SEQUENCE LISTING (20 sequences)

SEQ ID NO: 1 - protein, 5 aa, synthetic construct
SRSSY

SEQ ID NO: 2 - protein, 16 aa, synthetic construct
GIYHSGNTYDNPSLKS

SEQ ID NO: 3 - protein, 10 aa, synthetic construct
IVPGGDAFDI

SEQ ID NO: 4 - protein, 11 aa, synthetic construct
RASQSVGTYLN

SEQ ID NO: 5 - protein, 7 aa, synthetic construct
AASSLQS

SEQ ID NO: 6 - protein, 9 aa, synthetic construct
QQSYSSPPT

SEQ ID NO: 7 - protein, 120 aa, synthetic construct
QLQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSS

SEQ ID NO: 8 - protein, 107 aa, synthetic construct
DIQMTQSPSSLSASVGDRVTIACRASQSVGTYLNWYQQKRGKAPKLLIFAASSLQSGVPSRFSGSGSGTDFTLTISSLQPEDFATYYCQQSYSSPPTFGQGTKVEIK

SEQ ID NO: 9 - protein, 450 aa, synthetic construct
QLQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTIACRASQSVGTYLNWYQQKRGKAPKLLIFAASSLQSGVPSRFSGSGSGTDFTLTISSLQPEDFATYYCQQSYSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 119 aa, synthetic construct
LQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSS

SEQ ID NO: 12 - protein, 106 aa, synthetic construct
DIQMTQSPSSLSASVGDRVTIACRASQSVGTYLNWYQQKRGKAPKLLIFAASSLQSGVPSRFSGSGSGTDFTLTISSLQPEDFATYYCQQSYSSPPTFGQGTKVEI

SEQ ID NO: 13 - protein, 449 aa, synthetic construct
QLQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 449 aa, synthetic construct
LQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 448 aa, synthetic construct
LQLQESGPGLVKPSETLSLTCSVSGGSIISRSSYWGWIRQPPGKGLEWIGGIYHSGNTYDNPSLKSRLTMSVDTSKNQFSLNLRSVTAADTAVYYCARIVPGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 13 aa, synthetic construct
QLQLQESGPGLVK

SEQ ID NO: 17 - protein, 7 aa, synthetic construct
DTLMISR

SEQ ID NO: 18 - protein, 16 aa, synthetic construct
VVSVLTVLHQDWLNGK

SEQ ID NO: 19 - protein, 22 aa, synthetic construct
GFYPSDIAVEWESNGQPENNYK

SEQ ID NO: 20 - protein, 8 aa, synthetic construct
SLSLSPGK

(19) United States
(12) Patent Application Publication
(10) Pub. No.: US 2026/0250367 A1
(43) Pub. Date: Aug. 27, 2026

(54) METHODS FOR TREATING OR PREVENTING TRANSTHYRETIN-MEDIATED AMYLOIDOSIS

(71) Applicants: Neurimmune AG, Schlieren (CH); Alexion Pharmaceuticals, Inc., Boston, MA (US)

(72) Inventors: Aubin MICHALON, Baden (CH); Christoph HOCK, Erlenbach (CH); Peter KAHR, Zûrich (CH); Chandrasekhar UDATA, Boston, MA (US); Cristina Candida QUARTA, Boston, MA (US); Michele MERCURI, Boston, MA (US)

(21) Appl. No.: 19/129,766

(22) PCT Filed: Nov. 15, 2023
(86) PCT No.: PCT/EP2023/081809

§ 371 Date: May 14, 2025

Related U.S. Application Data
(60) Provisional application No. 63/383,807, filed on Nov. 15, 2022.

(30) Foreign Application Priority Data
Nov. 15, 2022 (EP) 22207651.5
Apr. 6, 2023 (EP) 23020175.8

(51) Int. Cl. C07K 16/18; A61J 1/10; A61P 9/00

(52) U.S. Cl. CPC C07K 16/18; A61P 9/00; A61J 1/10

(57) ABSTRACT

Provided herein are anti-TTR antibody dosing regimens useful for treatment of transthyretin amyloid cardiomyopathy (ATTR-CM) in an adult human patient. In embodiments, the patient is treated with an anti-TTR antibody comprising NI006/ALXN2220.

### TECHNICAL FIELD

This disclosure relates to methods of treating or preventing transthyretin-mediated amyloidosis (ATTR).

### BACKGROUND

Systemic amyloidosis is an infiltrative disease caused by progressive deposition of amyloid fibrilles in organs such as the heart, liver and pancreas. For heart amyloidosis, the most common forms include immunoglobulin light-chain and transthyretin amyloidosis. Current approved drugs either stabilize or block the production of amyloidogenic precursors, preventing further amyloid deposition. This approach, while reducing cell damage and disease progression, does not remove existing amyloid deposits and leads to functional recovery of the affected organ, thus improving quality of life and survival. A therapeutic strategy based on monoclonal antibodies capable of selectively binding amyloid deposits and inducing their removal could represent a key treatment for systemic amyloidosis such as of the heart.

There have been attempts to develop drugs that promote the degradation and re-absorption of amyloid deposits at tissue level. For example, antibodies binding to serum amyloid protein (SAP, a plasma glycoprotein produced by hepatocytes that represents one of the scaffold proteins of amyloid deposits in all tissues) have been investigated. However, a Phase II study (NCT03044353) in patients with cardiac amyloidosis was terminated prematurely due to an apparent change in the benefit/risk profile in this type of patient.

Transthyretin (TTR) is a soluble protein involved in thyroxin and retinol transport in the body. TTR is secreted in the blood by the liver and in the cerebrospinal fluid by the choroid plexus and is also expressed in specific tissues like the pancreatic alpha cells or retinal epithelium.

Under specific conditions which have been poorly elucidated and may include acidic pH, oxidative stress and local factors, the TTR protein adopts misfolded, misassembled and/or aggregated TTR conformations and becomes toxic, which can lead to transthyretin-mediated amyloidosis (ATTR).

Antibodies (e.g., human antibodies) that target misfolded, misassembled and/or aggregated TTR have been developed. There exists a need for improved methods (e.g., antibody dosing regimens) suitable for treating or preventing ATTR in a subject, with an acceptable benefit/risk profile and preferably dosing/administration as much convenient to the subject and the physician as possible.

In accordance with the present invention, the solutions to the above-described problems are provided by the embodiments characterized in the claims and as disclosed in the description and recited in the items preceding the claims.

### SUMMARY OF THE DISCLOSURE

Provided herein are, inter alia, methods and related dosing regimens for treating or preventing wild-type or hereditary transthyretin-mediated amyloidosis (ATTR), such as ATTR-CM, which is ATTR amyloidosis that leads to Cardiomyopathy (CM). More particularly, the present invention provides an anti-transthyretin (TTR) antibody for use in a method of treating ATTR in a subject in need of said treatment, wherein the method comprises administering the antibody at a dose of: 2000 mg to 2500 mg to a patient weighing equal or more than 40 kg (≥40 kg) to under 60 kg (<60 kg); (b) 3000 mg to 3500 mg to a patient weighing equal or more than 60 kg (≥60 kg) to under 100 kg (<100 kg); or (c) 4000 mg to 5000 mg to a patient weighing equal or more than 100 kg (≥100 kg).

As shown in Example 1, the anti-TTR antibody as used in accordance with the present invention, exemplarily shown for ALXN2220, also known as NI006, was shown to be safe and well tolerated by the human patient and depleted amyloid transthyretin deposits from cardiac tissue by antibody-dependent cellular phagocytosis (ADCP) in a dose- and time-dependent manner, with most pronounced effects at doses greater than 10 mg/kg when administered once every four weeks. In addition, the antibody has been applied with a maximum dose of 60 mg per kilogram of body weight every 28 days without any drug-related serious adverse events; see ClinicalTrials.gov ID NCT04360434 and Garcia-Pavia et al., Phase 1 Trial of Antibody NI006 for Depletion of Cardiac Transthyretin Amyloid. N. Engl. J. Med. 389 (2023), 239-250, each of which is incorporated herein by reference. As further shown in Example 1, data from bone scintigraphy and MRI indicated that doses of 30 mg/kg and 60 mg/kg decreased the cardiac amyloid deposition by a median of 12.8% and 25.6% compared to baseline at 4 months; at 12 months, the median reductions were 30.7% and 50.7%, respectively. In the same patients, NT-proBNP was reduced by 78.2% and 72.2% at 12 months. Since NT-ProBNP concentrations typically increase progressively in untreated ATTR-CM patients and are strongly correlated with patient mortality, the data point to usefulness of the anti-TTR antibodies of the present disclosure in treating and/or preventing ATTR-CM in human patients.

While it could be shown that immunotherapy of ATTR is possible with an anti-TTR antibody, dosing regimen used in the Clinical Trials by body weight-adjusted doses prove inconvenient in clinical practice since the administration of body weight-adjusted doses is complex and prone to mistakes, for example in the measurement of the body weight, calculation of the appropriate dose to administer, and extraction of the corresponding drug volume from the vials. In addition, this approach generates substantial drug wastage corresponding to the amount of drug remaining in the vial that is not administered to the patient.

Therefore, based on the results of the Phase 1 Clinical Trials, 14-day repeat-dose GLP toxicity studies and PK modelling, a new dose regimen, i.e., body weight-bracketed flat dose, has been developed to overcome the above-mentioned drawbacks.

In this context, a PK/PD model was established and flat doses in the range of 2000 mg to 5000 mg have been calculated to be safe and effective; see Example 5. In this context, single and repeated dose PK studies in rats confirm the pharmacokinetic (PK) profile for a IgG1 molecule. Taking all this into account, body weight adjusted flat doses have been calculated, wherein doses ranging from 2000 mg to 2500 mg for a patient weighing equal or more than 40 kg (≥40 kg) to under 60 kg (<60 kg), doses ranging from 3000 mg to 3500 mg for a patient weighing equal or more than 60 kg (≥60 kg) to under 100 kg (<100 kg), and doses ranging from 4000 mg to 5000 mg for a patient weighing equal or more than 100 kg (≥100 kg) have been calculated to be safe and efficient. Thus, the present invention relates to an anti-transthyretin (TTR) antibody for use in a method of treating transthyretin-mediated amyloidosis (ATTR) in a subject in need of said treatment, wherein the method comprises administering the antibody at a dose of: 2000 mg to 2500 mg to a patient weighing equal or more than 40 kg (≥40 kg) to under 60 kg (<60 kg); (b) 3000 mg to 3500 mg to a patient weighing equal or more than 60 kg (≥60 kg) to under 100 kg (<100 kg); or (c) 4000 mg to 5000 mg to a patient weighing equal or more than 100 kg (≥100 kg).

More specially, in one initial embodiment the following doses are proposed: 5000 mg if body weight ≥100 kg; 3500 mg if 60 kg≤body weight <100 kg; 2500 mg if body weight <60 kg to be administered via IV every 28 days (q4w). Those weight-bracketed flat doses were further optimized to ensure administration of the complete amount of drug present in each vial and eliminate residual volume and corresponding drug wastage, while maintaining the same predicted efficacy, and calculated to result: 2400 mg to a patient weighing equal or more than 40 kg (≥40 kg) to under 60 kg (<60 kg); 3200 mg to a patient weighing equal or more than 60 kg (≥60 kg) to under 100 kg (<100 kg); or 4800 mg to a patient weighing equal or more than 100 kg (≥100 kg); see Example 6. Accordingly, in a preferred embodiment, the antibody is administered at a dose of 2400 mg to a patient weighing equal or more than 40 kg (≥40 kg) to under 60 kg (<60 kg), at a dose of 3200 mg to a patient weighing equal or more than 60 kg (≥60 kg) to under 100 kg (<100 kg), or at a dose of 4800 mg to a patient weighing equal or more than 100 kg (≥100 kg). These flat doses are used in the Phase 3 Clinical Trial Protocol (CTP) as outlined in Example 8. As illustrated in the CTP, one concept of monitoring during administration of study drug and during the post-infusion observation period includes monitoring of heart rate, blood pressure and oxygen saturation and ECG assessments at screening, and both pre- and post-infusion at week 1, week 3, week 12, and approximately every 12 weeks thereafter.

As mentioned above, in the Clinical Trial referred to in Example 1, antibody ALXN2220/NI006 has been used as drug substance. The parent antibody of NI006 has been first described in WO 2015/092077 A1 (designated as antibody NI-301.37F1) and in Michalon et al., Nat. Commun. 12 (2021), 3142 (designated as antibody NI301A). As disclosed in WO 2015/092077 A1, NI006 (NI-301.37F1) is inter alia characterized by binding to aggregated human wild-type transthyretin (wtATTR), which is shown in FIGS. 2 to 4 and 7 and described in Examples 3 to 6, and further described at page 46, last paragraph. In addition, WO 2015/092077 A1 discloses that NI006 (NI-301.37F1) does not bind to monomers and dimers of the human native transthyretin (TTR) as shown in Example 5 and FIG. 4. This binding profile is advantageous since the antibody binds selectively to aggregated wtTTR and thus allows prima facie to consider not only the treatment of hereditary transthyretin amyloidosis (hATTR) with polyneuropathy (formerly known as Familial Amyloid Polyneuropathy, FAP), which is due to mutations in the gene encoding TTR, but also the treatment of wild-type transthyretin amyloidosis (wtATTR), known as senile systemic amyloidosis (SSA). Moreover, the antibody is not at risk to interfere with native monomer assembly into physiological tetramers. Said antibody has been described by comprising in its variable region or binding domain the complementary determining regions (CDRs) and variable heavy (V_{H}) and variable light (V_{L}) chain having the amino acid sequences depicted in FIGS. 1C and 1M, respectively, of WO 2015/092077 A1. The disclosure in U.S. Pat. No. 10,344,080 is incorporated by reference in parts pertinent thereto (e.g., sequences of VHCDR1-3 & VLCDR1-3, including, sequences of VH and VL chains).

In principle, any anti-TTR antibody, which recognizes the amyloidogenic form of TTR, i.e., aggregated TTR species, and preferably human aggregated TTR, but does not bind to physiological TTR species can be used in accordance with the present invention. Preferably, the anti-TTR antibody as used in accordance with the present invention is NI006/ALXN2220 or an equivalent antibody that substantially has the TTR binding profile of NI006 and preferably is of human origin. For example, WO 2015/092077 A1 discloses two further human antibodies which show the mentioned bind profile, i.e., antibodies NI-301.59F1 and NI-301.35G11, and two human antibodies, NI-301.28B3 and NI301.12D3 which have the substantially same epitope as NI006 (NI-301.37F7). More preferably, the equivalent antibody is derived from human antibody NI-301.37F1 as characterized in WO 2015/092077 A1 and in Michalon et al., Nat Commun. 12 (2021), 3142; see also supra.

Accordingly, the antibody or antigen-binding fragment as used in accordance with the present invention comprises in one embodiment a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody or the antigen-binding fragment thereof comprises a heavy chain variable region comprising at least 80% sequence identity to SEQ ID NO: 7 and light chain variable region comprising an amino acid sequence having at least 80% sequence identity to SEQ ID NO: 8.

In some embodiments, the VH region includes an amino acid sequence having at least 80% sequence identity (e.g., at least 85%, 90%, 95%, 97%, 99%, or 100% sequence identity) to SEQ ID NO: 7 and the VL includes an amino acid sequence having at least 80% sequence identity (e.g., at least 85%, 90%, 95%, 97%, 99%, or 100% sequence identity) to SEQ ID NO: 8.

In some embodiments, the VH region includes an amino acid sequence having at least 80% sequence identity (e.g., at least 85%, 90%, 95%, 97%, 99%, or 100% sequence identity) to SEQ ID NO: 11 and the VL includes an amino acid sequence having at least 80% sequence identity (e.g., at least 85%, 90%, 95%, 97%, 99%, or 100% sequence identity) to SEQ ID NO: 12.

Preferably, the VH region includes the amino acid sequence of SEQ ID NO: 7 and the VL includes the amino acid of SEQ ID NO: 8, or the VH region includes the amino acid sequence of SEQ ID NO: 11 and the VL includes the amino acid of SEQ ID NO: 8 or 12, preferably SEQ ID NO: 8.

To avoid generation of “anti-drug antibodies” (ADA) by a subject administered an antibody described herein, the antibody is preferably a human or humanized antibody, typically human IgG and most preferably a human IgG1. In a preferred embodiment, the antibody is a human IgG1m3 allotype.

Antibody NI006/ALXN2220 as used in accordance with the present invention is a fully human IgG1m3 allotype antibody and thus comprises the human constant heavy chain (HC) amino acid sequence as exemplified in SEQ ID NO: 9 and a corresponding human constant light chain (LC), here kappa light chain, as exemplified in SEQ ID NO: 10. As explained further below, IgG antibodies are made up as tetramers consisting of HC and two light LC chains linked by disulfide bridges. The theoretical molecular weight of antibody NI006/ALXN2220 is 144.2 kDa, and the weight determined by mass spectrometry (MS) is 144.2 kDa (deglycosylated) and between 147.0 and 147.6 kDa (intact IgG1), respectively.

Antibody NI006/ALXN2220 has been produced in Chinese hamster ovary (CHO)-K1 cells. CHO cells are the most widely used mammalian cells for the production of recombinant monoclonal antibodies due to their ability to perform post-translational modifications (PTMs) on the antibody molecules, which typically take place in the human body as well. Through genetic manipulation by mutagenesis, different CHO daughter cells with improved qualities have been established. Among those variants are CHO-K1, CHO-S, CHO-DXB11 and CHO-DG44. Thus, in one embodiment, the antibody for use in accordance with the present invention is produced in CHO cells, preferably in a CHO-K1 cell line and is purified from the cell culture medium for further use.

As shown in Example 7, the major PTMs that have been identified in antibody NI006/ALXN2220 are the modification in the HC of glutamine at the N-terminus to pyro-glutamic acid, the loss of C-terminal lysine, and N-glycosylation. In this context, the N-glycosylation site was identified at position 300 (HC N300, SEQ ID NO: 9). Thus, in one embodiment, the antibody for use in accordance with the present invention has lost the C-terminal lysine, i.e., the antibody has undergone C-terminal lysine clipping. In particular, the C-terminal lysine as shown in SEQ ID NO: 9 is chopped off the heavy chain of the antibody, preferably of each heavy chain of the antibody. Alternatively, the glutamine at the N-terminal is modified as pyro-glutamic acid, i.e., the heavy chain of the antibody as shown in SEQ ID NO: 9 has undergone N-terminal glutaminyl cyclization. Said sequence, i.e., the sequence of the heavy chain which comprises cyclic pyroglutamic acid and no N-terminal glutamate is set forth in SEQ ID NO: 14.

Alternatively, the heavy chain of the antibody for use in accordance with the present invention has lost the C-terminal lysine and the glutamine at the N-terminal is modified as pyro-glutamic acid. Said sequence, i.e., the sequence of the heavy chain with a clipped off C-terminal lysine and which comprises cyclic pyroglutamic acid and no N-terminal glutamate is set forth in SEQ ID NO: 15.

In addition, or alternatively, the antibody is glycosylated, in particular N-glycosylated. More particularly, the heavy chain of the antibody is glycosylated and even more particularly N300 of the heavy chain.

In a preferred embodiment, the anti-TTR antibody for use in accordance with the present invention lacks the C-terminal cysteine, has a modified glutamine at the N-terminal as pyro-glutamic acid and comprises at least one N-glycosylation site.

Thus, in one preferred embodiment, the antibody as used in accordance with the present invention is composed of two heavy chains having SEQ ID NO: 9, and two light chains having SEQ ID: 10, and wherein in the heavy chain the glutamine at the N-terminus is modified as pyro-glutamic acid, the C-terminal lysine is lost, and the heavy chain is N-glycosylated. In other words, the antibody as used in accordance with the present invention is preferably composed of two heavy chains having SEQ ID NO: 15, and two light chains having SEQ ID NO: 10, and wherein the heavy chain is N-glycosylated.

As mentioned above, the anti-TTR antibody, as used in accordance with the present invention, depleted amyloid transthyretin from cardiac tissue in a dose- and time-dependent manner. Thus, in some embodiments, the ATTR amyloidosis leads to Cardiomyopathy (CM) and thus, in a preferred embodiment, the subject to be treated in accordance with the present invention has ATTR amyloidosis with CM (ATTR-CM). In a preferred embodiment, the subject to be treated has either variant ATTR (ATTRv/hATTR) or wild-type ATTR-CM ((wATTR). Even more preferred, the subject to be treated has either variant ATTR-CM (ATTRv-CM/hATTR-CM) or wild-type ATTR-CM ((wATTR-CM).

In some embodiments, the subject has ATTR polyneuropathy (ATTR-PN). In some embodiments, the subject has Familial Amyloid Polyneuropathy (FAP). In some embodiments, the subject has Familial Amyloid Cardiomyopathy (FAC). In some embodiments, the subject has Senile Systemic Amyloidosis (SSA). In some embodiments, the subject has systemic familial amyloidosis. In some embodiments, the subject has leptomeningeal/Central Nervous System (CNS) amyloidosis. In some embodiments, the subject has Alzheimer disease. In some embodiments, the subject has TTR-related ocular amyloidosis. In some embodiments, the subject has TTR-related renal amyloidosis. In some embodiments, the subject has TTR-related hyperthyroxinemia. In some embodiments, the subject has TTR-related ligament amyloidosis including carpal tunnel syndrome. In some embodiments, the subject has rotator cuff tears and lumbar spinal stenosis. In some embodiments, the subject has preeclampsia.

In some embodiments, the subject has been diagnosed with hereditary ATTR-CM for a known pathogenic TTR mutation.

In some embodiments, the subject has sporadic, wild-type-ATTR-CM (WT-ATTR-CM) (e.g., a wild type ATTR gene that codes for TTR proteins that form deposits in the heart) and a negative genetic testing for a TTR mutation.

Preferably, the diagnosis is based either on the presence of symptomatic ATTR, preferably ATTR-CM or on an NT-proBNP level of >2000 pg/mL The use of NT-proBNP as biomarker in ATTR is recognized in the art and based on the level of said biomarker (either in combination with the level of cardiac troponin T (cTnT) (Grogan et al., J Am Coll Cardiol 68 (2016), 1014-1020) or in combination with estimated glomerular filtration rate (eGFR) (Gillmore et al., European Heart Journal 39 (2018), 2799-2806)) staging systems have been developed with a cut off for NT-proBNP of 3000 pg/mL; see also Perfetto et al., Internal and Emergency Medicine 17 (2022), 957-969. Thus, dependent on the level of cTnT and eGFR, and only with a view to the level of NT-proBNP, the subject has Grade I, II and III cardiac ATTR. Thus, the subject to be treated is preferably either symptomatic for ATTR, preferably ATTR-CM and/or has an NT-proBNP level of >2000 pg/mL.

In addition, or alternatively, the subject is an adult subject, in particular an adult human subject, preferably, a subject who is 18 years or older but less than 90 years old ((≥18 to ≤90 years of age).

Thus, the treatment regime of the present invention can be used for treating subjects having any one or all of the mentioned indications/characteristics.

Treatment of human subjects with the antibody as used in accordance with the present invention results, in one embodiment, in a lower cardiac amyloid load and/or composite of all-cause mortality (ACM) and total cardiovascular (CV) clinical events and/or heart failure (HF) events. In some embodiments, treatment with the anti-TTR antibody in accordance with the present method(s) results in a dose- and time-dependent reduction, in the patient, cardiac amyloid load of at least about 25%, about 30%, about 35%, about 40%, about 45%, about 50%, about 55%, about 60%, or more, e.g., about 70%, about 80%, about 90%, after a specified duration of treatment, e.g., 4 months, 6 months, 8 months, 10 months, 12 months, 15 months, 18 months, 21 months, or 24 months, or more, e.g., 48 months.

Preferably, the treatment, i.e., the administration of the dosing regimen of the present invention improves at least one of the following:
(a) symptoms, functionality, and health-related quality-of-life (QoL) as measured by the change from baseline in Kansas City Cardiomyopathy Questionnaire overall summary (KCCQ-OS) score;
(b) time to cardiovascular (CV)-related mortality;
(c) six-minute walk test (6MWT) score compared to baseline;
(d) rate of cardiovascular (CV) clinical events; and
(e) time to all-cause mortality (ACM).

In one embodiment, the treatment
(a) reduces NT-proBNP levels in the subject compared to baseline;
(b) reduces rate of heart failure (HF) events;
(c) reduces incidence of intensification of oral diuretic therapy, which optionally includes outpatient augmentation of oral diuretic therapy;
(d) reduces incidence of changes in disease modifying therapy;
(e) reduces incidence of hospitalization for atrial fibrillation;
(f) induces change from baseline in Kansas City Cardiomyopathy Questionnaire overall summary (KCCQ-OS) score beyond 24 months of study treatment;
(g) induces change from baseline in six-minute walk test (6MWT) beyond 24 months of study treatment;
(h) induces change from baseline in ATTR-CM disease severity based on the Mayo, NAC, and Columbia disease stage, and NYHA classification;
(i) induces change from baseline in GLS;
(j) induces change from baseline in stroke volume;
(k) induces change from baseline in echocardiography parameters of interest;
(l) induces change from baseline in hs-cTnT;
(m) induces change from baseline in DPD/PYP/HMDP cardiac scintigraphy cardiac uptake and/or cMRI-derived ECV, T1 and T2 mapping;
(n) induces change from baseline in eGFR
(o) induces change from baseline in EQ-5D-5L score and/or induces change from baseline in SF-36 scores;
(p) induces change from baseline in a marker selected from CRP, IL IL1b, IL6, IL8, IFNg, TNF-α, IL10, IL1RA, C3 and C4, serum amyloid A, and ferritin; preferably a marker which is CRP;
(q) induces change from baseline in a marker selected from serum carboxy-terminal PICP, PIIINP, serum CITP, and plasma PRO-C6;
(r) induces change from baseline in a marker selected from TTR (prealbumin), TSH, RBP and fT4;
(s) induces change from baseline in (1) PND score and FAP stage; (2) Norfolk QoL-DN total score; and/or sNFL levels; and/or
(t) induces change from baseline in NIS and/or induces a change in NC studies.

In some embodiments, the antibody for use in accordance with the present invention is administered to the subject once every four weeks (q4w).

In some embodiments, the antibody for use in accordance with the present invention is administered to the subject intravenously (IV), preferably via IV infusion, i.e., the method of treating as defined hereinbefore comprises administering the antibody to the subject intravenously (IV), preferably via IV infusion.

In some embodiments, the antibody for use in accordance with the present invention is administered for at least 24 months, preferably at least 48 months, i.e., the method of treating comprises administering the antibody for at least 24 months, preferably at least 48 months.

In some embodiments, the antibody for use in accordance with the present invention is administered in a body weight-bracketed flat dose based on the patient's recorded body weight, preferably in the flat doses as defined hereinbefore, wherein the recording of the body weight is made within 30 days of a scheduled administration, wherein said dose is administered intravenously (IV) via infusion every four weeks (q4w), i.e., the method comprises administering the antibody in a body weight-bracketed flat dose based on the patient's recorded body weight, preferably in the flat doses as defined hereinbefore, wherein the recording of the body weight is made within 30 days of a scheduled administration, wherein said dose is administered intravenously (IV) via infusion every four weeks (q4w).

In pharmacology studies (Michalon, 2021), NI006/ALXN2220 was shown to activate immune cells via binding to Fc gamma receptors. It has been demonstrated using different in vitro and in vivo models, that NI006/ALXN2220 triggers the elimination of ATTR fibrils from patient samples through immune driven phagocytosis clearance in a dose- and time-dependent manner. Thus, NI006/ALXN2220 induces antibody-mediated phagocytosis of ATTR fibrils by phagocytic immune cells such as macrophages, resulting in the clearance of ATTR deposits from tissues.

Accordingly, in some embodiments, the antibody for use in accordance with the present invention triggers the elimination of ATTR fibrils from patient samples through immune driven phagocytosis clearance in a dose- and time-dependent manner.

In the Phase 1 study NI006-101, monthly NI006/ALXN2220 treatment was generally safe and well-tolerated in adult patients with ATTR-CM up to the highest dose tested (i.e., 60 mg/kg IV q4w). NI006/ALXN2220 PK profiles were seen to be dose-proportional, provide sustained antibody levels and to be compatible with monthly dosing. In this study, the amount of ATTR deposits in the heart was estimated using 2 different methods: quantification of cardiac tracer uptake in the heart by scintigraphy or quantification of ECV by cMRI. These 2 PD measurements are proxies for cardiac amyloid load and served to estimate baseline amyloid load and change over time during the clinical study. NI006/ALXN2220 showed dose- and time-dependent reductions in the cardiac amyloid load up to approximately 51% at 60 mg/kg at 12 months. Thus, preferably, the elimination of ATTR fibrils by the anti-TTR antibody treatment is measured via cardiac tracer uptake scintigraphy or quantification of ECV with cMRI; see the Phase 1 study NI006-101. More preferably, the patient's treatment with the anti-TTR antibody results in a dose- and time-dependent reduction in the cardiac amyloid load up to approximately 51% at 12 months, preferably wherein the dose administered to the patient corresponds to about 60 mg/kg. Accordingly, in some embodiments, treatment with the anti-TTR antibody in accordance with the present method(s) results, in the patient, a dose- and time-dependent reduction in the cardiac amyloid load of at least about 25%, about 30%, about 35%, about 40%, about 45%, about 50%, about 55%, about 60%, or more, e.g., about 70%, about 80%, about 90%, after a specified duration of treatment, e.g., 4 months, 6 months, 8 months, 10 months, 12 months, 15 months, 18 months, 21 months, or 24 months, or more, e.g., 48 months.

In some embodiment, the patients to be treated with the antibody in accordance with the present invention is a male or a female subject who
(1) has a centrally confirmed diagnosis of ATTR-CM with either wild-type or variant TTR genotype based on evidence of cardiac amyloidosis by echocardiography or cMRI and one of the following:
   a. endomyocardial biopsy with confirmatory TTR amyloid typing by either immunohistochemistry or mass spectrometry; or
   b. grade 2 or 3 cardiac uptake on 99mTc scintigraphy (99mTc DPD, 99mTc PYP, or 99mTc HMDP) in the absence of monoclonal gammopathy; or
   c. grade 2 or 3 cardiac uptake on 99mTc scintigraphy (99mTc DPD, 99mTc PYP, or 99mTc HMDP) AND confirmatory TTR amyloid typing by either immunohistochemistry or mass spectrometry in non-cardiac tissue in the presence of monoclonal gammopathy;
(2) is willing to be genetically tested for mutations in the TTR gene during screening, if genetic testing was not previously performed or if genetic results are not available
(3) has end-diastolic interventricular septal wall thickness ≥11 mm for women or ≥12 mm for men on echocardiography measured at screening
(4) has NT-proBNP>2000 pg/mL, as measured by a central laboratory at screening
(5) has treatment with a loop diuretic for at least 30 days prior to screening;
(6) has a history of heart failure as documented by one of the following events within 1 year prior to screening:
   a. heart failure hospitalization
   b. urgent heart failure visit
   c. episode of volume overload documented by NT-proBNP>2000 μg/mL (or equivalent BNP)
(7) is New York Heart Association (NYHA) classification Class II-IV at screening; and/or
(8) has a life expectancy of at least 6 months as per the clinician's judgment.

As outlined in European patent application EP 22 207 645.7 and U.S. provisional application Nos. 63/383,803 and 63/503,286, as well as in the international application with the title “Pharmaceutical compositions for treating or preventing transthyretin-mediated amyloidosis” (Attorney docket number: NE30A100/P-WO), filed on Nov. 15, 2023 in detail, which content is herein incorporated by reference, a formulation comprising 50 mg/mL of the antibody, i.e., ALXN2220/NI006, in 20 mM histidine buffer, 80 mg/mL or 65 mg/mL sucrose, and 0.3 mg/mL polysorbate 80, at pH 5.8 has been found to be particularly suitable to ensure long-term stability of the drug product. Thus, a formulation comprising 50 mg/mL of the antibody, i.e., ALXN2220/NI006, in 20 mM histidine buffer, 80 mg/mL sucrose, and 0.3 mg/mL polysorbate 80, at pH 5.8 has been used in the Clinical Trials as described in the Examples, below.

Thus, in some embodiments, the antibody for use in accordance with the present invention is administered in a pharmaceutical formulation at 50 mg/mL in 20 mM histidine buffer (e.g., L-histidine and L-histidine monohydrochloride), 80 mg/mL sucrose, 0.3 mg/mL polysorbate 80, at pH 5.8. Alternatively, the antibody for use in accordance with the present invention is administered in a pharmaceutical formulation at 50 mg/mL in 20 mM histidine buffer (e.g., L-histidine and L-histidine monohydrochloride), 65 mg/mL sucrose, 0.3 mg/mL polysorbate 80, at pH 5.8.

Preferably, the antibody for use in accordance with the present invention is administered as an intravenous (IV) infusion to the patient over 2 hours for initial administration and over 1 hour for subsequent administrations, i.e., the method comprises administering the antibody as an intravenous (IV) infusion to the patient over 2 hours for initial administration and over 1 hour for subsequent administrations.

In some embodiments, the subject has been previously treated with and/or is concurrently receiving a disease modifying agent selected from TTR silencer and TTR stabilizer. For example, the TTR tetramer stabilizer can be diflunisal, tafamidis (VYNDAQEL® or VYNDAMAX®), or Acoramidis (AG10). Presently, tafamidis, oral disease-modifying treatment acting, is the only approved pharmacologic treatment specifically targeting ATTR-CM, both wild-type and hereditary forms. In accordance with the present invention, it was shown that ALXN2220 activity is maintained in presence of tafamidis.

In some embodiments, the antibody as used in accordance with the present invention is administered for up to 24 months in accordance with the intervention infusion schedule provided in Table 25. Preferably, as a follow-up, after the 24-month treatment period, the antibody as used in accordance with the present invention is administered in accordance with the intervention infusion schedule of Table 26. Thus, the method comprises in one embodiment administration of the antibody for up to 24 months in accordance with the intervention infusion schedule provided in Table 25, and preferably as a follow-up, after the 24-month treatment period, in accordance with the intervention infusion schedule of Table 26.

As shown in the Examples, the treatment with the anti-TTR antibody results in a median amyloid reduction when the antibody is administered at a dose 30 and 60 mg/kg in the patient and based on the PK modelling, same amyloid reduction is expected to occur with the flat doses. Accordingly, in some embodiments, the treatment with the anti-TTR antibody when used in accordance with the present invention, results in a median amyloid reduction when the antibody is administered at a dose which corresponds to the dose between 30 and 60 mg/kg in the patient. Preferably, the treatment efficacy is measured with endomyocardial biopsy comprising intra-epidermal nerve fiber density (IENFD) and/or sweat gland nerve fiber density (SGNFD).

In some embodiments, the patient is stratified based on:
(1) prior treatment with a disease modifying agent, wherein the disease modifying agent is selected from
(a) TTR silencer optionally together with a TTR stabilizer; (b) TTR stabilizer alone; and (c) no TTR stabilizer or TTR silencer treatment;
(2) TTR genotype comprising ATTR variant (ATTRv) or ATTR wild-type (ATTRwt); or
(3) disease severity based on cardiac biomarker levels comprising NT-proBNP>3000 pg/mL vs NT-proBNP≤3000 pg/mL, optionally together with levels of high sensitivity cardiac troponin C (hs-cTnT) pre- and post-treatment.

Furthermore, to monitor the treatment efficiency, serum biomarker levels may be monitored pre and post treatment with the antibody as used in accordance with the present invention. In particular, in some embodiments, the biomarkers are selected from: (a) complement factors selected from C3 and C4, together with CRP; (b) pro-inflammatory cytokines selected from IL1b, IL6, IL8, IFNg, and TNF-α; (c) anti-inflammatory cytokines selected from IL10, IL1RA; (d) positive acute phase proteins selected from SAA and ferritin; (e) PICP; (f) PIIINP; (g) CITP; (h) PRO-C6; and (i) plasma NNTTR, or a combination thereof.

### Definitions

For the avoidance of any doubt, it is emphasized that the expressions “in some embodiments”, “in a certain embodiments,” “in certain instances,” “in some instances,” “in some aspects,” “in a further embodiment,” “in one embodiment,” “in a further aspect,” “in a first aspect,” “in a second aspect,” etc., and the like are used and meant such that any of the embodiments described therein are to be read with a mind to combine each of the features of those embodiments and that the disclosure has to be treated in the same way as if the combination of the features of those embodiments and aspects would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices. In this context, the person skilled in the art will appreciate that the embodiments and features disclosed in the Examples are intended to be generalized to any anti-TTR antibody and equivalents having substantially the same properties.

As used herein, the term “about,” as used herein, refers to a value that is ±10% of a recited value; preferably ±5%.

In connection with the present invention, the term “and/or” is understood to mean that all members of a group which are connected by the term “and/or” are disclosed cumulatively in any combination, both alternatively to each other and in each case to each other. This means for the expression “A, B and/or C” that the following disclosure content is to be understood thereunder: a) A or B or C; or b) (A and B); or c) (A and C); or d) (B and C); or e) (A and B and C).

As used herein, the term “antibody” includes corresponding binding fragments thereof and the doses mentioned herein refer to a molecular weight of NI006 with approximately 147 kDa for the intact IgG1 antibody. Thus, for the use of antibodies which significantly differ in their MW the dose may be adjusted accordingly. Similarly, if an antibody is used with a lower or longer serum half-life, for example because of altered glycosylation and/or modification such as PEGylation, the dose and dosing interval, respectively, may be recalculated.

As used herein, the term “pharmaceutical composition” refers to a mixture containing a therapeutic agent (e.g., an anti-TTR antibody described herein), optionally in combination with one or more pharmaceutically acceptable excipients, diluents, and/or carriers. The pharmaceutical composition is, for example, formulated for administration to a subject, such as a mammal, e.g., a human, in order to prevent, treat or control a particular disease or condition affecting, or that may affect, the subject (e.g., ATTR, such as ATTR-CM, ATTR polyneuropathy (ATTR-PN), Familial Amyloid Polyneuropathy (FAP), Familial Amyloid Cardiomyopathy (FAC), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia).

As used herein, the term “pharmaceutically acceptable” refers to those compounds, materials, compositions, and/or dosage forms which are, within the scope of sound medical judgment, suitable for use in contact with the tissues a subject, such as a mammal (e.g., a human) without excessive toxicity, irritation, allergic response, or other problem or complication, commensurate with a reasonable benefit/risk ratio.

As used herein, the expressions “is capable of binding” and “binds to” refers to the capability of the antibody to bind to, for example aggregated TTR, under experimental conditions, for example in an ELISA assay.

The term “between” as used herein include the endpoints.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the terms “treat” or “treatment” refer to both therapeutic treatment and prophylactic or preventative measures, in which the object is to prevent or slow down (lessen) an undesired physiological change or disorder, such as the development of cardiac deficiency. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival (e.g., prolonging survival of a human subject having ATTR for at least 1, 2, 3, 4, 5, 6, 7, 8, 9, 10, 11, 12, 13, 14, 15, 16, 17, 18, 19, 20, or more years, e.g., for the lifetime of the subject) as compared to expected survival if not receiving treatment. Those in need of treatment include those already with the condition or disorder as well as those prone to have the condition or disorder or those in which the manifestation of the condition or disorder is to be prevented.

As used herein, the term “loading dose”, refers to a dose of an antibody of about 600 mg to 4000 mg (e.g., 2400 mg, 2500 mg, 3000 mg, 3200 mg, or 3500 mg) that increases the blood (e.g., serum or plasma) concentration in a subject to a desired therapeutic level (e.g., ≥1 μg/mL e.g., ≥1 μg/mL, ≥2.5 μg/mL, ≥5 μg/mL, ≥10 μg/mL, ≥20 μg/mL, ≥30 μg/mL, ≥40 μg/mL, ≥50 μg/mL, ≥60 μg/mL, ≥70 μg/mL, ≥80 μg/mL, ≥90 μg/mL, ≥100 μg/mL, ≥110 μg/mL, ≥120 μg/mL, ≥130 μg/mL, ≥140 μg/mL, 2 150 μg/mL, ≥160 μg/mL, ≥170 μg/mL, ≥180 μg/mL, ≥190 μg/mL, ≥200 μg/mL, ≥210 μg/mL, ≥220 μg/mL, ≥230 μg/mL, ≥240 μg/mL, ≥250 μg/mL, ≥260 μg/mL, ≥270 μg/mL, ≥280 μg/mL, ≥290 μg/mL, ≥300 μg/mL, ≥310 μg/mL, ≥320 μg/mL, ≥330 μg/mL, ≥340 μg/mL, ≥350 μg/mL, ≥360 μg/mL, ≥370 μg/mL, ≥380 μg/mL, ≥390 μg/mL, ≥400 μg/mL, ≥410 μg/mL, ≥420 μg/mL, ≥430 μg/mL, ≥440 μg/mL, ≥450 μg/mL, ≥460 μg/mL, ≥470 μg/mL, ≥480 μg/mL, ≥490 μg/mL, ≥500 μg/mL, ≥510 μg/mL, ≥520 μg/mL, ≥530 μg/mL, ≥540 μg/mL, ≥550 μg/mL, ≥560 μg/mL, ≥570 μg/mL, ≥580 μg/mL, ≥590 μg/mL, 2 600 μg/mL, ≥610 μg/mL, ≥620 μg/mL, ≥630 μg/mL, ≥640 μg/mL, ≥650 μg/mL, ≥660 μg/mL, ≥670 μg/mL, ≥680 μg/mL, ≥690 μg/mL, ≥700 μg/mL, ≥710 μg/mL, ≥720 μg/mL, ≥730 μg/mL, ≥740 μg/mL, ≥750 μg/mL, ≥760 μg/mL, ≥770 μg/mL, ≥780 μg/mL, ≥790 μg/mL, ≥800 μg/mL, ≥810 μg/mL, ≥820 μg/mL, ≥830 μg/mL, ≥840 μg/mL, ≥850 μg/mL, ≥860 μg/mL, ≥870 μg/mL, ≥880 μg/mL, ≥890 μg/mL, ≥900 μg/mL, ≥910 μg/mL, ≥920 μg/mL, ≥930 μg/mL, ≥940 μg/mL, ≥950 μg/mL, ≥960 μg/mL, ≥970 μg/mL, ≥980 μg/mL, ≥990 μg/mL, ≥1000 μg/mL, or more) in the subject being treated. By way of example, one or more (e.g., 1, 2, 3, 4, 5, 6, 7, 8, 9 10, or more) loading doses may be administered to a subject once every week (e.g., every 7 days), once every other week (e.g., biweekly or every 14 days), once every month (e.g., every 28±7 days), or once every other month (e.g., bimonthly, e.g., every 56±7 days) before a maintenance dose (e.g., a first maintenance dose or any subsequent maintenance doses) is administered to the subject.

As used herein, the term “maintenance dose” refers to a dose of an antibody of about 600 mg to 6000 mg (e.g., 2500 mg, 3000 mg, 3500 mg, or 5000 mg, and 2400 mg, 3200 mg, or 4800 mg, respectively) or 10-60 mg/kg (e.g., 10 mg/kg, 30 mg/kg, or 60 mg/kg) that maintains a desired minimum blood (e.g., serum or plasma) concentration of the antibody. For example, it is desirable to maintain a minimum concentration of ≥1 μg/mL (e.g., ≥1 μg/mL, ≥2.5 μg/mL, ≥_5 μg/mL, ≥10 μg/mL, ≥20 μg/mL, ≥30 μg/mL, ≥40 μg/mL, ≥50 μg/mL, ≥60 μg/mL, ≥70 μg/mL, ≥80 μg/mL, ≥90 μg/mL, ≥100 μg/mL, ≥110 μg/mL, ≥120 μg/mL, ≥130 μg/mL, ≥140 μg/mL, ≥150 μg/mL, ≥160 μg/mL, ≥170 μg/mL, ≥180 μg/mL, ≥190 μg/mL, ≥200 μg/mL, ≥210 μg/mL, ≥220 μg/mL, ≥230 μg/mL, ≥240 μg/mL, ≥250 μg/mL, ≥260 μg/mL, ≥270 μg/mL, ≥280 μg/mL, ≥290 μg/mL, ≥300 μg/mL, ≥310 μg/mL, ≥320 μg/mL, ≥330 μg/mL, ≥340 μg/mL, ≥350 μg/mL, ≥360 μg/mL, ≥370 μg/mL, ≥380 μg/mL, ≥390 μg/mL, ≥400 μg/mL, ≥410 μg/mL, ≥420 μg/mL, ≥430 μg/mL, ≥440 μg/mL, ≥450 μg/mL, ≥460 μg/mL, ≥470 μg/mL, ≥480 μg/mL, ≥490 μg/mL, ≥500 μg/mL, ≥510 μg/mL, ≥520 μg/mL, ≥530 μg/mL, ≥540 μg/mL, ≥550 μg/mL, ≥560 μg/mL, ≥570 μg/mL, ≥580 μg/mL, ≥590 μg/mL, ≥600 μg/mL, ≥610 μg/mL, ≥620 μg/mL, ≥630 μg/mL, 2 640 μg/mL, ≥650 μg/mL, ≥660 μg/mL, ≥670 μg/mL, ≥680 μg/mL, ≥690 μg/mL, ≥700 μg/mL, ≥710 μg/mL, ≥720 μg/mL, ≥730 μg/mL, ≥740 μg/mL, ≥750 μg/mL, ≥760 μg/mL, ≥770 μg/mL, ≥780 μg/mL, ≥790 μg/mL, ≥800 μg/mL, ≥810 μg/mL, ≥820 μg/mL, ≥830 μg/mL, ≥840 μg/mL, ≥850 μg/mL, ≥860 μg/mL, ≥870 μg/mL, ≥880 μg/mL, ≥890 μg/mL, ≥900 μg/mL, ≥910 μg/mL, ≥920 μg/mL, ≥930 μg/mL, ≥940 μg/mL, ≥950 μg/mL, ≥960 μg/mL, ≥970 μg/mL, ≥980 μg/mL, ≥990 μg/mL, ≥1000 μg/mL, or more) in the blood (e.g., serum or plasma) of a subject being treated. A maintenance dose may be administered to a subject at a concentration (e.g., 10-60 mg/kg, e.g., 10 mg/kg, 30 mg/kg, or 60 mg/kg) or flat dose (e.g., about 600 mg to 4000 mg, e.g., 2400 mg, 2500 mg, 3000 mg, or 3200 mg), preferably, a dose that is lower than a previously administered loading dose to the same subject or a subject of the same weight class.

### BRIEF DESCRIPTION OF THE DRAWINGS

FIG. 1 is a schematic diagram showing an exemplary dosing regimen. Red outlined boxes indicate the single-ascending dose (SAD) phases for each of cohorts 1 through 6, as described further in Example 1. Blue outlined boxes indicate the multiple-ascending dose (MAD) phases for each of cohorts 1 through 6, as described further in Example 1. Green-filled cells within the table, labeled DEC, are exemplary time periods for a Data Evaluation Committee, or the like, to examine a subject for safety and/or treatment efficacy before and after the indicated administration.

FIG. 2 is a schematic diagram showing an exemplary dosing regimen for an open-label extension OLE phase for each of cohorts 1 through 6, as described further in Example 1. Green-filled cells within the table, labeled DEC, are exemplary time periods for a Data Evaluation Committee, or the like, to examine a subject for safety and/or treatment efficacy before and after the indicated administration.

FIG. 3 is a schematic diagram showing an exemplary dosing regimen. Red outlined box indicates the single-ascending dose (SAD) phase for cohort 7, as described further in Example 1. Blue outlined box indicates the multiple-ascending dose (MAD) phase for cohort 7, as described further in Example 1. Green outlined box indicates the open-label extension OLE phase for cohort 7, as described further in Example 1. Green-filled cells within the table, labeled DEC, are exemplary time periods for a Data Evaluation Committee, or the like, to examine a subject for safety and/or treatment efficacy before and after the indicated administration.

FIG. 4 shows scintographies (A) and serial mid-ventricular ECV maps (B) visualizing amyloid transthyretin depletion in ATTR-CM by NI006/ALXN2220. FIG. 4 (A) shows serial bone scintigraphies of one patient randomized to NI006/ALXN2220 and one patient randomized to placebo, at baseline, at 4 months (after double-blinded SAD/MAD completion) and at 12 months (after OLE completion). Quantification of cardiac tracer uptake is indicated by heart/whole body ratio (H/WB ratio in %). Individual cumulative administered NI006/ALXN2220 dose (in g) as well as NI006/ALXN2220 exposure (AUC in day*mg/mL) are provided for the post-baseline imaging timepoints. FIG. 4 (B) shows serial mid-ventricular ECV maps of one patient randomized to NI006/ALXN2220 and one patient randomized to placebo at baseline, at 4 months and at 12 months Quantification of cardiac amyloid deposition is indicated by ECV measurement (in %). Individual cumulative administered NI006/ALXN2220 dose (in g) as well as NI006/ALXN2220 exposure (in day*mg/mL) are provided for the post-baseline imaging time points.

FIG. 5 shows graphs visualizing the changes in cardiac amyloid load, in particular the relative changes from baseline (RCFB) in cardiac amyloid derived from quantification of serial bone scintigraphies (triangles) and MRIs (points). FIG. 5 (A) shows RCFB per assigned NI006/ALXN2220 dose cohort and placebo at 4 months and 12 months. FIG. 5 (B) shows RCFB in patients assigned to NI006/ALXN2220 versus their individual cumulative NI006/ALXN2220 exposure at 4 months and at 12 months.

FIG. 6 shows graphs visualizing the changes in cardia amyloid in placebo switchers at 12 months, in particular the relative changes from baseline (RCFB) in cardiac amyloid derived from quantification of serial bone scintigraphies (triangles) and MRIs (points) for patients randomized to placebo. FIG. 6 (A) shows RCFB at 4 months and after switch to NI006/ALXN2220 during the open-label extension phase at 12 months. FIG. 6 (B) shows RCFB at 12 months in patients randomized to placebo but switched to NI006/ALXN2220 versus their individual cumulative NI006/ALXN2220 exposure at 12 months.

FIG. 7 shows graphs visualizing the changes in cardia biomarkers and echocardiographic parameters, in particular the relative changes from baseline in cardiac biomarkers and echocardiographic parameters after 12 months of treatment with NI006/ALXN2220. FIG. 7 (A) shows relative change from baseline (RCFB) of NT-proBNP and Troponin T per assigned NI006/ALXN2220 dose cohort (top) and versus each patient's individual cumulative NI006/ALXN2220 exposure at 12 months (bottom). FIG. 7 (B) shows absolute changes from baseline (ACFB) in echocardiographic indicators of cardiac structure (end-diastolic volume, ED-IVS), systolic function (left ventricular ejection fraction, LVEF, left systolic and diastolic volume, LVESV and LVEDV), and diastolic function (left atrial volume, LAV, and E/e′ ratio).

FIG. 8 shows graphs visualizing the predicted serum NI006/ALXN2220 95^{th} percentile Cₘₐₓ (FIG. 8 (A)) and median AUC_{28 days} (FIG. 8 (B)) after single dose. 1000 simulations by dose and body weight. Observed individual Cₘₐₓ and simulated individual AUC_{28 days}.

FIG. 9 shows graphs visualizing the predicted serum NI006/ALXN2220 95^{th} percentile Cₘₐₓ (FIG. 9 (A)) and median AUC_{28 days} (FIG. 9 (B)) at steady-state (9 q4w doses). 1000 simulations by dose and body weight. Observed individual Cₘₐₓ and simulated individual AUC^{28 days}.

FIG. 10 shows study schematic for the A Phase 3, Randomized, Double-blind, Placebo-controlled, Multicenter Study to Evaluate the Efficacy and Safety of Amyloid Depleter ALXN2220 in Adult Participants with Transthyretin Amyloid Cardiomyopathy (ATTR-CM). ^{a}Participants will be on standard therapy, as determined by treating and study physician(s), which may include conventional heart failure therapies and approved disease modifying agents for ATTR amyloidosis; ^{b}Randomization will be stratified by 3 factors: current treatment with a disease modifying agent (TTR silencer±TTR stabilizer vs TTR stabilizer alone vs none), TTR genotype (variant vs wild-type), and disease severity based on cardiac biomarker (NT-proBNP>3000 pg/mL vs NT-proBNP s 3000 pg/mL); ° Safety Follow-up Visits will be conducted up to 120 days after the last dose of study intervention.

### DETAILED DESCRIPTION

The present invention relates to methods of treating or preventing diseases associated with transthyretin-mediated amyloidosis (ATTR). Specifically, the present invention relates to a human anti-transthyretin (TTR) antibody, which is capable of binding mutated, misfolded, misassembled and/or aggregated TTR species and/or fragments thereof and does not substantially recognize physiological TTR species, for use in a method of treating or effecting prophylaxis of a subject having or at risk of having a transthyretin-mediated amyloidosis (ATTR), e.g., ATTR-CM, ATTR polyneuropathy (ATTR-PN), Familial Amyloid Polyneuropathy (FAP), Familial Amyloid Cardiomyopathy (FAC), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia, wherein the method includes the administration of the an anti-TTR antibody or pharmaceutical composition thereof in a dosing regimen described herein. The methods provide a reduction in cardiac amyloid deposits in the heart. The dosing regimens described herein may provide advantageous pharmacokinetic (PK) and pharmacodynamic (PD) properties, e.g., that allow for a dosing frequency of the anti-TTR antibody to be about once per month (e.g., once every 28±7 days, and once every 4 weeks, respectively) and reduce the level of cardiac amyloid (e.g., misfolded TTR protein) deposits in the heart. Accordingly, the treatment regimens described herein may impart surprisingly beneficial therapeutic and prophylactic effects on subjects having or who are at risk of having a disease associated with ATTR (e.g., ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, and preeclampsia).

In particular, the method comprises in one embodiment administering the antibody in a dosing regimen that results in a sustained (e.g., maintained for a period of time such as about 1 day to about 1 week, about 1 day to about 30 days, e.g., about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days) plasma concentration of the antibody at ≥1 μg/mL (e.g., from about 1 μg/mL to about 1000 μg/mL) in the subject.

In some embodiments, the antibody is for administration in a dosing regimen that results in a sustained plasma concentration of the antibody of 1 μg/mL to about 1000 μg/mL (e.g., about 1 μg/mL, about 2.5 μg/mL, about 5 μg/mL, about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, or about 1000 μg/mL) in the subject. In some embodiments, the antibody is for administration in a dosing regimen that results in a sustained plasma concentration of the antibody of 10 μg/mL to about 75 μg/mL (e.g., about 10 μg/mL to about 50 μg/mL, about 20 μg/mL to about 60 μg/mL, or about 50 μg/mL to about 75 μg/mL). Alternatively, the antibody is for administration in a dosing regimen that results in a sustained plasma concentration of the antibody of 20 μg/mL to about 200 μg/mL (e.g., about 20 μg/mL to about 100 μg/mL, about 50 μg/mL to about 150 μg/mL, or about 75 μg/mL to about 175 μg/mL). Alternatively, the antibody is for administration in a dosing regimen that results in a sustained plasma concentration of the antibody of 100 μg/mL to about 800 μg/mL (e.g., about 140 μg/mL to about 700 μg/mL, about 200 μg/mL to about 600 μg/mL, or about 400 μg/mL to about 800 μg/mL). For example, a trough concentration of the antibody in a subject of about 40-140 kg that receives about 10 mg/kg of the antibody once per month is about 20-100 μg/mL. In another embodiment, a trough concentration of the antibody in a subject of about 40-140 kg that receives about 60 mg/kg of the antibody once per month is about 140-700 μg/mL.

The present invention further relates to a human anti-transthyretin (TTR) antibody, which is capable of binding mutated, misfolded, misassembled and/or aggregated TTR species and/or fragments thereof and does not substantially recognize physiological TTR species, for use in treating or effecting prophylaxis of a subject having or at risk of having a transthyretin-mediated amyloidosis (ATTR), by administering the antibody in a dosing regimen that results in a sustained plasma concentration of the antibody at an area under the curve (AUC) (e.g., an area under a plasma drug concentration-time curve in a subject after, e.g., 17-50 weeks of treatment/dosing) of about 2,000 μg*day/mL to about 100,000 μg*day/mL, e.g., at least 30,000 μg*day/mL in the subject.

In some embodiments, the anti-TTR antibody is for administration in a dosing regimen that results in a sustained plasma concentration of the antibody at an AUC (e.g., an area under a plasma drug concentration-time curve in a subject after 17 weeks of treatment/dosing) of about 2,000 μg*day/mL to about 50,000 μg*day/mL (e.g., about 2,000 μg*day/mL, about 2,500 μg*day/mL, about 3,000 μg*day/mL, about 3,500 μg*day/mL, about 4,000 μg*day/mL, about 5,000 μg*day/mL, about 6,000 μg*day/mL, about 7,000 μg*day/mL, about 8,000 μg*day/mL, about 9,000 μg*day/mL, about 10,000 μg*day/mL, about, about 15,000 μg*day/mL, about, about 20,000 μg*day/mL, about 30,000 μg*day/mL, about 40,000 μg*day/mL, or about 50,000 μg*day/mL in the subject).

Alternatively, the anti-TTR antibody is for administration in a dosing regimen that results in a sustained plasma concentration of the antibody at an AUC (e.g., an area under a plasma drug concentration-time curve in a subject after 50 weeks of treatment/dosing) of about 2,500 μg*day/mL to about 100,000 μg*day/mL, about 5,000 μg*day/mL to about 100,000 μg*day/mL, or about 10,000 μg*day/mL to about 100,000 μg*day/mL (e.g., about 2,500 μg*day/mL, about 3,000 μg*day/mL, about 3,500 μg*day/mL, about 4,000 μg*day/mL, about 5,000 μg*day/mL, about 6,000 μg*day/mL, about 7,000 μg*day/mL, about 8,000 μg*day/mL, about 9,000 μg*day/mL, about 10,000 μg*day/mL, about 15,000 μg*day/mL, about 20,000 μg*day/mL, about 30,000 μg*day/mL, about 40,000 μg*day/mL, about 50,000 μg*day/mL, about 60,000 μg*day/mL, about 70,000 μg*day/mL, about 80,000 μg*day/mL, about 90,000 μg*day/mL, or about 100,000 μg*day/mL in the subject.

In some embodiments, administration of the anti-TTR antibody in accordance with the present invention is useful for long term treatment and/or for follow up treatment (e.g., at a dosage sufficient to achieve a sustained plasma concentration of about 1 μg/mL, 2.5 μg/mL, or 5 μg/mL) after initial higher dosing and amyloid removal.

In a preferred embodiment, the anti-TTR antibody for use in accordance with the present invention is administered at a dose of about 0.3 mg/kg to about 60 mg/kg (e.g., 0.3 mg/kg to 60 mg/kg, 0.3 mg/kg to 30 mg/kg, 0.3 mg/kg to 1 mg/kg, 1 mg/kg to 10 mg/kg, 10 mg/kg to 20 mg/kg, 20 mg/kg to 30 mg/kg, 30 mg/kg to 40 mg/kg, 40 mg/kg to 50 mg/kg, or 50 mg/kg to 60 mg/kg).

Alternatively, the anti-TTR antibody for use in accordance with the present invention is administered at a dose of about 0.3 mg/kg to about 30 mg/kg (e.g., 0.3 mg/kg to 20 mg/kg, 0.3 mg/kg to 10 mg/kg, 1 mg/kg to 15 mg/kg, 5 mg/kg to 15 mg/kg, 15 mg/kg to 20 mg/kg, 15 mg/kg to 25 mg/kg, or 25 mg/kg to 30 mg/kg).

In some embodiments, the anti-TTR antibody is administered at a dose of about 0.3 mg/kg.

In some embodiments, the anti-TTR antibody is administered at a dose of about 1 mg/kg.

In some embodiments, the anti-TTR antibody is administered at a dose of about 3 mg/kg.

In some embodiments, the anti-TTR antibody is administered at a dose of about 10 mg/kg.

In some embodiments, the anti-TTR antibody thereof is administered at a dose of about 30 mg/kg.

In some embodiments, the anti-TTR antibody is administered at a dose of about 30 mg/kg to about 80 mg/kg (e.g., 30 mg/kg to 40 mg/kg, 30 mg/kg to 50 mg/kg, 30 mg/kg to 60 mg/kg, 40 mg/kg to 50 mg/kg, 50 mg/kg to 60 mg/kg, 40 mg/kg to 60 mg/kg, 40 mg/kg to 70 mg/kg, 40 mg/kg to 80 mg/kg, 50 mg/kg to 60 mg/kg, 50 mg/kg to 70 mg/kg, or 50 mg/kg to 80 mg/kg).

In some embodiments, the anti-TTR antibody is administered at a dose of about 30 mg/kg.

In some embodiments, the anti-TTR antibody is administered at a dose of about 40 mg/kg.

In some embodiments, the anti-TTR antibody is administered at a dose of about 50 mg/kg.

In some embodiments, the anti-TTR antibody is administered at a dose of about 60 mg/kg.

In some embodiments, the anti-TTR antibody is administered at a dose of about 30 mg/kg and results in a sustained plasma concentration of the antibody of about 50 μg/mL in the subject.

In some embodiments, the anti-TTR antibody is administered at a dose of about 10 mg/kg and results in a sustained plasma concentration of the antibody of about 20 μg/mL to about 100 μg/mL in the subject.

In some embodiments, the anti-TTR antibody is administered at a dose of about 30 mg/kg and results in a sustained plasma concentration of the antibody of about 65 μg/mL to about 350 μg/mL in the subject.

In some embodiments, the anti-TTR antibody is administered at a dose of about 60 mg/kg and results in a sustained plasma concentration of the antibody of about 140 μg/mL to about 700 μg/mL in the subject.

In some embodiments, the anti-TTR antibody is administered at a dose of about 60 mg/kg.

In some embodiments, the anti-TTR is first administered at a dose of about 0.3 mg/kg to about 10 mg/kg (e.g., about 0.3 mg/kg to 1 mg/kg, 0.5 mg/kg to 2 mg/kg, 1 mg/kg to 5 mg/kg, 1 mg/kg to 10 mg/kg, 3 mg/kg to 8 mg/kg, 5 mg/kg to 10 mg/kg, or 8 mg/kg to 10 mg/kg.

In some embodiments, the anti-TTR antibody is further administered at a maintenance dose of about 10 mg/kg to about 60 mg/kg (e.g., 10 mg/kg to 20 mg/kg, 20 mg/kg to 30 mg/kg, 30 mg/kg to 40 mg/kg, 40 mg/kg to 50 mg/kg, 50 mg/kg to 60 mg/kg, 20 mg/kg to 50 mg/kg, or 30 mg/kg to 60 mg/kg). In a preferred embodiment of the present invention, the anti-TTR antibody is first administered at a dose of about 0.3 mg/kg to about 10 mg/kg (e.g., about 0.3 mg/kg to 1 mg/kg, 0.5 mg/kg to 2 mg/kg, 1 mg/kg to 5 mg/kg, 1 mg/kg to 10 mg/kg, 3 mg/kg to 8 mg/kg, 5 mg/kg to 10 mg/kg, or 8 mg/kg to 10 mg/kg, and further administered at a maintenance dose of about 10 mg/kg to about 60 mg/kg (e.g., 10 mg/kg to 20 mg/kg, 20 mg/kg to 30 mg/kg, 30 mg/kg to 40 mg/kg, 40 mg/kg to 50 mg/kg, 50 mg/kg to 60 mg/kg, 20 mg/kg to 50 mg/kg, or 30 mg/kg to 60 mg/kg).

As mentioned above, the anti-TTR antibody as used in accordance with the present invention depleted amyloid transthyretin from cardiac tissue in a dose- and time-dependent manner, with most pronounced effects at doses greater than 10 mg/kg when administered once every four weeks. Accordingly, in a preferred embodiment, the anti-TTR antibody is administered at a dose of greater than 10 mg/kg, for example between 10 mg/kg and 100 mg/kg, preferably between 10 mg/kg and 60 mg/kg, more preferably at a dose of 30 mg/kg or 60 mg/kg, preferably wherein the antibody is administered once every 4 weeks (q4w). Furthermore, data from bone scintigraphy and MRI indicated that doses of 30 mg/kg and 60 mg/kg decreased the cardiac amyloid deposition by a median of 12.8% and 25.6% compared to baseline at 4 months; at 12 months, the median reductions were 30.7% and 50.7%, respectively. In the same patients, NT-proBNP was reduced by 78.2% and 72.2% at 12 months.

Thus, in a further preferred embodiment of the present invention, the anti-TTR antibody is administered at a dose of about 30 mg/kg.

In another preferred embodiment of the present invention, the anti-TTR antibody is administered at a dose of about 60 mg/kg.

Accordingly, the method of treating or effecting prophylaxis in accordance with the present invention preferably comprises administration of the antibody at a dose of greater than 10 mg/kg, for example between 10 mg/kg and 100 mg/kg, preferably between 10 mg/kg and 60 mg/kg, more preferably at a dose of 30 mg/kg or 60 mg/kg, preferably wherein the antibody is administered once every 4 weeks (q4w).

Based on the results for the mg/kg doses in the safety and efficacy studies, modeling was performed to calculate flat doses, which are more convenient for administration and thus, a preferred embodiment of the present invention. In particular, a PK/PD model was established and flat doses in the range of 600 mg to 7500 mg have been calculated to be safe and effective; see Examples 5 and 6.

Accordingly, in one embodiment of the present invention, the anti-TTR antibody is administered at a maintenance dose of about 600 mg to about 7500 mg (e.g., 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6050 mg, 6100 mg, 6150 mg, 6200 mg, 6210 mg, 6220 mg, 6230 mg, 6240 mg, 6250 mg, 6260 mg, 6270 mg, 6280 mg, 6290 mg, 6300 mg, 6350 mg, 6400 mg, 6450 mg, 6500 mg, 6550 mg, 6600 mg, 6650 mg, 6700 mg, 6750 mg, 6800 mg, 6850 mg, 6900 mg, 6950 mg, 7000 mg, 7050 mg, 7100 mg, 7150 mg, 7200 mg, 7250 mg, 7300 mg, 7350 mg, 7400 mg, 7450 mg, or 7500 mg). In a preferred embodiment, the maintenance dose is about 2500 mg, e.g., preferably 2400 mg. In another preferred embodiment, the maintenance dose is about 3000 mg. In a most preferred embodiment, the maintenance dose is about 3500 mg, e.g., preferably 3200 mg.

In some embodiments, the anti-TTR antibody is further administered at a loading dose of about 600 mg to about 7500 mg (e.g., 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, or 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6050 mg, 6100 mg, 6150 mg, 6200 mg, 6210 mg, 6220 mg, 6230 mg, 6240 mg, 6250 mg, 6260 mg, 6270 mg, 6280 mg, 6290 mg, 6300 mg, 6350 mg, 6400 mg, 6450 mg, 6500 mg, 6550 mg, 6600 mg, 6650 mg, 6700 mg, 6750 mg, 6800 mg, 6850 mg, 6900 mg, 6950 mg, 7000 mg, 7050 mg, 7100 mg, 7150 mg, 7200 mg, 7250 mg, 7300 mg, 7350 mg, 7400 mg, 7450 mg, or 7500 mg).

Preferably, the anti-TTR antibody administered at a loading dose of about 600 mg to about 4000 mg (e.g., 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, or 4000 mg).

In a preferred embodiment, the loading dose is about 2500 mg, e.g., preferably 2400 mg. In another preferred embodiment, the loading dose is about 3000 mg. In a most preferred embodiment, the loading dose is about 3500 mg, e.g., preferably 2400 mg.

Preferably, the anti-TTR antibody is in accordance with the present invention administered at the above-mentioned maintenance dose of about 600 mg to about 7500 mg, which is preceded by administration of the anti-TTR antibody at the above-mentioned loading dose of about 600 mg to about 7500 mg, preferably of about 600 mg to about 4000 mg. In an optional embodiment, the loading dose is administered once every other week for up to two months before administration of a maintenance dose.

In a further preferred embodiment, the maintenance dose or the loading dose of the anti-TTR antibody is about 3000 mg, more preferably of about 3500 mg, and most preferably of about 3200 mg.

In a further preferred embodiment, the maintenance dose and the loading dose of the anti-TTR antibody is about 3000 mg, more preferably of about 3500 mg, and most preferably of about 3200 mg.

In some embodiments of the present invention, the anti-TTR is administered at a dose of 600 mg to 7500 mg (e.g., 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6050 mg, 6100 mg, 6150 mg, 6200 mg, 6210 mg, 6220 mg, 6230 mg, 6240 mg, 6250 mg, 6260 mg, 6270 mg, 6280 mg, 6290 mg, 6300 mg, 6350 mg, 6400 mg, 6450 mg, 6500 mg, 6550 mg, 6600 mg, 6650 mg, 6700 mg, 6750 mg, 6800 mg, 6850 mg, 6900 mg, 6950 mg, 7000 mg, 7050 mg, 7100 mg, 7150 mg, 7200 mg, 7250 mg, 7300 mg, 7350 mg, 7400 mg, 7450 mg, or 7500 mg). In a preferred embodiment, the dose is about 2500 mg, 3000 mg, 3500 mg, or 5000 mg, i.e., preferably the dose is 2200 mg, 3200 mg, or 4800 mg.

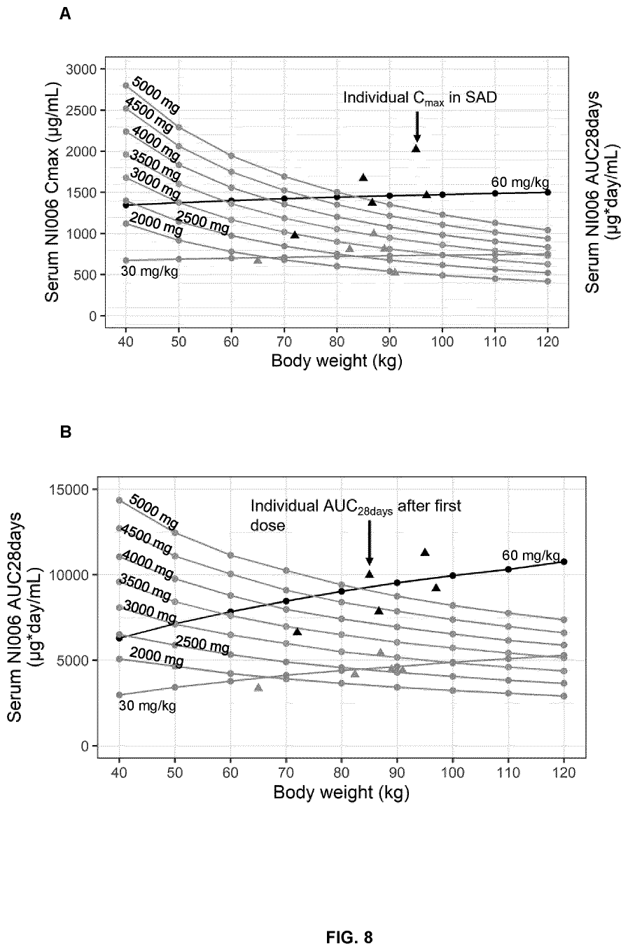

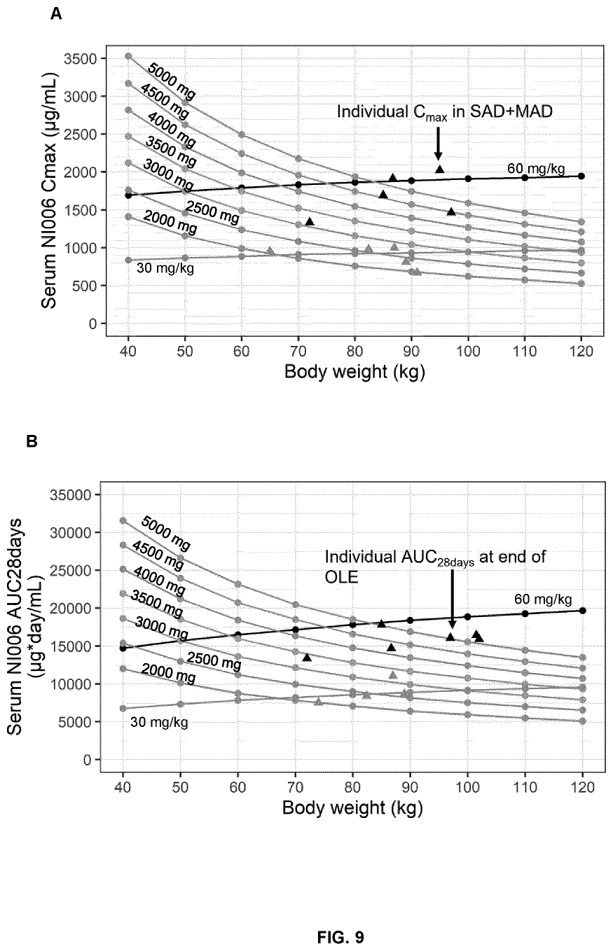

Using the above-mentioned PK/PD model, it has been further found that flat doses, but dependent on the patient's body weight, are most beneficial to achieve the safest and most effective treatment; see FIG. 8 and FIG. 9 and Example 5.

Accordingly, in one embodiment of the present invention, the anti-TTR antibody is administered at a dose of between 2000 mg and 2500 mg to patients with a body weight of about 40 to about ≤60 kg. In one embodiment, the anti-TTR antibody is administered at a dose of 2000 mg or 2500 mg to patients with a body weight of about 40 to about ≤60 kg. In preferred embodiment, the anti-TTR antibody is administered at a dose of 2500 mg to patients with a body weight of about 40 to about ≤60 kg. In one embodiment of the present invention, the anti-TTR antibody is administered at a dose of between 3000 mg and 3500 mg to patients with a body weight of about ≥60 kg to about 100 kg. In one embodiment, the anti-TTR antibody is administered at a dose of 3000 mg or 3500 mg to patients with a body weight of about ≥60 kg to about 100 kg. In a preferred embodiment, the anti-TTR antibody is administered at a dose of 3500 mg to patients with a body weight of about ≥60 kg to about 100 kg. In one embodiment of the present invention, the anti-TTR antibody is administered at a dose of between 4000 mg and 5000 mg to patients with a body weight of about ≤100 kg. In one embodiment, the anti-TTR antibody is administered at a dose of 4000 mg, 4500 mg, or 5000 mg to patients with a body weight of about ≤100 kg. In a preferred embodiment, the anti-TTR antibody is administered at a dose of 4000 mg or 5000 mg to patients with a body weight of about ≤100 kg, and most preferably the anti-TTR antibody is administered at a dose of 5000 mg to patients with a body weight of about ≤100 kg.

As explained in Example 6, based on the data of the Phase I Clinical Trial, the above-mentioned PK/PD model has been further refined to ensure administration of the complete amount of drug present in each vial and eliminate residual volume and corresponding drug wastage, while maintaining the same desired levels of efficacy. In particular, slightly lower flat doses have been developed using the PK/PD model, which also improve patient convenience since a lower amount of drug is administered and infused, respectively. These flat doses are used in the Phase III Clinical Trial as outlined in Example 8.

Accordingly, in a preferred embodiment of the present invention, the anti-TTR antibody is administered at a dose of in the range of 2200 mg and 2700 mg (inclusive of the endpoints) to patients with a body weight of about ≥40 kg to <60 kg. In a preferred embodiment, the anti-TTR antibody is administered at a dose of 2400 mg to patients with a body weight of about ≥40 kg to <60 kg. In one embodiment of the present invention, the anti-TTR antibody is administered at a dose of between 3000 mg and 3500 mg to patients with a body weight of about ≥60 kg to <100 kg. In a preferred embodiment, the anti-TTR antibody is administered at a dose of 3200 mg to patients with a body weight of about ≥60 kg to <100 kg. In one embodiment of the present invention, the anti-TTR antibody is administered at a dose of between 4400 mg and 5200 mg to patients with a body weight of about ≤100 kg. In a preferred embodiment, the anti-TTR antibody is administered at a dose of 4800 mg to patients with a body weight of about ≤100 kg.

In some embodiments, the anti-TTR antibody is administered to the subject once every 3 to 56 days, preferably once every 3 to 35 days, e.g., once every 7 to 35 days, e.g., once every 21 to 35 days (e.g., 22 to 34 days, 23 to 33 days, 24 to 32 days, 25 to 31 days, 23 to 33 days, or 27 days to 35 days). In some embodiments, the anti-TTR antibody is administered once weekly via subcutaneous or continuous infusion via a pump.

In some embodiments, the anti-TTR antibody is administered to the subject once every 28 to 35 days (e.g., 28 to 30 days, 29 to 34 days, 30 to 33 days, or 30 to 32 days).

In some embodiments, the anti-TTR antibody is administered to the subject once every 28 days.

In some embodiments, the anti-TTR antibody is administered to the subject once every 35 days. In preferred embodiment, the anti-TTR antibody is administered to the subject once every 28 days. Preferably, the anti-TTR antibody is administered once every 4 weeks (q4w).

Accordingly, in one embodiment, the anti-TTR antibody is administered at a dose of 30 mg/kg once every 28 days.

In one embodiment, the anti-TTR antibody is administered at a dose of 30 mg/kg once every 35 days.

In one embodiment, the anti-TTR antibody is administered at a dose of 30 mg/kg once every 4 weeks (q4w).

In one embodiment, the anti-TTR antibody is administered at a dose of 60 mg/kg once every 28 days.

In one embodiment, the anti-TTR antibody is administered at a dose of 60 mg/kg once every 35 days.

In one embodiment, the anti-TTR antibody is administered at a dose of 60 mg/kg once every 4 weeks (q4w).

In one embodiment, the anti-TTR antibody is administered at a dose of 2500 mg to patients with a body weight of 40 kg to ≤60 kg once every 28 days.

In one embodiment, the anti-TTR antibody is administered at a dose of 2500 mg to patients with a body weight of 40 kg to ≤60 kg once every 35 days.

In one embodiment, the anti-TTR antibody is administered at a dose of 2500 mg to patients with a body weight of 40 kg to ≤60 kg once every 4 weeks (q4w).

In one embodiment, the anti-TTR antibody is administered at a dose of 2400 mg to patients with a body weight of 40 kg to ≤60 kg once every 28 days.

In one embodiment, the anti-TTR antibody is administered at a dose of 2400 mg to patients with a body weight of 40 kg to ≤60 kg once every 35 days.

In one embodiment, the anti-TTR antibody is administered at a dose of 2400 mg to patients with a body weight of 40 kg to ≤60 kg once every 4 weeks (q4w).

In one embodiment, the anti-TTR antibody is administered at a dose of 3500 mg to patients with a body weight of ≥60 kg to 100 kg once every 28 days.

In one embodiment, the anti-TTR antibody is administered at a dose of 3500 mg to patients with a body weight of 60 kg to 100 kg once every 35 days.

In one embodiment, the anti-TTR antibody is administered at a dose of 3500 mg to patients with a body weight of 60 kg to 100 kg once every 4 weeks (q4w).

In one embodiment, the anti-TTR antibody is administered at a dose of 3200 mg to patients with a body weight of 60 kg to 100 kg once every 28 days.

In one embodiment, the anti-TTR antibody is administered at a dose of 3200 mg to patients with a body weight of 60 kg to 100 kg once every 35 days.

In one embodiment, the anti-TTR antibody is administered at a dose of 3200 mg to patients with a body weight of 60 kg to 100 kg once every 4 weeks (q4w).

In one embodiment, the anti-TTR antibody is administered at a dose of 5000 mg to patients with a body weight of 100 kg once every 28 days.

In one embodiment, the anti-TTR antibody is administered at a dose of 5000 mg to patients with a body weight of 100 kg once every 35 days.

In one embodiment, the anti-TTR antibody is administered at a dose of 5000 mg to patients with a body weight of 100 kg once every 4 weeks (q4w).

In one embodiment, the anti-TTR antibody is administered at a dose of 4800 mg to patients with a body weight of 100 kg once every 28 days.

In one embodiment, the anti-TTR antibody is administered at a dose of 4800 mg to patients with a body weight of 100 kg once every 35 days.

In one embodiment, the anti-TTR antibody is administered at a dose of 4800 mg to patients with a body weight of 100 kg once every 4 weeks (q4w).

In some embodiments, the anti-TTR antibody is administered to the subject for about 4-30 months (e.g., 4-12 months, 8-16 months, 12-20 months, 16-24 months, or 20-30 months).

In some embodiments, the anti-TTR antibody is administered to the subject for about 12-18 months (e.g., 12-15 months, 13-16 months, 14-17 months, or 15-18 months).

In some embodiments, the anti-TTR antibody is administered to the subject for about 4 months.

In some embodiments, the anti-TTR antibody is administered to the subject for about 11 months.

In some embodiments, the anti-TTR antibody is administered to the subject for about 24 to 48 months, but at least for 24 months.

In some embodiments, the anti-TTR antibody is administered to the subject at a dose of about 30 mg/kg or 60 mg/kg for about 12-18 months (e.g., 12, 13, 14, 15, 16, 17, or 18 months).

In some embodiments, the anti-TTR antibody is administered to the subject at a dose of about 30 mg/kg to 60 mg/kg once every 21 to 35 days for about 12-18 months (e.g., for about 12, 13, 14, 15, 16, 17, or 18 months).

In some embodiments, the loading dose is administered once every other week for up to two months before administration of a maintenance dose. In some embodiments, the maintenance dose is lower than the loading dose.

In some embodiments, the anti-TTR antibody is administered for up to 24 months in accordance with the intervention infusion (i.e., intravenous administration of the anti-ATTR antibody) schedule provided in Table 25. In further embodiments, the anti-TTR antibody is administered, as follow-up, after the 24-month treatment period in accordance with the intervention infusion schedule of Table 26.

In some embodiments, the plasma concentration of the anti-TTR antibody is sustained at about 1 μg/mL to about 1000 μg/mL (e.g., about 1 μg/mL to about 100 μg/mL, about 2.5 μg/mL to about 125 μg/mL, about 5 μg/mL to about 150 μg/mL, about 10 μg/mL to about 200 μg/mL, about 65 μg/mL to about 350 μg/mL, about 20 μg/mL to about 200 μg/mL, about 20 μg/mL to about 100 μg/mL, about 50 μg/mL to about 150 μg/mL, or about 75 μg/mL to about 175 μg/mL, about 100 μg/mL to about 800 μg/mL, about 140 μg/mL to about 700 μg/mL, about 200 μg/mL to about 600 μg/mL, or about 400 μg/mL to about 800 μg/mL, e.g., about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, or about 1000 μg/mL) for about 1 day to about 30 days (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days).

In some embodiments, the plasma concentration is sustained at about 1 μg/mL to about 1000 μg/mL (e.g., about 1 μg/mL to about 100 μg/mL, about 2.5 μg/mL to about 125 μg/mL, about 5 μg/mL to about 150 μg/mL, about 10 μg/mL to about 200 μg/mL, about 65 μg/mL to about 350 μg/mL, about 20 μg/mL to about 200 μg/mL, about 20 μg/mL to about 100 μg/mL, about 50 μg/mL to about 150 μg/mL, or about 75 μg/mL to about 175 μg/mL, about 100 μg/mL to about 800 μg/mL, about 140 μg/mL to about 700 μg/mL, about 200 μg/mL to about 600 μg/mL, or about 400 μg/mL to about 800 μg/mL, e.g., about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, or about 1000 μg/mL) for about 1 day to about 1 week.

In some embodiments, administration of the anti-TTR antibody or an antigen-binding fragment thereof is monitored by determining a level of at least one biomarker, e.g., N-terminal pro-B-type natriuretic peptide (NT-proBNP) or a fragment thereof. In addition, or alternatively, administration of the anti-TTR antibody is monitored by determining a level of one or more biomarkers, such as, e.g., cardiac troponin T (TnT) and N-terminal pro-B-type natriuretic peptide (NT-proBNP) or a fragment thereof. These biomarkers, and in particular NT-proBNP and/or its fragments are recognized in the art for being useful for monitoring the progress in ATTR treatment; see for example Perfetto et al., Internal and Emergency Medicine 17 (2022), 957-969 (incorporated herein by reference).

Thus, in an embodiment, a decrease in the level of the biomarker(s) is indicative of treatment efficacy.

In some embodiments, the subject has an N-terminal pro b-type natriuretic peptide (NT-proBNP) level of about 300 pg/mL to about 20,000 pg/mL before undergoing the treatment methods described herein. In some embodiments, the subject has an N-terminal pro b-type natriuretic peptide (NT-proBNP) level of about 0 pg/mL to about 300 pg/mL after undergoing the treatment methods described herein.

In some embodiments, the subject has a cardiac troponin T (TnT) level of about 10 pg/mL to about 200 pg/mL before undergoing the treatment methods described herein. In some embodiments, the subject has a cardiac troponin T (TnT) level of 0 pg/mL to about 10 pg/mL after undergoing the treatment methods described herein. In a preferred embodiment, the subject has an N-terminal pro b-type natriuretic peptide (NT-proBNP) level of >2000 pg/mL.

In one embodiment of the present invention, administration of the anti-TTR antibody in accordance with the present invention further comes along with determining a level of C-reactive protein (CRP), wherein a dose-dependent, transient (e.g., for about 1 to about 14 days, e.g., 1, 2, 3, 4, 5, 6, 7, 8, 9, 10, 11, 12, 13, or 14 days) increase of CRP level is indicative of on-target immune activation and efficacy of the treatment. Accordingly, CRP can be used as biomarker and a dose dependent transient increase of CRP level is indicative for on-target immune activation and efficacy of the treatment.

In some embodiments, administration of the anti-TTR antibody leads to reduction of cardiac amyloid burden, which is for example determined by magnetic resonance imaging (MRI).

In some embodiments, administration of the anti-TTR antibody leads to cardiac mass reduction, e.g., as measured by echocardiography or cardiac MRI.

In some embodiments, administration of the anti-TTR antibody leads to improved cardiovascular function (e.g., diastolic myocardial relaxation, atrioventricular (AV) flow, global longitudinal strain (GLS), ventricular ejection fraction, or end diastolic volume), e.g., as assessed by echocardiography. For example, echocardiography may be used to measure the ratio of early diastolic mitral inflow velocity to early diastolic mitral annulus velocity (E/e′ ratio) to assess cardiovascular function.

Analysis of the Clinical Trials performed within the scope of the present invention surprisingly revealed that after administration of 30 mg/kg or 60 mg/kg of the anti-TTR antibody to the patient, the cardiac amyloid deposition decreased (see Example 1) and the CRP levels transiently increased in a dose dependent manner. It has been even shown for the first time that a therapeutic intervention resulted in substantial and sustained amyloid depletion, which support the preclinical data that demonstrated the ability of NI006's/ALXN2220 to induce degradation of transthyretin amyloid. While not being bound to a particular mechanism or theory, this TTR amyloid degrading effect of NI006/ALXN2220 may be mediated via recruitment of phagocytic cells through its Fc-effector function.

Accordingly, due to this observation CRP can serve as a biomarker for monitoring the success of the treatment of an amyloidosis, in particular for monitoring the treatment of an amyloidosis by depletion of TTR via phagocytosis. This effect was particularly pronounced after the first administration of the antibody. The transient CRP responses observed with the anti-TTR antibody were mostly asymptomatic. CRP is an annular pentameric protein found in blood plasma, whose circulating concentrations rise for example in response to inflammation. The amino acid sequence of CRP is publicity available, see for example UniProt Reference: P02741 CRP_HUMAN. Early CRP kinetics have been described to be a possible implement on-treatment biomarker to predict response to 1st-line immune checkpoint blockade (IO) combination therapy of metastatic renal cell carcinoma (mRCC); see Klümper et al., Clinical & Translational Immunology 10 (2021), e1358. However, the observation that CRP kinetics accompany amyloid removal by phagocytosis in a transient and dose dependent manner has not been reported or suggested before.

Accordingly, in a further aspect, the present invention relates to CRP as biomarker for on-target immune activation and/or for monitoring the efficacy of the treatment of an amyloidosis, preferably of a systemic amyloidosis, preferably of ATTR, most preferably of ATTR-CM. In a further preferred embodiment, CRP as a biomarker is used for monitoring the treatment of an amyloidosis, preferably of a systemic amyloidosis, preferably of ATTR, most preferably of ATTR-CM, wherein the treatment is performed by depleting TTR by depletion of TTR via phagocytosis. More particularly, the present invention relates to the use of CRP as biomarker for on-target immune activation and/or for monitoring the efficacy of the treatment of the above-mentioned amyloidosis, e.g., of ATTR, preferably of ATTR-CM with an amyloid depleter, for example a therapeutic agent which is capable of depleting amyloid deposition, preferably via phagocytosis, preferably of cardiac amyloid deposition. In a preferred embodiment, the therapeutic agent/the amyloid depleter is an anti-TTR antibody as defined herein.

In this aspect, the present invention further relates to a method for monitoring on-target immune activation and/or the efficacy of the treatment of an amyloidosis, preferably of a systemic amyloidosis, more preferably of ATTR, and most preferably of ATTR-CM as mentioned above, with an amyloid deplete, for example a therapeutic agent which is capable of depleting amyloid deposition, preferably via phagocytosis, preferably of cardiac amyloid deposition, wherein the method comprises determining the level of CRP in a sample from the subject undergoing treatment, preferably wherein the level of CRP is determined at a first timepoint, e.g., before administration of the amyloid deplete and at least one second timepoint, e.g., one timepoint after administration of the amyloid depleter, wherein an increased level of CRP in the sample of the second timepoint in comparison to the sample of the first timepoint is indicative for the treatment efficacy. In a preferred embodiment, the second timepoint is after the first administration of the amyloid depleter. In a preferred embodiment, the amyloid depleter is an anti-TTR, preferably as defined herein and most preferably formulated as described herein and provided in the specific doses as described herein. The sample can be any tissue or preferably body fluid in which CRP is present, for example blood.

The present invention further relates to a kit comprising reagents for detection and measuring the level of CRP in a patient's sample, which is particularly useful for monitoring the treatment efficacy of the therapeutic agent/the amyloid depleter as explained above. In a preferred embodiment, the kit comprises non-immunological and/or immunological reagents for detecting CRP, for example anti-CRP antibodies, nucleic acid probes, or a PC-conjugate that contains multiple copies of covalently coupled phosphorylcholine (PC) moieties. The kit may further comprise reagents and/or instructions for use. Preferably, the amyloid depleter/therapeutic agent which efficacy is monitored, is the anti-TTR antibody as described herein, and which is administered in the indicated doses. Means and methods for the detection of CRP are known to the person skilled in the art.

The present invention further relates to an article of manufacture, e.g., a kit, preferably a therapeutic kit, providing the effective doses of the anti-TTR antibody as described herein, preferably the 30 mg/kg or 60 mg/kg doses, and the 2500 mg, 3500 mg, and 5000 mg flat doses, respectively, optionally including reagents for delivery of said doses, like in the form of a formulation. Most preferably, the therapeutic kit of the present invention provides the 2400 mg, 3200 mg, and/or 4800 mg flat doses. Thus, the kit preferably comprises one or more containers, wherein said containers comprise a formulation of the anti-TTR antibody as defined hereinbefore, preferably of antibody NI006/ALXN2022 as defined by its CDRs, VH and VL regions, and by its heavy chain and light chain, respectively as defined hereinbefore, wherein the containers comprise 2400 mg, 3200 mg, or 4800 mg of the antibody, or multiple doses of the antibody, for example 4800 mg, 6400 mg, 9600 mg, or 7200 mg, 9600 mg, or 14400 mg, etc. The kit preferably further comprises means for delivery of the antibody to the subject, wherein the means preferably comprise an infusion bag and/or a syringe.

Thus, the kit may comprise the anti-TTR antibody, for example provided in a container like a vial, optionally an infusion bag to which the antibody is added, optionally an infusion solution and/or dilution material (e.g., the formulation, and more preferably glucose). The kit may further comprise means to detect biomarkers for monitoring the efficacy of the treatment, preferably cardiac troponin T (TnT), N-terminal pro-B-type natriuretic peptide (NT-proBNP), and/or C-reactive protein (CRP). Alternatively, the kit of the present invention may further comprise means to detect at least the N-terminal pro-B-type natriuretic peptide (NT-proBNP). In one embodiment, the article of manufacture may comprise the anti-TTR antibody and the above-described kit for detecting CRP.

The present invention also relates to an article of manufacture which comprises on one more containers as defined with regard to the kit above, and a label which prescribes that the antibody is indicated for the treatment of the cardiomyopathy of wild-type or hereditary transthyretin-mediated amyloidosis in adults. Alternatively, the label indicates the dosing regime in accordance with the present invention, preferably the flat doses regimen.

In some embodiments, the anti-TTR antibody or antigen-binding fragment thereof is provided in an aqueous formulation at a concentration of about 25 to 200 mg/mL (e.g., 25 mg/mL, 50 mg/mL, 75 mg/mL, 100 mg/mL, 125 mg/mL, 150 mg/mL, 175 mg/mL, or 200 mg/mL), which may be diluted prior to administration. In some embodiments, the anti-TTR antibody is administered to a subject in an aqueous formulation at a diluted concentration of about 1 mg/mL to about 50 mg/mL (e.g., about 1 mg/mL to about 42 mg/mL, about 1 mg/mL to about 30 mg/mL, about 1 mg/mL to about 20 mg/mL, or about 1 mg/mL to about 10 mg/mL, e.g., about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL).

In some embodiments, the subject has been previously treated with and/or is concurrently receiving a TTR tetramer stabilizer. For example, the TTR tetramer stabilizer is selected from the group consisting of diflunisal, Tafamidis, and Acoramidis (AG10).

In some embodiments, the anti-TTR antibody is administered to the subject by intravenous infusion in an aqueous formulation, in which the aqueous formulation has a pH of about 5.0 to 6.5, includes a histidine buffer, a sugar such as sucrose, and a surfactant such as polysorbate 80 (PS80), and in which the anti-TTR antibody is present in the aqueous formulation at a concentration of about 25 to 125 mg/mL.

In some embodiments, the aqueous formulation has a pH of about 5.8 and includes 20 mM histidine (e.g., histidine hydrochloride, e.g., L-histidine and/or L-histidine monohydrochloride), 6.5% weight per volume (w/v) sucrose, and 0.03% PS80 w/v, and in which the antibody is present in the aqueous formulation at a concentration of about 50 mg/mL or about 100 mg/mL.

In some embodiment, the aqueous formulation has a pH of about 5.8 and includes 20 mM histidine (e.g., histidine hydrochloride, e.g., L-histidine and/or L-histidine monohydrochloride), 8% weight per volume (w/v) sucrose, and 0.03% PS80 w/v, and in which the anti-TTR antibody is present in the aqueous formulation at a concentration of about 50 mg/mL or about 100 mg/mL, preferably of about 50 mg/mL.

In some embodiments, the anti-TTR antibody is administered to the subject in a diluted form including a diluent.

In some embodiments, the diluent is glucose or a polymer thereof (e.g., the polymer is dextran). The glucose or polymer thereof (e.g., dextran) may be used as a diluent at a concentration of about 1%, 2%, 3%, 4%, 5%, 6%, 7%, 8%, 9%, or 10% w/v.

In some embodiments, an infusion line is flushed with the diluent before and after the intravenous infusion.

In some embodiments, the intravenous infusion is performed with a syringe pump with an infusion syringe or an infusion pump. In an embodiment, a total antibody dose of up to 100 mg is administered by infusion, for example, a syringe pump with an infusion syringe. In an embodiment, a total antibody dose greater than 100 mg is administered with an infusion pump. In an embodiment, the anti-TTR antibody is diluted into an infusion bag prefilled with the diluent.

In some embodiments, the infusion syringe includes a minimum administration volume of 10 mL of the aqueous formulation.

In some embodiments, a total volume of the diluted form administered to the subject does not exceed 200 mL (e.g., about 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 105 mL, 110 mL, 115 mL, 120 mL, 125 mL, 130 mL, 135 mL, 140 mL, 145 mL, 150 mL, 155 mL, 160 mL, 165 mL, 170 mL, 175 mL, 180 mL, 185 mL, 190 mL, 195 mL, or 200 mL), including a flushing volume. For example, in some embodiments, the total volume of a single administration does not exceed 200 mL.

In some embodiments, at least for a first infusion, the aqueous formulation is administered over approximately 2 hours (±10 minutes).

In some embodiments, the aqueous formulation is administered over approximately 1 hour (±10 minutes).

In some embodiments, the ATTR amyloidosis leads to Cardiomyopathy (CM) and thus, in a preferred embodiment, the subject to be treated in accordance with the present invention has ATTR amyloidosis with CM (ATTR-CM). In some embodiments, the subject has ATTR polyneuropathy (ATTR-PN). In some embodiments, the subject has Familial Amyloid Polyneuropathy (FAP). In some embodiments, the subject has Familial Amyloid Cardiomyopathy (FAC). In some embodiments, the subject has Senile Systemic Amyloidosis (SSA). In some embodiments, the subject has systemic familial amyloidosis. In some embodiments, the subject has leptomeningeal/Central Nervous System (CNS) amyloidosis. In some embodiments, the subject has Alzheimer disease. In some embodiments, the subject has TTR-related ocular amyloidosis. In some embodiments, the subject has TTR-related renal amyloidosis. In some embodiments, the subject has TTR-related hyperthyroxinemia. In some embodiments, the subject has TTR-related ligament amyloidosis including carpal tunnel syndrome. In some embodiments, the subject has rotator cuff tears and lumbar spinal stenosis. In some embodiments, the subject has preeclampsia.

In some embodiments, the subject has been diagnosed with hereditary ATTR-CM for a known pathogenic TTR mutation.

In some embodiments, the subject has sporadic, wild-type-ATTR-CM (WT-ATTR-CM) (e.g., a wild type ATTR gene that codes for TTR proteins that form deposits in the heart) and a negative genetic testing for a TTR mutation.

Preferably, the diagnosis is based on evidence of cardiac amyloidosis by echocardiography or cMRI and one of the following: (a) endomyocardial biopsy with confirmatory TTR amyloid typing by either immunohistochemistry or mass spectrometry; or (b) grade 2 or 3 cardiac uptake on 99mTc scintigraphy (99mTc DPD, 99mTc PYP, or 99mTc HMDP) in the absence of monoclonal gammopathy; or (c) grade 2 or 3 cardiac uptake on 99mTc scintigraphy (99mTc DPD, 99mTc PYP, or 99mTc HMDP), and confirmatory TTR amyloid typing by either immunohistochemistry or mass spectrometry in non-cardiac tissue in the presence of monoclonal gammopathy

In addition, or alternatively, the subject has left ventricular ejection fraction (LVEF) of 220% (e.g., about 20% to about 60%, e.g., 20%, 25%, 30%, 35%, 40%, 45%, 50%, 55%, or 60%) before undergoing the treatment methods described herein, e.g., as measured by echocardiography. In an embodiment, the subject has a LVEF of 50% to 70% (e.g., 50%, 55%, 60%, 65%, or 70%) after undergoing the treatment methods described herein (e.g., the subject exhibits an increase in LVEF after treatment relative to LVEF prior to treatment).

In addition, or alternatively, the subject has left ventricular wall thickness (LVWT) of ≥12 mm (e.g., about 12 mm to about 30 mm, e.g., about 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm) before undergoing the treatment methods described herein, e.g., as measured by echocardiography. In an embodiment, the subject has a LVWT of <12 mm (e.g., about 8 mm to about 12 mm, e.g., about 8 mm, 9 mm, 10 mm, 11 mm, or 12 mm) after undergoing the treatment methods described herein.

In addition, or alternatively, the subject, in particular a woman, has an end-diastolic interventricular septal wall thickness of 211 mm (e.g., about 11 mm to about 30 mm, e.g., about 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm), or the subject, in particular a man, has an end-diastolic interventricular septal wall thickness 212 mm (e.g., about 12 mm to about 30 mm, e.g., about 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm) before undergoing the treatment methods described herein, e.g., as measured by echocardiography.

In addition, or alternatively, the subject (e.g., a woman) has an end-diastolic interventricular septal wall thickness of <11 mm (e.g., about 8 mm to about 11 mm, e.g., about 8 mm, 9 mm, 10 mm, or 11 mm) after undergoing the treatment methods described herein; or the subject (e.g., a man) has an end-diastolic interventricular septal wall thickness of <12 mm (e.g., about 8 mm to about 12 mm, e.g., about 8 mm, 9 mm, 10 mm, 11 mm, or 12 mm) after undergoing the treatment methods described herein.

In addition, or alternatively, the subject has an NT-proBNP level of about 300 pg/mL to about 20,000 pg/mL before undergoing the treatment methods described herein, preferably the subject has a NT-proBNP level of >2000 pg/mL before undergoing the treatment methods described herein. The use of NT-proBNP as biomarker in ATTR is recognized in the art and based on the level of said biomarker (either in combination with the level of cardiac troponin T (cTnT) (Grogan et al., J Am Coll Cardiol 68 (2016), 1014-1020) or in combination with estimated glomerular filtration rate (eGFR) (Gillmore et al., European Heart Journal 39 (2018), 2799-2806)) staging systems have been developed with a cut off for NT-proBNP of 3000 pg/mL; see also Perfetto et al., Internal and Emergency Medicine 17 (2022), 957-969. Thus, dependent on the level of cTnT and eGFR, and only with a view to the level of NT-proBNP, the subject has Grade I, II and III cardiac ATTR.

In addition, or alternatively, the subject has a history of heart failure as documented by one of the following events within about 1 year before undergoing the treatment methods described herein: (i) heart failure hospitalization, (ii) urgent heart failure visit, and (iii) episode of volume overload documented by NT-proBNP>2000 pg/mL (or equivalent BNP).

Preferably, the subject is an adult subject.

Thus, the treatment regime of the present invention can be used for treating subjects having any one or all of the mentioned indications/characteristics. In an embodiment of the present invention, the heavy chain variable region of the anti-TTR antibody or antigen-binding fragment thereof as used in accordance with the present invention includes an amino acid sequence with about 85% sequence identity (e.g., about 86%, 87%, 88%, 89%, 90%, 91%, 92%, 93%, 94%, 95%, 96%, 97%, 98%, 99%, or 100% sequence identity) to the amino acid sequence of SEQ ID NO: 7 and the light chain variable region of the anti-TTR antibody or antigen-binding fragment thereof includes an amino acid sequence with about 85% sequence identity (e.g., about 86%, 87%, 88%, 89%, 90%, 91%, 92%, 93%, 94%, 95%, 96%, 97%, 98%, 99%, or 100% sequence identity) to the amino acid of SEQ ID NO: 8.

In some embodiments, the heavy chain variable region of the anti-TTR antibody or antigen-binding fragment thereof includes the amino acid sequence of SEQ ID NO: 7 and the light chain variable region of the anti-TTR antibody or antigen-binding fragment thereof includes the amino acid of SEQ ID NO: 8.

In some embodiments, the heavy chain variable region of the anti-TTR antibody or antigen-binding fragment thereof includes the amino acid sequence of SEQ ID NO: 11 and the light chain variable region of the anti-TTR antibody or antigen-binding fragment thereof includes the amino acid of SEQ ID NO: 8.

In some embodiments, the heavy chain variable region of the anti-TTR antibody or antigen-binding fragment thereof includes the amino acid sequence of SEQ ID NO: 11 and the light chain variable region of the anti-TTR antibody or antigen-binding fragment thereof includes the amino acid of SEQ ID NO: 12.

As mentioned above, the parent antibody NI006 is also described in WO 2015/092077 A1 (designated as antibody NI-301.37F1) and in Michalon et al., Nat Commun. 12 (2021), 3142 (designated as antibody NI301A) and is capable of binding a human TTR epitope which comprises or consists of the amino acid sequence TTR41-45 (SEQ ID NO: 51 of WO 2015/092077 A1). Characterization of the binding properties of the antibody demonstrated that it presents high binding affinity to misfolded TTR in the sub-nanomolar range, is highly selective for the amyloid conformation of TTR, i.e., binds selectively with high affinity to the disease associated ATTR aggregates, exerts similar binding to wild type TTR and variant TTR. Related to sporadic or hereditary disease, respectively, and does not bind physiological TTR monomers. Furthermore, the anti-TTR antibody binds ATTR deposits in cardiac tissues obtained at autopsy from ATTR-CM patients. Accordingly, in one embodiment, the antibody as used in accordance with the present invention is an antibody, which is equivalent to the above-characterized antibody having a heavy chain and a light chain variable region including the amino acid of SEQ ID NO: 7 and SEQ ID NO: 8 meaning that the equivalent antibody has substantially the same binding characteristics than the above-characterized antibody having a heavy chain and a light chain variable region including the amino acid of SEQ ID NO: 7 and SEQ ID NO: 8. In particular, the equivalent antibody
(i) binds a human TTR epitope which comprises or consists of the amino acid sequence TTR41-45,
(ii) presents high binding affinity to misfolded TTR in the sub-nanomolar range,
(iii) is highly selective for the amyloid conformation of TTR, i.e., binds selectively with high affinity to the disease associated ATTR aggregates
(iv) exerts similar binding to wild type TTR and variant TTR. Related to sporadic or hereditary disease,
(v) does not bind physiological TTR monomers, and/or
(vi) binds ATTR deposits in cardiac tissues obtained at autopsy from ATTR-CM patients.

In one embodiment, the equivalent antibody shows one of the binding characteristics (i) to (vi). In one embodiment, the equivalent antibody shows at least two of the listed binding characteristics. In one embodiment, the equivalent antibody shows at least three of the listed binding characteristics. In one embodiment, the equivalent antibody shows at least four of the listed binding characteristics. In one embodiment, the equivalent antibody shows at least five of the listed binding characteristics. In a preferred embodiment, the equivalent antibody shows all of the binding characteristics (i) to (vi).

Antibody NI006/ALXN2220 is a fully human IgG1m3 allotype antibody and thus comprises the human constant heavy chain (HC) amino acid sequence as exemplified in SEQ ID NO: 9 and a corresponding human constant light chain (LC), here kappa light chain, as exemplified in SEQ ID NO: 10.

As shown in Example 7 and referred to in detail above, the major PTMs that have been identified in antibody NI006/ALXN2220 are the modification in the HC of glutamine at the N-terminus to pyro-glutamic acid, the loss of C-terminal lysine, and N-glycosylation. Thus, in a preferred embodiment, the heavy chain of the anti-TTR antibody for use in accordance with the present invention lacks the C-terminal cysteine, has a modified glutamine at the N-terminal as pyro-glutamic acid and comprises at least one N-glycosylation site.

Another class of anti-TTR antibodies that could be used in accordance with the present invention is described in international applications by Prothena Biosciences Limited (Prothena). In particular, embodiments of the disclosure relate to the use of anti-TTR antibody NN-6019 of Novo Nordisk (formerly known as PRX004 from Prothena Biosciences) in the treatment of a human subject in need of such treatment, as provided herein. NN-6019 (PRX004) corresponds to and is the humanized version of antibody 14G8 described in Higaki et al., Amyloid 23 (2016) 86-97 and which is disclosed in WO 2016/120810 A1 and WO 2018/007922A2 and more specifically in WO 2019/108689 A1, the disclosure in these documents being incorporated by reference. NN-6019 (PRX-004) is an investigational monoclonal antibody designed to specifically target and clear the misfolded (toxic) forms of the TTR amyloid protein found in ATTR. Accordingly, antibody PRX004 would be another preferred anti-TTR antibody for use in the treatment method in accordance with the present invention among others which recognize the same epitope as PRX004, i.e. amino acids TTR89-97 or an epitope comprising amino acids TTR₁₀₁₋₁₀₉, and which are humanized versions of the originally cloned mouse monoclonal antibodies 14G8, 9D5, 5A1, 6C1 disclosed in WO 2016/120810 A1, WO 2018/007924 A2, WO 2018/007924 A2 and WO 2018/007923 A1, the disclosure in these references, including, the antibody sequences, e.g., full-length and/or CDRs thereof, relating to these antibody clones and/or deposits thereof are incorporated herein by reference.

Thus, in one embodiment, the antibody for use in accordance with the present invention is a monoclonal antibody comprising a mature heavy chain variable region comprising three CDRs of SEQ ID NO:61 as set forth in WO 2019/108689 A1 and a mature light chain variable region comprising three CDRs of SEQ ID NO:70 as set forth in WO 2019/108689 A1, except that positions H52 and L26 by Kabat numbering can each be independently N or S, or a monoclonal antibody comprising a mature heavy chain variable region comprising three CDRs of SEQ ID NO: 1 as set forth in WO 2019/108689 A1 and a mature light chain variable region comprising three CDRs of SEQ ID NO:16 as set forth in WO 2019/108689 A1, In one embodiment, the antibody is characterized by comprising a mature heavy chain variable region comprising the amino acid sequence of SEQ ID NO:65 as set forth in WO 2019/108689 A1, and a mature light chain variable region comprising the amino acid sequence of SEQ ID NO:76 as set forth in WO 2019/108689 A1. More preferably, the antibody is characterized as in claims 26 to 35 of WO 2019/108689 A1, which content is herein incorporated by reference.

In a further aspect, the present invention relates to a TTR tetramer stabilizer for use in a method of treating or effecting prophylaxis of a subject having or at risk of ATTR (e.g., ATTR-CM, ATTR polyneuropathy (ATTR-PN), Familial Amyloid Polyneuropathy (FAP), Familial Amyloid Cardiomyopathy (FAC), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia), in which the subject has previously been and/or is concomitantly treated with an anti-TTR antibody in accordance with the present invention.

In some embodiments, the TTR tetramer stabilizer is selected from the group consisting of diflunisal, tafamidis, and acoramidis (AG10).

In a further aspect, the present invention relates to a method of treating ATTR (e.g., ATTR-CM, ATTR polyneuropathy (ATTR-PN), Familial Amyloid Polyneuropathy (FAP), Familial Amyloid Cardiomyopathy (FAC), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia) by administering to a human subject a human anti-TTR antibody at a dosage providing about 0.3 to about 100 mg/kg to the subject once every 3 to 42 days. The antibody is preferably the antibody as defined hereinbefore. Thus, preferably, the antibody or antigen-binding fragment thereof includes a heavy chain variable region with at least 80% sequence identity (e.g., at least 85%, 90%, 95%, 97%, 99%, or 100% sequence identity) to SEQ ID NO: 7 and a light chain variable region including an amino acid sequence with at least 80% sequence identity (e.g., at least 85%, 90%, 95%, 97%, 99%, or 100% sequence identity) to SEQ ID NO: 8, or the antibody can be an equivalent antibody with substantially the same binding characteristics as defined, infra. The heavy chain variable region includes the CDRs set forth in SEQ ID NOs: 1-3 and the light chain variable region includes the CDRs set forth in SEQ ID NOs: 4-6. Most preferably, the antibody is a fully human IgG1m3 allotype antibody and thus comprises the human constant heavy chain (HC) amino acid sequence as exemplified in SEQ ID NO: 9 and a corresponding human constant light chain (LC) as exemplified in SEQ ID NO: 10, preferably having the above-identified posttranslational modifications. The antibody is formulated in a pharmaceutical composition at a concentration of about 10 mg/mL to about 100 mg/mL.

In some embodiments, the anti-TTR antibody is administered at a dose of about 0.3 mg/kg to about 80 mg/kg (e.g., 0.3 mg/kg to 60 mg/kg, 0.3 mg/kg to 30 mg/kg, 0.3 mg/kg to 1 mg/kg, 1 mg/kg to 10 mg/kg, 10 mg/kg to 20 mg/kg, 20 mg/kg to 30 mg/kg, 30 mg/kg to 40 mg/kg, 30 mg/kg to 50 mg/kg, 30 mg/kg to 60 mg/kg, 40 mg/kg to 50 mg/kg, 40 mg/kg to 60 mg/kg, 40 mg/kg to 70 mg/kg, 40 mg/kg to 80 mg/kg, 50 mg/kg to 60 mg/kg, 50 mg/kg to 70 mg/kg, 50 mg/kg to 80 mg/kg, 60 mg/kg to 70 mg/kg, 60 mg/kg to 80 mg/kg, or 70 mg/kg to 80 mg/kg).

In some embodiments, the anti-TTR antibody is administered at a dose of about 0.3 mg/kg to about 30 mg/kg (e.g., 0.3 mg/kg to 20 mg/kg, 0.3 mg/kg to 10 mg/kg, 1 mg/kg to 15 mg/kg, 5 mg/kg to 15 mg/kg, 15 mg/kg to 20 mg/kg, 15 mg/kg to 25 mg/kg, or 25 mg/kg to 30 mg/kg).

In some embodiments, the anti-TTR antibody is administered at a dose of about 0.3 mg/kg.

In some embodiments, the anti-TTR antibody is administered at a dose of about 1 mg/kg.

In some embodiments, the anti-TTR antibody is administered at a dose of about 3 mg/kg.

In some embodiments, the anti-TTR antibody is administered at a dose of about 10 mg/kg.

In some embodiments, the anti-TTR antibody is administered at a dose of about 30 mg/kg.

In some embodiments, the anti-TTR antibody is administered at a dose of about 30 mg/kg to about 60 mg/kg (e.g., 30 mg/kg to 40 mg/kg, 40 mg/kg to 50 mg/kg, 50 mg/kg to 60 mg/kg, 30 mg/kg to 50 mg/kg, or 40 mg/kg to 60 mg/kg).

In some embodiments, the anti-TTR antibody is administered at a dose of about 30 mg/kg and results in a sustained plasma concentration of the antibody of about 50 μg/mL in the subject.

In some embodiments, the anti-TTR antibody is administered at a dose of about 60 mg/kg.

In some embodiments, the anti-TTR antibody is first administered at a dose of about 0.3 mg/kg to about 10 mg/kg (e.g., about 0.3 mg/kg to 1 mg/kg, 0.5 mg/kg to 2 mg/kg, 1 mg/kg to 5 mg/kg, 1 mg/kg to 10 mg/kg, 3 mg/kg to 8 mg/kg, 5 mg/kg to 10 mg/kg, or 8 mg/kg to 10 mg/kg.

In some embodiments, the anti-TTR antibody is further administered at a maintenance dose of about 10 mg/kg to about 60 mg/kg (e.g., 10 mg/kg to 20 mg/kg, 20 mg/kg to 30 mg/kg, 30 mg/kg to 40 mg/kg, 40 mg/kg to 50 mg/kg, 50 mg/kg to 60 mg/kg, 20 mg/kg to 50 mg/kg, or 30 mg/kg to 60 mg/kg).

In a preferred embodiment of the present invention, the anti-TTR antibody is first administered at a dose of about 0.3 mg/kg to about 10 mg/kg (e.g., about 0.3 mg/kg to 1 mg/kg, 0.5 mg/kg to 2 mg/kg, 1 mg/kg to 5 mg/kg, 1 mg/kg to 10 mg/kg, 3 mg/kg to 8 mg/kg, 5 mg/kg to 10 mg/kg, or 8 mg/kg to 10 mg/kg, and further administered at a maintenance dose of about 10 mg/kg to about 60 mg/kg (e.g., 10 mg/kg to 20 mg/kg, 20 mg/kg to 30 mg/kg, 30 mg/kg to 40 mg/kg, 40 mg/kg to 50 mg/kg, 50 mg/kg to 60 mg/kg, 20 mg/kg to 50 mg/kg, or 30 mg/kg to 60 mg/kg).

In a further preferred embodiment, the anti-TTR antibody is administered at a dose of greater than 10 mg/kg, for example between 10 mg/kg and 100 mg/kg, preferably between 10 mg/kg and 60 mg/kg, preferably wherein the antibody is administered once every 4 weeks.

In a further preferred embodiment, the anti-TTR antibody is administered at a dose of about 30 mg/kg, preferably wherein the antibody is administered once every 4 weeks.

In another preferred embodiment, the anti-TTR antibody is administered at a dose of about 60 mg/kg, preferably wherein the antibody is administered once every 4 weeks.

In some embodiments, the anti-TTR antibody is administered at a maintenance dose of about 600 mg to about 7500 mg (e.g., 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6050 mg, 6100 mg, 6150 mg, 6200 mg, 6210 mg, 6220 mg, 6230 mg, 6240 mg, 6250 mg, 6260 mg, 6270 mg, 6280 mg, 6290 mg, 6300 mg, 6350 mg, 6400 mg, 6450 mg, 6500 mg, 6550 mg, 6600 mg, 6650 mg, 6700 mg, 6750 mg, 6800 mg, 6850 mg, 6900 mg, 6950 mg, 7000 mg, 7050 mg, 7100 mg, 7150 mg, 7200 mg, 7250 mg, 7300 mg, 7350 mg, 7400 mg, 7450 mg, or 7500 mg).

In a preferred embodiment, the maintenance dose is about 2500 mg, e.g., 2400 mg, about 3000 mg, or about 3500 mg, e.g., 3200 mg.

In one embodiment of the present invention, the anti-TTR antibody is further administered at a loading dose of about 600 mg to about 4000 mg (e.g., 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, or 4000 mg.

In a preferred embodiment, the loading dose is about 2500 mg, e.g., 2400 mg, about 3000 mg, or about 3500 mg, e.g., 3200 mg.

Preferably, the anti-TTR antibody is administered at the above-mentioned maintenance dose of about 600 mg to about 7500 mg, which is preceded by administration of the anti-TTR antibody at the above-mentioned loading dose of about 600 mg to about 4000 mg. In an optional embodiment, the loading dose is administered once every other week for up to two months before administration of a maintenance dose.

In one embodiment of the present invention, the maintenance dose or the loading dose of the anti-TTR antibody is about 3000 mg.

In one embodiment of the present invention, the anti-TTR antibody is administered at a dose of between 2000 mg and 2500 mg to patients with a body weight of about 40 to about <60 kg. In one embodiment, the anti-TTR antibody is administered at a dose of 2000 mg or 2500 mg to patients with a body weight of about 40 to about ≤60 kg. In preferred embodiment, the anti-TTR antibody is administered at a dose of 2500 mg to patients with a body weight of about 40 to about ≤60 kg. In one embodiment of the present invention, the anti-TTR antibody is administered at a dose of between 3000 mg and 3500 mg to patients with a body weight of about 60 kg to about 100 kg. In one embodiment, the anti-TTR antibody is administered at a dose of 3000 mg or 3500 mg to patients with a body weight of about 60 kg to about 100 kg. In a preferred embodiment, the anti-TTR antibody is administered at a dose of 3500 mg to patients with a body weight of about 60 kg to about 100 kg. In one embodiment of the present invention, the anti-TTR antibody is administered at a dose of between 4000 mg and 5000 mg to patients with a body weight of about ≤100 kg. In one embodiment, the anti-TTR is administered at a dose of 4000 mg, 4500 mg, or 5000 mg to patients with a body weight of about ≤100 kg. In a preferred embodiment, the anti-TTR antibody is administered at a dose of 4000 mg or 5000 mg to patients with a body weight of about ≤100 kg, and most preferably the anti-TTR antibody is administered at a dose of 5000 mg to patients with a body weight of about ≤100 kg.

In a preferred embodiment of the present invention, the anti-TTR antibody is administered at a dose in the range of 2200 mg and 2700 mg (inclusive of the endpoints) to a subject with a body weight of about ≥40 kg to <60 kg. In a preferred embodiment, the anti-TTR antibody is administered at a dose of 2400 mg to patients with a body weight of about ≥40 kg to <60 kg. In one embodiment of the present invention, the anti-TTR antibody is administered at a dose in the range of 3000 mg and 3500 mg (inclusive of the endpoints) to patients with a body weight of about ≥60 kg to <100 kg. In a preferred embodiment, the anti-TTR antibody is administered at a dose of 3200 mg to patients with a body weight of about ≥60 kg to <100 kg. In one embodiment of the present invention, the anti-TTR antibody is administered at a dose in the range of 4400 mg and 5200 mg (inclusive of the endpoints) to patients with a body weight of about ≤100 kg. In a preferred embodiment, the anti-TTR antibody is administered at a dose of 4800 mg to patients with a body weight of about ≤100 kg.

In some embodiments, the anti-TTR antibody is administered to the subject once every 21 to 35 days (e.g., 22 to 34 days, 23 to 33 days, 24 to 32 days, 25 to 31 days, 23 to 33 days, or 27 days to 35 days).

In some embodiments, the anti-TTR antibody is administered to the subject once every 28 to 35 days (e.g., 28 to 30 days, 29 to 34 days, 30 to 33 days, or 30 to 32 days).

In some embodiments, the anti-TTR antibody is administered to the subject once every 28 days.

In some embodiments, the anti-TTR antibody is administered to the subject once every 35 days.

In some embodiments, the anti-TTR antibody is administered to the subject once every 4 weeks (q4w).

In some embodiments, the anti-TTR antibody is administered to the subject for about 4-30 months (e.g., 4-12 months, 8-16 months, 12-20 months, 16-24 months, or 20-30 months).

In some embodiments, the anti-TTR antibody is administered to the subject for about 12-18 months (e.g., 12-15 months, 13-16 months, 14-17 months, or 15-18 months).

In some embodiments, the anti-TTR antibody is administered to the subject for about 4 months.

In some embodiments, the anti-TTR antibody is administered to the subject for about 11 months.

In some embodiments, the anti-TTR antibody is administered to the subject at a dose of about 30 mg/kg for about 12-18 months (e.g., 12, 13, 14, 15, 16, 17, or 18 months).

In some embodiments, the loading dose is administered once every other week for up to two months before administration of a maintenance dose.

In some embodiments, administration of the anti-TTR antibody results in a sustained plasma concentration of the antibody at about 1 μg/mL to about 1000 μg/mL (e.g., about 1 μg/mL to about 100 μg/mL, about 2.5 μg/mL to about 125 μg/mL, about 5 μg/mL to about 150 μg/mL, about 10 μg/mL to about 200 μg/mL, about 10 μg/mL to about 50 μg/mL, about 25 μg/mL to about 75 μg/mL, about 50 μg/mL to about 1000 μg/mL) in the subject.

In some embodiments, the plasma concentration is sustained at about 1 μg/mL to about 1000 μg/mL (e.g., about 1 μg/mL to about 100 μg/mL, about 2.5 μg/mL to about 125 μg/mL, about 5 μg/mL to about 150 μg/mL, about 10 μg/mL to about 200 μg/mL, about 65 μg/mL to about 350 μg/mL, about 20 μg/mL to about 200 μg/mL, about 20 μg/mL to about 100 μg/mL, about 50 μg/mL to about 150 μg/mL, about 75 μg/mL to about 175 μg/mL, about 100 μg/mL to about 800 μg/mL, about 140 μg/mL to about 700 μg/mL, about 200 μg/mL to about 600 μg/mL, or about 400 μg/mL to about 800 μg/mL, e.g., about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, or about 1000 μg/mL) for about 1 day to about 30 days (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days).

In some embodiments, the plasma concentration is sustained at about 1 μg/mL to about 1000 μg/mL (e.g., about 1 μg/mL to about 100 μg/mL, about 2.5 μg/mL to about 125 μg/mL, about 5 μg/mL to about 150 μg/mL, about 10 μg/mL to about 200 μg/mL, about 65 μg/mL to about 350 μg/mL, about 20 μg/mL to about 200 μg/mL, about 20 μg/mL to about 100 μg/mL, about 50 μg/mL to about 150 μg/mL, about 75 μg/mL to about 175 μg/mL, about 100 μg/mL to about 800 μg/mL, about 140 μg/mL to about 700 μg/mL, about 200 μg/mL to about 600 μg/mL, or about 400 μg/mL to about 800 μg/mL, e.g., about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, or about 1000 μg/mL) for about 1 day to about 1 week.

In some embodiments, administration of the anti-TTR antibody is monitored by determining the level of one or more biomarkers.

In some embodiments, the marker is a protein marker obtained from serum or plasma of a subject, e.g., patient with ATTR-CM.

In some embodiments, the biomarkers include cardiac troponin T (TnT) and N-terminal pro-B-type natriuretic peptide (NT-proBNP), in which a decrease of the level of the biomarkers is indicative for efficacy of the treatment. In a preferred embodiment, the biomarker is at least NT-proBNP. In addition, or alternatively, the biomarkers include C-reactive protein (CRP), wherein an increase of CRP level, preferably in a dose-dependent manner, transient (e.g., about 1 to about 14 days, e.g., 1, 2, 3, 4, 5, 6, 7, 8, 9, 10, 11, 12, 13, or 14 days) increase of CRP level, is indicative for on-target immune activation and efficacy of the treatment.

In some embodiments, administration of the anti-TTR antibody or an antigen-binding fragment thereof leads to reduction of cardiac amyloid burden and/or composite of all-cause mortality (ACM) and total cardiovascular (CV) clinical events and/or heart failure (HF) events.

In some embodiments, administration of the anti-TTR antibody leads to cardiac mass reduction as measured by cardiac magnetic resonance imaging (MRI).

In some embodiments, treatment with the anti-TTR antibody in accordance with the present method(s) results in a dose- and time-dependent reduction, in the patient, cardiac amyloid load of at least about 25%, about 30%, about 35%, about 40%, about 45%, about 50%, about 55%, about 60%, or more, e.g., about 70%, about 80%, about 90%, after a specified duration of treatment, e.g., 4 months, 6 months, 8 months, 10 months, 12 months, 15 months, 18 months, 21 months, or 24 months, or more, e.g., 48 months.

Preferably, the treatment, i.e., the administration of the dosing regimen of the present invention improves at least one of the following:
(a) symptoms, functionality, and health-related quality-of-life (QoL) as measured by the change from baseline in Kansas City Cardiomyopathy Questionnaire overall summary (KCCQ-OS) score;
(b) time to cardiovascular (CV)-related mortality;
(c) six-minute walk test (6MWT) score compared to baseline;
(d) rate of cardiovascular (CV) clinical events; and
(e) time to all-cause mortality (ACM).

In one embodiment, the treatment
(a) reduces NT-proBNP levels in the subject compared to baseline;
(b) reduces rate of heart failure (HF) events;
(c) reduces incidence of intensification of oral diuretic therapy, which optionally includes outpatient augmentation of oral diuretic therapy;
(d) reduces incidence of changes in disease modifying therapy;
(e) reduces incidence of hospitalization for atrial fibrillation;
(f) induces change from baseline in Kansas City Cardiomyopathy Questionnaire overall summary (KCCQ-OS) score beyond 24 months of study treatment;
(g) induces change from baseline in six-minute walk test (6MWT) beyond 24 months of study treatment;
(h) induces change from baseline in ATTR-CM disease severity based on the Mayo, NAC, and Columbia disease stage, and NYHA classification;
(i) induces change from baseline in GLS;
(j) induces change from baseline in stroke volume;
(k) induces change from baseline in echocardiography parameters of interest;
(l) induces change from baseline in hs-cTnT;
(m) induces change from baseline in DPD/PYP/HMDP cardiac scintigraphy cardiac uptake and/or cMRI-derived ECV, T1 and T2 mapping;
(n) induces change from baseline in eGFR
(o) induces change from baseline in EQ-5D-5L score and/or induces change from baseline in SF-36 scores;
(p) induces change from baseline in a marker selected from CRP, IL IL1b, IL6, IL8, IFNg, TNF-α, IL10, IL1RA, C3 and C4, serum amyloid A, and ferritin; preferably a marker which is CRP;
(q) induces change from baseline in a marker selected from serum carboxy-terminal PICP, PIIINP, serum CITP, and plasma PRO-C6;
(r) induces change from baseline in a marker selected from TTR (prealbumin), TSH, RBP and fT4;
(s) induces change from baseline in (1) PND score and FAP stage; (2) Norfolk QoL-DN total score; and/or sNFL levels; and/or
(t) induces change from baseline in NIS and/or induces a change in NC studies.

In some embodiments, administration of the anti-TTR antibody leads to improved cardiovascular function (e.g., diastolic myocardial relaxation, atrioventricular (AV) flow, global longitudinal strain (GLS), ventricular ejection fraction, or end diastolic volume), e.g., as assessed by echocardiography. For example, echocardiography may be used to measure the ratio of early diastolic mitral inflow velocity to early diastolic mitral annulus velocity (E/e′ ratio) to assess cardiovascular function.

In some embodiments, the anti-TTR antibody is provided in an aqueous formulation at a concentration of about 25 to 125 mg/mL, which is diluted prior to administration. In some embodiments, the anti-TTR antibody is administered to a subject in an aqueous formulation at a diluted concentration of about 1 mg/mL to about 50 mg/mL (e.g., about 1 mg/mL to about 42 mg/mL, about 1 mg/mL to about 30 mg/mL, about 1 mg/mL to about 20 mg/mL, or about 1 mg/mL to about 10 mg/mL, e.g., about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL).

In some embodiments, the subject has been previously treated with and/or is concurrently receiving a TTR tetramer stabilizer.

In some embodiments, the TTR tetramer stabilizer is selected from the group consisting of diflunisal, Tafamidis, and Acoramidis (AG10).

In some embodiments, the antibody is administered to the subject by intravenous infusion in an aqueous formulation, in which the aqueous formulation has a pH of about 5.0 to 6.5, includes a histidine buffer, a sugar such as sucrose, and a surfactant such as polysorbate 80 (PS80), and the antibody is present in the aqueous formulation at a concentration of about 25 to 125 mg/mL.

In some embodiments, the aqueous formulation has a pH of about 5.8 and includes 20 mM histidine (e.g., L-histidine and/or L-histidine monohydrochloride), 6.5% weight per volume (w/v) sucrose, 0.03% PS80 w/v, and the antibody is present in the aqueous formulation at a concentration of about 50 mg/mL or 100 mg/mL.

In some embodiments, the aqueous formulation has a pH of about 5.8 and includes 20 mM histidine (e.g., L-histidine and/or L-histidine monohydrochloride), 8% weight per volume (w/v) sucrose, 0.03% PS80 w/v, and the antibody is present in the aqueous formulation at a concentration of about 50 mg/mL or 100 mg/mL.

In some embodiments, the antibody is administered to the subject in a diluted form including a diluent.

In some embodiments, the diluent is glucose or a polymer thereof (e.g., the polymer is dextran). The glucose or polymer thereof (e.g., dextran) may be used as a diluent at a concentration of about 1%, 2%, 3%, 4%, 5%, 6%, 7%, 8%, 9%, or 10% w/v.

In some embodiments, an infusion line is flushed with the diluent before and after the intravenous infusion.

In some embodiments, the intravenous infusion is performed with a syringe pump with an infusion syringe or an infusion pump. In an embodiment, a total antibody dose of up to 100 mg is administered by infusion, for example, a syringe pump with an infusion syringe. In an embodiment, a total antibody dose greater than 100 mg is administered with an infusion pump. In an embodiment, the anti-TTR antibody is diluted into an infusion bag prefilled with the diluent.

In some embodiments, the infusion syringe includes a minimum administration volume of 10 mL of the aqueous formulation.

In some embodiments, a total volume of the diluted form administered to the subject does not exceed 200 mL (including a flushing volume).

In some embodiments, at least for a first infusion, the aqueous formulation is administered over approximately 2 hours (±10 minutes).

In some embodiments, the aqueous formulation is administered over approximately 1 hour (±10 minutes).

In some embodiments, the ATTR amyloidosis leads to Cardiomyopathy (CM).

In some embodiments, the subject has ATTR amyloidosis with CM (ATTR-CM). In some embodiments, the subject has ATTR polyneuropathy (ATTR-PN). In some embodiments, the subject has Familial Amyloid Polyneuropathy (FAP). In some embodiments, the subject has Familial Amyloid Cardiomyopathy (FAC). In some embodiments, the subject has Senile Systemic Amyloidosis (SSA). In some embodiments, the subject has systemic familial amyloidosis. In some embodiments, the subject has leptomeningeal/Central Nervous System (CNS) amyloidosis. In some embodiments, the subject has Alzheimer disease. In some embodiments, the subject has TTR-related ocular amyloidosis. In some embodiments, the subject has TTR-related renal amyloidosis. In some embodiments, the subject has TTR-related hyperthyroxinemia. In some embodiments, the subject has TTR-related ligament amyloidosis including carpal tunnel syndrome. In some embodiments, the subject has rotator cuff tears and lumbar spinal stenosis. In some embodiments, the subject has preeclampsia.

In some embodiments, the subject has been diagnosed with hereditary ATTR-CM for a known pathogenic TTR mutation.

In some embodiments, the subject has sporadic, WT-ATTR-CM and a negative genetic testing for a TTR mutation.

Preferably, the diagnosis is based on evidence of cardiac amyloidosis by echocardiography or cMRI and one of the following: a) endomyocardial biopsy with confirmatory TTR amyloid typing by either immunohistochemistry or mass spectrometry; or (b) grade 2 or 3 cardiac uptake on 99mTc scintigraphy (99mTc DPD, 99mTc PYP, or 99mTc HMDP) in the absence of monoclonal gammopathy; or (c) grade 2 or 3 cardiac uptake on 99mTc scintigraphy (99mTc DPD, 99mTc PYP, or 99mTc HMDP), and confirmatory TTR amyloid typing by either immunohistochemistry or mass spectrometry in non-cardiac tissue in the presence of monoclonal gammopathy

In some embodiments, the subject has left ventricular ejection fraction (LVEF) of 220% (e.g., about 20% to about 60%, e.g., 20%, 25%, 30%, 35%, 40%, 45%, 50%, 55%, or 60%) before undergoing the treatment methods described herein, e.g., as measured by echocardiography. In an embodiment, the subject has a LVEF of 50% to 70% (e.g., 50%, 55%, 60%, 65%, or 70%) after undergoing the treatment methods described herein (e.g., the subject exhibits an increase in LVEF after treatment relative to LVEF prior to treatment).

In some embodiments, the subject has left ventricular wall thickness (LVWT) of 212 mm (e.g., about 12 mm to about 30 mm, e.g., about 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm) before undergoing the treatment methods described here, e.g., as measured by echocardiography. In an embodiment, the subject has a LVWT of <12 mm (e.g., about 8 mm to about 12 mm, e.g., about 8 mm, 9 mm, 10 mm, 11 mm, or 12 mm) after undergoing the treatment methods described herein.

In some embodiments, the subject, in particular a woman, has an end-diastolic interventricular septal wall thickness of 211 mm (e.g., about 11 mm to about 30 mm, e.g., about 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm), or the subject, in particular a man, has an end-diastolic interventricular septal wall thickness 212 mm (e.g., about 12 mm to about 30 mm, e.g., about 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm) before undergoing the treatment methods described herein, e.g., as measured by echocardiography.

In some embodiments, the subject (e.g., a woman) has an end-diastolic interventricular septal wall thickness of <11 mm (e.g., about 8 mm to about 11 mm, e.g., about 8 mm, 9 mm, 10 mm, or 11 mm) after undergoing the treatment methods described herein; or the subject (e.g., a man) has an end-diastolic interventricular septal wall thickness of <12 mm (e.g., about 8 mm to about 12 mm, e.g., about 8 mm, 9 mm, 10 mm, 11 mm, or 12 mm) after undergoing the treatment methods described herein.

In some embodiments, the subject has an N-terminal pro b-type natriuretic peptide (NT-proBNP) level of about 300 μg/mL to about 20,000 μg/mL before undergoing the treatment methods described herein. In some embodiments, the subject has an N-terminal pro b-type natriuretic peptide (NT-proBNP) level of about 0 μg/mL to about 300 μg/mL after undergoing the treatment methods described herein, preferably the subject has a NT-proBNP level of >2000 μg/mL before undergoing the treatment methods described herein.

In some embodiments, the subject has a cardiac troponin T (TnT) level of about 10 pg/mL to about 200 pg/mL before undergoing the treatment methods described herein. In some embodiments, the subject has a cardiac troponin T (TnT) level of about 0 pg/mL to about 10 pg/mL after undergoing the treatment methods described herein.

In some embodiments, the subject has a history of heart failure as documented by one of the following events within about 1 year before undergoing the treatment methods described herein: (i) heart failure hospitalization, (ii) urgent heart failure visit, and (iii) episode of volume overload documented by NT-proBNP>2000 pg/mL (or equivalent BNP).

In some embodiments, the heavy chain variable region of the anti-TTR antibody as used in accordance with the present invention includes the amino acid sequence of SEQ ID NO: 7 or 11, preferably of SEQ ID NO: 11, and the light chain variable region includes the amino acid of SEQ ID NO: 8, or the antibody can be an equivalent antibody with substantially the same binding characteristics as defined, infra. Most preferably, the antibody is a fully human IgG1m3 allotype antibody and thus comprises the human constant heavy chain (HC) amino acid sequence as exemplified in SEQ ID NO: 9 and a corresponding human constant light chain (LC) as exemplified in SEQ ID NO: 10, preferably having the above-identified posttranslational modifications, e.g., preferably a HC amino acid sequence present in SEQ ID NO: 15.

In some embodiments, the method further includes administering a TTR tetramer stabilizer. In some embodiments, the TTR tetramer stabilizer is selected from the group consisting of diflunisal, Tafamidis, and Acoramidis (AG10), preferably wherein tafamidis is administered as specified above.

In a further aspect, the present invention relates to the use of a human anti-TTR antibody, which is capable of binding mutated, misfolded, misassembled and/or aggregated TTR species and/or fragments thereof and does not substantially recognize physiological TTR species, in the manufacture of a medicament for treating or effecting prophylaxis of a subject having or at risk of having ATTR (e.g., ATTR-CM, ATTR polyneuropathy (ATTR-PN), Familial Amyloid Polyneuropathy (FAP), Familial Amyloid Cardiomyopathy (FAC), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia). As outlined in more detail above, in one embodiment, the anti-TTR antibody is capable of binding wild type and mutant TTR aggregates. In addition, or alternatively, the anti-TTR antibody does not bind to the physiological TTR tetramer and more preferably also not the wild type TTR monomer and preferably also not to the wild type TTR dimer. Preferably, the antibody is the antibody as defined hereinbefore.

The use includes administration of the medicament in a dosing regimen that results in a sustained plasma concentration of the antibody at about 1 μg/mL to about 1000 μg/mL (e.g., about 1 μg/mL to about 100 μg/mL, about 2.5 μg/mL to about 125 μg/mL, about 5 μg/mL to about 150 μg/mL, about 10 μg/mL to about 200 μg/mL, about 65 μg/mL to about 350 μg/mL, about 20 μg/mL to about 200 μg/mL, about 20 μg/mL to about 100 μg/mL, about 50 μg/mL to about 150 μg/mL, about 75 μg/mL to about 175 μg/mL, about 100 μg/mL to about 800 μg/mL, about 140 μg/mL to about 700 μg/mL, about 200 μg/mL to about 600 μg/mL, or about 400 μg/mL to about 800 μg/mL, e.g., about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, or about 1000 μg/mL) in the subject.

The present invention further relates to the use of a human anti-TTR antibody, which is capable of binding mutated, misfolded, misassembled and/or aggregated TTR species and/or fragments thereof and does not substantially recognize physiological TTR species, in the manufacture of a medicament for treating or effecting prophylaxis of a subject having or at risk of having ATTR (e.g., ATTR-CM, ATTR polyneuropathy (ATTR-PN), Familial Amyloid Polyneuropathy (FAP), Familial Amyloid Cardiomyopathy (FAC), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia). As outlined in more detail above, in one embodiment, the anti-TTR antibody is capable of binding wild type and mutant TTR aggregates. In addition, or alternatively, the anti-TTR antibody does not bind to the physiological TTR tetramer and more preferably also not the wild type TTR monomer and preferably also not to the wild type TTR dimer. Preferably, the antibody is the antibody as defined hereinbefore.

The use includes administration of the medicament in a dosing regimen that results in a sustained plasma concentration of the antibody at an AUC (e.g., an area under a plasma drug concentration-time curve in a subject after, e.g., 17-50 weeks of treatment/dosing) of about 2,000 μg*day/mL to about 100,000 μg*day/mL, about 2,500 μg*day/mL to about 100,000 μg*day/mL, about 5,000 μg*day/mL to about 100,000 μg*day/mL, or about 10,000 μg*day/mL to about 100,000 μg*day/mL (e.g., about 2,000 μg*day/mL, about 2,500 μg*day/mL, about 3,000 μg*day/mL, about 3,500 μg*day/mL, about 4,000 μg*day/mL, about 5,000 μg*day/mL, about 6,000 μg*day/mL, about 7,000 μg*day/mL, about 8,000 μg*day/mL, about 9,000 μg*day/mL, about 10,000 μg*day/mL, about 15,000 μg*day/mL, about 20,000 μg*day/mL, about 30,000 μg*day/mL, about 40,000 μg*day/mL, about 50,000 μg*day/mL, about 60,000 μg*day/mL, about 70,000 μg*day/mL, about 80,000 μg*day/mL, about 90,000 μg*day/mL, or about 100,000 μg*day/mL), preferably at least 30,000 μg*day/mL in the subject.

In some embodiments, the anti-TTR antibody is for administration in a dosing regimen that results in a sustained plasma concentration of the antibody at an AUC (e.g., an area under a plasma drug concentration-time curve in a subject after 17 weeks of treatment/dosing) of about 2,000 μg*day/mL to about 50,000 μg*day/mL (e.g., about 2,000 μg*day/mL, about 2,500 μg*day/mL, about 3,000 μg*day/mL, about 3,500 μg*day/mL, about 4,000 μg*day/mL, about 5,000 μg*day/mL, about 6,000 μg*day/mL, about 7,000 μg*day/mL, about 8,000 μg*day/mL, about 9,000 μg*day/mL, about 10,000 μg*day/mL, about 15,000 μg*day/mL, about 20,000 μg*day/mL, about 30,000 μg*day/mL, about 40,000 μg*day/mL, or about 50,000 μg*day/mL in the subject).

In some embodiments, the anti-TTR antibody is for administration in a dosing regimen that results in a sustained plasma concentration of the antibody at an AUC (e.g., an area under a plasma drug concentration-time curve in a subject after 50 weeks of treatment/dosing) of about 2,500 μg*day/mL to about 100,000 μg*day/mL, about 5,000 μg*day/mL to about 100,000 μg*day/mL, or about 10,000 μg*day/mL to about 100,000 μg*day/mL (e.g., about 2,500 μg*day/mL, about 3,000 μg*day/mL, about 3,500 μg*day/mL, about 4,000 μg*day/mL, about 5,000 μg*day/mL, about 6,000 μg*day/mL, about 7,000 μg*day/mL, about 8,000 μg*day/mL, about 9,000 μg*day/mL, about 10,000 μg*day/mL, about 15,000 μg*day/mL, about 20,000 μg*day/mL, about 30,000 μg*day/mL, about 40,000 μg*day/mL, about 50,000 μg*day/mL, about 60,000 μg*day/mL, about 70,000 μg*day/mL, about 80,000 μg*day/mL, about 90,000 μg*day/mL, or about 100,000 μg*day/mL in the subject.

In some embodiments, administration of the anti-TTR antibody is useful for long term treatment and/or for follow up treatment (e.g., at a dosage sufficient to achieve a sustained plasma concentration of about 1 μg/mL, 2.5 μg/mL, or 5 μg/mL) after initial higher dosing and amyloid removal.

The present invention further relates to the use of a human anti-TTR antibody in the manufacture of a medicament for treating ATTR (e.g., ATTR-CM, ATTR polyneuropathy (ATTR-PN), Familial Amyloid Polyneuropathy (FAP), Familial Amyloid Cardiomyopathy (FAC), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia) in a subject in need thereof, in which:
(a) the use includes administering the medicament at a dosage of about 0.3 to about 100 mg/kg to the subject once every 3 to 56 days (e.g., once evert 3 to about 35 days, e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 days); or the use includes administering the medicament at a dosage about 600 mg to about 7500 mg, preferably of about 2500 mg, 3500 mg, or 5000 mg, or of about 2400 mg, 3200 mg, or 4800 mg, respectively, dependent of the patient's body weight once every 3 to 56 days (e.g., once evert 3 to about 35 days, e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 days), preferably once every 4 weeks (q4w) and
(b) the antibody or antigen-binding fragment thereof includes a heavy chain variable region with at least 80% (e.g., 85%, 90%, 91%, 92%, 93%, 94%, 95%, 96%, 97%, 98%, 99%, or 100%) sequence identity to SEQ ID NO: 7 and a light chain variable region includes an amino acid sequence with at least 80% (e.g., 85%, 90%, 91%, 92%, 93%, 94%, 95%, 96%, 97%, 98%, 99%, or 100%) sequence identity to SEQ ID NO: 8, or wherein the antibody is an equivalent antibody with substantially the same binding characteristics as defined herein; see also infra. The heavy chain variable region includes the CDRs set forth in SEQ ID NOs: 1-3 and the light chain variable region includes the CDRs set forth in SEQ ID NOs: 4-6. Most preferably, the antibody is a fully human IgG1m3 allotype antibody and thus comprises the human constant heavy chain (HC) amino acid sequence as exemplified in SEQ ID NO: 9 and a corresponding human constant light chain (LC) as exemplified in SEQ ID NO: 10, preferably having the above-identified posttranslational modifications. The antibody or antigen-binding fragment thereof is present in the medicament at a concentration of about 10 mg/mL to about 125 mg/mL. In some embodiments, the antibody is diluted from a concentration stock prior to administration. The concentration of the medicament may be diluted, e.g., into a dilution bag, prior to administration to the subject.

### Methods of Treatment

Described herein are methods of preventing or treating subjects having or at risk of having a disease associated with ATTR (e.g., ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, and preeclampsia) by administering an anti-TTR antibody.

The methods of treatment described herein provide improved signs of cardiac function (e.g., diastolic myocardial relaxation, atrioventricular (AV) flow, global longitudinal strain (GLS), ventricular ejection fraction, or end diastolic volume) as well as a reduction (e.g., ≥5%, ≥10%, ≥15%, ≥20%, ≥25%, ≥30%, ≥35%, ≥40%, ≥45%, ≥50%, ≥55%, ≥60%, ≥65%, ≥70%, ≥75%, ≥80%, ≥85%, ≥90%, ≥95%, or 100% reduction) in cardiac amyloid deposits (e.g., misfolded TTR protein) in the heart of a subject within 4-30 months (e.g., within 5-25 months, within 10-20 months, or within 12-18 months) following treatment with the methods described herein.

The methods described herein include administration of a recombinant anti-TTR antibody, which is capable of binding mutated, misfolded, misassembled and/or aggregated TTR species and/or fragments thereof and does not substantially recognize physiological TTR species as outlined in more detail above. Such methods are capable of treating or effecting prophylaxis of a subject having or at risk of having a disease associated with ATTR (e.g., ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, and preeclampsia). The antibody can be administered in a dosing regimen that results in a sustained (e.g., maintained for a period) blood (e.g., serum or plasma) concentration in the subject for a period of time (e.g., about 1 day to about 1 week, about 1 day to about 30 days, e.g., about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days) as described herein. The sustained blood (e.g., serum or plasma) concentration may be, e.g., at least 1 μg/mL, at least 2.5 μg/mL, at least 5 μg/mL, at least 10 μg/mL, at least 20 μg/mL, at least 30 μg/mL, at least 40 μg/mL, at least 50 μg/mL, at least 60 μg/mL, at least 70 μg/mL, at least 80 μg/mL, at least 90 μg/mL, at least 100 μg/mL, at least 110 μg/mL, at least 120 μg/mL, at least 130 μg/mL, at least 140 μg/mL, at least 150 μg/mL, at least 160 μg/mL, at least 170 μg/mL, at least 180 μg/mL, at least 190 μg/mL, at least 200 μg/mL, at least 210 μg/mL, at least 220 μg/mL, at least 230 μg/mL, at least 240 μg/mL, at least 250 μg/mL, at least 260 μg/mL, at least 270 μg/mL, at least 280 μg/mL, at least 290 μg/mL, at least 300 μg/mL, at least 310 μg/mL, at least 320 μg/mL, at least 330 μg/mL, at least 340 μg/mL, at least 350 μg/mL, at least 360 μg/mL, at least 370 μg/mL, at least 380 μg/mL, at least 390 μg/mL, at least 400 μg/mL, at least 410 μg/mL, at least 420 μg/mL, at least 430 μg/mL, at least 440 μg/mL, at least 450 μg/mL, at least 460 μg/mL, at least 470 μg/mL, at least 480 μg/mL, at least 490 μg/mL, at least 500 μg/mL, at least 510 μg/mL, at least 520 μg/mL, at least 530 μg/mL, at least 540 μg/mL, at least 550 μg/mL, at least 560 μg/mL, at least 570 μg/mL, at least 580 μg/mL, at least 590 μg/mL, at least 600 μg/mL, at least 610 μg/mL, at least 620 μg/mL, at least 630 μg/mL, at least 640 μg/mL, at least 650 μg/mL, at least 660 μg/mL, at least 670 μg/mL, at least 680 μg/mL, at least 690 μg/mL, at least 700 μg/mL, at least 710 μg/mL, at least 720 μg/mL, at least 730 μg/mL, at least 740 μg/mL, at least 750 μg/mL, at least 760 μg/mL, at least 770 μg/mL, at least 780 μg/mL, at least 790 μg/mL, at least 800 μg/mL, at least 810 μg/mL, at least 820 μg/mL, at least 830 μg/mL, at least 840 μg/mL, at least 850 μg/mL, at least 860 μg/mL, at least 870 μg/mL, at least 880 μg/mL, at least 890 μg/mL, at least 900 μg/mL, at least 910 μg/mL, at least 920 μg/mL, at least 930 μg/mL, at least 940 μg/mL, at least 950 μg/mL, at least 960 μg/mL, at least 970 μg/mL, at least 980 μg/mL, at least 990 μg/mL, or 1000 μg/mL and may last for at least 3 days to about 42 days (e.g., about 3 to about 35 days, about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 days).

The antibody can be also administered in a dosing regimen that results in a sustained plasma concentration of the antibody at an AUC (e.g., an area under a plasma drug concentration-time curve in a subject after treatment/dosing) of about 2,000 μg*day/mL to about 100,000 μg*day/mL, about 2,500 μg*day/mL to about 100,000 μg*day/mL, about 3,000 μg*day/mL to about 100,000 μg*day/mL, about 5,000 μg*day/mL to about 100,000 μg*day/mL, or about 10,000 μg*day/mL to about 100,000 μg*day/mL, or of about 2,000 μg*day/mL to about 50,000 μg*day/mL, about 5,000 μg*day/mL to about 50,000 μg*day/mL, or about 10,000 μg*day/mL to about 50,000 μg*day/mL (e.g., about 2,000 μg*day/mL, about 2,500 μg*day/mL, about 3,000 μg*day/mL, about 3,500 μg*day/mL, about 4,000 μg*day/mL, about 5,000 μg*day/mL, about 6,000 μg*day/mL, about 7,000 μg*day/mL, about 8,000 μg*day/mL, about 9,000 μg*day/mL, about 10,000 μg*day/mL, about 15,000 μg*day/mL, about 20,000 μg*day/mL, about 30,000 μg*day/mL, about 40,000 μg*day/mL, about 50,000 μg*day/mL, about 60,000 μg*day/mL, about 70,000 μg*day/mL, about 80,000 μg*day/mL, about 90,000 μg*day/mL, or about 100,000 μg*day/mL in the subject. The sustained plasma concentration of the antibody as determined by an AUC may differ depending on the treatment duration. For example, after 17 weeks treatment, the AUC is rather about 2,000 μg*day/mL to about 50,000 μg*day/mL and after 50 weeks of treatment, the AUC is rather about 2,500 μg*day/mL to about 100,000 μg*day/mL.

The dosing regimen may include administering a dose of from about 0.3 mg/kg to about 100 mg/kg (e.g., 0.3 mg/kg to 60 mg/kg, 0.3 mg/kg to 30 mg/kg, 0.3 mg/kg to about 10 mg/kg, 0.3 mg/kg to 1 mg/kg, 1 mg/kg to 10 mg/kg, 10 mg/kg to 20 mg/kg, 10 mg/kg to 30 mg/kg, 10 mg/kg to 60 mg/kg, 20 mg/kg to 30 mg/kg, 30 mg/kg to 40 mg/kg, 40 mg/kg to 50 mg/kg, 50 mg/kg to 60 mg/kg, 60 mg/kg to 70 mg/kg, 70 mg/kg to 80 mg/kg, 80 mg/kg to 90 mg/kg, or 90 mg/kg to 100 mg/kg) of the antibody or antigen-biding fragment thereof. For example, the antibody may be administered at a dosage of about 0.3 mg/kg, 1 mg/kg, 3 mg/kg, 10 mg/kg, 30 mg/kg, or 60 mg/kg of the anti-TTR antibody. More particularly, the dosing regimen may include administering a first starting dose of about 0.3 mg/kg to about 60 mg/kg, of about 0.3 mg/kg to about 30 mg/kg, and preferably of about 0.3 mg/kg to about 10 mg/kg, and further maintenance dose of about 10 mg/kg to about 100 mg/kg, of about 10 mg/kg to about 80 mg/kg, and preferably of about 10 mg/kg to about 60 mg/kg, and most preferably of about 30 mg/kg or 60 mg/kg.

In one embodiment, only the maintenance dose is administered.

The dosing regimen may also include administering a dose of from about 300 mg to about 10,000 mg (e.g., about 600 mg to about 7500 mg, 600 mg to 1000 mg, 600 mg to 2000 mg, 600 mg to 4000 mg, 600 mg to 6000 mg, 1000 mg to 2000 mg, 2000 mg to 3000 mg, 3000 mg to 4000 mg, 4000 mg to 5000 mg, 5000 mg to 6000 mg, 6000 mg to 8000 mg, 8000 mg to 9000 mg, 9000 mg to 10000 mg) of the antibody. For example, the antibody may be administered at a dosage of about 3000 mg, or 3500 mg of the anti-TTR antibody. More particularly, the dosing regimen may include administering a maintenance dose of about 300 mg to about 10000 mg, about 600 mg to about 10000 mg, and preferably about 600 mg to about 7500 mg. The dosing regimen may further include administering a loading dose of about 300 mg to 6000 mg, about 600 mg to 5000 mg, and preferably about 600 mg to 4000 mg, wherein preferably the loading dose is administered before administration of the maintenance dose, more preferably for up to two months before the maintenance dose, and most preferably once every other week for up to two months before administration of the maintenance dose.

The dosing regimen may include administering a flat dose dependent on the subject's body weight. Thus, the dosing regimen may include administering for example 2500 mg of the anti-TTR antibody to a subject with a body weight of about 40 to about ≤60 kg, for example 3500 mg of the anti-TTR antibody to a subject with a body weight of about ≥60 kg to about 100 kg, and for example 5000 mg to a subject with a body weight of about ≤100 kg.

Preferably, the dosing regimen may include administering for example 2400 mg of the anti-TTR antibody to a subject with a body weight of about 40 to about ≤60 kg, for example 3200 mg of the anti-TTR antibody to a subject with a body weight of about ≥60 kg to about 100 kg, and for example 4800 mg to a subject with a body weight of about ≤100 kg.

The anti-TTR antibody may be administered to the subject once every 3 to 42 days (e.g., about once every 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 days), and preferably once every 21 to 35 days, preferably about once every 28 to 35 days, most preferably about once every 28 days or about once every 35 days, most preferably once every 4 weeks The anti-TTR antibody may be administered via subcutaneous or continuous infusion via a pump, for example once weekly.

The anti-TTR antibody may be administered to the subject at a dose of about 30 mg/kg for at least 12 months (e.g., about 12-18 months, e.g., about 12 months).

Additional details on the anti-TTR antibodies, dosing regimens (e.g., dosage of antibody, frequency of administration, and/or duration of treatment), antibody formulations (e.g., pharmaceutical compositions), subjects to be treated, and how to monitor and assess the efficacy of treatment are described herein.

### Antibodies

An anti-TTR antibody may be administered to a subject (e.g., a human) to treat or prevent (e.g., to effect prophylaxis of) a disease or disorder (e.g., ATTR or ATTR-CM), as described herein. The methods of treating or preventing a disease associated with ATTR (e.g., ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, and preeclampsia) described herein utilize an anti-TTR antibody, e.g., a human anti-TTR antibody. The anti-TTR antibodies herein may be any antibody capable of binding (e.g., as determined by a dissociation constant (K_{D})) mutated, misfolded, misassembled and/or aggregated TTR species and/or fragments thereof and may not substantially recognize physiological TTR species.

Preferably, the antibody may be any antibody which presents high binding affinity to misfolded TTR in the sub-nanomolar range, is highly selective for the amyloid conformation of TTR, i.e., binds selectively with high affinity to the disease associated ATTR aggregates, exerts similar binding to wild type TTR and variant TTR. related to sporadic or hereditary disease, does not bind physiological TTR monomers, and/or binds ATTR deposits in cardiac tissues obtained at autopsy from ATTR-CM patients. The binding characteristics can be determined with conventional methods in the art, for example ELISA assays, Surface plasmon resonance (SPR) analyses, dot blot analyses, time-course aggregation studies, immunoprecipitation experiments, and immunohistochemistry (IHC) as for example described in Michalon et al., Nat Commun. 12 (2021), 3142.

For example, the anti-TTR antibody described herein may have a different K_{D} for different TTR isoforms, such as a K_{D} of >300 nM for wild-type native TTR, and/or a K_{D} of ≤15 nM, e.g., ≤5 nM, e.g., ≤2 nM for denaturated TTR, and/or a K_{D} of ≤35 nM, e.g., ≤20 nM for native TTR-V30M, ≤5 nM for native TTR-V122K, and/or a K_{D} of ≤150 nM, e.g., of ≤5 nM, such as ≤2 nM for native TTR-L55P.

Exemplary heavy chain variable (VH) regions, light chain variable (VL) region, and complimentary determining regions (CDRs) of anti-TTR antibodies and antigen-binding fragments thereof are shown in Table 1 below. CDR sequences were defined by the Kabat system (bioinf.org.uk/abs/). SEQ ID NO: 1 (VH-CDR1) represents residues 31-35 (Kabat numbering) of SEQ ID NO: 7 (VH). SEQ ID NO: 2 (VH-CDR2) represents residues 52-67 (Kabat numbering) of SEQ ID NO: 7 (VH). SEQ ID NO: 3 (VH-CDR3) represents residues 100-109 (Kabat numbering) of SEQ ID NO: 7 (VH).

SEQ ID NO: 4 (VL-CDR1) represents residues 31-35 (Kabat numbering) of SEQ ID NO: 8 (VL). SEQ ID NO: 5 (VL-CDR2) represents residues 52-67 (Kabat numbering) of SEQ ID NO: 8 (VL). SEQ ID NO: 6 (VL-CDR3) represents residues 100-109 (Kabat numbering) of SEQ ID NO: 8 (VL).

TABLE 1
Anti-TTR Antibody Sequences

| | SEQ | |
|---|---|---|
| Domain/ | ID | |
| Region | NO: | Amino Acid Sequence |
| VH-CDR1 | 1 | SRSSY |
| VH-CDR2 | 2 | GIYHSGNTYDNPSLKS |
| VH-CDR3 | 3 | IVPGGDAFDI |
| VL-CDR1 | 4 | RASQSVGTYLN |
| VL-CDR2 | 5 | AASSLOS |
| VL-CDR3 | 6 | QQSYSSPPT |
| VH | 7 | QLQLQESGPGLVKPSETLSLTCSVSGGSIISRSSYWGWIRQPPGKGLEWIGGI |
| | | YHSGNTYDNPSLKSRLTMSVDTSKNQFSLNLRSVTAADTAVYYCARIVPGGD |
| | | AFDIWGQGTMVTVSS |
| VL | 8 | DIQMTQSPSSLSASVGDRVTIACRASQSVGTYLNWYQQKRGKAPKLLIFAASS |
| | | LQSGVPSRFSGSGSGTDFTLTISSLOPEDFATYYCQQSYSSPPTFGQGTKVEI |
| | | K |
| HC | 9 | Q*LQLQESGPGLVKPSETLSLTCSVSGGSIISRSSYWGWIRQPPGKGL |
| | | EWIGGIYHSGNTYDNPSLKSRLTMSVDTSKNQFSLNLRSVTAADTAVYYCARI |
| | | VPGGDAFDIWGQGTMVTVSSASTKGPSVFPLAPSSKSTSGGTAALGCLVKDY |
| | | FPEPVTVSWNSGALTSGVHTFPAVLQSSGLYSLSSVVTVPSSSLGTQTYICNV |
| | | NHKPSNTKVDKRVEPKSCDKTHTCPPCPAPELLGGPSVFLFPPKPKDTLMISR |
| | | TPEVTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYSKLTVD |
| | | KSRWQQGNVFSCSVMHEALHNHYTQKSLSLSPGK* |
| | | *the C-terminal lysine (K) is optional and/or the N-terminal glutamine (Q) |
| | | undergoes intramolecular cyclization, resulting in the formation of pyroglutamic |
| | | acid |
| LC | 10 | DIQMTQSPSSLSASVGDRVTIACRASQSVGTYLNWYQQKRGKAPKLLIFAASS |
| | | LQSGVPSRFSGSGSGTDFTLTISSLQPEDFATYYCQQSYSSPPTFGQGTKVEI |
| | | KRTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSFN |
| | | RGEC |
| VH | 11 | LQLQESGPGLVKPSETLSLTCSVSGGSIISRSSYWGWIRQPPGKGLEWIGGIY |
| | | HSGNTYDNPSLKSRLTMSVDTSKNQFSLNLRSVTAADTAVYYCARIVPGGDA |
| | | FDIWGQGTMVTVSS |
| VL | 12 | DIQMTQSPSSLSASVGDRVTIACRASQSVGTYLNWYQQKRGKAPKLLIFAASS |
| | | LQSGVPSRFSGSGSGTDFTLTISSLQPEDFATYYCQQSYSSPPTFGQGTKVEI |
| HC - | 13 | QLQLQESGPGLVKPSETLSLTCSVSGGSIISRSSYWGWIRQPPGKGLEWIGGI |
| without C- | | YHSGNTYDNPSLKSRLTMSVDTSKNQFSLNLRSVTAADTAVYYCARIVPGGD |
| terminal | | AFDIWGQGTMVTVSSASTKGPSVFPLAPSSKSTSGGTAALGCLVKDYFPEPV |
| lysine | | TVSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQV |
| | | SLTCLVKGFYPSDIAVEWESNGQPENNYKTTPPVLDSDGSFFLYSKLTVDKSR |
| | | WQQGNVFSCSVMHEALHNHYTQKSLSLSPG |
| HC - | 14 | LQLQESGPGLVKPSETLSLTCSVSGGSIISRSSYWGWIRQPPGKGLEWIGGIY |
| without N- | | HSGNTYDNPSLKSRLTMSVDTSKNQFSLNLRSVTAADTAVYYCARIVPGGDA |
| terminal | | FDIWGQGTMVTVSSASTKGPSVFPLAPSSKSTSGGTAALGCLVKDYFPEPVT |
| glutamate** | | VSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQV |
| | | SLTCLVKGFYPSDIAVEWESNGQPENNYKTTPPVLDSDGSFFLYSKLTVDKSR |
| | | WQQGNVFSCSVMHEALHNHYTQKSLSLSPGK |
| HC - | 15 | LQLQESGPGLVKPSETLSLTCSVSGGSIISRSSYWGWIRQPPGKGLEWIGGIY |
| without C- | | HSGNTYDNPSLKSRLTMSVDTSKNQFSLNLRSVTAADTAVYYCARIVPGGDA |
| terminal | | FDIWGQGTMVTVSSASTKGPSVFPLAPSSKSTSGGTAALGCLVKDYFPEPVT |
| lysine and | | VSWNSGALTSGVHTFPAVLQSSGLYSLSSVVTVPSSSLGTQTYICNVNHKPS |
| N-terminal | | NTKVDKRVEPKSCDKTHTCPPCPAPELLGGPSVFLFPPKPKDTLMISRTPEVT |
| glutamate** | | CVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSREEMTKNQV |
| | | SLTCLVKGFYPSDIAVEWESNGQPENNYKTTPPVLDSDGSFFLYSKLTVDKSR |
| | | WQQGNVFSCSVMHEALHNHYTQKSLSLSPG |

CDR = complimentary determining region; VH = heavy chain variable region; VL = light chain variable region, HC = heavy chain; LC = light chain.
**the N-terminal glutamate has been modified to pyroglutamic acid

The anti-TTR antibody or antigen binding fragment thereof may include one or more CDR sequences including an amino acid sequence having about 80%, about 85%, about 86%, about 87%, about 88%, about 89%, about 90%, about 91%, about 92%, about 93%, about 94%, about 95%, about 96%, about 97%, about 98%, about 99% or about 100% sequence identity to the amino acid sequence of SEQ ID NO: 1, SEQ ID NO: 2, SEQ ID NO: 3, SEQ ID NO: 4, SEQ ID NO: 5, and/or SEQ ID NO: 6. Further, the anti-TTR antibody or antigen binding fragment thereof may include one or more CDR sequences having an amino acid sequence with 1, 2, or 3 mismatches relative to the amino acid sequence of SEQ ID NO: 1, SEQ ID NO: 2, SEQ ID NO: 3, SEQ ID NO: 4, SEQ ID NO: 5, and/or SEQ ID NO: 6. In a particular example, the anti-TTR antibody or antigen binding fragment thereof includes six CDR amino acid sequences with 100% sequence identity to the amino acid sequence of SEQ ID NO: 1, SEQ ID NO: 2, SEQ ID NO: 3, SEQ ID NO: 4, SEQ ID NO: 5, and SEQ ID NO: 6.

The anti-TTR antibody or antigen-binding fragment thereof may have a VH region including an amino acid sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 7. In a particular example, the anti-TTR antibody or antigen-binding fragment thereof has a VH region including an amino acid sequence with 100% sequence identity to the amino acid sequence of SEQ ID NO: 7.

The anti-TTR antibody or antigen-binding fragment thereof may have a VL region including an amino acid sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 8. In a particular example, the anti-TTR antibody or antigen-binding fragment thereof has a VL region including an amino acid sequence with 100% sequence identity to the amino acid sequence of SEQ ID NO: 8.

The anti-TTR antibody or antigen-binding fragment thereof may have a VH region including an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the amino acid sequence of SEQ ID NO: 11. In a particular example, the anti-TTR antibody or antigen-binding fragment thereof has a VH region including an amino acid sequence with 100% sequence identity to the amino acid sequence of SEQ ID NO: 11.

The anti-TTR antibody or antigen-binding fragment thereof may have a VL region including an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the amino acid sequence of SEQ ID NO: 12. In a particular example, the anti-TTR antibody or antigen-binding fragment thereof has a VL region including an amino acid sequence with 100% sequence identity to the amino acid sequence of SEQ ID NO: 12.

In some embodiments, the anti-TTR antibody is an anti-TTR antibody described in U.S. Pat. No. 10,344,080 or 11,180,545 (each of which is incorporated herein by reference in its entirety). The anti-TTR antibody may be an anti-TTR antibody described in U.S. Publication Number US 20220144928 (which is incorporated herein by reference in its entirety).

Also, within the scope of the present invention are anti-TTR antibodies and antigen-binding fragments thereof, in which specific amino acids have been substituted, deleted, or added. These modifications do not have a substantial effect on the anti-TTR antibody's biological properties such as binding activity; see also infra. For example, antibodies may have amino acid substitutions in the framework region (FR), so as to improve binding to the antigen. In another example, a number of acceptor framework residues can be replaced by the corresponding donor amino acids. The donor framework can be a mature or germline human antibody framework sequence or a consensus sequence. Guidance concerning how to make phenotypically silent amino acid substitutions is provided in, e.g., Bowie et al. (Science, 247: 1306-1310, 1990), Cunningham et al. (Science, 244: 1081-1085, 1989), Ausubel (ed.) (Current Protocols in Molecular Biology, John Wiley and Sons, Inc., 1994), T. Maniatis, E. F. Fritsch and J. Sambrook (Molecular Cloning: A Laboratory Manual, Cold Spring Harbor laboratory, Cold Spring Harbor, N.Y., 1989), Pearson (Methods Mol. Biol. 243:307-31, 1994), and Gonnet et al. (Science 256:1443-45, 1992); each of which is incorporated herein by reference.

The variant antibodies or antigen-binding fragments thereof are functionally active and may have, e.g., fewer than about 30%, about 25%, about 20%, about 15%, about 10%, about 5% or about 1% amino acid of the number of residues substituted or deleted while retaining essentially the same immunological properties including, but not limited to, binding to TTR, as described herein, i.e., equivalent antibodies having substantially the same binding properties to TTR as the exemplarity antibody comprising a heavy chain variable region with the amino acid sequence of SEQ ID NO: 7 and the light chain variable region with the amino acid of SEQ ID NO: 8, which is characterized in U.S. Pat. Nos. 10,344,080 B2 and 11,180,545 B2; see also supra, (corresponding to international application WO 2015/092077 A1, where the antibody is named NI-301.37F1) as well as in Michalon et al., Nat. Commun. 12 (2021), 3142 (designated as antibody NI301A) by (i) binding selectively with high affinity to the disease associated ATTR aggregates, (ii) binding to misfolded/aggregated wild type TTR and variant TTR related to sporadic and hereditary disease, respectively, as well as to ATTR deposits in cardiac tissues obtained at autopsy from ATTR-CM patients, but not substantially binding native TTR monomers, and, importantly, being capable of removing ATTR fibrils by macrophage-mediated phagocytosis. The latter property can be easily tested as described in international application WO 2020/094883 A1; see also Michalon et al. (2021), supra.

The antibodies or antigen-binding fragments thereof may also include variants, including, e.g., humanized or chimeric antibodies or antigen-binding fragments thereof, analogs, orthologs, homologs and derivatives of the exemplified antibody, that exhibit a biological activity, e.g., binding of an antigen such as TTR. The antibodies may contain one or more analogs of an amino acid (including, for example, non-naturally occurring amino acids, amino acids which only occur naturally in an unrelated biological system, modified amino acids from mammalian systems etc.), antibodies with substituted linkages, as well as other modifications known in the art.

The anti-TTR antibody fragment can be selected from the group consisting of bis-Fab, Fab, Fab′-SH, Fv, scFv, and (Fab′)₂ fragments.

In certain embodiments, the anti-TTR antibody is a monoclonal antibody (mAb). The anti-TTR antibody may be a human or chimeric antibody. The anti-TTR antibody may be an IgG antibody. The anti-TTR antibody may be a recombinant human IgG1 antibody.

In an embodiment, the human anti-TTR antibody or antigen-binding fragment thereof does not elicit an anti-drug antibody (ADA) response in a human subject. The anti-TTR antibody or antigen-binding fragment thereof may have a VH region including an amino acid sequence with 100% sequence identity to the amino acid sequence of SEQ ID NO: 7 and a VL region including an amino acid sequence with 100% sequence identity to the amino acid sequence of SEQ ID NO: 8.

In this context, the person skilled in the art is aware that effector function and intensity can, inter alia, depend on the IgG class or isotype and that IgG2 and IgG4 have only attenuated effector functions compared to IgG1 or IgG3. Therefore, in an embodiment, the anti-TTR antibody described herein can be of the IgG1 or IgG3 class or isotype, for example, IgG1. Of course, besides using native IgG immunoglobulins corresponding effector functions can be genetically engineered; see, e.g., Saunders K O (2019) Conceptual Approaches to Modulating Antibody Effector Functions and Circulation Half-Life. Front. Immunol. 10: 1296. doi: 10.3389/fimmu.2019.01296.

The five primary classes of immunoglobulins are IgG, IgM, IgA, IgD and IgE. These are distinguished by the type of heavy chain found in the molecule. IgG molecules have heavy chains known as gamma-chains; IgMs have mu-chains; IgAs have alpha-chains; IgEs have epsilon-chains; and IgDs have delta-chains; see for review, e.g., Schroeder et al., J. Allergy Clin. Immunol. 125 (2010), S41-S52. Furthermore, different subclasses exist, wherein the IgAs are further divided into subclasses IgAQ1 and IgA2, and wherein IgGs are further divided into subclasses IgG1, IgG2, IgG3, and IgG4. Furthermore, two types of light chain, kappa (κ) and lambda (λ) exist.

In principle, the antibody as used in accordance with the present invention may be of any kind of class and subclass, respectively, and may comprise any kind of light chain, as long as the antibody binds to misfolded and preferably aggregated forms of TTR, and preferably as long as binding specificity towards TTR as indicated in the Examples of WO 2015/092077 A1 for antibody NI-301.37F1 remains unaffected in kind and as long as no adverse effects occur when administering said antibody to a patient, wherein the adverse effects can be determined as described in Example 1. However, preferably complete IgG antibodies are used, wherein the antibody comprises a constant domain. Accordingly, in one embodiment, the immunoglobulin heavy and/or light chain constant domain present in the antibody as used in accordance with the present invention is of the IgG type, the IgM type, the IgA type, the IgD type or the IgE type, preferably of the IgG type. In one embodiment, the immunoglobulin heavy and/or light chain constant domain present in the antibody as used in accordance with the present invention is of the IgAQ1, IgAQ1, IgG1, IgG2, IgG3, or IgG4 subclass, preferably of the IgG1, IgG2, IgG3, or IgG4 subclass and most preferably of the IgG1 subclass.

As mentioned above, NI006/ALXN2220 induces antibody-mediated phagocytosis of ATTR fibrils by phagocytic immune cells such as macrophages, resulting in the clearance of ATTR deposits from tissues. Thus, in one embodiment, the antibody comprises a region equivalent to the human IgG constant region, and which is capable of mediating phagocytosis, like a IgA subclass or engineered Fc regions as for example described in Liu et al., Antibodies 9 (2020), 64.

Recombinant expression of complete human IgG1 antibodies with a human or mouse constant domain can be performed substantially as described in the Examples of WO 2015/092077 A1. Preferably, the antibody is a monoclonal antibody or derived from a monoclonal antibody.

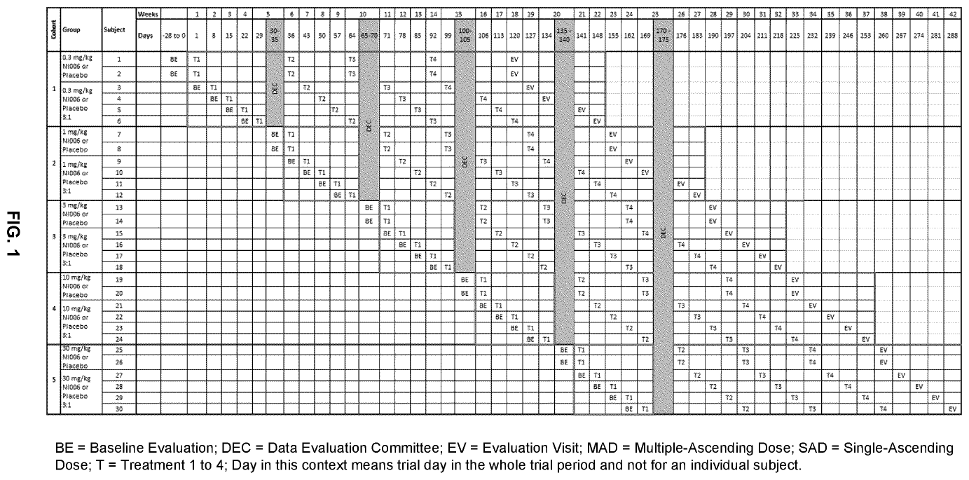

There are not only the above-mentioned four subclasses of IgGs but human heavy and light chain genes also exhibit extensive structural polymorphism(s) and, being closely linked, are inherited as a haplotype. Allotypic variants can be immunogenic and provoke antibody responses as a result of allo-immunization. Thus, switching the allotype can be of particular interest to provide non-immunogenic antibody therapeutics. So far, extensive allotypes (polymorphisms) are known, but focus is put on the serologically defined allotypes. Allotypes of IgG proteins are defined by the expression of unique epitope(s) recognized by unique serologic reagent(s). Allotypes expressed on the constant region of IgG heavy chain are designated as Gm (Genetic marker) together with the subclass, e.g., G1m, and the allotype number (or letter), e.g., G1m1 [or G1m(a)], G3m5 [or G3m(b1)]. Human immunoglobulin allotypes are listed in Table 1 of Jefferis and Lefrance, mAbs 1 (2009), 1-7 and in FIG. 1A of Irani et al., Molecular Immunology 67 (2015), 171-182, which content is herein incorporated by reference. Accordingly, in one embodiment, the antibody as used in accordance with the present invention is of any one of the following allotypes, but not limited thereto: G1m1, G1m2, G1m3, G1m17, G2m23, G3m21, G3m28, G3m11, G3m5, G3m13, G3m14, G3m10, G3m15, G3m16, G3m6, G3m24, G3m26, G3m27, A2m1, A2m2, A2m3, Emi, Km1, Km2, and Km3, but preferably of G1m2, G1m3, or G1m17, and most preferably of G1m3.

As explained above, antibody NI006/ALXN2220 is a fully human IgG1m3 allotype antibody and composed of two identical heavy chains of the IgG1 subclass and the IgG1m3 allotype. In addition, as mentioned above, original human antibody NI-301.37F1 is of the kappa type and thus, NI006/ALXN2220 is composed of two identical light chains of the kappa subclass. The sequences of the variable heavy (VH) and variable light (VL) chains of NI006/ALXN2220 are set forth in SEQ ID NOs: 2 and 6, and the sequences of the corresponding human constant regions are known in the art. For example, each isotype and like the IgG1m3 isotype has a unique amino acid sequence of the constant regions of their heavy chains; see Jefferis and Lefrance (2009), supra. Thus, in one embodiment, the antibody present in the pharmaceutical formulation of the present invention is characterized by two heavy chains, wherein each heavy chain (HC) comprises an amino acid sequence set forth in SEQ ID NO: 9, and by two light chains, wherein each light chain (LC) comprises an amino acid sequence set forth in SEQ ID NO: 10. Each heavy chain is comprised of 450 amino-acid residues, and each light chain consists of 214 amino acid residues. The four chains are stabilized by intra-chain and inter-chain disulfide bonds, wherein the positions of the disulfide bridges, which have been identified per Lys-C and trypsin digestion and subsequent LC-MS (see Example 7) are the following:
LC:C23-LC:C88
LC:C134-LC:C194
LC:C214-HC:C223
HC:C22-HC:C97
HC:C147-HC:C203
HC1:229-HC2:229 and HC1:232-HC2:232
HC:C264-HC:C324
HC:C370-HC:C428.
(amino acid numbering corresponds to the heavy chain sequence set forth in SEQ ID NO: 9)

Thus, the antibody as used in accordance with the present invention may be characterized to comprise at least 8 disulfide bridges, preferably at the above-identified positions.

Furthermore, each heavy chain of antibody NI006/ALXN2220 contains a single N-linked glycosylation site at Asn300. The N-linked glycosylation structure is predominantly a fucosylated, complex biantennary glycan with 0 galactose residues (G0F) (about 49%) or with 1 galactose residue (G1F) (about 25%). The detailed glycosylation profile is shown in Example 7. Glycosylation plays a vital role in the stability, in vivo activity, solubility, serum half-life and immunogenicity of many therapeutic proteins. N-glycan analysis determines the relative distribution of N-glycans released from the glycoprotein, and provides insightful information on the safety and efficacy of bio-therapeutics.

Thus, in a preferred embodiment, the antibody as used in accordance with the present invention is an IgG antibody and has a heavy chain which is N-glycosylated, preferably wherein the N-linked glycosylation site is Asn300, preferably, if for example expressed in CHO cells, wherein the antibody comprises a N-linked glycosylation structure which is predominantly a glycan with 0 galactose residues (G0F) (about 49%) or with 1 galactose residue (G1F) (about 25%). Most preferably, the antibody has the glycosylation profile as shown in Example 7.

Furthermore, one or several amino acids at the amino or carboxy terminus of the light and/or heavy chain, such as the C-terminal lysine of the heavy chain, if present, may be missing or derivatized in a proportion or all of the molecules.

Thus, in one embodiment, the antibody present in the pharmaceutical composition of the present invention has a heavy chain that does not comprise a C-terminal lysine. For example, in such embodiment, the C-terminal lysine included in SEQ ID NO: 9 is missing. The sequence of such a heavy chain is set forth in SEQ ID NO: 13.

In addition, or alternatively, the antibody has a heavy chain, in which the glutamine at the N-terminal is derivatized, preferably substituted with pyroglutamate. This pyroglutamate formation is also referred to as N-terminal cyclization. The sequence of such a heavy chain is set forth in SEQ ID NO: 14 or SEQ ID NO: 15.

Most preferably, the antibody has a heavy chain that does not comprise a C-terminal lysine, i.e., which C-terminal lysine has undergone C-terminal lysine clipping, in which the glutamine at the N-terminal is substituted with pyroglutamate, i.e., which has undergone N-terminal glutaminyl cyclization (see SEQ ID NO: 15), and which is N-glycosylated.

The amino acid sequences of the heavy and light chains are shown below:

QLQLQESGPGLVKPSETLSLTCSVSGGSIISRSSYWGWIRQPPGKGLEWI
GGIYHSGNTYDNPSLKSRLTMSVDTSKNQFSLNLRSVTAADTAVYYCARI
VPGGDAFDIWGQGTMVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

(SEQ ID NO: 9, NI006/ALXN2220, heavy chain amino acid sequence, wherein the amino acids of the constant region are underlined, and wherein the C-terminal lysine (K) is optional and/or the N-terminal glutamine (Q) undergoes intramolecular cyclization, resulting in the formation of pyroglutamic acid)

DIQMTQSPSSLSASVGDRVTIACRASQSVGTYLNWYQQKRGKAPKLLIFA
ASSLQSGVPSRFSGSGSGTDFTLTISSLOPEDFATYYCQQSYSSPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

(SEQ ID NO: 10, NI006/ALXN2220, light chain amino acid sequence, wherein the amino acids of the constant region are underlined).

Furthermore, the theoretical molecular weight of antibody NI006/ALXN2220 is 144.2 kDa, and the weight determined by mass spectrometry (MS) is 144.2 kDa (deglycosylated) and between 147.0 and 147.6 kDa (intact IgG1), respectively. Thus, in one embodiment, the antibody comprised in the pharmaceutical composition of the present invention has a molecular weight of about 150 kDa, preferably of about 147 kDa.

### Antibody Formulations

Any of the anti-TTR antibodies or antigen-binding fragments thereof described herein and used in accordance with the present invention (e.g., the anti-TTR antibody with a VH region having the amino acid sequence of SEQ ID NO: 7 or SEQ ID NO: 11, and a VL region having the amino acid sequence of SEQ ID NO: 8 or SEQ ID NO: 12, preferably the anti-TTR antibody with a VH region having the amino acid sequence of SEQ ID NO: 11, and a VL region having the amino acid sequence of SEQ ID NO: 8, or an equivalent antibody having substantially the same binding properties, and an anti-TTR antibody with a heavy chain having the amino acid sequence of SEQ ID NO: 9, preferably including the PTMs mentioned hereinbefore, e.g., an anti-TTR antibody with a heavy chain including an amino acid sequence having the amino acid sequence of SEQ ID NO: 13, 14, or 15, preferably of SEQ ID NO: 15, preferably wherein the N-terminus is cyclized and wherein the HC is N-glycosylated, and a light chain having the amino acid sequence of SEQ ID NO: 10, respectively) may be formulated at a concentration of about 1 mg/mL to about 500 mg/mL (e.g., 25 mg/mL to 200 mg/mL (e.g., 25 mg/mL, 50 mg/mL, 75 mg/mL, 100 mg/mL, 125 mg/mL, 150 mg/mL, 175 mg/mL, or 200 mg/mL). For example, the anti-TTR antibody may be provided in the form of an aqueous formulation (e.g., a pharmaceutical composition) that is at a concentration of about 10 mg/mL to about 125 mg/mL, e.g., about 10 mg/mL to about 90 mg/mL, e.g., about 20 mg/mL to about 80 mg/mL, e.g., about 25 mg/mL to about 75 mg/mL, e.g., about 25 mg/mL to about 125 mg/mL. Exemplarily antibodies having the same binding specificities, in particular strong binding to misfolded-aggregated TTR but no binding to the physiological TTR monomers, are for example provided in WO 2015/092077 A1, e.g., antibodies NI-301.59F1 and NI-301.35G11, which content is herein incorporated by reference.

For example, the anti-TTR antibody is formulated into an aqueous solution (e.g., a pharmaceutical composition) at about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, or about 125 mg/mL of the anti-TTR antibody described herein.

In an example, the anti-TTR antibody is provided as an aqueous solution in a vial (e.g., a glass vial) at a concentration of 50 mg/mL (±12%) or 100 mg/mL (±12%). The total volume of the aqueous solution in a vial may be about 1 mL to about 200 mL, about 1 mL to about 150 mL, about 1 mL to about 100 mL, about 1 mL to about 50 mL, about 5 mL to about 25 mL, about 18 mL to about 22 mL, or about 1 mL to about 10 mL (e.g., about 1 mL to about 2 mL, about 1 mL to about 1.8 mL, about 1 mL to about 1.6 mL, about 1 mL to about 1.4 mL, about 1 mL to about 1.2 mL, about 1.5 mL to about 1.25 mL, about 1.5 mL to about 2 mL, about 1.9 mL to about 1.2 mL, about 2.1 mL to about 2.25 mL) or about 1 mL to about 100 mL (e.g., about 1 mL, about 1.8 mL, about 1.9 mL, about 2 mL, about 2.1 mL, about 2.2 mL about 2.25 mL, about 2.3 mL, about 2.4 mL, about 2.5 mL about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 40 mL, about 50 mL, about 60 mL, about 70 mL, about 80 mL, about 90 mL, or about 100 mL In another example, the total volume of an aqueous solution in the vial will be 2 mL (±12%), 5 mL (±12%), 10 mL (±12%), 15 mL (±12%), 20 mL (±12%), 25 mL (±12%), or 30 mL (±12%). The subject may receive a quantity of vials sufficient to provide a desired total dose.

### Subjects

The methods provided herein may be used to treat a subject that has ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, preeclampsia, or a known pathogenic TTR mutation (e.g., one that causes amyloidosis). The subject may have sporadic, WT-ATTR-CM and a negative genetic testing for a TTR mutation. Genetic testing may be performed by standard laboratory techniques, for example, DNA or RNA sequencing or protein sequencing using mass spectrometry.

In some examples, the subject to be treated has been previously treated with a TTR tetramer stabilizer (e.g., diflunisal, Tafamidis, and Acoramidis (AG10)). Alternatively, the subject may concurrently receive treatment with a TTR tetramer stabilizer (e.g., diflunisal, Tafamidis, and Acoramidis (AG10)) while receiving treatment with an anti-TTR antibody, as described herein.

The administration of the anti-TTR antibody (e.g., at a particular dosage, frequency, and/or duration of treatment described below) may provide a therapeutic or prophylactic effect to the subject. Such a therapeutic of prophylactic effect of the antibody may result in a sustained blood (e.g., plasma or serum) concentration of the antibody at ≥1 μg/mL (e.g., ≥1 μg/mL, ≥2.5 μg/mL, ≥5 μg/mL, ≥10 μg/mL, ≥20 μg/mL, ≥30 μg/mL, ≥40 μg/mL, ≥50 μg/mL, ≥60 μg/mL, ≥70 μg/mL, ≥80 μg/mL, ≥90 μg/mL, 2 100 μg/mL, ≥110 μg/mL, ≥120 μg/mL, ≥130 μg/mL, ≥140 μg/mL, ≥150 μg/mL, ≥160 μg/mL, ≥170 μg/mL, ≥180 μg/mL, ≥190 μg/mL, ≥200 μg/mL, ≥210 μg/mL, ≥220 μg/mL, ≥230 μg/mL, ≥240 μg/mL, ≥250 μg/mL, ≥260 μg/mL, ≥270 μg/mL, ≥280 μg/mL, ≥290 μg/mL, ≥300 μg/mL, ≥310 μg/mL, ≥320 μg/mL, ≥330 μg/mL, ≥340 μg/mL, ≥350 μg/mL, ≥360 μg/mL, ≥370 μg/mL, ≥380 μg/mL, ≥390 μg/mL, ≥400 μg/mL, ≥410 μg/mL, ≥420 μg/mL, ≥430 μg/mL, ≥440 μg/mL, ≥450 μg/mL, ≥460 μg/mL, ≥470 μg/mL, ≥480 μg/mL, ≥490 μg/mL, ≥500 μg/mL, ≥510 μg/mL, ≥520 μg/mL, ≥530 μg/mL, ≥540 μg/mL, ≥550 μg/mL, ≥560 μg/mL, ≥570 μg/mL, ≥580 μg/mL, ≥590 μg/mL, ≥600 μg/mL, ≥610 μg/mL, ≥620 μg/mL, ≥630 μg/mL, ≥640 μg/mL, ≥650 μg/mL, ≥660 μg/mL, ≥670 μg/mL, ≥680 μg/mL, ≥690 μg/mL, ≥700 μg/mL, ≥710 μg/mL, ≥720 μg/mL, ≥730 μg/mL, ≥740 μg/mL, ≥750 μg/mL, ≥760 μg/mL, ≥770 μg/mL, ≥780 μg/mL, ≥790 μg/mL, ≥800 μg/mL, ≥810 μg/mL, ≥820 μg/mL, ≥830 μg/mL, ≥840 μg/mL, ≥850 μg/mL, ≥860 μg/mL, ≥870 μg/mL, ≥880 μg/mL, ≥890 μg/mL, ≥900 μg/mL, ≥910 μg/mL, ≥920 μg/mL, ≥930 μg/mL, ≥940 μg/mL, ≥950 μg/mL, ≥960 μg/mL, ≥970 μg/mL, ≥980 μg/mL, ≥990 μg/mL, ≥1000 μg/mL, or more) in the subject. Moreover, such a plasma concentration may be sustained for any period of time (e.g., about 1 day to about 1 week, about 1 day to about 30 days, e.g., about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days). For example, the plasma concentration may be sustained at ≥1 μg/mL for 21 days to 35 days. For example, the plasma concentration may be sustained at ≥2.5 μg/mL for 21 days to 35 days. For example, the plasma concentration may be sustained at ≥_5 μg/mL for 21 days to 35 days. For example, the plasma concentration may be sustained at ≥10 μg/mL for 21 days to 35 days. Preferably, the indicated plasma concentrations may be sustained for 21 days to 35 days, preferably for 28 days to 35 days, most preferably for 28 days or 35 days.

Alternatively, the therapeutic or prophylactic effect of the antibody may result in a sustained plasma concentration of the antibody at an area under the curve (AUC) of ≥2,000 μg*day/mL (e.g., 2 2,000 μg*day/mL, ≥4,000 μg*day/mL, ≥6,000 μg*day/mL, ≥8,000 μg*day/mL, ≥10,000 μg*day/mL, 12,000 μg*day/mL, ≥14,000 μg*day/mL, ≥16,000 μg*day/mL, ≥18,000 μg*day/mL, ≥20,000 μg*day/mL, ≥22,000 μg*day/mL, ≥24,000 μg*day/mL, ≥26,000 μg*day/mL, ≥28,000 μg*day/mL, ≥30,000 μg*day/mL, ≥32,000 μg*day/mL, ≥34,000 μg*day/mL, ≥36,000 μg*day/mL, ≥38,000 μg*day/mL, 40,000 μg*day/mL, ≥42,000 μg*day/mL, ≥44,000 μg*day/mL, ≥46,000 μg*day/mL, ≥48,000 μg*day/mL, ≥50,000 μg*day/mL, ≥52,000 μg*day/mL, ≥54,000 μg*day/mL, ≥56,000 μg*day/mL, ≥58,000 μg*day/mL, ≥60,000 μg*day/mL, ≥62,000 μg*day/mL, ≥64,000 μg*day/mL, ≥66,000 μg*day/mL, 68,000 μg*day/mL, ≥70,000 μg*day/mL, ≥72,000 μg*day/mL, ≥74,000 μg*day/mL, ≥76,000 μg*day/mL, ≥78,000 μg*day/mL, ≥80,000 μg*day/mL, ≥82,000 μg*day/mL, ≥84,000 μg*day/mL, ≥86,000 μg*day/mL, ≥88,000 μg*day/mL, ≥90,000 μg*day/mL, ≥92,000 μg*day/mL, ≥94,000 μg*day/mL, 96,000 μg*day/mL, ≥98,000 μg*day/mL, ≥100,000 μg*day/mL.

### Monitoring and Assessing Treatment

Those skilled in the art (e.g., a clinical practitioner) can monitor and assess the efficacy of treatment. Methods for monitoring and assessing treatment efficacy are described below. For any of the methods described herein, efficacy of treatment may be assessed or monitored according to standard techniques known in the art. For example, administration of the anti-TTR antibody or pharmaceutical composition thereof may be monitored by determining the level (e.g., mRNA or protein level) of one or more biomarkers (e.g., a biomarker of ATTR). Exemplary biomarkers include cardiac troponin T (TnT), N-terminal pro-B-type natriuretic peptide (NT-proBNP), C-reactive protein (CRP), C3, C4, IL1 b, IL6, IL8, IFNg, TNF-α, IL110, IL1RA, serum amyloid A (SAA), ferritin, ATTR, galectin 3 (Gal-3), soluble suppression of tumorigenicity 2 (sST2), carboxy-terminal propeptide of procollagen type 1 (PICP), and propeptide of procollagen type III (PIIINP). In some embodiments, administration of the anti-TTR antibody is monitored by determining a level of one or more biomarkers, such as, e.g., cardiac troponin T (TnT) and N-terminal pro-B-type natriuretic peptide (NT-proBNP). In a preferred embodiment, administration of the anti-TTR antibody or pharmaceutical composition thereof may be monitored by determining the level (e.g., mRNA or protein level) of N-terminal pro-B-type natriuretic peptide (NT-proBNP).

In an embodiment, a decrease in the level of the biomarker(s) is indicative of treatment efficacy. For example, a subject may have an NT-proBNP level of about 300 pg/mL to about 20,000 pg/mL before undergoing the treatment methods described herein, and a level of about 0 pg/mL to about 300 pg/mL after undergoing the treatment methods described herein. Preferably, a subject may have an NT-proBNP level of >2000 pg/mL before undergoing the treatment methods described herein.

In another example, a subject may have a TnT level of about 10 pg/mL to about 200 pg/mL before undergoing the treatment methods described herein, and a level of 0 pg/mL to about 10 pg/mL after undergoing the treatment methods described herein.

Any decrease (e.g., a 30%, 40%, 50%, 60%, 70% 80%, 90%, or 100% decrease) in the level of TnT or NT-proBNP (e.g., over time, such as over 1, 2, 3, 4, 5, 6, 7, 8, 9, 10, 11, or 12 months, or relative to an untreated ATTR subject) is indicative of treatment efficacy. For example, subjects with ATTR or ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, or preeclampsia may have high levels (e.g., about 300 pg/mL to about 20,000 pg/mL) of NT-proBNP, which can decrease over time in the blood (e.g., plasma or serum) of subject being treated with the methods described herein.

An increase of CRP level, in particular a dose dependent transient (e.g., about 1 to about 14 days, e.g., 1, 2, 3, 4, 5, 6, 7, 8, 9, 10, 11, 12, 13, or 14 days) increase of CRP level (e.g., over time or relative to an untreated ATTR subject) is also indicative of on-target immune activation and efficacy of the treatment.

Any suitable laboratory techniques for determining protein levels of a biomarker (e.g., TnT, NT-proBNP, and CRP) in a sample (e.g., blood, e.g., plasma or serum) can be used, including, but not limited, to flow cytometry (FC), fluorescence-activated cell sorting (FACS) Western blot, enzyme-linked immunosorbent assay (ELISA), mass spectrometry (MS), immunofluorescence (IF), immunoprecipitation (IP), radioimmunoassay, dot blotting, high performance liquid chromatography (HPLC), surface plasmon resonance, optical spectroscopy, and immunohistochemistry (IHC). Any suitable laboratory techniques for determining mRNA expression levels of a biomarker (e.g., TnT, NT-proBNP, and CRP) in a sample can be used, including, but not limited to, PCR, RT-PCR, qPCR, RT-qPCR, microarray analysis, Northern blot, MASSARRAY® technique, SAGE, and RNA-sequencing.

For detecting and measuring the levels of CRP also the above-described kit comprising reagents for detection and measuring the level of CRP in a patient's sample can be used. As mentioned, means and methods for the detection of CRP are known to the person skilled in the art; see for example US 2006/0246522 A1 as well as the literature cited in section [0010] to [0017] of US 2006/0246522 A1. In general, detection of CRP can be accomplished by any suitable method. Exemplary detection methods include immunodetection methods (e.g., by using antibodies that specifically bind CRP), optical methods (e.g., microscopy, both confocal and non-confocal, detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry)), electrochemical methods (voltametry and amperometry techniques), atomic force microscopy, and radio frequency methods, e.g., multipolar resonance spectroscopy. In this context, standard immunoassays can be used for the detection of CRP, wherein immunoassays can be conducted in a variety of different formats, and generally involve the detection of binding between an anti-biomarker antibody (e.g., an anti-CRP antibody) and its target biomarker antigen (e.g., CRP) in a biological sample obtained for a patient. Immunoassays can be conducted in any of a variety of formats and in general, the assay will measure the reactivity between an anti-biomarker antibody and a patient sample. For example, commercially available kits for the detection of CRP like ELISA kits as for example the CRP/C-Reactive Protein ELISA Kit from LifeSpan BioSciences, Seattle, Washington, USA are available to the skilled person.

Additional methods for monitoring or assessing treatment efficacy may include monitoring or assessing the level of cardiac amyloid burden and cardiac mass in the subject. Subjects with a disease associated with ATTR (e.g., ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, or preeclampsia) may have a left ventricular ejection fraction [LVEF) of ≥20% (e.g., about 20% to about 60%, e.g., 20%, 25%, 30%, 35%, 40%, 45%, 50%, 55%, or 60%) before undergoing the treatment methods described herein and/or a left ventricular wall thickness (LVWT) 212 mm (e.g., about 12 mm to about 30 mm, e.g., about 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, or 30 mm) before undergoing the treatment methods described here, e.g., as measured by echocardiography. A reduction (e.g., a 5% to 100% reduction, relative to a control) in the level of cardiac amyloid burden or cardiac mass indicates treatment efficacy in a subject for a treatment as described herein. For example, a decrease of about 5% to about 25% in left ventricular cardiac mass over a treatment period of about 17 weeks to about 50 weeks indicates treatment efficacy in a subject. In another example, a decrease of about 5% to about 75% in cardiac amyloid burden over a treatment period of about 17 weeks to about 50 weeks indicates treatment efficacy in a subject. Cardiac mass reduction and cardiac amyloid burden may be measured by cardiac magnetic resonance imaging (MRI).

Further examples for monitoring or assessing the efficacy of treatment may include monitoring and assessing cardiac function, which may be assessed by echocardiography. For example, an echocardiogram maybe used to measure contractile function (strain), thickness and filling pressure, and LVEF, left ventricular end-diastolic/end-systolic diameter, systolic function, left ventricular end systolic volume (LVESV), left ventricular end diastolic volume (LVEDV), left ventricular diastolic function, right ventricular function, global longitudinal strain, end diastolic-interventricular septum (ED-IVS) and/or end diastolic-posterior wall (ED-PW).

Other readouts for assessing the efficacy of treatment include the 6-minute walt test (6-MWT), the Kansas City Cardiomyopathy Questionnaire (KCCQ), magnetic resonance imaging (MRI), and bone cintigraphy. The 6-MWT is a sub-maximal exercise test used to assess walking endurance and aerobic capacity. Subjects will walk around the perimeter of a set circuit for a total of 6 minutes. The score of the test is the distance a subject walks on a flat, hard surface in a period of 6 minutes (measured in meters and can be rounded up to the nearest decimal point). The KCCQ is a 23-item self-administered questionnaire developed to independently measure the subject's perception of their health status, which includes heart failure symptoms, impact on physical and social function, and how their heart failure impacts their quality of life within a 2-week recall period. MRI may assess morphological (e.g., LV mass) and functional (e.g., LV ejection fraction, global longitudinal strain) characteristic. Bone scintigraphy may be used to assess a heart retention (HR)/whole-body retention (WBR) ratio and heart retention (HR)/skull retention (SR) ratio.

### Dosing Regimens

Any of the anti-TTR antibodies or pharmaceutical compositions thereof as used in accordance with the present invention may be administered to a subject in a dosing regimen to treat or prevent ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, or preeclampsia in a subject. The dosage of the anti-TTR antibody (or fragment or pharmaceutical composition thereof), frequency of administration, and/or duration of treatment are described below.

### Dosage

For any of the methods described herein, the anti-TTR antibody or pharmaceutical composition thereof may be administered at a dose (e.g., a maintenance dose and/or loading dose) of about 0.3 milligrams per kilogram (mg/kg) of body weight to about 100 mg/kg (e.g., 0.3 mg/kg, 1 mg/kg, 3 mg/kg, 10 mg/kg, 30 mg/kg, 60 mg/kg, or 100 mg/kg) to the subject. For example, the anti-TTR antibody as used in accordance with the present invention may be administered at a dose (e.g., a maintenance dose and/or loading dose) of about 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, 80 mg/kg, 81 mg/kg, 82 mg/kg, 83 mg/kg, 84 mg/kg, 85 mg/kg, 86 mg/kg, 87 mg/kg, 88 mg/kg, 89 mg/kg, 90 mg/kg, 91 mg/kg, 92 mg/kg, 93 mg/kg, 94 mg/kg, 95 mg/kg, 96 mg/kg, 97 mg/kg, 98 mg/kg, 99 mg/kg, or 100 mg/kg.

More particularly, the anti-TTR antibody as used in accordance with the present invention may be preferably administered at a starting dose of about 0.3 mg/kg to 10 mg/kg to the subject, e.g., of about 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 6 mg/kg, 7 mg/kg, 8 mg/kg, 9 mg/kg, or 10 mg/kg. Furthermore, the anti-TTR antibody as used in accordance with the present invention may further and preferably be administered at a maintenance dose of about 10 mg/kg to about 60 mg/kg to the subject, e.g., of about 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, or 60 mg/kg.

For example, and preferably, the anti-TTR antibody or pharmaceutical composition thereof may be administered at a dose of 30 mg/kg-60 mg/kg, such as 30 mg/kg or 60 mg/kg. The anti-TTR antibody may be administered at a dose of 30 mg/kg. The anti-TTR antibody may be administered at a dose of 60 mg/kg.

For any of the methods described herein, the anti-TTR antibody or pharmaceutical composition thereof may be administered (e.g., in a dosing regimen) at a dose (e.g., a maintenance dose and/or loading dose) of about 600 mg to about 7500 mg (e.g., 600 mg, 2500 mg, 3000 mg, 3500 mg, 4000 mg, 5000 mg, 6000 mg, 6240 mg, or 7500 mg). For example, the anti-TTR antibody may be administered at a dose (e.g., a maintenance dose and/or loading dose) of about 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, 4800 mg, 4850 mg, 4900 mg, 4950 mg, 5000 mg, 5050 mg, 5100 mg, 5150 mg, 5200 mg, 5250 mg, 5300 mg, 5350 mg, 5400 mg, 5450 mg, 5500 mg, 5550 mg, 5600 mg, 5650 mg, 5700 mg, 5750 mg, 5800 mg, 5850 mg, 5900 mg, 5950 mg, 6000 mg, 6050 mg, 6100 mg, 6150 mg, 6200 mg, 6210 mg, 6220 mg, 6230 mg, 6240 mg, 6250 mg, 6260 mg, 6270 mg, 6280 mg, 6290 mg, 6300 mg, 6350 mg, 6400 mg, 6450 mg, 6500 mg, 6550 mg, 6600 mg, 6650 mg, 6700 mg, 6750 mg, 6800 mg, 6850 mg, 6900 mg, 6950 mg, 7000 mg, 7050 mg, 7100 mg, 7150 mg, 7200 mg, 7250 mg, 7300 mg, 7350 mg, 7400 mg, 7450 mg, or 7500 mg.

More particularly, the anti-TTR antibody as used in accordance with the present invention may be preferably administered at a loading dose for about 600 mg to about 4000 mg (e.g., a loading dose) of about 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg. In one embodiment, preferably after administration of the loading dose, the anti-TTR antibody as used in accordance with the present invention may be preferably administered at a maintenance dose for about 600 mg to about 7500 mg (e.g., a maintenance dose) of about 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, 4800 mg, 4850 mg, 4900 mg, 4950 mg, 5000 mg, 5050 mg, 5100 mg, 5150 mg, 5200 mg, 5250 mg, 5300 mg, 5350 mg, 5400 mg, 5450 mg, 5500 mg, 5550 mg, 5600 mg, 5650 mg, 5700 mg, 5750 mg, 5800 mg, 5850 mg, 5900 mg, 5950 mg, 6000 mg, 6050 mg, 6100 mg, 6150 mg, 6200 mg, 6210 mg, 6220 mg, 6230 mg, 6240 mg, 6250 mg, 6260 mg, 6270 mg, 6280 mg, 6290 mg, 6300 mg, 6350 mg, 6400 mg, 6450 mg, 6500 mg, 6550 mg, 6600 mg, 6650 mg, 6700 mg, 6750 mg, 6800 mg, 6850 mg, 6900 mg, 6950 mg, 7000 mg, 7050 mg, 7100 mg, 7150 mg, 7200 mg, 7250 mg, 7300 mg, 7350 mg, 7400 mg, 7450 mg, or 7500 mg.

For example, the anti-TTR antibody or pharmaceutical composition thereof may be administered at a dose of 2000-5000 mg, such as 2400 mg, 2500 mg, 3000 mg, 3200 mg, or 3500 mg, preferably 3500 mg or 3200 mg, most preferably 3200 mg.

The anti-TTR antibody may be administered at a dose (e.g., a maintenance dose and/or loading dose) of 2000 mg, 2010 mg, 2020 mg, 2030 mg, 2040 mg, 2050 mg, 2060 mg, 2070 mg, 2080 mg, 2090 mg, 2100 mg, 2110 mg, 2120 mg, 2130 mg, 2140 mg, 2150 mg, 2160 mg, 2170 mg, 2180 mg, 2190 mg, 2200 mg, 2210 mg, 2220 mg, 2230 mg, 2240 mg, 2250 mg, 2260 mg, 2270 mg, 2280 mg, 2290 mg, 2300 mg, 2310 mg, 2320 mg, 2330 mg, 2340 mg, 2350 mg, 2360 mg, 2370 mg, 2380 mg, 2390 mg, 2400 mg, 2410 mg, 2420 mg, 2430 mg, 2440 mg, 2450 mg, 2460 mg, 2470 mg, 2480 mg, 2490 mg, 2500 mg, 2510 mg, 2520 mg, 2530 mg, 2540 mg, 2550 mg, 2560 mg, 2570 mg, 2580 mg, 2590 mg, 2600 mg, 2610 mg, 2620 mg, 2630 mg, 2640 mg, 2650 mg, 2660 mg, 2670 mg, 2680 mg, 2690 mg, 2700 mg, 2710 mg, 2720 mg, 2730 mg, 2740 mg, 2750 mg, 2760 mg, 2770 mg, 2780 mg, 2790 mg, 2800 mg, 2810 mg, 2820 mg, 2830 mg, 2840 mg, 2850 mg, 2860 mg, 2870 mg, 2880 mg, 2890 mg, 2900 mg, 2910 mg, 2920 mg, 2930 mg, 2940 mg, 2950 mg, 2960 mg, 2970 mg, 2980 mg, 2990 mg, 3000 mg, 3010 mg, 3020 mg, 3030 mg, 3040 mg, 3050 mg, 3060 mg, 3070 mg, 3080 mg, 3090 mg, 3100 mg, 3110 mg, 3120 mg, 3130 mg, 3140 mg, 3150 mg, 3160 mg, 3170 mg, 3180 mg, 3190 mg, 3200 mg, 3210 mg, 3220 mg, 3230 mg, 3240 mg, 3250 mg, 3260 mg, 3270 mg, 3280 mg, 3290 mg, 3300 mg, 3310 mg, 3320 mg, 3330 mg, 3340 mg, 3350 mg, 3360 mg, 3370 mg, 3380 mg, 3390 mg, 3400 mg, 3410 mg, 3420 mg, 3430 mg, 3440 mg, 3450 mg, 3460 mg, 3470 mg, 3480 mg, 3490 mg, 3500 mg, 3510 mg, 3520 mg, 3530 mg, 3540 mg, 3550 mg, 3560 mg, 3570 mg, 3580 mg, 3590 mg, 3600 mg, 3610 mg, 3620 mg, 3630 mg, 3640 mg, 3650 mg, 3660 mg, 3670 mg, 3680 mg, 3690 mg, 3700 mg, 3710 mg, 3720 mg, 3730 mg, 3740 mg, 3750 mg, 3760 mg, 3770 mg, 3780 mg, 3790 mg, 3800 mg, 3810 mg, 3820 mg, 3830 mg, 3840 mg, 3850 mg, 3860 mg, 3870 mg, 3880 mg, 3890 mg, 3900 mg, 3910 mg, 3920 mg, 3930 mg, 3940 mg, 3950 mg, 3960 mg, 3970 mg, 3980 mg, 3990 mg, 4000 mg, 4010 mg, 4020 mg, 4030 mg, 4040 mg, 4050 mg, 4060 mg, 4070 mg, 4080 mg, 4090 mg, 4100 mg, 4110 mg, 4120 mg, 4130 mg, 4140 mg, 4150 mg, 4160 mg, 4170 mg, 4180 mg, 4190 mg, 4200 mg, 4210 mg, 4220 mg, 4230 mg, 4240 mg, 4250 mg, 4260 mg, 4270 mg, 4280 mg, 4290 mg, 4300 mg, 4310 mg, 4320 mg, 4330 mg, 4340 mg, 4350 mg, 4360 mg, 4370 mg, 4380 mg, 4390 mg, 4400 mg, 4410 mg, 4420 mg, 4430 mg, 4440 mg, 4450 mg, 4460 mg, 4470 mg, 4480 mg, 4490 mg, 4500 mg, 4510 mg, 4520 mg, 4530 mg, 4540 mg, 4550 mg, 4560 mg, 4570 mg, 4580 mg, 4590 mg, 4600 mg, 4610 mg, 4620 mg, 4630 mg, 4640 mg, 4650 mg, 4660 mg, 4670 mg, 4680 mg, 4690 mg, 4700 mg, 4710 mg, 4720 mg, 4730 mg, 4740 mg, 4750 mg, 4760 mg, 4770 mg, 4780 mg, 4790 mg, 4800 mg, 4810 mg, 4820 mg, 4830 mg, 4840 mg, 4850 mg, 4860 mg, 4870 mg, 4880 mg, 4890 mg, 4900 mg, 4910 mg, 4920 mg, 4930 mg, 4940 mg, 4950 mg, 4960 mg, 4970 mg, 4980 mg, 4990 mg, or 5000 mg.

Most preferably, the flat doses are administered depending on the body weight of the subject to be treated as explained further above.

Any of the dosages described herein may be administered to the subject as a maintenance dose and/or loading dose. For example, the method of treating or preventing a disease associated with ATTR (e.g., ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, or preeclampsia) may include the step of administering the anti-TTR antibody at an initial dose of about 0.3 mg/kg to about 10 mg/kg (e.g., 0.3 mg/kg, 1 mg/kg, 3 mg/kg, or 10 mg/kg). In a further example, the method may further include the step of administering the anti-TTR antibody at a maintenance dose of about 10 mg/kg to about 60 mg/kg (e.g., 10 mg/kg, 30 mg/kg, or 60 mg/kg), or at a total dose of about 600 mg to about 4000 mg (e.g., 2500 mg or 3000 mg).

The methods described herein contemplate the administration of the anti-TTR antibody to a subject more than once, e.g., in a plurality of doses. For example, the administration of two or more (e.g., 2, 3, 4, 5, 6, 7, 8, 9, 10, 11, 12, 13, 14, 15, 16, 17, 18, 19, 20, 21, 22, 23, 24, 25, or more) doses of the anti-TTR antibody may occur. Further contemplated herein is the up-titration (e.g., increase) of the administered doses. For example, the administration of two or more doses may include one, two three, four, five, or six up-titrations (e.g., increases) of the dosage (e.g., if a subject is not responsive or is insufficiently responsive to the prior dose administered). For example, an up-titration may include an increase from 0.3 mg/kg to 1 mg/kg, 1 mg/kg to 3 mg/kg, 3 mg/kg to 10 mg/kg, 10 mg/kg to 30 mg/kg, or 30 mg/kg to 60 mg/kg. The up-titration may occur at any point during the subject's treatment. Up-titration may be performed when a blood (e.g., serum or plasma level) of the antibody is determined to be below a desired threshold (e.g., less than 10 μg/mL).

In a preferred embodiment, the anti-TTR antibody is administered in a weight based, flat dosing regimen comprising: (a) 2400 mg to a patient weighing equal or more than 40 kg (≥40 kg) to under 60 kg (<60 kg); (b) 3200 mg to a patient weighing equal or more than 60 kg (≥60 kg) to under 100 kg (<100 kg); or (c) 4800 mg to a patient weighing equal or more than 100 kg (≥100 kg). Preferably, the weight-based dosing regimen is administered intravenously (IV) to an adult patient.

### Frequency and Duration of Treatment

For any of the methods described herein, the anti-TTR antibody or pharmaceutical composition thereof may be administered to the subject at a frequency of about once every 3 to about once every 42 days (e.g., about once every 21 days to about once every 35 days or about once every 28 days to about once every 35 days). For example, the anti-TTR antibody may be administered at a dose described herein (e.g., 0.3 mg/kg, 1 mg/kg, 3 mg/kg, 10 mg/kg, 30 mg/kg, or 60 mg/kg, preferably 30 mg/kg or 60 mg/kg, or 2000 mg, 2400 mg, 2500 mg, 3000 mg, 3200 mg, 3500 mg, 4000 mg, 4800 mg, or 5000 mg, preferably 2500 mg, 3500 mg, or 5000 mg, and most preferably 2400 mg, 3200 mg, or 4800 mg) about once every 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

Additionally, treatment with the anti-TTR antibody may be administered to the subject for any duration of time, such as for at least 6 months, 12 months, or 18 months (e.g., from 1 week to 1 year). For example, the anti-TTR antibody may be administered to the subject for about 4-30 months (e.g., about 4-30 months or 12-18 months). For example, the anti-TTR antibody may be administered to the subject for 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, or 30 months. Alternatively, the anti-TTR antibody may be administered to the subject at a frequency described above (e.g., about once every 28 days to about once every 35 days) for the lifetime of the subject.

A method of treating, or preventing, a subject having, or at risk of having, ATTR ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, or preeclampsia may utilize any combination of the above dosages, frequencies, and/or duration of treatment.

In a preferred embodiment, the anti-TTR antibody (e.g., ALXN2220) is administered every four weeks (q4w).

### Routes of Administration

Administration of the anti-TTR antibody to the subject may be, for example, by subcutaneous or intravenous routes (e.g., by intravenous infusion). For example, the anti-TTR antibody may be administered intravenously using a dosing syringe in a syringe pump or an infusion bag. If an infusion line is utilized for administration, the infusion line may be flushed before and/or after the infusion of the anti-TTR antibody. An infusion syringe may be used for administration of a total antibody dose of up to 100 mg, while an infusion pump is used for administration of a total antibody dose exceeding 100 mg, optionally using an infusion bag prefilled with the diluent. Glucose or a polymer thereof, such as dextran, may be used as a diluent at a concentration of about 1%, 2%, 3%, 4%, 5%, 6%, 7%, 8%, 9%, or 10% w/v. A diluted form (e.g., an aqueous solution) of the anti-TTR antibody may be administered to a subject with an infusion syringe at a volume of about 10 mL to about 200 mL (e.g., about 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 105 mL, 110 mL, 115 mL, 120 mL, 125 mL, 130 mL, 135 mL, 140 mL, 145 mL, 150 mL, 155 mL, 160 mL, 165 mL, 170 mL, 175 mL, 180 mL, 185 mL, 190 mL, 195 mL, or 200 mL). Administration of the anti-TTR antibody as an aqueous solution may occur over 1-5 hours, such as 1, 2, 3, 4, or 5 hours, e.g., 2 hours±10 minutes. The first administration of the anti-TTR antibody may occur over, e.g., 2 hours±10 minutes while subsequent administrations occur over, e.g., approximately 1 hour±10 minutes.

Preferably, the anti-TTR antibody (e.g., ALXN2220) is administered to the patient intravenously (IV) in the form of a pharmaceutical formulation at 50 mg/ml using an infusion, e.g., syringe.

### Pharmaceutical Compositions

The methods of treating or preventing a disease associated with ATTR (e.g., ATTR, ATTR-CM, ATTR-PN, FAP, FAC, SSA, systemic familial amyloidosis, CNS, Alzheimer disease, TTR-related ocular amyloidosis, TTR-related renal amyloidosis, TTR-related hyperthyroxinemia, TTR-related ligament amyloidosis, carpal tunnel syndrome, rotator cuff tears, lumbar spinal stenosis, or preeclampsia) described herein may utilize any anti-TTR antibody as described herein that is formulated into a pharmaceutical composition. For example, a pharmaceutical composition containing the anti-TTR antibody may be formulated with sucrose, polysorbate 80, and/or a polar excipient, e.g., a buffer agent (e.g., histidine). Furthermore, the pharmaceutical composition containing the anti-TTR antibody may be formulated at a desired pH described herein (e.g., pH 5.8). The pharmaceutical composition containing the anti-TTR antibody may further include a pharmaceutically acceptable excipient or diluent, as described herein.

The pharmaceutical composition may also include sucrose, for example, in an amount of about 6% to about 9%, about 6% to about 7%, or about 7.5% to about 8.5% weight per volume (w/v) (e.g., about 6%, 6.5%, 7%, 7.5%, 8%, 8.5%, or 9% weight per volume (w/v) sucrose).

The pharmaceutical composition may also include polysorbate 80 (PS80), for example, in an amount of about 0.001% to about 0.1% w/v (e.g., about 0.001%, 0.005%, 0.01%, 0.05% or 0.1% w/v PS80).

The pharmaceutical composition may also include a polar excipient. The polar excipient may be or include, for example, a sugar, a polyol, or an amino acid. The sugar may be, for example, sucrose, trehalose, fructose, lactose, dextrose, or mannitol. The polyol may be, for example, polyethylene glycol or sorbitol. The amino acid may be, for example, one or more of alanine, arginine, aspartic acid, asparagine, carnitine, citrulline, ornithine, glycine, glutamic acid, glutamine, glycine, histidine, isoleucine, leucine, lysine, methionine, phenylalanine, proline, serine, threonine, tyrosine, and valine. In some embodiments, the polar excipient is histidine (e.g., L-histidine and/or L-histidine monohydrochloride, or a pharmaceutically acceptable salt thereof). For example, the polar excipient is L-histidine and/or L-histidine monohydrochloride, or a pharmaceutically acceptable salt thereof.

The pharmaceutical composition may include a polar excipient (e.g., histidine) in an amount of about, for example, about 1 mM to about 100 mM (e.g., about 10 mM, 20 mM, 30 mM, 40 mM, 50 mM, 60 mM, 70 mM, 80 mM, mM, 90 mM, or 100 mM).

The pharmaceutical composition may have a pH of from about 5.0 to about 8.0 (e.g., about 5.5, 6.0, 6.5, 7.0, 7.5, or 8.0).

The pharmaceutical composition may contain a pharmaceutically acceptable excipient (e.g., a buffer, carrier, stabilizer, or preservative) or diluent (e.g., saline and aqueous buffer solutions). The pharmaceutical composition thereof may be provided (e.g., in a vial or other container, as described herein) as an aqueous solution in a volume of about 1 mL to about 200 mL, about 1 mL to about 150 mL, about 1 mL to about 100 mL, about 1 mL to about 50 mL, about 5 mL to about 25 mL, about 18 mL to about 22 mL, or about 1 mL to about 10 mL (e.g., about 1 mL to about 2 mL, about 1 mL to about 1.8 mL, about 1 mL to about 1.6 mL, about 1 mL to about 1.4 mL, about 1 mL to about 1.2 mL, about 1.5 mL to about 1.25 mL, about 1.5 mL to about 2 mL, about 1.9 mL to about 1.2 mL, about 2.1 mL to about 2.25 mL) or about 1 mL to about 100 mL (e.g., about 1 mL, about 1.8 mL, about 1.9 mL, about 2 mL, about 2.1 mL, about 2.2 mL about 2.25 mL, about 2.3 mL, about 2.4 mL, about 2.5 mL about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 40 mL, about 50 mL, about 60 mL, about 70 mL, about 80 mL, about 90 mL, or about 100 mL).

The pharmaceutical composition may be any pharmaceutical composition described in the patent application entitled Pharmaceutical Compositions for Treating or Preventing Transthyretin-Mediated Amyloidosis, filed on Nov. 15, 2022, and has the application number EP 22 207 645.7 and the attorney docket number NE30A100/P-EP (herein incorporated by reference).

In one example, the pharmaceutical composition containing the antibody is formulated for intravenous or infusion injection and contains the antibody at a concentration of about 50 mg/m or 100 mg/mL. The pharmaceutical composition includes 6.5% or 8% weight per unit volume (w/v) sucrose, 0.03% w/v polysorbate 80, 20 mM histidine, and a pH of 5.8.

In one example, the pharmaceutical composition containing the antibody is formulated for intravenous or infusion injection and contains the antibody at a concentration of about 50 mg/mL. The pharmaceutical composition includes 6.5% weight per unit volume (w/v) sucrose (65 mg/ml sucrose), 0.03% w/v polysorbate 80 (0.3 mg/ml polysorbate 80), 20 mM histidine (L-Histidine 1.06 mg/mL and L-Histidine monohydrochloride 2.78 mg/mL), and a pH of 5.8. The composition can be present in a container at a volume of 2 mL.

In another example, the pharmaceutical composition containing the antibody is formulated for intravenous or infusion injection and contains the antibody at a concentration of about 50 mg/mL. The pharmaceutical composition includes 6.5% weight per unit volume (w/v) sucrose, 0.03% w/v polysorbate 80, 20 mM histidine, and a pH of 5.8. The composition can be present in a container at a volume of 20 mL.

In another example, the pharmaceutical composition containing the antibody is formulated for intravenous injection or infusion and contains the antibody at a concentration of about 50 mg/mL. The pharmaceutical composition includes 8% w/v sucrose (80 mg/ml sucrose), 0.03% w/v polysorbate 80 (0.3 mg/ml polysorbate 80), 20 mM histidine (L-Histidine 1.06 mg/mL and L-Histidine monohydrochloride 2.78 mg/mL), and a pH of 5.8. The composition can be present in a container at a volume of 2 mL.

In another example, the pharmaceutical composition containing the antibody is formulated for intravenous injection or infusion and contains the antibody at a concentration of about 50 mg/mL. The pharmaceutical composition includes 8% w/v sucrose, 0.03% w/v polysorbate 80, 20 mM histidine, and a pH of 5.8. The composition can be present in a container at a volume of 20 mL.

In another example, the pharmaceutical composition containing the antibody is formulated for intravenous or infusion injection and contains the antibody at a concentration of about 100 mg/mL. The pharmaceutical composition includes 6.5% weight per unit volume (w/v) sucrose, 0.03% w/v polysorbate 80, 20 mM histidine, and a pH of 5.8. The composition can be present in a container at a volume of 2 mL.

In another example, the pharmaceutical composition containing the antibody is formulated for intravenous or infusion injection and contains the antibody at a concentration of about 100 mg/mL. The pharmaceutical composition includes 6.5% weight per unit volume (w/v) sucrose, 0.03% w/v polysorbate 80, 20 mM histidine, and a pH of 5.8. The composition can be present in a container at a volume of 20 mL.

In another example, the pharmaceutical composition containing the antibody is formulated for intravenous injection or infusion and contains the antibody at a concentration of about 100 mg/mL. The pharmaceutical composition includes 8% w/v sucrose, 0.03% w/v polysorbate 80, 20 mM histidine, and a pH of 5.8. The composition can be present in a container at a volume of 2 mL.

In another example, the pharmaceutical composition containing the antibody is formulated for intravenous injection or infusion and contains the antibody at a concentration of about 100 mg/mL. The pharmaceutical composition includes 8% w/v sucrose, 0.03% w/v polysorbate 80, 20 mM histidine, and a pH of 5.8. The composition can be present in a container at a volume of 20 mL.

### EXAMPLES

### Example 1. A Phase 1, First-in-Human, Double-Blind, Placebo-Controlled, Multicenter, Single and Multiple Ascending Dose Study of NI006 in Patients with Amyloid Transthyretin Cardiomyopathy Followed by an Open-Label Extension

### Overall Design

This example describes a randomized, placebo-controlled, double-blind trial combining a single-ascending dose (SAD) phase and multiple-ascending dose (MAD) phase, followed by an open-label extension OLE phase in subjects with amyloid transthyretin (ATTR)-cardiomyopathy (CM). The study is designed as the first-in-human investigation of the safety, tolerability and exploratory efficacy profile of single and multiple doses of antibody NI006, i.e., an anti-TTR antibody having a heavy chain variable (VH) region having an amino acid sequence of SEQ ID NO: 7 and a light chain variable (VL) region having an amino acid sequence of SEQ ID NO: 8, from 0.3 mg/kg up to 60 mg/kg. NI006 is a recombinant human anti-ATTR monoclonal IgG1 antibody that was generated based on a comprehensive immune repertoire analysis of memory B-cell complements of healthy elderly human subjects as described for example in WO 2015/092077 A1 (U.S. Pat. Nos. 10,344,080; and 11,180,545), where the antibody is named NI-301.37F1.

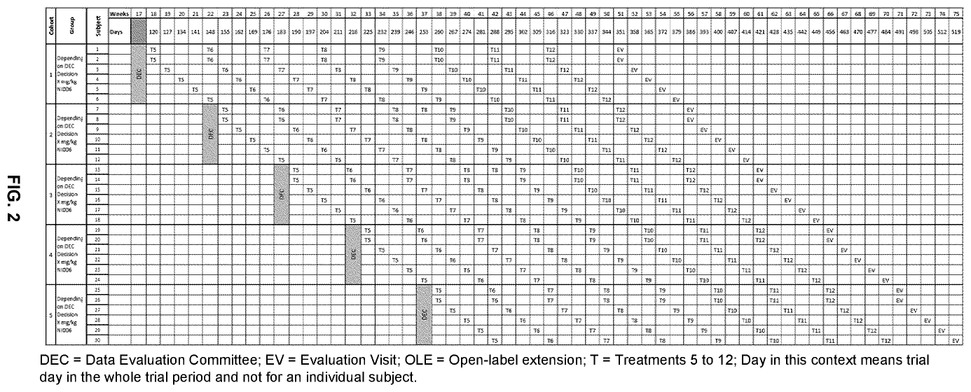

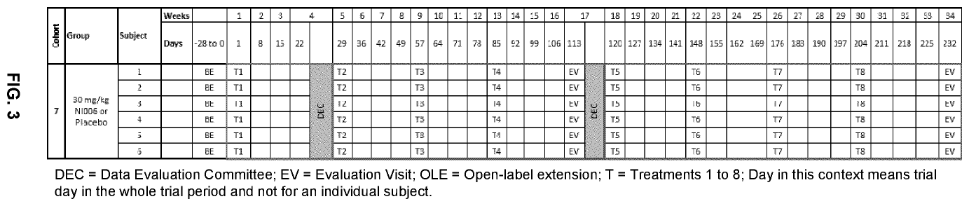

This trials objects and endpoint are presented in Table 2. NI006 or placebo (e.g., a control) will be administered to subjects during each phase. Subjects completing the SAD phase will be enrolled in the MAD phase. Subjects completing the MAD phase will have the possibility to continue in an OLE phase with dose up-titrations. A second open-label extension (OLE2) phase is optional to allow longer-term treatment at a maximum dose of 30 mg/kg or lower. There are several exemplary cohorts (e.g., cohort 1 through 7) of subjects described herein, each receiving varying dosages of the anti-TTR antibody or placebo. Exemplary dosages of the anti-TTR antibody or placebo in milligram per kilogram (mg/kg) body weight are: 0.3 mg/kg (cohort 1), 1 mg/kg (cohort 2), 3 mg/kg (cohort 3), 10 mg/kg (cohort 4), 30 mg/kg (cohorts 5 and 7), and 60 mg/kg (cohort 6). An exemplary trial schema is provided in FIG. 1 (e.g., SAD/MAD Cohorts 1-6), FIG. 2 (OLE Cohorts 1-6), and FIG. 3 (SAD/MAD and OLE cohort 7).

Subjects of cohorts 1 to 5 have the possibility for a second OLE phase (OLE2) by up to 10 months of treatment at a maximal dose of 30 mg/kg.

The detailed study design as performed in this dose study as finally performed is described in the following:

The first 2 patients in each dose cohort (sentinels) were randomized 1:1 to receive NI006 or placebo. The 4 subsequent patients in each cohort were randomized in a 3:1 ratio to NI006 or placebo if no relevant safety signals occurred in the sentinels. After favorable review of sufficient SAD safety data, the next higher dose cohort was opened. Patients received a total of 4 administrations q4w of NI006 or placebo during the combined SAD/MAD phase. At baseline and at 4 months, cardiac imaging was performed to evaluate changes in NI006 and placebo during the SAD/MAD phase. In case of discontinuation during the SAD/MAD phase for reasons other than suspected drug-toxicity, replacement patients were recruited. Patients rolling over into the OLE phase were entitled to receive 8 administrations of NI006 q4w, irrespective of the treatment assignment in the SAD/MAD phase, which remained blinded at that time. As new safety information became available from the SAD/MAD phase of higher dose cohorts, patients who started on lower dose levels were up-titrated to the maximal safe dose level at the time of each dosing. At the end of the OLE phase, at 12 months, cardiac imaging was performed.

NI006 was administered as IV infusion over approximately 2 hours (±10 minutes; except for up to 3 hr at 60 mg/kg) at first infusion. Subsequent infusions were administered over approximately 50-70 minutes. Patients were hospitalized for 4 nights after the SAD administration and the first OLE administration (i.e., first NI006 administration in patients randomized to placebo), and for 1 to 2 nights after each of the three MAD administrations. All further administrations in the OLE phase were performed as outpatient visits.

TABLE 2
Trial Objectives and Endpoints
Single-Ascending Dose (SAD), Multiple-Ascending
Dose (MAD) and Open-Label Extension (OLE) Phases:

| Primary Objective | Endpoints |
|---|---|
| Determine the short and longer- | Number and proportion of treatment |
| term safety and tolerability of | emergent adverse events (TEAEs). |
| single and multiple doses of the | Number and proportion of SAEs. |
| antibody in subjects with | Changes in clinical laboratory |
| ATTR-CM by evaluating | parameters (continuous parameters). |
| adverse events (AE) and serious | Change from baseline in cardiac |
| adverse events (SAE) and | biomarkers. |
| changes in laboratory | Changes by visit for vital signs. |
| parameters (hematology, clinical | Changes by visit for ECG parameters |
| chemistry, immunology, and | (continuous parameters). |
| urinalysis), cardiac biomarkers, | Summary of echocardiogram results. |
| vital signs, electrocardiogram | |
| (ECG), and echocardiogram. | |
| Secondary Objective | Endpoints |
| Determine the pharmacokinetic | PK parameters for the SAD phase: |
| (PK) profile and PK parameters | Maximum observed serum |
| of the antibody in subjects with | concentration (Cmax), time to |
| ATTR-CM. | maximum observed serum |
| | concentration (Tmax), area under the |
| | serum concentration-time curve from |
| | zero to infinity (AUCinf), serum |
| | clearance (CL), apparent volume of |
| | distribution during terminal phase (Vz), |
| | apparent volume of distribution at |
| | steady state (Vss), and/or terminal |
| | elimination half-life (t½). |
| | PK parameters for the MAD phase: |
| | Cmax, Tmax, area under the serum |
| | concentration-time curve from time |
| | zero to the end of the dosing interval |
| | after the first dose (AUC_{T}), CL, Vz, |
| | Vss, t½, accumulation ratio for |
| | maximum concentration (RaccCmax), |
| | and/or accumulation ratio calculated |
| | from AUC (RaccAUC). |
| | PK parameters for the OLE and OLE2 |
| | phase: |
| | Minimum observed serum |
| | concentration (Ctrough), dose- |
| | normalized Ctrough, and/or |
| | accumulation ratio compared to SAD/ |
| | MAD calculated from dose-normalized |
| | Ctrough. |
| Exploratory Objectives | Endpoints |
| Explore the efficacy of multiple | 6-Minute Walk Test (6-MWT). |
| doses of the antibody in | Kansas City Cardiomyopathy |
| subjects with ATTR-CM on | Questionnaire (KCCQ). |
| clinical, imaging and laboratory | Echocardiography (contractile function |
| variables. | [strain], thickness and filling pressure, |
| Confirm absence of effect on | left ventricular ejection fraction |
| physiological TTR. | (LVEF). |
| Evaluate the immunogenicity of | Estimation of amyloid load by imaging |
| the antibody. | method in a subset of subjects. |
| Characterize target engagement | Changes in biomarkers. |
| and immune activation on | Determination of TTR, free thyroxine |
| available tissue samples. | (T4), Vitamin A levels and Retinol- |
| | binding protein (RBP). |
| | Determination of anti-drug antibody |
| | (ADA) response. |
| | Immunohistochemistry (IHC) of tissue |
| | samples in a subset of subjects as |
| | available. |

### Patient Population

40 patients with wild-type or hereditary ATTR-CM (median age 72 years, 97.5% male), chronic heart failure and baseline NT-proBNP between of 766 and 5892 μg/mL were enrolled in this study. More particularly, the study population was comprised of patients with a confirmed diagnosis of ATTR-CM established according to current guidelines (Garcia-Pavia et al., Eur Heart J. 42 (2021), 1554-68; Kittleson et al., Circulation 142 (2020), e7-e22), irrespective of the underlying genotype (ATTRv and ATTRwt), with a left ventricular wall thickness ≥14 mm, left ventricular ejection fraction (LVEF)≥40%, NYHA stages I to III, NT-proBNP between 600 pg/mL and 6000 pg/mL, and eGFR>30 mL/min/1.73 m2. Concomitant treatment with tafamidis was accepted if the dose was stable for at least 30 days prior to screening. Treatment with other ATTR-specific drugs, including gene silencers was not permitted. Patients were recruited at 6 specialized amyloidosis centers in 4 European countries (Germany, France, Spain, Netherlands). The demographic and clinical characteristics of the patients are shown in Table 3.

TABLE 3
Demographic and Clinical Characteristics of the Patients at Baseline

| | NI006 | | Placebo | |
|---|---|---|---|---|
| Characteristic | (N = 27) | | (N = 13) | |
| Age—yr | | | | |
| Mean | 73 ± 8 | | 68 ± 14 | |
| Median (range) | 74 (70-77) | | 68 (67-74) | |
| Sex—no (%) | | | | |
| Male | 26 | (96.3) | 13 | (100) |
| Female | 1 | (3.7) | 0 | |
| TTR genotype—no (%) | | | | |
| ATTRV | 4 | (14.8) | 3 | (23.1) |
| ATTRwt | 23 | (85.2) | 10 | (76.9) |
| Weight—kg | | | | |
| Mean | 85 ± 10 | | 80 ± 12 | |
| Median (range) | 85 (79-92) | | 75 (71-80) | |
| Ethnicity | | | | |
| Asian | 0 | | 1 | |
| Caucasian | 14 | | 7 | |
| Not reported* | 13 | | 5 | |
| NYHA Class—no (%) | | | | |
| Class I | 3 | (11.1) | 3 | (23.1) |
| Class II | 19 | (70.4) | 10 | (76.9) |
| Class III | 5 | (18.5) | 0 | |
| NT-proBNP level—pg/ml | | | | |
| Mean | 2551 ± 1549 | | 1840 ± 876 | |
| Median | 2029 | | 1591 | |
| Interquartile range | (1433-3674) | | (1310-2107) | |
| >3000 pg/mL—no (%) | 9 | (33.3) | 1 | (7.7) |
| Troponin T | | | | |
| Mean | 78 ± 131 | | 43 ± 34 | |
| Median | 52 | | 33 | |
| Interquartile range | (38-71) | | (22-43) | |
| >50 pg/mL—no (%) | 14 | (51.9) | 3 | (23.1) |
| Estimated GFR | | | | |
| Mean | 65 ± 22 | | 75 ± 21 | |
| Median | 63 | | 79 | |
| Interquartile range | (44-86) | | (62-86) | |
| <45 ml/min * 1.73 m2 | 8 | (29.6) | 1 | (7.7) |

*At French trial sites, collection of ethnical background information was not permitted.
Plus-minus values are means ± standard deviation.
NT-proBNP denotes N-terminal pro-B-type natriuretic peptide,
GFR glomerular filtration rate, and
NYHA New York Heart Association.

### SAD Phase for Cohorts 1 to 6

The treatment regimen described herein begins on the first day the anti-TTR antibody is administered to a subject, which will be considered day 1 of the treatment regimen. Day 1 to day 29 of the treatment regimen is considered the SAD phase for cohorts 1 to 6 (e.g., see FIG. 1, red boxes). Subjects from cohorts 1 to 6 will receive treatment with the anti-TTR antibody or a placebo on day 1 of the SAD phase. Cohort 1 will receive 0.3 mg/kg of the anti-TTR antibody or a placebo on day 1 of the SAD phase. Cohort 2 will receive 1 mg/kg of the anti-TTR antibody or a placebo on day 1 of the SAD phase. Cohort 3 will receive 3 mg/kg of the anti-TTR antibody or a placebo on day 1 of the SAD phase. Cohort 4 will receive 10 mg/kg of the anti-TTR antibody or a placebo on day 1 of the SAD phase. Cohort 5 will receive 30 mg/kg of the anti-TTR antibody or a placebo on day 1 of the SAD phase. Cohort 6 will receive 60 mg/kg of the anti-TTR antibody or a placebo on day 1 of the SAD phase.

### MAD Phase for Cohorts 1 to 6

Day 30 to day 148 of the treatment regimen is considered the MAD phase (e.g., see FIG. 1, blue boxes). Subjects from cohort 1 to 6 completing the SAD phase will continue in the MAD phase of the trial, which includes 3 additional administrations of the anti-TTR antibody or a placebo starting 35 days after the subject's first treatment with the anti-TTR antibody.

For example, the anti-TTR antibody or a placebo will be administered to the subject at the subject's assigned dose (e.g., based on their cohort) on day 36 of the treatment regimen, followed by an inter-treatment regimen of 28±2 days. For example, cohort 1 will receive 0.3 mg/kg of the anti-TTR antibody or a placebo on day 36, on any one of days 62-66 (e.g., day 64), and on any one of days 90-94 (e.g., day 92) of the treatment regimen; cohort 2 will receive 1 mg/kg of the anti-TTR antibody or a placebo on day 36, on any one of days 62-66 (e.g., day 64), and on any one of days 90-94 (e.g., day 92) of the treatment regimen; cohort 3 will receive 3 mg/kg of the anti-TTR antibody or a placebo on day 36, on any one of days 62-66 (e.g., day 64), and on any one of days 90-94 (e.g., day 92) of the treatment regimen; cohort 4 will receive 10 mg/kg of the anti-TTR antibody or a placebo on day 36, on any one of days 62-66 (e.g., day 64), and any one of days 90-94 (e.g., day 92) of the treatment regimen; cohort 5 will receive 30 mg/kg of the anti-TTR antibody or a placebo on day 36, on any one of days 62-66 (e.g., day 64), and on any one of days 90-94 (e.g., day 92) of the treatment regimen; and cohort 6 will receive 60 mg/kg of the anti-TTR antibody or a placebo on day 36, on any one of days 62-66 (e.g., day 64), and on any one of days 90-94 (e.g., day 92) of the treatment regimen.

In another example, the anti-TTR antibody or a placebo will be administered to the subject at the subject's assigned dose (e.g., based on their cohort) on day 36 of the treatment regimen, followed by an inter-treatment regimen of 28±7 days. For example, cohort 1 will receive 0.3 mg/kg of the anti-TTR antibody or a placebo on day 36, on any one of days 57-71 (e.g., day 64), and on any one of days 85-99 (e.g., day 92) of the treatment regimen; cohort 2 will receive 1 mg/kg of the anti-TTR antibody or a placebo on day 36, on any one of days 57-71 (e.g., day 64), and on any one of days 85-99 (e.g., day 92) of the treatment regimen; cohort 3 will receive 3 mg/kg of the anti-TTR antibody or a placebo on day 36, on any one of days 57-71 (e.g., day 64), and on any one of days 85-99 (e.g., day 92) of the treatment regimen; cohort 4 will receive 10 mg/kg of the anti-TTR antibody or a placebo on day 36, on any one of days 57-71 (e.g., day 64), and any one of days 85-99 (e.g., day 92) of the treatment regimen; cohort 5 will receive 30 mg/kg of the anti-TTR antibody or a placebo on day 36, on any one of days 57-71 (e.g., day 64), and on any one of days 85-99 (e.g., day 92) of the treatment regimen; and cohort 6 will receive 60 mg/kg of the anti-TTR antibody or a placebo on day 36, on any one of days 57-71 (e.g., day 64), and on any one of days 85-99 (e.g., day 92) of the treatment regimen.

### SAD/MAD Phase for Cohort 7

The treatment regimen described herein (e.g., see FIG. 3) begins on the first day the anti-TTR antibody is administered to a subject, which will be considered day 1 of the treatment regimen. Day 1 to day 28 of the treatment regimen is considered the SAD phase for cohort 7. Subjects from cohort 7 will receive treatment with the anti-TTR antibody or a placebo on day 1 of the SAD phase. For example, cohort 7 may receive 30 mg/kg of the anti-TTR antibody or a placebo on day 1 of the SAD phase.

Day 29 to day 119 of the treatment regimen is considered the MAD phase for cohort 7. Subjects from cohort 7 completing the SAD phase will continue in the MAD phase of the trial, which includes 3 additional administrations of the anti-TTR antibody or a placebo starting 28 days after the subject's first treatment with the anti-TTR antibody.

For example, the anti-TTR antibody or a placebo will be administered to the subject at 30 mg/kg on day 29 of the treatment regimen, followed by an inter-treatment regimen of 28±2 days. For example, cohort 7 may receive 30 mg/kg of the anti-TTR antibody or a placebo on day 29, on any one of days 55-59 (e.g., day 57), and any one of days 83-87 (e.g., day 85) of the treatment regimen.

In another example, the anti-TTR antibody or a placebo will be administered to the subject at 30 mg/kg on day 29 of the treatment regimen, followed by an inter-treatment regimen of 28±7 days. For example, cohort 7 may receive 30 mg/kg of the anti-TTR antibody or a placebo on day 29, on any one of days 50-64 (e.g., day 57), and any one of days 78-92 (e.g., day 85) of the treatment regimen.

### OLE Phase for Cohorts 1 to 6

Day 120 to day 351 of the treatment regimen is considered the OLE phase (e.g., see FIG. 2). Subjects from cohort 1 to 6 completing the SAD phase will continue in the OLE phase of the trial. Subjects who received placebo during SAD and MAD phases may now receive the anti-TTR antibody during the OLE phase.

The starting dose of the anti-TTR antibody for a subject who received placebo in the SAD and MAD phases will either be at the same dose level as on the subject's SAD and MAD phases (e.g., if subject received 1 mg/kg in the SAD and MAD phases, then the subject will receive 1 mg/kg of the anti-TTR antibody as the starting dose in the OLE phase) or the highest dose of the anti-TTR antibody which at the time point of this subject's first treatment in the OLE phase is deemed safe and well tolerated (e.g., 0.3 mg/kg, 1 mg/kg, 3 mg/kg, 10 mg/kg, 30 mg/kg, or 60 mg/kg).

The OLE phase includes 8 additional administrations of the anti-TTR antibody or a placebo starting 119 days after the subject's first treatment with the anti-TTR antibody. The anti-TTR antibody or a placebo will be administered to the subject at 0.3 mg/kg, 1 mg/kg, 3 mg/kg, 10 mg/kg, 30 mg/kg, or 60 mg/kg on day 120 of the treatment regimen, followed by an inter-treatment regimen of 28±2 days or 28±7 days.

For example, cohort 7 will receive 0.3 mg/kg, 1 mg/kg, 3 mg/kg, 10 mg/kg, 30 mg/kg, or 60 mg/kg of the anti-TTR antibody or a placebo on day 120, on any one of days 146-150 (e.g., day 148), on any one of days 174-178 (e.g., day 176), on any one of days 202-206 (e.g., day 204), on any one of days 230-234 (e.g., day 232), on any one of days 258-262 (e.g., day 260), on any one of days 286-290 (e.g., day 288), and on any one of days 314-318 (e.g., day 316) of the treatment regimen.

In another example, cohort 7 will receive 0.3 mg/kg, 1 mg/kg, 3 mg/kg, 10 mg/kg, 30 mg/kg, or 60 mg/kg of the anti-TTR antibody or a placebo on day 120, on any one of days 141-155 (e.g., day 148), on any one of days 169-183 (e.g., day 176), on any one of days 197-211 (e.g., day 204), on any one of days 225-239 (e.g., day 232), on any one of days 253-267 (e.g., day 260), on any one of days 281-295 (e.g., day 288), and on any one of days 309-323 (e.g., day 316) of the treatment regimen

During the OLE phase for cohorts 1 to 6, administration of the anti-TTR antibody may be up-titrated one or more times. Up-titration of the antibody will occur at one or more inter-treatments: on any one of days 141-155 (e.g., on any one of days 146-150, e.g., day 148), on any one of days 169-183 (e.g., on any one of days 174-178, e.g., day 176), on any one of days 197-211 (e.g., on any one of days 202-206, e.g., day 204), on any one of days 225-239 (e.g., on any one of days 230-234, e.g., day 232), on any one of days 253-267 (e.g., on any one of days 258-262, e.g., day 260), on any one of days 281-295 (e.g., on any one of days 286-290, e.g., day 288), and/or and on any one of days 309-323 (e.g., 314-318, e.g., day 316).

Up-titration will include an increase from 0.3 mg/kg to 1 mg/kg, 1 mg/kg to 3 mg/kg, 3 mg/kg to 10 mg/kg, 10 mg/kg to 30 mg/kg, or 30 mg/kg to 60 mg/kg at each inter-treatment. For example, an up-titration may occur at each inter-treatment. In another example, an up-titration may occur at least one, two, three, four, five, or six times.

### OLE Phase for Cohort 7

Day 120 to day 232 of the treatment regimen is considered the OLE phase for cohort 7 (e.g., FIG. 3). Subjects from cohort 7 completing the MAD phase will continue in the OLE phase of the trial, which includes 4 additional administrations of the anti-TTR antibody or a placebo starting 119 days after the subject's first treatment with the anti-TTR antibody. For example, the anti-TTR antibody or a placebo will be administered to the subject at 30 mg/kg on day 120 of the treatment regimen, followed by an inter-treatment regimen of 28±2 days or 28±2 days later.

For example, cohort 7 will receive 30 mg/kg of the anti-TTR antibody or a placebo on day 120, on any one of days 146-150 (e.g., day 148), on any one of days 174-178 (e.g., day 176), and on any one of days 202-206 (e.g., day 204) of the treatment regimen.

In another example, cohort 7 will receive 30 mg/kg of the anti-TTR antibody or a placebo on day 120, on any one of days 141-155 (e.g., day 148), on any one of days 169-183 (e.g., day 176), and on any one of days 197-211 (e.g., day 204) of the treatment regimen.

### Expanded OLE (OLE+) Phase for Cohorts 1 to 2

If a subject from cohort 1 or cohort 2 received 1-7 doses in the OLE phase, and therefore did not complete the OLE phase, the subject will enter an expanded OLE (OLE+) phase. The OLE+ phase includes one or two additional administrations of the anti-TTR antibody starting 28±2 days since their last treatment with the anti-TTR antibody. For example, the anti-TTR antibody will be administered to the subject at 0.3 mg/kg, 1 mg/kg, 3 mg/kg, 10 mg/kg, 30 mg/kg, or 60 mg/kg 28±2 days since their last treatment with the anti-TTR antibody, followed by one additional treatment 28±2 days later or 28±7 days later. Up-titration of the anti-TTR antibody is possible, such as an increase from 0.3 mg/kg to 1 mg/kg, 1 mg/kg to 3 mg/kg, 3 mg/kg to 10 mg/kg, 10 mg/kg to 30 mg/kg, or 30 mg/kg to 60 mg/kg. For example, an up-titration may occur during the first administration of the OLE+ phase, the second administration of the OLE+ phase, or both.

### OLE2 Phase for Cohorts 1 to 5

Subject from any one of cohorts 1 to 5 receiving at least one (e.g., 1-7) dose in the OLE or OLE+ phase will enter an OLE2 phase, which includes up to 8 additional administrations of the anti-TTR antibody. Subjects will be treated with a maximal dose of 30 mg/kg in OLE2. Subjects that were treated with a dose lower than 30 mg/kg before starting OLE2 will have to start OLE2 with the last dose level at which they were treated in the OLE or OLE+ phase and can be up-titrated step-wise from one dose level to another dose level on a monthly (e.g., 28±2 days or 28±7 days) basis (e.g., if a subject's previous dose was 3 mg/kg, then the subject will have to start OLE2 at that dose, which can then be up-titrated to 10 mg/kg at the second OLE2 treatment and then to 30 mg/kg at the third OLE2 treatment). Up-titration will include an increase from 0.3 mg/kg to 1 mg/kg, 1 mg/kg to 3 mg/kg, 3 mg/kg to 10 mg/kg, or 10 mg/kg to 30 mg/kg at each inter-treatment. For example, if a subject had no interruption between OLE and OLE2 and was already at a dose of 10 mg/kg or higher at the end of OLE, the subject can be directly up-titrated to/dosed at 30 mg/kg. Up-titration may occur at each inter-treatment. In other instances, an up-titration may occur at least one, two, three, four, five, six, or seven times.

### Administration

The anti-TTR antibody will be administered intravenously using a dosing syringe in a syringe pump or an infusion bag. An aqueous formulation of the anti-TTR antibody should be brought to room temperature before use. Immediate use after opening a single-use vial containing anti-TTR antibody is recommended. The anti-TTR antibody should be clear or slightly opalescent and colorless or slightly yellow. Aqueous anti-TTR antibody formulations which are cloudy or with deposits must not be used.

A subject's initial dose (e.g., day 1 of the treatment regimen) will be delivered over approximately 2 hours (±10 minutes). A subject's initial dose in Cohort 6 will be delivered up to a maximum duration of 3 hours. If the first infusion is tolerated without a hypersensitivity-associated adverse event, subsequent infusions will be delivered over approximately 1 hour (±10 minutes) for cohort 1 to 5, or 60-70 minutes for cohort 6 and 7.

A subject's initial OLE dose (e.g., day 120 of the treatment regimen) will be delivered over approximately 2 hours (±10 minutes). A subject's initial OLE dose in Cohort 6 can be delivered up to a maximum duration of 3 hours.

A subject's initial dose in the OLE2 phase will be delivered over approximately 2 hours (±10 minutes). All subsequent infusions can be delivered over approximately 1 hour (±10 minutes).

In case of an infusion site reaction or report of similar adverse event the infusion duration of any infusion may be increased up to 3 hours.

End of infusion (EOI) is defined as completing the whole anti-TTR infusion plus flushing the entire infusion line.

An adverse event is defined as any untoward medical occurrence in a subject administered a pharmaceutical product (e.g., an anti-TTR antibody) and which does not necessarily have a causal relationship with this treatment. An adverse event can therefore be any unfavorable or unintended sign (including an abnormal laboratory finding), symptom, or disease temporally associated with the use of a medicinal product (e.g., an anti-TTR antibody, whether or not related to the medicinal product. The adverse event may be any of the following:
A new illness;
An exacerbation of a sign or symptom or the underlying condition or of a concomitant illness under treatment;
Unrelated to participation in the clinical trial or an effect of the medicinal product (e.g., anti-TTR antibody) or comparator drug;
A combination of 1 or more of the above factors

### Dose-Limiting Toxicity

The dose limiting toxicity (DLT) period for safety assessment is defined as 28 calendar days after the subject's first infusion with the anti-TTR antibody.

The DLT is defined as an adverse reaction (based on the National Cancer Institute [NCI] Common Terminology Criteria for Adverse Events (CTCAE) v5.0 (e.g., Table 4) captured within 28 days of a subject's first dosing and fulfilling any of the below defined criteria.

The DLTs will include:
1. Hypersensitivity reaction including cytokine release syndrome Grade 3 and higher;
2. Injection site reaction Grade 3 or higher; or
3. New/acute onset of the following symptoms that are most likely attributable to cardiac inflammation because of the exposure to the anti-TTR antibody:
   Any of the below:
      Sinus tachycardia Grade 3 and higher
      Arrhythmia (atrial fibrillation, supraventricular tachycardia) Grade 3 or higher
      Mobitz II
      Signs and symptoms of myocarditis/cardiac inflammation Grade 3 or higher
         New onset of acute heart failure
         Syncope
         Cardiogenic shock
         Dyspnea
      Significant ECG abnormalities (ST elevation, complete atrioventricular (AV) block, ventricular tachycardia); and
   any of the following:
   a) A sudden drop in LVEF by more than 10 points from baseline with an absolute value <40%; and/or
   b) Increase in troponins (increase by 100% from the previous value in the absence of acute renal failure).

Cardiac toxicity should be assessed in great detail and with special attention to the findings and/or its severity that are unexpected and unpredictable in the context of subject's disease history and concomitant medication.

Any other procedure assumed as necessary by a treating physician may be performed to clarify the presence of a DLT.

TABLE 4
Common Terminology Criteria for Adverse Events [CTCAE] v5.0

| | Common Terminology Criteria |
|---|---|
| Grade | for Adverse Events (CTCAE) v5.0 |
| Grade 1 | Mild; asymptomatic or mild symptoms; clinical or diagnostic |
| | observations only; intervention not indicated. |
| Grade 2 | Moderate; minimal, local or noninvasive intervention |
| | indicated; limiting age appropriate instrumental Activities of |
| | Daily Living (ADL)*. |
| Grade 3 | Severe or medically significant but not immediately life- |
| | threatening; hospitalization or prolongation of hospitalization |
| | indicated; disabling; limiting self-care ADL**. |
| Grade 4 | Life-threatening consequences; urgent intervention indicated. |
| Grade 5 | Death related to AE. |

ADL = Activities of Daily Living;
*Instrumental ADL refer to preparing meals, shopping for groceries or clothes, using the telephone, managing money, etc;
**Self-care ADL refer to bathing, dressing and undressing, feeding self, using the toilet, taking medications, and not bedridden.

The following are guidelines for the treatment of infusion-related reactions:
Mild Reaction (flushing, mild headache, nausea, mild discomfort, mild weakness)
   Slow infusion to ½ of previous rate (maximum infusion time of 3 hours cannot be exceeded)
   Assess vital signs (blood pressure, pulse rate, oxygen saturation [SpO2]) every 15 minutes
   Observe 30 minutes
   If reaction resolving, begin increasing rate every 15 minutes until reaching original target rate
      If reaction occurs again, reduce infusion to highest previously tolerated rate
      If the reaction persists or worsens see Moderate Reaction below.
Moderate Reaction (fever, severe headache, wheezing, urticaria, SpO2<95%, vomiting)
   Stop infusion
   Withdraw 3 mL of blood from cannula (dispose of blood)
   Connect new 0.9% NaCl infusion bag with new tubing to cannula and initiate infusion at maintenance rate per subject weight, per local guidelines
   Assess vital signs every 5 minutes (blood pressure, pulse rate, SpO2)
      Start supplemental 02 (mask or nasal according to subject age and local guidelines) if SpO2<95%
   Give diphenhydramine 1 mg/kg body weight IV (or other antihistamine according to hospital practice and recommended dose)
   Give acetaminophen 500 mg orally (or other non-steroidal anti-inflammatory drug [NSAID] according to hospital practice and recommended dose) for fever or headache
   Additional medications recommended to be used if necessary and as needed (or equivalents/similar medications according to hospital practice and recommended dose):
      Hydrocortisone 2 mg/kg body weight IV
      Ondansetron 4 mg IV for nausea and vomiting
      Albuterol 5 mg inhalation for wheezing
   If reaction resolving:
      Wait 30 minutes after IV medications given, then restart infusion at ½ rate at which infusion reaction occurred (maximum total infusion time of 3 hours cannot be exceeded)
      Increase rate every 15 minutes (as tolerated) until reaching original target rate. If reaction occurs again, repeat reaction procedure and increase only to highest previously tolerated rate
   If reaction persists:
      Consider additional medications listed above
   If reaction worsens:
      See Section ‘Severe Reaction’ below
Severe Reaction (anaphylaxis)

Anaphylaxis is a life-threatening medical emergency and should always be handled according to local guidelines and procedures to ensure the quickest, safest and most efficient resolution of the reaction. The following are general recommendations:
Stop infusion
Withdraw 3 mL of blood from cannula (dispose of blood)
Connect new 0.9% NaCl infusion bag with new tubing to cannula and initiate infusion at maintenance rate per subject weight, per local guidelines
Assess vital signs every 5 minutes (blood pressure, pulse rate, SpO2)
   Start supplemental 02 (mask or nasal according to subject age and local guidelines) if SpO2<95%
Give epinephrine 0.01 mg/kg body weight (using 1:1000 dilution or 1 mg/mL, maximum 0.5 mL) intramuscular
Give diphenhydramine 1 mg/kg body weight IV (or other antihistamine according to hospital practice and recommended dose)
Additional medications recommended to be used if necessary and as needed (or equivalents/similar medications according to hospital practice and recommended dose):
   Hydrocortisone 2 mg/kg body weight IV
   Ondansetron 4 mg IV for nausea and vomiting
   Albuterol 5 mg inhalation for wheezing
   Acetaminophen 500 mg PO (or other NSAID according to hospital practice and recommended dose) for fever or headache
Reaction resolving:
   Do not restart infusion
   Monitor as medically indicated
Reaction persists or worsens:
   Repeat above medications as needed and according to label/hospital practice
   Continue treating until reaction resolves and then monitor as medically indicated.

### Infusion Pre-Medications

In case subjects experience any type of infusion-related reaction during the trial, premedication 30-60 minutes prior to infusion with antihistamines (e.g., diphenhydramine) and/or antipyretics (e.g., acetaminophen), and/or corticosteroids (e.g., hydrocortisone 2-4 mg/kg body weight, or equivalent) is recommended. Because the specific medications used may vary from country to country and institution to institution, this is left to the discretion of the medical professional. As an example, it is recommended to give diphenhydramine and acetaminophen prior to infusion in any subject who previously had even a mild infusion reaction.

### Efficacy Assessments

The following efficacy assessments may be performed for this trial as exploratory read-outs.

### 6-Minute Walk Test

The 6-minute walt test (6-MWT) is a sub-maximal exercise test used to assess walking endurance and aerobic capacity. Subjects will walk around the perimeter of a set circuit for a total of 6 minutes. The score of the test is the distance a subject walks on a flat, hard surface in a period of 6 minutes (measured in meters and can be rounded up to the nearest decimal point).

### Kansas City Cardiomyopathy Questionnaire

The Kansas City Cardiomyopathy Questionnaire (KCCQ) is a 23-item self-administered questionnaire developed to independently measure the subject's perception of their health status, which includes heart failure symptoms, impact on physical and social function, and how their heart failure impacts their quality of life within a 2-week recall period.

The KCCQ tool quantifies the following 6 distinct domains and 2 summary scores:
KCCQ Symptom Domain quantifies the frequency and burden of clinical symptoms in heart failure, including fatigue, shortness of breath, paroxysmal nocturnal dyspnea and subjects' edema/swelling. An overall symptom score is generally used in analyses; subscale scores for both frequency and severity are also available.
KCCQ Physical Function Domain measures the limitations subjects experience, due to their heart failure symptoms, in performing routine activities. Activities are common, gender-neutral, and generalizable across cultures, while also capturing a range of exertional requirements.
KCCQ Quality of Life Domain is designed to reflect subjects' assessment of their quality of life, given the current status of their heart failure.
KCCQ Social Limitation Domain quantifies the extent to which heart failure symptoms impair subjects' ability to interact in a number of gender-neutral social activities.
KCCQ Self-efficacy Domain quantifies subjects' perceptions of how to prevent heart failure exacerbations and manage complications when they arise. This scale is not included in the summary scores.
KCCQ Symptom Stability Domain measures recent changes in subjects' symptoms; their shortness of breath, fatigue or swelling. It compares subject's frequency of heart failure symptoms at the time of completing the KCCQ with their frequency 2 weeks ago. As a measure of change, it is most interpretable as a baseline assessment of the stability of subjects' symptoms at the start of a trial and shortly thereafter, as a measure of the acute response to treatment. This domain is not included in the summary scores.
Clinical Summary Score includes total symptom and physical function scores to correspond with NYHA Classification.
Overall Summary Score includes the total symptom, physical function, social limitations and quality of life scores.

### Echocardiography

A full echocardiogram to record the contractile function (strain), thickness and filling pressure, and LVEF may be performed. Central echocardiography may contain:
Left ventricular end-diastolic/end-systolic diameter and systolic function (LVEF by Simpson), left ventricular end systolic volume (LVESV), left ventricular end diastolic volume (LVEDV);
Left ventricular diastolic function—transmitral flow-mitral E/A ratio, E/e′, maximal left atrial volume or left atrial volume index;
Right ventricular function—TAPSE and trans-tricuspid regurgitation systolic gradient
Global longitudinal strain; and/or
End diastolic-interventricular septum (ED-IVS) and End diastolic-posterior wall (ED-PW).

### Magnetic Resonance Imaging, Bone Scintigraphy

One of the following assessments may be performed per subject.
Magnetic resonance imaging (MRI) in a subset of subjects including:
   Morphological (e.g., LV mass) and functional assessments (e.g., LV ejection fraction, global longitudinal strain);
   Native T1 mapping, assessment of extracellular volume after Gadolinium administration; and
   T2 (inflammation).
Bone scintigraphy using DPD or HMDP in a subset of subjects including:
   Heart retention (HR)/whole-body retention (WBR) ratio and
   Heart retention (HR)/skull retention (SR) ratio
   The data of these imaging methods will be sent for central reading to the central reading provider.

### Biomarkers

Biomarkers may be evaluated at pre-infusion and at specific timepoints after infusion during the course of the trial. Serum samples (approximately 12 mL blood per sample) may be collected during this trial and may be used for the analysis of novel biomarkers to identify the influence on the course of amyloidosis or other medically related conditions as well as the clinical and biological responses to the anti-TTR antibody.

The biomarkers during SAD, MAD and OLE phases may include, but not limited to:
Indicative for cardiac function and cardiac stress:
   Plasma NT-proBNP
   Troponin-T
Acute phase proteins, complement activation and inflammation markers:
   C3, C4 and C-reactive protein (CRP)
   IL1b, IL6, IL8, IFNg, TNF-α (Pro-inflammatory cytokines)
   IL10, IL1RA (Anti-inflammatory cytokines)
   Serum amyloid A (SAA), Ferritin (positive acute phase proteins)
      The biomarkers during OLE2 may include, but are not limited to:
Circulating levels of ATTR
Indicative for cardiac function, cardiac stress
   Plasma NT-proBNP, Troponin T or Galectin 3 (Gal-3), soluble suppression of tumorigenicity 2 (sST2)
Cardiac fibrosis biomarkers:
   Carboxy-terminal propeptide of procollagen type 1 (PICP)
   Propeptide of procollagen type III (PIIINP)
Acute phase proteins, complement activation and inflammation markers:
   C3, C4 and C-reactive protein (CRP)
   IL1b, IL6, IL8, IFNg, TNF-α (Pro-inflammatory cytokines)
   IL10, IL1RA (Anti-inflammatory cytokines)
   Serum amyloid A (SAA), Ferritin (positive acute phase proteins)

### Immunohistochemistry of Tissue Samples

The immunohistochemistry (IHC) of cardiac, fat or salivary gland biopsies may be performed in a subset of subjects. In OLE2, in case of salivary gland biopsy, a saliva sample will be taken at the time of biopsy.

### Study Results

After completing the Phase 1 study, the safety and efficacy of antibody NI006 were evaluated. The primary results were generated after all patients in the highest dose cohort had completed the placebo-controlled SAD/MAD phase and, unless indicated otherwise, results are presented for the safety population, i.e., including all patients that received at least one dose of investigational product (NI006 or placebo). All available data from the OLE phase at the time of analysis data-cut was included in the analysis. There were no formal statistical hypotheses tested and no imputations were performed for missing data. For descriptive statistics, patients randomized to NI006 were grouped according to their nominal dose cohort at enrollment, while patients on placebo were pooled from all dose cohorts. Irrespective of potential up-titrations, OLE data was aggregated using the same, nominal group allocation. Absolute (ACFB) or relative change from baseline (RCFB) were calculated. A pre-OLE baseline was used for the calculation of change during the OLE for patients receiving placebo during the SAD/MAD phase before switching to NI006 treatment during the OLE. Kruskal Wallis test was utilized to compare ACFB/RCFB either between the 6 dose cohorts and placebo or between high dose cohorts (10-60 mg/kg), low dose cohorts (0.3-3 mg/kg) and placebo. Data analysis was performed by a contract research organization using Statistical Analysis System® (SAS®) version 9.4 (or higher), and GGplot2 package for R was used for plotting of data.

The following results were obtained.

As mentioned above, 40 patients with wild-type or hereditary ATTR-CM (median age 72 years, 97.5% male), chronic heart failure and baseline NT-proBNP between of 766 and 5892 μg/mL were randomized 4:2 to intravenous infusions of NI006 or placebo every four weeks in six ascending dose cohorts (0.3 to 60 mg/kg). After 4 double-blinded infusions, participants of the open-label extension phase received 8 further administrations of NI006 with stepwise dose up-titration.

NI006 was found to be generally safe and well-tolerated up to the highest dose tested. No dose-limiting toxicity and no serious adverse reactions occurred. The PK profile was typical for an IgG antibody, and no anti-NI006 antibodies were identified. Data from bone scintigraphy and MRI indicated that doses of 30 mg/kg and 60 mg/kg decreased the cardiac amyloid deposition by a median of 12.8% and 25.6% compared to baseline at 4 months; at 12 months, the median reductions were 30.7% and 50.7%, respectively. In the same patients, NT-proBNP was reduced by 78.2% and 72.2% at 12 months, and echocardiographic parameters indicated improved systolic and diastolic function. Accordingly, this study is the first to demonstrate that substantial depletion of cardiac transthyretin amyloid, accompanied by signs of NT-proBNP lowering and improved cardiac function, is possible with NI006, an anti-TTR antibody.

In more detail:

### Patient Characteristics

In total, 40 participants (median age 72 years, range 28 to 87 years, 97.5% male) were sequentially enrolled at dose levels between 0.3 and 60 mg/kg, including 4 replacement participants; 27 were randomized to NI006 and 13 to placebo (Table 3). The majority of patients presented with ATTRwt-CM (33/40, 82.5%) and were on concomitant therapy with tafamidis (36/40, 90%). Some degree of polyneuropathic symptoms was documented in 15 of 40 patients (37.5%). According to established markers of ATTR-CM disease severity (NYHA, NAC and MAYO stages), patients with mild to advanced disease status were enrolled, with a tendency towards a higher number of advanced patients randomized to NI006 than placebo (Table 3). Otherwise, baseline parameters were well balanced between the different NI006 dose cohorts and placebo, including echocardiographic parameters and estimates of baseline amyloid load on scintigraphy and cMRI. Trial adherence was high: 85% of patients (34/40, including the replaced patients) received all four scheduled placebo-controlled doses in the SAD/MAD phase and 97% of eligible patients (34/35) enrolled in the OLE phase after SAD/MAD completion.

### Safety and Tolerability of NI006

The primary objective of the trial was the determination of NI006's safety and tolerability profile at a planned dose range between 0.3 mg/kg and 60 mg/kg by investigation of both clinically relevant treatment-emergent adverse events (AEs) reported by the investigators and evaluation of changes in safety markers, including orientational echocardiograms, close ECG monitoring during administrations and hospitalizations, and laboratory safety parameters measured locally at the trial sites. Blinded safety data was continuously monitored by both investigators and sponsor and reviewed by a Data Evaluation Committee (DEC).

The results showed that NI006 was generally safe and well-tolerated up to the highest dose level tested; no patient experienced a dose-limiting toxicity and no serious AE (SAE) occurred that was considered related to NI006 (i.e., no serious adverse reaction). Most patients experienced ≥1 AE (38/40, 95%) during the SAD/MAD phase, the majority of which were mild to moderate in intensity (CTCAE Grade 1 and 2, 64.9% and 31.4%, respectively) and without dose-dependency in the overall incidence of non-serious AEs or SAEs (see Tables 5 and 6). Deaths were reported in two patients (1 placebo, 1 in 3 mg/kg cohort) during their participation in the OLE phase, both of which were attributed to progression of amyloidosis (see Table 7).

As expected in this patient population, cardiac disorders, in particular events of heart failure and arrhythmias, were numerically the most frequently observed system organ class (SOC) for AEs. Frequency and type of those AEs were similar across dose cohorts without a trend for a dose-dependent increase in severity of events (see Tables 6 and 8). Non-serious AEs of cytokine release syndrome with associated increases in cardiac biomarkers were reported in 3 patients in the 10 mg/kg and 30 mg/kg cohorts during the SAD/MAD phase. All 3 patients continued dosing throughout the SAD/MAD and OLE phases without recurrence of the AE. A trend towards a dose-dependent increase in the incidence of musculoskeletal events, mainly consisting of events of arthralgia and arthritis, was observed with ascending doses in the SAD/MAD phase and in patients who switched from placebo in the OLE phase (see Table 7). The majority of these events were of Grade 1-Grade 2 intensity and did not result in discontinuations. Two subjects experienced transient asymptomatic decreases in platelet count, one of whom was discontinued due to a non-serious Grade 3 event of thrombocytopenia. No infusion reactions (IR) and no other clinically significant values or changes from baseline in safety laboratory parameters, vital signs, or FOG parameters were observed.

TABLE 5
AE summary for SAD/MAD phase

| | 0.3 mg/kg | 1 mg/kg | 3 mg/kg | 10 mg/kg | 30 mg/kg | 60 mg/kg | All NI006 | Placebo |
|---|---|---|---|---|---|---|---|---|
| | n = 4 | n = 4 | n = 4 | n = 5 | n = 5 | n = 5 | n = 27 | n = 13 |
| Subjects with at least | 4 (100) | 4 (100) | 3 (75.0) | 5 (100) | 5 (100) | 5 (100) | 26 (96.3) | 11 (84.6) |
| one AE * | [24] | [14] | [22 | [30 | [37] | [25] | [152] | [39] |
| Mild | 2 (50.0) | 2 (50.0) | 1 (25.0) | 2 (40.0) | 2 (40.0) | 2 (40.0) | 11 (40.7) | 6 (46.2) |
| | [20] | [12] | [14] | [17] | [21] | [17] | [101] | [23] |
| Moderate | 1 (25.0) | 0 | 2 (50.0) | 2 (40.0) | 2 (40.0) | 3 (60.0) | 10 (37.0) | 3 (23.1) |
| | [3] | | [8] | [12] | [15] | [8] | [46] | [14] |
| Severe | 1 (25.0) | 2 (50.0) | 0 | 1 (20.0) | 1 (20.0) | 0 | 5 (18.5) | 2 (15.4) |
| | [1] | [2] | | [1] | [1] | | [5] | [2] |
| Life Threatening | 0 | 0 | 0 | 0 | 0 | 0 | 0 | 0 |
| Death | 0 | 0 | 0 | 0 | 0 | 0 | 0 | 0 |
| Subjects with at least | 1 (25.0) | 3 (75.0) | 0 | 1 (20.0) | 1 (20.0) | 0 | 6 (22.2) | 3 (23.1) |
| one SAE | [1] | [3] | | [3] | [1] | | [8] | [3] |
| Subjects with at least | 0 | 1 (25.0) | 1 (25.0) | 1 (20.0) | 2 (40.0) | 1 (20.0) | 6 (22.2) | 0 |
| one related AE | | [1] | [2] | [5] | [13] | [4] | [22] | |
| Subjects with at least | 0 | 0 | 0 | 0 | 0 | 0 | 0 | 0 |
| one related SAE | | | | | | | | |
| Subjects with any AE | 0 | 0 | 0 | 0 | 0 | 0 | 0 | 1 (7.7) |
| leading to d/c of IMP | | | | | | | | |
| Subjects with any AE | 0 | 0 | 0 | 2 (40.0) | 1 (20.0) | 1 (20.0) | 4 (14.8) | 0 |
| leading to study d/c | | | | | | | | |

Numbers represent n of patients (% within group) and [n of events].
* subjects are only counted once at the highest CTCAE grade.
d/c discontinuation

TABLE 6
AE summary for SAD/MAD phase by SOC

| | 0.3 mg/kg | 1 mg/kg | 3 mg/kg | 10 mg/kg | 30 mg/kg | 60 mg/kg | All NI006 | Placebo |
|---|---|---|---|---|---|---|---|---|
| | n = 4 | n = 4 | n = 4 | n = 5 | n = 5 | n = 5 | n = 27 | n = 13 |
| Blood and | 0 | 0 | 1 (25.0) | 0 | 2 (40.0) | 2 (40.0) | 5 (18.5) | 2 (15.4) |
| lymphatic system | | | [1] | | [2] | [5] | [8] | [2] |
| disorders | | | | | | | | |
| Cardiac disorders | 3 (75.0) | 3 (75.0) | 3 (75.0) | 4 (80.0) | 4 (80.0) | 2 (40.0) | 19 (70.4) | 5 (38.5) |
| | [7] | [6] | [3] | [8] | [7] | [4] | [35] | [9] |
| Gastrointestinal | 0 | 1 (25.0) | 1 (25.0) | 0 | 0 | 2 (40.0) | 4 (14.8) | 3 (23.1) |
| disorders | | [1] | [1] | | | [3] | [5] | [3] |
| General disorders | 3 (75.0) | 0 | 1 (25.0) | 1 (20.0) | 3 (60.0) | 1 (20.0) | 9 (33.3) | 2 (15.4) |
| and administration | [4] | | [1] | [1] | [5] | [1] | [12] | [2] |
| site conditions | | | | | | | | |
| Immune system | 0 | 0 | 0 | 1 (20.0) | 2 (40.0) | 0 | 3 (11.1) | 0 |
| disorders | | | | [1] | [2] | | [3] | |
| Infections and | 2 (50.0) | 0 | 1 (25.0) | 1 (20.0) | 1 (20.0) | 1 (20.0) | 6 (22.2) | 3 (23.1) |
| infestations | [2] | | [1] | [2] | [1] | [1] | [7] | [3] |
| Injury, poisoning | 1 (25.0) | 1 (25.0) | 1 (25.0) | 0 | 0 | 0 | 3 (11.1) | 1 (7.7) |
| and procedural | [1] | [1] | [1] | | | | [3] | [1] |
| complications | | | | | | | | |
| Investigations | 2 (50.0) | 0 | 0 | 2 (40.0) | 2 (40.0) | 0 | 6 (22.2) | 1 (7.7) |
| | [2] | | | [5] | [6] | | [13] | [1] |
| Metabolism and | 2 (50.0) | 2 (50.0) | 1 (25.0) | 1 (20.0) | 1 (20.0) | 2 (40.0) | 9 (33.3) | 1 (7.7) |
| nutrition disorders | [2] | [2] | [2] | [1] | [1] | [2] | [10] | [2] |
| Musculoskeletal | 0 | 0 | 2 (50.0) | 3 (60.0) | 2 (40.0) | 1 (20.0) | 8 (29.6) | 2 (15.4) |
| and connective | | | [4] | [8] | [9] | [3] | [24] | [2] |
| tissue disorders | | | | | | | | |
| Nervous system | 1 (25.0) | 0 | 1 (25.0) | 2 (40.0) | 1 (20.0) | 2 (40.0) | 7 (25.9) | 4 (30.8) |
| disorders | [1] | | [1] | [2] | [1] | [2] | [7] | [5] |
| Psychiatric | 0 | 1 (25.0) | 0 | 0 | 0 | 2 (40.0) | 3 (11.1) | 1 (7.7) |
| disorders | | [1] | | | | [2] | [3] | [2] |
| Respiratory, | 0 | 1 (25.0) | 2 (50.0) | 0 | 1 (20.0) | 1 (20.0) | 5 (18.5) | 1 (7.7) |
| thoracic and | | [1] | [2] | | [1] | [1] | [5] | [1] |
| mediastinal | | | | | | | | |
| disorders | | | | | | | | |
| Skin and | 1 (25.0) | 1 (25.0) | 2 (50.0) | 1 (20.0) | 0 | 1 (20.0) | 6 (22.2) | 0 |
| subcutaneous | [2] | [1] | [3] | [1] | | [1] | [8] | |
| tissue disorders | | | | | | | | |
| Vascular | 1 (25.0) | 1 (25.0) | 0 | 0 | 1 (20.0) | 0 | 3 (11.1) | 0 |
| disorders | [1] | [1] | | | [1] | | [3] | |

TABLE 7
AE summary for combined SAD/MAD and OLE phase

| | | | | | | | All patients | Placebo, |
|---|---|---|---|---|---|---|---|---|
| | | | | | | | randomized | switched |
| | 0.3 mg/kg | 1 mg/kg | 3 mg/kg | 10 mg/kg | 30 mg/kg | 60 mg/kg | to NI006 | to NI006 |
| | n = 4 | n = 4 | n = 4 | n = 5 | n = 5 | n = 5 | n = 27 | n = 13 |
| Subjects with at | 4 (100) | 4 (100) | 3 (75.0) | 5 (100) | 5 (100) | 5 (100) | 26 (96.3) | 12 (92.3) |
| least one AE * | [41] | [25] | [37] | [39] | [57] | [41] | [240] | [117] |
| Mild | 0 | 2 (50.0) | 0 | 2 (40.0) | 1 (20.0) | 0 | 5 (18.5) | 2 (15.4) |
| | [29] | [22] | [25] | [25] | [36] | [27] | [164] | [65] |
| Moderate | 3 (75.0) | 0 | 1 (25.0) | 2 (40.0) | 2 (40.0) | 4 (80.0) | 12 (44.4) | 6 (46.2) |
| | [11] | [1] | [9] | [13] | [19] | [13] | [66] | [41] |
| Severe | 1 (25.0) | 2 (50.0) | 1 (25.0) | 1 (20.0) | 2 (40.0) | 1 (20.0) | 8 (29.6) | 3 (23.1) |
| | [1] | [2] | [1] | [1] | [2] | [1] | [8] | [10] |
| Life Threatening | 0 | 0 | 0 | 0 | 0 | 0 | 0 | 0 |
| | | | [1] | | | | [1] | |
| Death | 0 | 0 | 1 (25.0) | 0 | 0 | 0 | 1 (3.7) | 1 (7.7) |
| | | | [1] | | | | [1] | [1] |
| Subjects with at | 2(50.0) | 3(75.0) | 1(25.0) | 1(20.0) | 1(20.0) | 1(20.0) | 9 (33.3) | 5 (38.5) |
| least one SAE | [2] | [3] | [1] | [3] | [1] | [1] | [11] | [13] |
| Subjects with at | 1 (25.0) | 2 (50.0) | 2 (50.0) | 2 (40.0) | 2 (40.0) | 1 (20.0) | 10 (37.0) | 3 (23.1) |
| least one related | [1] | [3] | [5] | [7] | [20] | [4] | [40] | [12] |
| AE | | | | | | | | |
| Subjects with at | 0 | 0 | 0 | 0 | 0 | 0 | 0 | 0 |
| least one related | | | | | | | | |
| SAE | | | | | | | | |
| Subjects with any | 0 | 0 | 0 | 0 | 1(20.0) | 0 | 1(3.7) | 1(7.7) |
| AE leading to d/c | | | | | | | | |
| of IMP | | | | | | | | |
| Subjects with any | 0 | 0 | 1(25.0) | 2(40.0) | 1(20.0) | 1(20.0) | 5(18.5) | 1(7.7) |
| AE leading to | | | | | | | | |
| study d/c | | | | | | | | |

Numbers represent n of patients (% within group) and [n of events].
* subjects are only counted once at the highest CTCAE grade.
d/c discontinuation

TABLE 8
AE summary for combined SAD/MAD and OLE phase by SOC

| | | | | | | | All patients | Placebo, |
|---|---|---|---|---|---|---|---|---|
| | | | | | | | randomized | switched |
| | 0.3 mg/kg | 1 mg/kg | 3 mg/kg | 10 mg/kg | 30 mg/kg | 60 mg/kg | to NI006 | to NI006 |
| | n = 4 | n = 4 | n = 4 | n = 5 | n = 5 | n = 5 | n = 27 | n = 13 |
| Blood and lymphatic | 0 | 0 | 1 (25.0) | 0 | 2 (40.0) | 2 (40.0) | 5 (18.5) | 3 (23.1) |
| system disorders | | | [1] | | [3] | [5] | [9] | [3] |
| Cardiac disorders | 4 (100) | 3 (75.0) | 3 (75.0) | 4 (80.0) | 5 (100) | 3 (60.0) | 22 (81.5) | 8 (61.5) |
| | [11] | [6] | [6] | [10] | [10] | [8] | [51] | [29] |
| Eye disorders | 1 (25.0) | 0 | 0 | 0 | 0 | 0 | 1 (3.7) | 4 (30.8) |
| | [1] | | | | | | [1] | [7] |
| Gastrointestinal | 0 | 3 (75.0) | 2 (50.0) | 0 | 0 | 4 (80.0) | 9 (33.3) | 5 (38.5) |
| disorders | | [3] | [3] | | | [6] | [12] | [7] |
| General disorders | 3 (75.0) | 1 (25.0) | 2 (50.0) | 1 (20.0) | 4 (80.0) | 2 (40.0) | 13 (48.1) | 4 (30.8) |
| and administration | [5] | [1] | [5] | [1] | [6] | [2] | [20] | [13] |
| site conditions | | | | | | | | |
| Immune system | 0 | 0 | 0 | 1 (20.0) | 2 (40.0) | 0 | 3 (11.1) | 0 |
| disorders | | | | [1] | [2] | | [3] | |
| Infections and | 3 (75.0) | 0 | 1 (25.0) | 1 (20.0) | 3 (60.0) | 3 (60.0) | 11 (40.7) | 6 (46.2) |
| infestations | [4] | | [1] | [2] | [3] | [4] | [14] | [6] |
| Injury, poisoning and | 4 (100) | 2 (50.0) | 1 (25.0) | 0 | 1 (20.0) | 0 | 8 (29.6) | 2 (15.4) |
| procedural | [5] | [2] | [2] | | [1] | | [10] | [2] |
| complications | | | | | | | | |
| Investigations | 2 (50.0) | 0 | 0 | 2 (40.0) | 2 (40.0) | 0 | 6 (22.2) | 1 (7.7) |
| | [4] | | | [5] | [6] | | [15] | [1] |
| Metabolism and | 2 (50.0) | 2 (50.0) | 1 (25.0) | 1 (20.0) | 1 (20.0) | 2 (40.0) | 9 (33.3) | 4 (30.8) |
| nutrition disorders | [2] | [4] | [2] | [1] | [2] | [2] | [13] | [7] |
| Musculoskeletal and | 0 | 2 (50.0) | 3 (75.0) | 4 (80.0) | 3 (60.0) | 2 (40.0) | 14 (51.9) | 5 (38.5) |
| connective tissue | | [2] | [7] | [10] | [16] | [5] | [40] | [17] |
| disorders | | | | | | | | |
| Nervous system | 1 (25.0) | 1 (25.0) | 1 (25.0) | 2 (40.0) | 2 (40.0) | 2 (40.0) | 9 (33.3) | 7 (53.8) |
| disorders | [1] | [2] | [1] | [2] | [3] | [2] | [11] | [10] |
| Psychiatric disorders | 0 | 1 (25.0) | 0 | 0 | 0 | 2 (40.0) | 3 (11.1) | 1 (7.7) |
| | | [1] | | | | [2] | [3] | [2] |
| Respiratory, thoracic | 0 | 0 | 1 (25.0) | 0 | 0 | 1 (20.0) | 2 (7.4) | 2 (15.4) |
| and mediastinal | | | [1] | | | [1] | [2] | [2] |
| disorders | | | | | | | | |
| Skin and | 1 (25.0) | 1 (25.0) | 2 (50.0) | 3 (60.0) | 0 | 1 (20.0) | 8 (29.6) | 2 (15.4) |
| subcutaneous tissue | [2] | [1] | [3] | [6] | | [1] | [13] | [3] |
| disorders | | | | | | | | |
| Vascular disorders | 2 (50.0) | 1 (25.0) | 0 | 0 | 2 (40.0) | 0 | 5 (18.5) | 0 |
| | [2] | [1] | | | [2] | | [5] | |

### PK and Immunogenicity Profile

Serum NI006 concentration was measured serially throughout the trial in all patients using a validated assay. Individual total serum NI006 exposure was calculated as the area under the curve (AUC) from the simulated PK profiles using individual parameter estimates. Monitoring for occurrence of anti-drug antibodies was performed throughout the trial. As regards the immunogenicity analysis, the presence of potential anti-drug antibodies was assessed using biotinylated- and sulfotag-labeled NI006 as detection reagents in an electrochemiluminescence immunoassay validated by QPS (the Netherlands). For PK analysis, serum NI006 concentrations were measured using a validated sandwich ELISA assay built on two anti-idiotypic Fab fragments binding selectively to NI006. The assay lower limit of quantification (LLOQ) was 0.17 ug/mL. A non-compartmental analysis (NCA) was performed by Nuventra (Durham, NC, USA) on serum NI006 concentration versus time data for calculation of standard PK parameters, with a validated installation of Phoenix WinNonlin version 8.2.2 using actual blood sampling times and dosing levels and utilizing the intravenous (IV) infusion model. Population PK modeling was performed by LYO-X (Switzerland) using a two-compartmental model with linear antibody clearance from the central compartment, and antibody binding to its target ATTR and elimination of the NI006:ATTR complex in the peripheral compartment. Population PK parameters were estimated using the stochastic approximation of expectation maximization (SAEM) algorithm implemented in Monolix. Individual total serum NI006 exposure in patients were calculated from the simulated PK profiles using individual parameter estimates and the linear trapezoidal rule, at 4 months and at 12 months.

It has been shown that NI006 pharmacokinetic profile was typical for a human IgG with low to moderate inter-subject variability: following a single IV dose, serum NI006 concentrations declined in a biphasic manner with elimination half-life ranging from 15.5 to 19.2 days. Exposure, as measured by Cmax and AUC, increased with increasing dose in a dose-proportional manner. There was a progressive, dose-dependent drug accumulation that reached stable levels after repeated doses q4w. None of the patients developed anti-drug antibodies (ADA) throughout the study including the OLE.

### Amyloid Depletion

Amyloid depletion was determined by scintigraphy and cardia MRT. In particular, for cardiac amyloid imaging, all enrolled patients underwent either serial scintigraphy, or cardiac MRI. The selection of the imaging modality was independently selected by the investigator for each patient to allow accommodation of both individual patient characteristics (e.g., claustrophobia precluding MRI) and local standards at the trial site. For both imaging modalities, acquisition was performed according to harmonized protocols across the trial sites, and analysis was performed at a central imaging core lab by two independent, blinded readers. In planar scintigraphy, changes in cardiac tracer retention relative to the whole-body retention (Heart/Whole Body Retention ratio, H/WBR ratio) was used to estimate the effect of NI006 on the cardiac amyloid load. Correspondingly, using contrast-enhanced cardiac MRI, treatment effects of NI006 were estimated by changes in the quantity of extracellular volume (ECV) in the left ventricle. Relative changes at month 4 and month 12 were calculated separately for scintigraphy and MRI imaging and across results from both imaging modalities.

Cardiac MRIs were recorded using local scanners at the study sites. The acquisition protocol was based on latest guidelines [1-3] and standardized across all sites during the trial set-up phase and adherence to the laboratory manual was continuously monitored. MRI scans were interpreted by two independent readers (specialized radiologists or cardiologists) at the imaging core lab using Medis software (Medis, Leiden, The Netherlands). All MRIs were analyzed individually; central readers were blinded not only to the treatment allocation, but also to the patient identifier, dose cohort and acquisition timepoint. The MRI acquisition and analysis procedure was similar to the method described by Martinez-Naharro et al. (PMID 28728692): T1 and ECV measurements were performed before and after contrast administration defining the whole left ventricular myocardium at the basal and midventricular level as the region of interest. Both ventricular myocardium and blood T1 measurements were performed in short-axis views. Information on hematocrit was available. ECV was calculated according to the following formula:

$$ECV = ( 1 - hematocrit ) \times \frac{Δ R 1 myocardium}{Δ R 1 blood}$$

with R1=1/T1 and Δ being the difference between pre- and post-contrast measurements.

Consensus readings were performed in case of disagreement between both readers beyond a pre-defined range of ±10% for the key parameters.

Bone scintigraphies were acquired at trial sites using ^{99m}Tc-hydroxyl-methylene-diphosphonate (HMDP) or ^{99m}Tc-3,3-diphosphono-1,2-propanodicarboxylic acid (DPD) as tracers according to a harmonized acquisition protocol established in a laboratory manual during trial set-up. Adherence to the laboratory manual and image quality was monitored continuously. Planar whole-body images were acquired 3 h after tracer infusion. Reading of acquired images was performed centrally at the imaging core lab by two independent nuclear physicians using Syngovia software (Siemens). To calculate the Heart/Whole Body Retention ratio (H/WB ratio), potential areas of high tracer retention (e.g. bladder, kidneys, injection site) were considered as rejection areas in the following formula:

$$H / WB ratio = \frac{Heart Retention}{Whole Body Retention - Rejection Areas}$$

Consensus readings were performed in case of disagreement between both readers beyond a pre-defined range of ±10% for the key parameters.

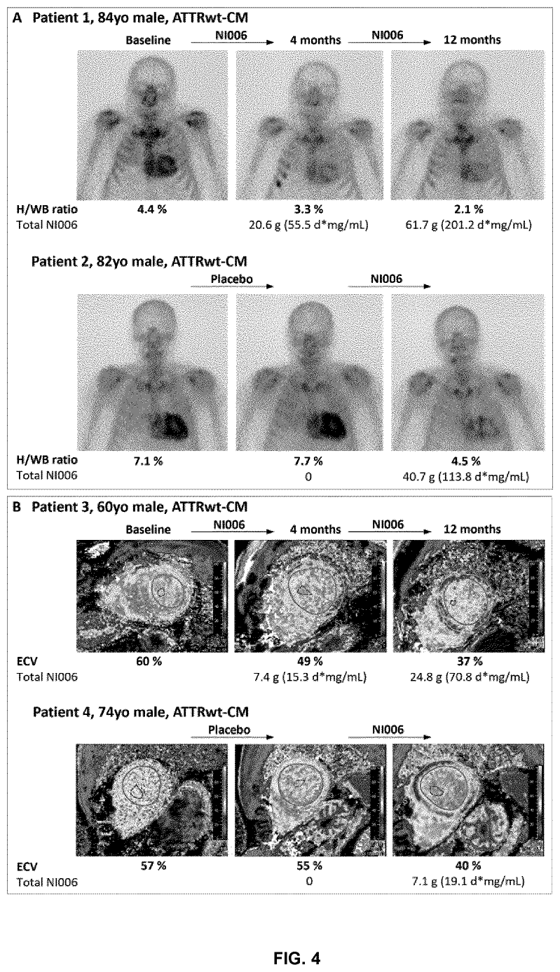

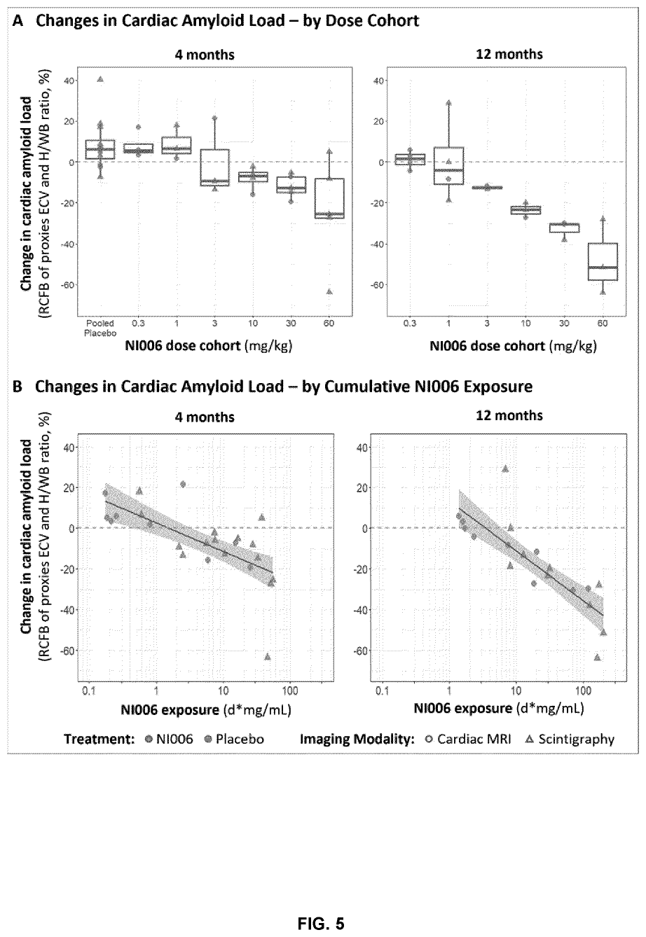

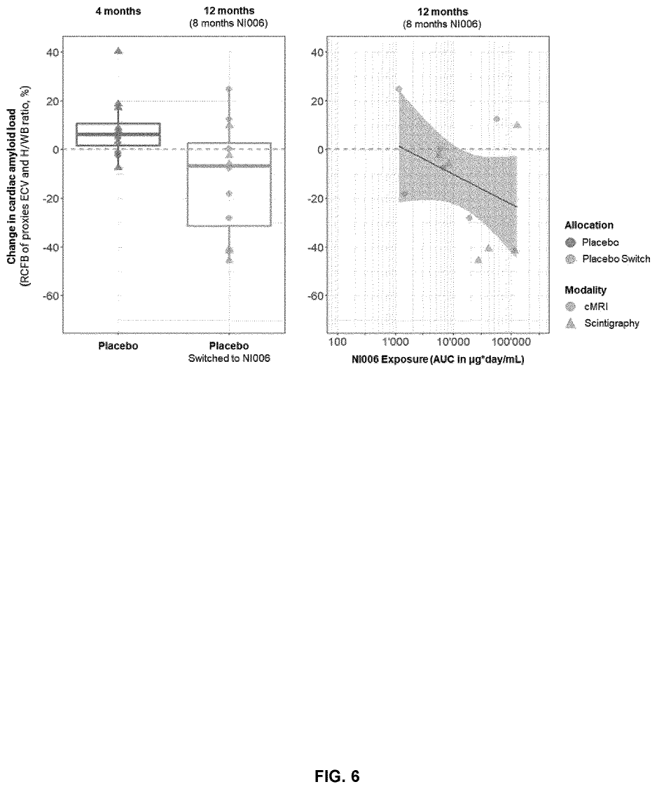

It was shown that treatment with NI006 reduced in a substantial manner cardiac ATTR amyloid deposition in patients with ATTR-CM as measured by both scintigraphy and cMRI compared to baseline as early as 4 months (FIG. 4). Continued treatment with NI006 up to 12 months resulted in further reduction of the amyloid deposition. In contrast, patients initially randomized to placebo showed an increase of cardiac amyloid deposition after 4 months and presented decreased amyloid load after switching to NI006 for 8 months of treatment in the OLE. Pooled data from both imaging techniques indicated that doses of 10 mg/kg, 30 mg/kg or 60 mg/kg decreased the cardiac amyloid deposition by a median of 6.9%, 12.8% and 25.6% compared to baseline at 4 months. After 8 additional months of treatment in the OLE including some dose up-titration from 10 mg/kg to 30 mg/kg and from 30 mg/kg to 60 mg/kg, the median reductions in cardiac amyloid load were 23.6%, 30.7% and 50.7% compared to baseline at 12 months, respectively (FIG. 5). In individual cases, amyloid reductions down to an absolute ECV of 35% or H/WB ratio of 2.1% were observed after 12 months of treatment with NI006. Relative changes from baseline were significantly different between low dose cohorts (0.3-3 mg/kg), high dose cohorts (10-60 mg/kg) and placebo for both for scintigraphy (p=0.011) and MRI (p=0.016) at 4 months. Higher total NI006 exposure, simulated from individual PK profiles to account for unforeseen treatment interruptions (e.g., pandemic-related) and individual up-titration schedules during the OLE, was correlated with higher reduction of cardiac amyloid at 4 months (r=−0.673) and 12 months (r=−0.651), across both imaging modalities (FIG. 5B). Data from placebo switchers were consistent with these observations and confirmed them (FIG. 6).

### Effects on Cardiac Biomarkers and Echocardiographic Parameters

Changes in cardiac structure and function were explored on echocardiogram and utilizing plasma biomarkers NT-proBNP and Troponin-T. In addition, even though the study was not powered for this, overall functional capacity and quality of life were explored using the 6-minute walk test (6mwt) and Kansas City Cardiomyopathy Questionnaire (KCCQ), respectively. Changes at month 4 and month 12 were analyzed. In more detail, standardized echocardiograms were acquired at 3 timepoints throughout the trial: Full echocardiograms for efficacy analyses were recorded at baseline prior to the first treatment, after MAD completion (4 months) and after OLE completion (12 months). Harmonized acquisition protocols were applied across all sites and analysis was performed centrally at the imaging core lab (Biotrial, Rennes, Francce) by an experienced cardiologist with demonstrated low intra-reader variability blinded to the pseudonymized patient ID, treatment allocation, dose cohort and acquisition timepoint. Measurements were performed in triplicate (sinus rhythm) or quintuplet (atrial fibrillation) using EchoPAC CE Medical software (GE Healthcare, Milwaukee, MI, USA) and the mean value of the triplicates/quintuplets was used for further analysis. If recording did not allow measurement of individual parameters (e.g., poor echogenicity, insufficient number of loops recorded, suboptimal section), measurements were reported as missing.

In addition, non-standardized orientational echocardiograms were performed by the investigators or sub-investigators according to local practice prior to each of the first 5 treatment administrations and prior to hospital discharge to exclude sudden deterioration of systolic left ventricular function or occurrence of pericardial effusion.

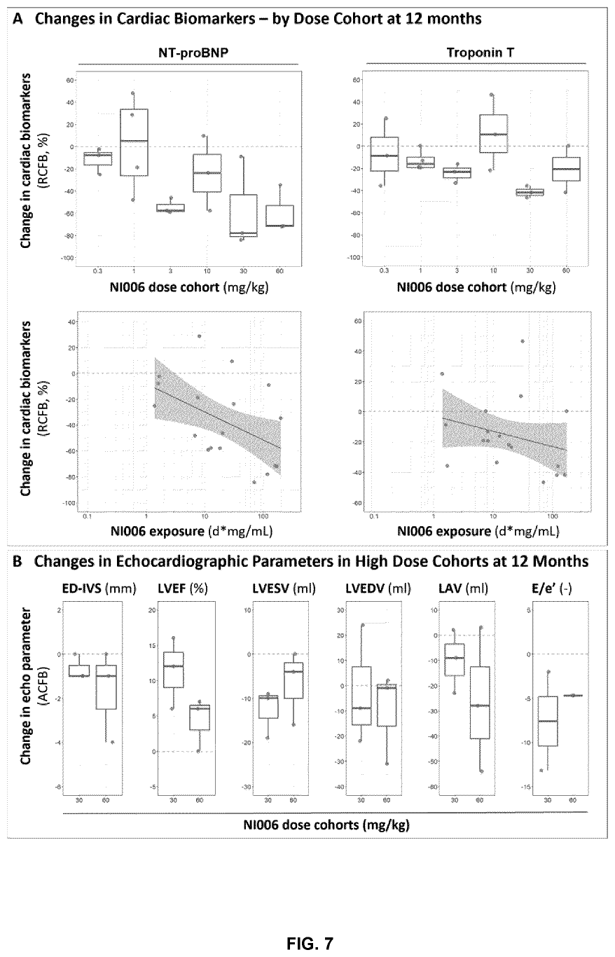

It was shown that plasma biomarkers of cardiac stress and function, NT-proBNP and Troponin-T, were reduced compared to baseline in a majority of patients at 12 months (FIG. 7A). NI006 reduced NT-proBNP in a dose and time dependent manner. In the dose cohorts treated at 30 mg/kg or 60 m/kg, with the highest observed reductions of cardiac amyloid load, the median relative reductions from baseline of NT-proBNP reached 78.2% and 72.2% at 12 months. In the 60 mg/kg cohort, median NT-proBNP was reduced from 1482 ng/L at baseline to 420 ng/L at 12 months, which is below the threshold of inclusion for the present study. Similarly, Troponin-T showed larger reductions from baseline with higher total NI006 exposure.

Patients in the two dose cohorts with highest amyloid removal, 30 mg/kg and 60 mg/kg, also demonstrated signs of treatment benefit at 12 months in echocardiographic parameters of cardiac structure, systolic and diastolic function (FIG. 7B): reduced left ventricular wall-thickness (1 mm median reduction of ED-IVS in both dose cohorts), increased LVEF (12% and 6% median absolute increase, respectively), which appeared to be mainly driven by a reduction of the end-systolic volume (LVESV), reduced left atrial volume (9 and 28 mL median reduction) and reduced E/e′ ratio (−7.6 and −4.7 median absolute change).

### Summary and Discussion

NI006 is useful for the treatment of transthyretin amyloidosis with cardiomyopathy, irrespective of the underlying genotype. In patients with a confirmed ATTR-CM diagnosis, this first-in-human study demonstrated that IV administration of NI006 every 4 weeks for 12 months was generally safe and well-tolerated up to the highest dose tested. As determined by two different proxies of cardiac amyloid load, bone scintigraphy and cardiac MRI, NI006 depleted amyloid transthyretin from cardiac tissue in a dose- and time-dependent manner, with most pronounced effects at doses greater than 10 mg/kg q4w. Echocardiographic and laboratory parameters indicative of the disease status in patients ATTR-CM showed signs of improvements in the highest dose cohorts.

Good tolerability of NI006, and absence of infusion reactions and dose-limiting toxicities may be attributed to the human origin of the amino acid sequence and the extraordinary selectivity for the misfolded amyloid transthyretin protein with no biding to the physiological TTR tetramer. Some adverse events, including reported cases of transient cytokine-release and arthralgias after the initial dose, may be linked to NI006's activation of phagocytic immune cells when bound to its target.

Disease-stabilizing therapies including TTR tetramer stabilization by tafamidis or acoramidis, or silencing of the TTR gene expression, are expected to slow the accumulation of new ATTR amyloid deposits, but, conceptionally, do not act on the removal of existing ATTR amyloid deposits. While published data from clinical trials and real-world settings indicate that disease stabilizing therapies are capable of improving amyloid load on imaging proxies compared to an untreated population (Chamling et al., Clin Res Cardiol. 112 (2023), 353-62; Fontana et al., JACC Cardiovasc Imaging. 14 (2021), 189-99), a substantial improvement compared to baseline is rare (Wu et al., ESC Heart Fail. 9 (2022), 4335-4339). In the present study, patients on tafamidis presented with high-degree cardiac amyloid deposition at baseline (overall median H/WB ratio 5.0%, median ECV 59.8%, a cut-off associated with increased mortality, and after four months of placebo treatment, further progression of amyloid deposition (median RCFB+6.1% across both imaging modalities), indicating the urgent need for the development of novel treatments capable of reverting this disease pathology (FIG. 5). In this patient population, treatment with NI006 depleted cardiac ATTR amyloid deposits dose- and time dependently. For the first time, a therapeutic intervention resulted in substantial and sustained amyloid depletion as evidenced by complementary imaging technologies. In some patients, nearly complete removal of cardiac amyloid was achieved during the treatment period and as early as 4 months. H/WB ratio of scintigraphy tracer uptake could be reduced to values close to 2.0% following 12 months of treatment, a threshold observed in heart failure (HF) patients without amyloidosis (Galat et al., J Nucl Cardiol. 22 (2015)853-857) or ECV to about 40% following 12 months, a threshold value that may also indicate nearly complete removal of cardiac ATTR amyloid and is associated with improved prognosis (Schelbert et al., JACC Cardiovasc Imaging. 12 (2019), 2305-2318). These findings further support the preclinical data that demonstrated NI006's ability to induce degradation of transthyretin amyloid by recruitment of phagocytic cells through its Fc-effector function, and mechanistically similar to antibodies aducanumab, lecanemab and donanemab for the depletion of amyloid beta in Alzheimer's disease.

In ATTR-CM, NT-pro-BNP is associated with mortality when assessed either at time of diagnosis or serially as change from baseline in patients with cardiac amyloidosis (see for example Kristen et al., PLoS One. 12 (2017), e0173086. Treatment with NI006 reduced NT-proBNP compared to baseline in a dose- and time dependent manner, in contrast to an expected increase when treated with tafamidis (Nativi-Nicolau et al., ESC Heart Fail. 8 (2021), 3875-3884; Rapezzi et al., JACC Heart Fail. 9 (2021), 115-123). Patients receiving treatment at 30 mg/kg or 60 mg/kg over 12 months had a NT-proBNP relative median reduction of >72% compared to baseline, median NT-proBNP down to 420 ng/L and echocardiographic changes suggestive of months improved systolic and diastolic function. These findings indicate a possible clinical benefit downstream of amyloid removal in ATTR-CM patients treated with NI006.

In conclusion, the present study is the first to demonstrate that substantial depletion of cardiac transthyretin amyloid is possible, the primary disease pathology in ATTR-CM. Amyloid depletion was accompanied by substantial lowering of plasma NT-proBNP and signs of improved cardiac function that could indicate that removal of cardiac amyloid results in improved cardiac function and survival.

### Example 2. Calculating Administration Volume

The following example describes how to calculate the volume needed for administration of the anti-TTR antibody to a subject. The subject's weight should be calculated in kilograms (kg) and dosage determined. The anti-TTR antibody will be provided in a vial (e.g., a glass vial) at a concentration of 50 mg/mL (±12%) or 100 mg/mL (±12%). The total volume of an aqueous solution in the vial will be 2 mL (±12%), 5 mL (±12%), 10 mL (±12%), 15 mL (±12%), 20 mL (±12%), 25 mL (±12%), or 30 mL (±12%).

Calculation of the subject's dosage, total volume, and number of vials needed for administration is as follows:

$$Body weight ( kg ) \times dosage ( mg / kg ) = dose ( mg )$$

$$dose ( mg ) / antibody concentration ( mg / mL ) = total volume ( mL )$$

$$total volume ( mL ) / mL per vial ( mL ) = number of vials needed$$

Always round up the number of vials to the next whole vial number to get the number of vials needed.

Tables 9 and 10 are examples of how to calculate the number of 2 mL vials needed for administrating the anti-TTR antibody to a subject. It will be understood by one of skill in the art how to calculate the number of vials when the vial contains a volume of 5 mL, 10 mL, 15 mL, 20 mL, 25 mL, or 30 mL.

TABLE 9
Calculating the number of vials for a 58 kg subject

| | |
|---|---|
| Subject's body weight: | 58 kg |
| Assigned dose group (cohort): | 0.3 mg/kg (=cohort 1) |
| Calculated antibody dose (mg): | 58 kg × 0.3 mg/kg = 17.4 mg |
| calculated total volume (mL): | 17.4 mg/50 mg/mL = 0.348 mL |
| Number of vials: | 0.348 mL/2 mL = 0.174 vials |
| Round up to the next whole vial | 1 vial |
| number: | |
| Conclusion: | If a subject weighs 58 kg and is |
| | assigned to cohort 1 (=dose group 0.3 |
| | mg/mL), then 1 vial is needed to |
| | prepare the subject's infusion and |
| | 0.348 mL need to be withdrawn from |
| | this vial. |

TABLE 10
Calculating the number of vials for a 93 kg subject

| | |
|---|---|
| Subject's body weight: | 93 kg |
| Assigned dose group (cohort): | 10 mg/kg (=cohort 4) |
| Calculated dose (mg): | 93 kg × 10 mg/kg = 930 mg |
| calculated total volume (mL): | 930 mg/50 mg/mL = 18.6 mL |
| Number of vials: | 18.6 mL/2 mL = 9.3 vials |
| Round up to the next whole vial | 10 vials |
| number: | |
| Conclusion: | If a subject weighs 93 kg and is |
| | assigned to cohort 4 (=dose group 10 |
| | mg/mL), then 10 vials are needed to |
| | prepare the subject's infusion and |
| | 18.6 mL need to be withdrawn from |
| | this vials. |

### Example 3. Administration of an Anti-TTR Antibody Based on the Subject's Weight

A 60 kg subject may be administered 30 mg/kg of an anti-TTR antibody to in order to treat ATTR. A 20 mL glass vial containing a pharmaceutical composition with an anti-TTR antibody (e.g., an anti-TTR antibody having a heavy chain variable (VH) region having an amino acid sequence of SEQ ID NO: 7 and a light chain variable (VL) region having an amino acid sequence of SEQ ID NO: 8) at a concentration of 100 mg/mL may be prepared for administration to the subject. The pharmaceutical composition can be prepared for administration to the subject by diluting the pharmaceutical composition 5-fold with glucose into an infusion bag. After diluting 5-fold, the antibody is present in the dilution bag at a final concentration of 20 mg/mL and at a final volume of 100 mL. The total dose of the anti-TTR antibody in the infusion bag is 2000 mg. Next, the antibody may be administered by an infusion syringe to administer 30 mg/kg of the anti-TTR antibody intravenously to the subject. This results in a total volume of 90 mL and a total dose of 1800 mg being administered to the subject. This treatment may be repeated about once every 28 days for 15 months. Efficacy of this treatment may be evaluated by measuring the cardiac mass by magnetic resonance imaging (MRI) and assessing any cardiac mass reduction (e.g., reduction in amyloid) post treatment relative to the subject's cardiac mass pre-treatment.

### Example 4. Administration of a Flat Dose of an Anti-TTR Antibody to a Subject

A subject with ATTR may be treated by administration of 2000 mg of an anti-TTR antibody. A 20 mL glass vial containing a pharmaceutical composition with an anti-TTR antibody (e.g., an anti-TTR antibody having a heavy chain variable (VH) region having an amino acid sequence of SEQ ID NO: 7 and a light chain variable (VL) region having an amino acid sequence of SEQ ID NO: 8) at a concentration of 100 mg/mL may be prepared for administration to the subject. The pharmaceutical composition can be prepared for administration to the subject by diluting the pharmaceutical composition 10-fold with glucose into an infusion bag. After diluting 10-fold, the antibody is present in the dilution bag at a final concentration of 10 mg/mL and at a final volume of 200 mL. The total dose of the anti-TTR antibody in the infusion bag is 2000 mg. Next, the antibody may be administered by an infusion syringe so as to administer 2000 mg of the anti-TTR antibody to the subject. This results in a total volume of 200 mL and a total dose of 2000 mg being administered to the subject. This treatment may be repeated about once every 28 days for 12 months. Efficacy of this treatment may be evaluated by measuring the cardiac mass by magnetic resonance imaging (MRI) and assessing any cardiac mass reduction (e.g., reduction in amyloid) post treatment relative to the subject's cardiac mass pre-treatment.

### Example 5. Simulation of Serum NI006 Profiles for Body Weight Adjusted Doses of 30 and 60 mg/Kg, as Well as Flat Doses of 2000 to 5000 mg, q4w for Body Weights Ranging from 40 to 120 kg

A two-compartmental PK model with NI006 binding to ATTR in the peripheral compartment and NI006:ATTR complex elimination was fitted to serum NI006 concentrations measured in ATTR-CM patients participating in the clinical study. Patients were treated with NI006 doses ranging from 0.3 to 60 mg/kg q4w for different durations exceeding 1 year. The PK/PD model described well the observations, including between-patient variability.

The PK/PD model directly linked the total ATTR amount in the peripheral compartment with proxy measurements of cardiac amyloid load using MRI (ECV_Mid) or scintigraphy (HRWBR). The link between imaging readouts and amyloid load in the model relied on estimating individual proportionality factors on top of the physiological baseline ECV_Mid and HRWBR values found in the literature for individuals without cardiac amyloidosis. The PK/PD model fitted well the ECV_Mid and HRWBR observations.

A covariate search identified body weight as a significant covariate on the central and peripheral volumes of distribution, and age as a covariate on KD. A body weight covariate on clearance, which approached statistical significance in our limited data set, was also included in the model but using the theoretical covariate coefficient value. Simulations showed that age had no clinically relevant effect on dose-response predictions.

Predicted 95^{th} percentile Cₘₐₓ and median AUC_{28 days} vs body weight after the first dose and at steady-state are shown in FIG. 8 and FIG. 9 and as shown in these Figures, the PK/PD model was used to predict serum NI006 Cₘₐₓ and AUC_{28 days} after single dose and at steady-state after q4w dosing for the following bracketed flat doses:
5000 mg if body weight ≥100 kg.
3500 mg if 60 kg s body weight <100 kg.
2500 mg if body weight <60 kg.

The predicted Cₘₐₓ after a single dose was in the range of observed values in the 30 mg/kg group from the NI006-101 study for all body weight brackets. The predicted AUC_{28 days} after a single dose was in the range of observed values in the 30 and 60 mg/kg groups. The PK predictions were considered robust given the extensive PK data the model was fitted on.

The PK/PD model was further used to predict ATTR removal, ECV_Mid and HRWBR signals for 24 months of q4w NI006 doses. Simulations of the proposed bracketed flat doses predicted substantial ATTR removal and corresponding decreases in ECV_Mid and HRWBR similar to the ones achieved by dosing at 30 and 60 mg/kg in the trial. Because of limitations in the PD data set, the predictions for ECV_Mid at 60 mg/kg were fully extrapolated, and the predictions for HRWBR beyond 12 months at 60 mg/kg were also extrapolated.

### Example 6. Optimized Flat Doses for Body Weights Ranging from 40 to 120 kg

The flat doses simulated with the PK/PD model described in Example 5 have been further optimized. In particular, the PK/PD model was used for dose selection balancing safety & efficacy, e.g., to ensure administration of the complete amount of drug present in each vial and eliminate residual volume and corresponding drug wastage, while maintaining the same predicted efficacy. In particular, PK/PD modelling was performed as described in section 4.3 of Example 8, below, inter alia consider AUCtau between 30 mg/kg and 60 mg/kg, Cmax<60 mg/kg (95th percentile of modelled flat dose levels between 2000 mg and 5000 mg q4w). This approach along with dose formulation considerations resulted in the following flat dose weight-brackets.

TABLE 11
Flat dose calculations

| Body Weight Range | Dose |
|---|---|
| ≥40 kg to <60 kg | 2400 mg |
| ≥60 kg to <100 kg | 3200 mg |
| ≥100 kg | 4800 mg |

As already described in Example 5, the population PK/PD model was developed based on data from the Phase 1 study (NI006-101) of ALXN2220 in participants with ATTR-CM, in particular the based on the 30 mg/kg and 60 mg/kg treatment arm. These data indicate that with, compared to the initial proposed flat doses, the preferred reduced weight-bracketed dose, exposure (Cmax and AUC) will be within the range as observed with the 30 and 60 mg/kg q4w as investigated in the SAD/MAD dose. Thus, the target exposure range is justified by efficacy and safety considerations.

### Example 7. Characterization of Mature NI006/ALXN2220

Antibody NI006/ALXN2220 was produced in the CHO-K1 cell line (ATCC No. CCL 61) and obtained from the cell culture after culturing in a large-scale production bioreactor. The amino acid sequence of NI006/ALXN2220's mature heavy chain (HC) and light chain (LC) is set forth in SEQ ID NOs: 18 and 19, with the below-mentioned modifications. The total number of amino acids, number of amino acids of the heavy chain, and number of amino acids of the light chain are 1328, 450, and 214, respectively.

Further characterization of antibody NI006/ALXN2022 was mainly performed by standard procedures, for example by mass spectroscopy analysis. For example, liquid chromatography with tandem mass spectrometry (LC-MS/MS) analysis of fragments of NI006/ALXN2220 obtained from Lys-C and trypsin sequential digestion as well as free sulfhydryl analysis was used to identify post-translational modifications of NI006/ALXN2220. The characterization of antibody-based therapeutics via LC-MS analysis is a standard procedure and can be performed by a skilled artisan; see for example Robotham and Kelly, Approaches to the Purification, Analysis and Characterization of Antibody-Based Therapeutics (2020), 1-33.

N-glycan profiling was performed by releasing the N-glycans by using PNGase F and subsequent labelling with 2-AB, followed by HILIC (Hydrophilic Interaction Chromatography) separation and fluorescence detection (FLD) with a UPLC system. Individual N-glycans and unknown peaks were quantified by their peak area percentages relative to the total peak area.

The results are mentioned in the following:

The molecular weight of antibody NI006/ALXN2220 as determined by standard mass spectroscopy is approximately 147.1 kDa for the intact IgG1 and 144.2 kDa for the deglycosylated variant.

The monoclonal antibody NI006/ALXN2220 is an IgG1 subclass antibody, which is composed of two heavy chains of the IgG1 subclass and two light chains of the kappa subclass. The four chains are stabilized by multiple disulfide bonds. In particular, as determined by standard procedures, i.e., per Lys-C and trypsin digestion and subsequent LC-MS, at least the following disulfide bridges are present in
NI006/ALXN2220:
LC:C23-LC:C88
LC:C134-LC:C194
LC:C214-HC:C223
HC:C22-HC:C97
HC:C147-HC:C203
HC1:229-HC2:229 and HC1:232-HC2:232
HC:C264-HC:C324
HC:C370-HC:C428
(the corresponding amino acid sequences of the HC and LC are set forth in SEQ ID NOs: 9 and 10)
   NI006/ALXN2220 is a glycoprotein and the constant region of each heavy chain contains one N-linked glycan site at residue N300. During determination of the glycosylation profile, it was shown that the major N-glycan types are G0F (~49.0%) and G1F (~25.4%). In more detail, the following glycosylation profile (the types of sugar, the location of glycosylation site(s), etc.) has been determined for NI006/ALXN2220:

TABLE 12
Glycan Types

| Glycan | Glycan Type |
|---|---|
| 23100 | Man3+1F |
| 33000 | G0-GN |
| 33100 | G0F-GN |
| 43000 | G0 |
| 43100 | G0F |
| 25000 | Man5 |
| 34100/44000 | G1F-GN/G1a |
| 44000 | G1b |
| 44100 | G1Fa |
| 44100 | G1Fb |
| 45100 | G2F |
| 45110 | G2FS1 |
| 45120 | G2FS2 |

### Note:

1. The nomenclature of the glycans follows the order of HexNac-Hexose-Fucose-NeuAc-NeuGc. For example, 23000 is HexNac(2)-Hexose(3)-Fucose(0)-NeuAc(0)-NeuGc(0).
2. G1Fa and G1Fb are isomers and are grouped into G1F. G1F is calculated as the sum of G1Fa and G1Fb using the original unrounded numbers.

Furthermore, glutamine at N-terminal modified as pyro-glutamic (abundance in sample: 99.9) acid and C-terminal lysine clipping of the heavy chain (abundance in sample: 95.8%) have been identified as the major post-translational modifications. In addition, small ratio modifications such as methionine oxidation, asparagine deamidation and asparagine succinimide formation have been experimental determined as shown in the following Table:

TABLE 13
Post-translational Modifications

| | Sequence | | | |
|---|---|---|---|---|
| No. | No. | Sequence | SEQ ID NO: | PTMs |
| *1 | HC: 1-13 | QLQLQESGPGLVK | 16 | / |
| | | QLQLQESGPGLVK | 16 | pE(Q) |
| 2 | HC: 252- | DTLMISR | 17 | / |
| | 258 | DTLMISR | 17 | Oxidation(M) |
| 3 | HC: 305- | VVSVLTVLHQDWLNGK | 18 | / |
| | 320 | VVSVLTVLHQDWLNGK | 18 | Deamidation(N) |
| | | VVSVLTVLHQDWLNGK) | 18 | Succinimide |
| | | | | Formation(N) |
| 4 | HC: 374- | GFYPSDIAVEWESNGQPENNYK | 19 | / |
| | 395 | GFYPSDIAVEWESNGQPENNYK | 19 | Deamidation(N) |
| #5 | HC: 443- | SLSLSPGK | 20 | / |
| | 450 | SLSLSPGK | 20 | -K |
| | | SLSLSPGK | 20 | -K -G Amidation(P) |

Note:
1. HC refers to heavy chain and LC refers to light chain.
2. Peptide sequences in red and underlined font were identified as the site of the PTMs.
3. * refers to N-terminal related peptide of heavy chain and # refers to C-terminal related peptide of heavy chain.
4. / refers to no PTM reported.
5. pE(Q) refers to the N-terminal glutamine modified as pyro-glutamic acid.
6. -K refers to loss of the C-terminal lysine.
7. -K -G Amidation(P) refers to the amidation of the C-terminal proline after the loss of the C-terminal lysine and glycine.
In summary, N-linked glycosylation of the heavy chain, N-terminal pyro-glutamic acid modified from N-terminal glutamine, and C-terminal lysine clipping of the heavy chain are the major post-translational modifications of NI006/ALXN2220

### Example 8. a Phase 3, Randomized, Double-Blind, Placebo-Controlled, Multicenter Study to Evaluate the Efficacy and Safety of Amyloid Depleter ALXN2220 in Adult Participants with Transthyretin Amyloid Cardiomyopathy (ATTR-CM)

### 1. Protocol Summary

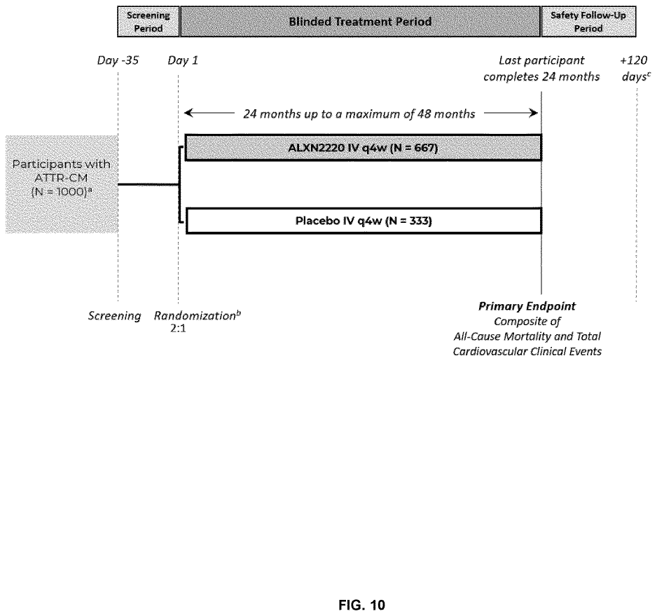

A Phase 3, Randomized, Double-blind, Placebo-controlled, Multicenter Study to Evaluate the Efficacy and Safety of Amyloid Depleter ALXN2220 in Adult Participants with Transthyretin Amyloid Cardiomyopathy (ATTR-CM). The study schematic is shown in FIG. 10.

### Background & Objective

ALXN2220 is proposed for the treatment of confirmed wild-type or hereditary TTR-mediated amyloidosis (ATTR amyloidosis) in adults with clinically established cardiomyopathy. ALXN2220, a recombinant human anti-ATTR IgG1 mAb, was developed for the removal of ATTR fibrils and depletion of ATTR deposits from tissues by phagocytic immune cells.

ATTR-CM is an increasingly recognized cause of heart failure with poor prognosis and limited therapeutic options. Despite advances in slowing disease progression, there is no available treatment that depletes ATTR from the heart for the amelioration of cardiac dysfunction and participants with a greater cardiac amyloid load and more advanced stages of the disease represent an unmet medical need.

In the Phase 1 study (NI006-101), treatment with ALXN2220 for over a year was associated with no apparent drug-related SAEs and surrogate markers indicated cardiac ATTR depletion and improvement of cardiac biomarkers and function (Garcia-Pavia, 2023). Based on data from the Study NI006-101, and population PK/PD model-based dose simulation, body weight bracketed flat doses of ALXN2220 administered q4w were identified for this study.

A primary objective of this clinical study is to evaluate the efficacy and safety of ALXN2220 in adult participants with ATTR-CM treated with standard therapy.

### Objectives, Endpoints, and Estimands

TABLE 14
Objectives, Endpoints, and Estimands of the Clinical Trial

| | |
|---|---|
| Objectives | Endpoints and Estimands |

Primary

| | |
|---|---|
| To assess the efficacy of ALXN2220 in the | Population: Adult (≥18 to ≤90 years of age) |
| treatment of adult participants with ATTR-CM | participants with symptomatic ATTR-CM (either |
| by evaluating the difference between the | variant or wild-type) and NT-proBNP >2000 pg/mL |
| ALXN2220 and placebo groups as assessed | Endpoint: Composite of ACM and total CV clinical |
| by the composite endpoint of ACM and total | events during the Blinded Treatment Period |
| CV clinical events | Treatment conditions: ALXN2220 or placebo IV q4w |
| | Handling of IEs: |
| | Study treatment discontinuation will be addressed |
| | based on treatment policy strategy with post-treatment |
| | discontinuation data included in the analysis. |
| | Receiving heart transplant, or permanent CMAD will |
| | be based on a composite strategy with the IE handled |
| | as death in the analysis. |
| | Population-level summary: Rate ratio from the |
| | Andersen-Gill model with robust variance estimation |

Secondary

| | |
|---|---|
| To assess the effects of ALXN2220 on the | Composite of ACM and total HF events |
| composite of ACM and total HF events | |
| To assess the effects of ALXN2220 on | Change from baseline in KCCQ-OS to 24 months |
| symptoms, functionality, and health-related | |
| QoL as measured by the change from | |
| baseline in KCCQ-OS | |
| To assess the effects of ALXN2220 on CV | Time to CV related mortality |
| related mortality | |
| To assess the effects of ALXN2220 on change | Change from baseline in 6MWT to 24 months |
| from baseline in 6MWT | |
| To assess the efficacy of ALXN2220 by the | Rate of CV clinical events |
| rate of CV clinical events | |
| To assess the efficacy of ALXN2220 in the | Time to ACM |
| treatment of ATTR-CM as assessed by time to | |
| ACM | |

Safety

| | |
|---|---|
| To assess the safety and tolerability of | Incidence of TEAEs and treatment-emergent SAEs |
| ALXN2220 | Changes from baseline in physical examination, vital |
| | signs, clinical laboratory tests, and 12-lead ECGs |

Immunogenicity

| | |
|---|---|
| To assess immunogenicity to ALXN2220 | ADA incidence, response categories and titer |

Pharmacokinetics

| | |
|---|---|
| To characterize the PK of ALXN2220 in | Serum ALXN2220 concentration |
| participants with ATTR-CM | |

This is a Phase 3, randomized, double-blind, placebo-controlled study to evaluate the efficacy and safety of amyloid depleter ALXN2220 compared to placebo in adult participants with ATTR-CM who are on standard therapy, as determined by treating and study physician(s), which may include conventional heart failure therapies and approved disease modifying agents for ATTR amyloidosis.

The primary purpose of this study is to measure if ALXN2220 improves mortality and CV morbidity in participants with ATTR-CM.

The study consists of a Screening Period (up to 35 days), followed by a Blinded Treatment Period. The Blinded Treatment Period will last a minimum of 24 months for each participant. It will end when the last participant completes 24 months of blinded treatment or after a participant completes 48 months of blinded treatment, whichever is earlier. Upon completion of the Blinded Treatment Period, participants will be followed in the Safety Follow-up Period (120 days). The primary objective of the study is the assessment of the efficacy of ALXN2220 through analysis of a composite endpoint of ACM and total CV clinical events.

Approximately 1000 participants will be enrolled and randomized to receive either ALXN2220 or placebo in a 2:1 ratio. The randomization will be stratified by 3 factors: treatment with a disease modifying agent at Screening (TTR silencer±TTR stabilizer vs TTR stabilizer alone vs none), TTR genotype (variant vs wild-type), and disease severity based on cardiac biomarker at Screening (NT-proBNP>3000 pg/mL vs NT-proBNP s 3000 pg/mL).

The number of participants in any of the strata may be capped depending on recruitment numbers.

During the Blinded Treatment Period, participants will receive study intervention (ALXN2220 or placebo) q4w via IV infusion for at least 24 months up to a maximum of 48 months according to the Schedule of Activities.

The total duration of the study from the first day of screening to the last visit is approximately 2.5 years for the last surviving participant. The total duration of the study for participants recruited earlier will be dependent on the overall recruitment duration and individual date of enrollment. The maximum total duration will be 4.5 years.

The treatment duration of surviving participants will be a minimum 24 months up to a maximum of 48 months.

For all participants, post-treatment Safety Follow-up Visits will be conducted approximately 60 days and 120 days after the last dose of study intervention to collect information on laboratory parameters, concomitant medications, non-pharmacologic therapies and procedures, and AEs.

An Open-Label-Extension (OLE) study is anticipated.

### Number of Participants

Approximately 1000 participants will be enrolled and randomized in a 2:1 ratio to receive either ALXN2220 or placebo.

### Treatment Groups and Duration

During the Blinded Treatment Period, participants will receive study intervention (ALXN2220 or placebo) q4w via IV infusion for at least 24 months up to a maximum of 48 months.

### 2. Introduction

### 2.1 Study Rationale

ALXN2220 is proposed for the treatment of confirmed wild-type or hereditary TTR mediated amyloidosis (ATTR amyloidosis) in adults with clinically established cardiomyopathy. ALXN2220, a recombinant human anti-ATTR IgG1 mAb, was developed for the removal of ATTR fibrils and depletion of ATTR deposits from tissues by phagocytic immune cells.

ATTR-CM is an increasingly recognized cause of heart failure with poor prognosis and limited therapeutic options. Despite advances in slowing disease progression, there is no available treatment that depletes ATTR from the heart for the amelioration of cardiac dysfunction and participants with a greater cardiac amyloid load and more advanced stages of the disease represent an unmet medical need.

The purpose of this study is to evaluate the efficacy and safety of ALXN2220 in adult participants with ATTR-CM treated with standard therapy.

### 2.2 ALXN2220

ALXN2220 (formerly known as NI006) is a recombinant human anti-ATTR IgG1 mAb that was generated based on a comprehensive immune repertoire analysis of memory B-cell complements of healthy elderly human subjects (Michalon, 2021).

ALXN2220 selectively binds ATTR, with no binding to the native (physiological) non-amyloid tetrameric protein conformation of TTR. ALXN2220 induces antibody-mediated phagocytosis of ATTR fibrils by phagocytic immune cells such as macrophages, resulting in the clearance of ATTR deposits from tissues. ALXN2220 targets in a similar manner wild-type and mutant ATTR deposits in various tissues. ALXN2220 also binds to misfolded oligomeric transthyretin proteins (Michalon, 2021).

The high selectivity of ALXN2220 results from its binding to the linear epitope with the sequence WEPFA hidden in TTR's naturally folded conformation, but accessible to antibody binding following unfolding and aggregation. The absence of binding to physiological TTR has relevant advantages, it preserves TTR's biological activity and avoids antibody sequestration on the physiological protein (Michalon, 2021).

By activating the elimination of ATTR deposits in patients suffering from ATTR amyloidosis, ALXN2220 is expected to restore organ function and structure. Specifically, in the proposed program, ALXN2220 is expected to decrease cardiac muscle stiffness, to improve heart contractility and elasticity, and to possibly improve peripheral neurological functions. This effect is expected to result in symptom stabilization or regression and to improve organ function and survival.

Safety, tolerability, PK, and efficacy of treatment with ALXN2220 were evaluated in a first-in-human Phase 1 study in adult participants with ATTR-CM (Study NI006-101, NCT04360434, conducted in 4 European countries). Primary efficacy data from Cohort 1 to Cohort 6 showed that ALXN2220 reduced cardiac ATTR amyloid deposits in a dose- and time dependent-manner as measured by 2 independent imaging proxies of cardiac amyloid load (Garcia-Pavia, 2023).

### 2.3 Benefit/Risk Assessment

### 2.3.1 Risk Assessment

TABLE 15
Study Intervention Risk Assessment

| | | |
|---|---|---|
| Potential Risk of | Summary of | |
| Clinical Significance | Data/Rationale for Risk | Mitigation Strategy |

Study Intervention ALXN2220

| | | |
|---|---|---|
| Immunogenicity | ALXN2220 is derived from the | Provisions are made in the |
| Treatment with any therapeutic | human immune repertoire and | protocol for the clinical |
| biologic has the potential to induce | has a 100% human sequence. | management of hypersensitivity |
| an immune response. Potential | On this basis, its | in the clinic. |
| clinical consequences may include | immunogenicity is expected to | Guidance is provided to |
| hypersensitivity, anaphylaxis, or | be very low or absent. | interrupt study intervention in |
| related type of reactions or loss of | Available results from | the event of any severe |
| efficacy. | Study NI006-101 indicate the | hypersensitivity reaction |
| | absence of ADAs for up to | (Section 7 and Section 10) that |
| | 30 months of treatment in all | occurs in the clinic and to |
| | participants. | instruct the participants to notify |
| | | the Investigator and seek |
| | | medical care urgently for |
| | | delayed reactions. |
| | | In case of suspected SAEs of |
| | | hypersensitivity or anaphylaxis, |
| | | additional ADA samples may |
| | | be collected during or in |
| | | proximity to the event. |
| | | Guidance for evaluation and |
| | | management of symptoms is |
| | | provided in Section 10.9. |
| Infusion-related reactions and | The ALXN2220 drug product | The Pharmacy Manual will |
| other hypersensitivity-related | is a human monoclonal | outline proper preparation of |
| events | antibody intended for IV | investigational material and |
| IRR and other | administration. | study personnel involved in the |
| hypersensitivity-related events are | Individual cases of IRR | administration of doses for this |
| a potential risk for IV administered | described as mild to moderate | study will be properly trained. |
| therapeutic agents including | cytokine release syndrome | Participants will be closely |
| monoclonal antibodies such as | were noted in the | monitored during infusions and |
| ALXN2220. | Study NI006-101 in | during a postinfusion |
| IRRs may present during the | association with the initial or | observation period. |
| infusion or may be delayed in onset | second dose of ALXN2220. | Guidance for evaluation and |
| and may or may not be causally | Clinical manifestations varied | management of IRR and other |
| related to the presence of specific | among the affected | hypersensitivity reactions and |
| ADA. | participants and included | symptoms is provided in |
| IRRs have been reported using a | elevated temperature, | Section 10.9 and as noted |
| variety of descriptors (e.g., IRR, | decreased oxygen saturation, | above immunogenicity potential |
| cytokine release syndrome, | decrease in blood pressure, | risk. |
| anaphylaxis, anaphylactoid | and arthralgias, and varied | |
| reactions) (Doessegger, 2015; | among the participants. | |
| Stewart, 2022). | One case of Grade 1 | |
| While IRRs are generally mild, | cutaneous blister (possible | |
| severe life threatening and fatal | drug reaction) was reported | |
| reactions may occur. | after long term exposure. | |
| Clinical manifestations can vary | | |
| from patient to patient and can | | |
| involve multiple body systems | | |
| (e.g., cardiovascular, pulmonary, | | |
| skin, gastrointestinal). | | |
| Tissue inflammation | The probability of ALXN2220 | Routine safety laboratory |
| Consistent with its mechanism of | binding to non-target | assessments, monthly |
| action, an ALXN2220 mediated | molecules is considered low. | physician examinations. |
| immune response against ATTR | However, the possibility for | Guidance for evaluation and |
| deposits in the heart or other | ALXN2220-mediated immune | management of reactions and |
| tissues may provoke a transient | activation to affect | sequelae are provided in |
| symptomatic inflammatory | surrounding tissues in amyloid | Section 10.10. |
| response (e.g., noninfectious | affected organs such as heart | |
| myocarditis, arthritis, | and joints is an acknowledged | |
| tenosynovitis). | potential risk. | |
| Mild transient tissue inflammation | In Study NI006-101, instances | |
| may be clinically asymptomatic. | of mild/moderate | |
| More severe symptomatic | (Grade 1/Grade 2) drug | |
| inflammation may lead to varying | related-increase in | |
| types of clinical sequelae and | Troponin T and myocardial | |
| depending on the tissue involved | inflammation were reported as | |
| could be life threatening or fatal. | myocarditis following the initial | |
| | dose of ALXN2220. Other | |
| | drug-related inflammatory | |
| | reactions affecting the | |
| | musculoskeletal system (e.g., | |
| | reactive arthritis, rheumatoid | |
| | and psoriatic arthritis, | |
| | polymyalgia rheumatica) were | |
| | also observed. | |
| Decreased platelet count | In the Study NI006-101, 2 of | Reduction of dose: majority of |
| Substantial decreases in platelet | 5 participants in the 60 mg/kg | participants (body weight range |
| count can increase the risk of | cohort (dosed with | ≥60 kg to <100 kg) will receive |
| bleeding, which may have severe | approximately 5800 mg [97 kg | 3200 mg ALXN2220 per |
| sequelae. | patient] and 5200 mg [87 kg | infusion (Section 4.3). |
| | patient]) experienced a | Exclusion criteria for minimum |
| | decrease in platelet count | screening platelet count, |
| | (decrease range 50,000 to | historical conditions associated |
| | 130,000 × 10⁹/L) within | with thrombocytopenia. |
| | several days of each of the | Routine safety laboratory |
| | first 2 to 3 infusions. No | assessments of complete blood |
| | clinical sequelae were | count and additional follow-up |
| | observed. | platelet count approximately |
| | | 48 hours after initial infusion. |
| | | Guidance for evaluation and |
| | | management of sequelae |
| | | (Section 10.11). |
| Pregnancy and lactation | Treatment-related effects | WOCBP and males must agree |
| | upon reproductive | to contraceptive requirements |
| | performance have not been | (Section 5.1 Inclusion |
| | evaluated in nonclinical | Criterion 10 and Section 10.5). |
| | toxicology studies. | Pregnancy testing will be |
| | | conducted predose for women |
| | | of childbearing potential. |
| | | Pregnancy outcome for |
| | | participants and female |
| | | partners of male participants |
| | | will be collected. |

Study Procedures

| | | |
|---|---|---|
| Placebo | Placebo treatment is not | Participants should be treated |
| Exposure of participants to a | expected to benefit | according to the local standard |
| nonactive treatment. | participants. | therapy at the treating |
| | Superiority of ALXN2220 over | physician's discretion which |
| | placebo is not established. | may include approved disease |
| | | modifying agents for ATTR |
| | | amyloidosis in addition to |
| | | ATTR-adapted conventional |
| | | heart failure therapies. |
| Disallowed Medication | No formal interaction studies | ALXN2220 should not be |
| Concomitant use of other | have been performed with | administered within 30 days or |
| medications may increase the risk | ALXN2220. | 5 half-lives of intake of another |
| of adverse events or may | Based on ALXN2220's mode | investigational drug, whichever |
| complicate the assessment of | of action, concomitant therapy | is longer. |
| clinical efficacy and cannot be | with immunosuppressive | Wash out periods must be |
| addressed with statistical | therapies may reduce its | respected for a list of drugs |
| methodology. | expected efficacy. | (including inotersen) defined in |
| | Concomitant therapy with | Section 6.9.2. |
| | inotersen may increase the | |
| | potential risk of platelet | |
| | decreases. | |
| Optional Tissue Biopsy | Complications are infrequent | Optional study procedure with |
| Salivary gland | with salivary gland and skin | separate informed consent. |
| Skin | punch biopsy but may include | Participants with pre-existing |
| Cardiac (right heart) | pain, bleeding or bruising, | risk factors for bleeding, |
| | infection, or allergic reaction to | infection or impaired wound |
| | local anesthetic, scarring. | healing are not eligible to |
| | Transient drainage of salivary | participate in these optional |
| | fluid from the puncture site | procedures. |
| | could occur after the salivary | In addition, for cardiac biopsy, |
| | gland biopsy. | institutional requirements for |
| | Cardiac biopsy may | biopsy eligibility need to be |
| | additionally be associated with | fulfilled. |
| | a risk of arrythmias, and less | |
| | common complications could | |
| | include conduction | |
| | abnormalities, pneumothorax, | |
| | hemothorax, cardiac | |
| | tamponade, tricuspid value | |
| | damage, thromboembolic | |
| | events, e.g., pulmonary | |
| | embolism, very infrequent | |
| | fatal complications have | |
| | occurred following cardiac | |
| | biopsy. | |

### 2.3.2 Benefit Assessment

ALXN2220 is a novel investigational drug in development for the treatment of participants with ATTR-CM, a debilitating, serious, and life-threatening orphan disease with limited treatment options. Current treatment approaches (approved or in late-stage clinical development) focus on eliminating or stabilizing the source of the amyloidogenic protein, either through TTR stabilization (tafamidis is currently the only approved drug for ATTR-CM) or TTR gene silencing (patisiran, vutrisiran, and eplontersen, all currently under investigation for ATTR-CM). However, there are no approved therapies that deplete or eliminate already deposited amyloid fibrils, which continues to cause progressive organ damage, especially the heart, and be a cause of death due to worsening heart failure despite treatment with TTR stabilizers or gene silencers.

Based on non-clinical data (Michalon, 2021) and the primary results of the Phase 1 study (Garcia-Pavia, 2023), administration of ALX2220 to patients with moderate to severe ATTR-CM in the Phase 3 study is hypothesized to remove deposits of ATTR amyloid in the heart as indicated by changes observed with either one of two imaging-based surrogate markers of cardiac amyloid load (ECV on cMRI and cardiac tracer uptake on scintigraphy). By depleting cardiac TTR amyloid deposits, ALXN2220 is expected to halt or reverse disease progression via cardiac reverse remodeling. These clinically meaningful improvements will be demonstrated and measured by specific clinical outcomes, including time to death (mortality), CV morbidity (CV hospitalizations or ex-hospital clinical handling of worsening heart failure) and endpoints of cardiac function and well-being (e.g., KCCQ-OS, NT-proBNP, and 6MWT). It is expected that ALXN2220 added to the current therapies to treat cardiac related ATTR conditions will improve ATTR-CM patients' survival and QoL.

In this study, ATTR patients with a mixed phenotype, defined as having cardiac amyloidosis coexistent with sensory or motor neurological involvement, will likely be enrolled. Of note, those ATTR-CM participants who only have symptoms of autonomic dysfunction and/or carpal tunnel syndrome are not considered to have mixed phenotype. Beneficial effects of ALXN2220 on neuropathy symptoms and signs in ATTRv participants will be explored in this pivotal Phase 3 study using PND score, NIS, QoL-DN and sNFL, which are assessments for measuring the progression of neuropathy disease in ATTR-PN.

### 2.3.3 Overall Benefit Risk Combination

This study's target population of ATTR-CM patients includes patients with moderate to severe cardiomyopathy, including cases of mixed phenotype with concomitant polyneuropathy and patients with very advanced cardiomyopathy that have been traditionally understudied or excluded in recent clinical studies. Existing data indicate that current approved therapies, and those under investigation, provide, or may provide, a clinical benefit that is inversely related to the clinical severity of the ATTR amyloidosis syndrome. Current and upcoming treatment options are directed to reduce the production of amyloidogenic TTR proteins, but scarce evidence exists about the possibility to eliminate ATTR deposits in tissues and organs. Hence, biological stabilization is expected, but organ remodeling or restoration of function is deemed unlikely, illustrating a therapeutic gap. Recent data indicate that spontaneous regression in ATTR affected patients may occur, but it is extremely rare (Fontana, 2023).

Treatment with ALXN2220, a mAb designed to deplete ATTR deposits, in addition to the best CV care, offers the opportunity to address important and unmet medical needs in patients with ATTR-CM. ALXN2220 is expected to decrease amyloid depositions, decrease hospitalizations, improve QoL, improve signs and symptoms of heart failure and polyneuropathy, and increase longevity.

Considering the experimental nature of ALXN2220, and the data from the Phase 1 study, several measures are being taken in this study to minimize the few identifiable, measurable, and treatable risks that participants may suffer. The potential risks identified in association with ALXN2220 are justified by the anticipated benefits that may be afforded to adult participants with ATTR-CM. Additionally, the potential risks associated with study design (placebo), and study procedures (optional tissue biopsy), are appropriately mitigated by the strategies outlined in Table 15, above).

### 3. Objectives, Endpoints, and Estimands

TABLE 16
Objectives and Endpoints

| | |
|---|---|
| Objectives | Endpoints and Estimands |

Primary

| | |
|---|---|
| To assess the efficacy of ALXN2220 in the | Population: Adult (≥18 to ≤90 years of age) |
| treatment of adult participants with ATTR- | participants symptomatic ATTR-CM (either variant |
| CM by evaluating the difference between the | or wild-type) and NT-proBNP >2000 pg/mL |
| ALXN2220 and placebo groups as assessed | Endpoint: Composite of ACM and total CV clinical |
| by the composite endpoint of ACM and total | events during the Blinded Treatment Period |
| CV clinical events | Treatment conditions: ALXN2220 or placebo IV |
| | q4w |
| | Handling of IEs: |
| | Study treatment discontinuation will be |
| | addressed based on treatment policy strategy |
| | with post-treatment discontinuation data included |
| | in the analysis. |
| | Receiving heart transplant, or permanent CMAD |
| | will be based on a composite strategy with the IE |
| | handled as death in the analysis. |
| | Population-level summary: Rate ratio from the |
| | Andersen-Gill model with robust variance |
| | estimation |

Secondary

| | |
|---|---|
| To assess the effects of ALXN2220 on the | Population: Adult (≥18 to ≤90 years of age) |
| composite of ACM and total HF events | participants symptomatic ATTR-CM (either variant |
| | or wild-type) and NT-proBNP >2000 pg/mL |
| | Endpoint: Composite of ACM and total HF events |
| | Treatment conditions: ALXN2220 or placebo IV |
| | q4w |
| | Handling of IEs: |
| | Study treatment discontinuation will be |
| | addressed based on treatment policy strategy |
| | with post-treatment discontinuation data included |
| | in the analysis. |
| | Receiving heart transplant, or permanent CMAD |
| | will be based on a composite strategy with the IE |
| | handled as death in the analysis. |
| | Population-level summary: Rate ratio from the |
| | Andersen-Gill model with robust variance |
| | estimation |
| To assess the effects of ALXN2220 on | Population: Adult (≥18 to ≤ 90 years of age) |
| symptoms, functionality, and health-related | participants symptomatic ATTR-CM (either variant |
| QoL as measured by the change from | or wild-type) and NT-proBNP >2000 pg/mL |
| baseline in KCCQ-OS | Endpoint: Change from baseline in KCCQ-OS to |
| | 24 months |
| | Treatment conditions: ALXN2220 or placebo IV |
| | q4w |
| | Handling of IEs: |
| | Study treatment discontinuation will be |
| | addressed based on treatment policy strategy |
| | with post-treatment discontinuation data included |
| | in the analysis. |
| | Death will be addressed based on composite |
| | strategy. Missing data post-death will use J2R |
| | imputation and sample from the bottom 50% of |
| | observed change from baseline values in the |
| | placebo participants, assuming MNAR. |
| | Receiving heart transplant, or permanent CMAD: |
| | treated as death and addressed based on |
| | composite strategy. Assessment after the IE will |
| | be censored and imputed by the same J2R |
| | approach as for death. |
| | Population-level summary: LS mean difference |
| | between ALXN2220 and placebo in change from |
| | baseline in KCCQ-OS to 24 months from the |
| | repeated measures mixed model |
| To assess the effects of ALXN2220 on CV | Population: Adult (≥18 to ≤ 90 years of age) |
| related mortality | participants symptomatic ATTR-CM (either variant |
| | or wild-type) and NT-proBNP >2000 pg/mL |
| | Endpoint: Time to CV related mortality |
| | Treatment conditions: ALXN2220 or placebo IV |
| | q4w |
| | Handling of IEs: |
| | Study treatment discontinuation will be |
| | addressed based on treatment policy strategy |
| | with post-treatment discontinuation data included |
| | in the analysis. |
| | Non-CV death will be addressed based on |
| | composite strategy with observed data until the |
| | IE will be analyzed. |
| | Receiving heart transplant, or permanent CMAD |
| | will be addressed on composite strategy with IE |
| | handled as CV death in the analysis. |
| | Population-level summary: Hazard ratio from Cox |
| | proportional hazards model |
| To assess the effects of ALXN2220 on | Population: Adult (≥18 to ≤90 years of age) |
| change from baseline in 6MWT | participants symptomatic ATTR-CM (either variant |
| | or wild-type) and NT-proBNP >2000 pg/mL |
| | Endpoint: Change from baseline in 6MWT to |
| | 24 months |
| | Treatment conditions: ALXN2220 or placebo IV |
| | q4w |
| | Handling of IEs: |
| | Study treatment discontinuation will be |
| | addressed based on treatment policy strategy |
| | with post-treatment discontinuation data included |
| | in the analysis. |
| | Death will be addressed based on composite |
| | strategy. Missing data post-death will use J2R |
| | imputation and sample from the bottom 50% of |
| | observed change from baseline values in the |
| | placebo participants, assuming MNAR. |
| | Receiving heart transplant, or permanent CMAD: |
| | treated as death and addressed based on |
| | composite strategy. Assessment after the IE will |
| | be censored and imputed by the same J2R |
| | approach as for death. |
| | Population-level summary: LS mean difference |
| | between ALXN2220 and placebo in change from |
| | baseline in 6MWT to 24 months from the repeated |
| | measures mixed model |
| To assess the efficacy of ALXN2220 in the | Population: Adult (≥18 to ≤90 years of age) |
| treatment of ATTR-CM as assessed by the | participants symptomatic ATTR-CM (either variant |
| rate of CV clinical events | or wild-type) and NT-proBNP >2000 pg/mL |
| | Endpoint: Rate of CV clinical events |
| | Treatment conditions: ALXN2220 or placebo IV |
| | q4w |
| | Handling of IEs: |
| | Study treatment discontinuation will be |
| | addressed based on treatment policy strategy |
| | with post-treatment discontinuation data included |
| | in the analysis. |
| | Death will be addressed based on composite |
| | strategy with observed data until the IE will be |
| | analyzed. |
| | Receiving heart transplant, or permanent CMAD |
| | will be based on a composite strategy with the IE |
| | handled as death in the analysis. |
| | Population-level summary: Rate ratio of CV clinical |
| | events from Andersen-Gill model with robust |
| | variance estimation |
| To assess the efficacy of ALXN2220 in the | Population: Adult (≥18 to ≤90 years of age) |
| treatment of ATTR-CM as assessed by time | participants symptomatic ATTR-CM (either variant |
| to ACM | or wild-type) and NT-proBNP >2000 pg/mL |
| | Endpoint: Time to ACM |
| | Treatment conditions: ALXN2220 or placebo IV |
| | q4w |
| | Handling of IEs: |
| | Study treatment discontinuation will be |
| | addressed based on treatment policy strategy |
| | with post-treatment discontinuation data included |
| | in the analysis. |
| | Receiving heart transplant, or permanent CMAD |
| | will be based on composite strategy with the IE |
| | handled as death in the analysis. |
| | Population-level summary: Hazard ratio from Cox |
| | proportional hazards model |

Safety

| | |
|---|---|
| To assess the safety and tolerability of | Incidence of TEAEs and treatment-emergent SAEs |
| ALXN2220 | Changes from baseline in physical examination, |
| | vital signs, clinical laboratory tests, and 12-lead |
| | ECGs |

Immunogenicity

| | |
|---|---|
| To assess immunogenicity to ALXN2220 | ADA incidence, response categories and titer |

Pharmacokinetics

| | |
|---|---|
| To characterize the PK of ALXN2220 in | Serum ALXN2220 concentration |
| participants with ATTR-CM | |

Exploratory

| | |
|---|---|
| To assess the effects of ALXN2220 on | Change from baseline in NT-proBNP |
| change from baseline in cardiac biomarker | |
| NT-proBNP | |
| To assess the efficacy of ALXN2220 as | Rate of HF events |
| assessed by the rate of HF events | |
| To assess the effects of ALXN2220 on the | Incidence of intensification of oral diuretic therapy |
| requirement for intensification of oral diuretic | (including outpatient augmentation of therapy) |
| therapy | |
| To assess the effects of ALXN2220 on the | Incidence of changes in disease modifying therapy |
| frequency of changes in disease modifying | |
| therapies | |
| To assess the effects of ALXN2220 on the | Incidence of hospitalization for atrial fibrillation |
| incidence of atrial fibrillation related events | |
| To assess the effects of ALXN2220 on | Change from baseline in KCCQ-OS beyond |
| symptoms, functionality, and health-related | 24 months of study treatment |
| QoL as measured by the change from | |
| baseline in KCCQ-OS | |
| To assess the effects of ALXN2220 on | Change from baseline in 6MWT beyond 24 months |
| change from baseline in 6MWT | of study treatment |
| To assess the effects of ALXN2220 on the | Change from baseline in ATTR-CM disease |
| disease severity of ATTR-CM utilizing | severity based on the Mayo, NAC, and Columbia |
| different staging systems | disease stage, and NYHA classification. Additional |
| | staging systems will be considered if developed |
| | and recommended by professional guidelines. |
| To assess the effects of ALXN2220 on left | Change from baseline in GLS |
| ventricular myocardial contractility | |
| To assess the effects of ALXN2220 on global | Change from baseline in stroke volume |
| left ventricular output | |
| To assess the effects of ALXN2220 on further | Change from baseline in echocardiography |
| echocardiography parameters of interest | parameters of interest |
| To assess the effects of ALXN2220 on | Change from baseline in hs-cTnT |
| cardiac biomarker troponin T | |
| To assess the effects of ALXN2220 on | Change from baseline in DPD/PYP/ HMDP cardiac |
| reducing imaging surrogate markers of | scintigraphy cardiac uptake |
| cardiac amyloid load | Changes from baseline in cMRI-derived ECV, T1 |
| | and T2 mapping |
| To assess the effects of ALXN2220 on renal | Changes from baseline in eGFR |
| function | |
| To assess the effects of ALXN2220 on QoL | Changes from baseline in EQ-5D-5L |
| using PROs | Change from baseline in SF-36 scores |
| To assess the effects of ALXN2220 on | Changes from baseline in CRP, IL IL1b, IL6, IL8, |
| circulating biomarkers for immune activation | IFNg, TNF-α, IL10, IL1RA, C3 and C4, and serum |
| | amyloid A, and ferritin |
| To assess the effects of ALXN2220 on | Changes from baseline in serum carboxy-terminal |
| circulating biomarkers for fibrosis | PICP, PIIINP, serum CITP, plasma PRO-C6 |
| To assess the effects of ALXN2220 on | Change from baseline in TTR (prealbumin), TSH, |
| physiological TTR function | RBP, fT4 |
| To assess the effects of ALXN2220 on | Change from baseline in PND score and FAP |
| neurological disability, QoL, and | stage |
| neurodegeneration measures among | Changes from baseline in Norfolk QoL-DN total |
| ATTRv-CM | score |
| | Change from baseline in sNFL levels |
| To assess the effects of ALXN2220 on | Change from baseline in NIS |
| polyneuropathy measures among ATTRv-CM | Change in NC studies |

### 4. Study Design

### 4.1 Overall Design

This is a Phase 3, randomized, double-blind, placebo-controlled study to evaluate the efficacy and safety of amyloid depleter ALXN2220 compared to placebo in adult participants with ATTR-CM who are on standard therapy, as determined by treating and study physician(s), which may include conventional heart failure therapies and approved disease modifying agents for ATTR amyloidosis.

The study consists of a Screening Period (up to 35 days), followed by a Blinded Treatment Period. The Blinded Treatment Period will last a minimum of 24 months for each participant. It will end when the last participant completes 24 months of blinded treatment or after a participant completes 48 months of blinded treatment, whichever is earlier. Upon completion of the Blinded Treatment Period, participants will be followed in the Safety Follow-up Period (120 days). The primary objective of the study is the assessment of the efficacy of ALXN2220 through analysis of a composite endpoint of ACM and total CV clinical events.

### Screening

Consenting participants will be screened for study eligibility for up to 35 days prior to Day 1. Participants who satisfy all the inclusion criteria and meet none of the exclusion criteria will be randomized.

### Randomization

Approximately 1000 participants will be enrolled and randomized to receive either ALXN2220 or placebo in a 2:1 ratio. The randomization will be stratified by 3 factors: treatment with a disease modifying agent at Screening (TTR silencer±TTR stabilizer vs TTR stabilizer alone vs none), TTR genotype (variant vs wild-type), and disease severity based on cardiac biomarker at Screening (NT-proBNP>3000 pg/mL vs NT-proBNP s 3000 pg/mL).

The number of participants in any of the strata may be capped depending on recruitment numbers.

### Blinded Treatment Period

During the Blinded Treatment Period, participants will receive study intervention (ALXN2220 or placebo) q4w via IV infusion for at least 24 months up to a maximum of 48 months according to the schedule summarized in Table 25 (up to 24 months) and Table 26 (after 24 months for all participants who complete 24 months of study treatment prior to the last participant).

### Early Discontinuation

Participants who prematurely discontinue study treatment should be encouraged to complete all scheduled study visits and assessments (except study intervention infusion). Participants withdrawing from the study prior to completion of the Blinded Treatment Period, regardless of cause, should be encouraged to complete the ED Visit as soon as possible and should be encouraged to attend the subsequent safety follow-up visits up to 120 days from the last dose (Tables 25 and 26).

### Safety Follow-Up Period

For all participants, post-treatment safety follow-up visits will be conducted 60 days and 120 days after the last dose of study intervention to collect information on laboratory parameters, concomitant medications, non-pharmacologic therapies and procedures, and AEs. Safety data will be reviewed by an IDMC during the study (Section 10.1.6.1).

### 4.2 Study Design

ATTR amyloidosis is a rare, progressive degenerative fatal disease caused by the misfolding of TTR, a tetrameric plasma protein that is secreted primarily by hepatic cells and is involved in the transport of T4 and vitamin A (retinol). The disease may arise from TTR genetic variants causing a hereditary disease (ATTRv) or from wild-type TTR, which manifests as a late-onset sporadic disease (ATTRwt). ATTR-CM is an increasingly recognized cause of heart failure with poor prognosis and limited therapeutic options. In ATTR-CM, ATTR deposits primarily accumulate in the cardiac extracellular space. ATTR deposits stiffen the myocardium and cause diastolic dysfunction with associated filling abnormalities and, as the disease progresses, conduction abnormalities, arrhythmias, and ultimately impaired systolic function.

Current treatment options for ATTR-CM include TTR-stabilizing therapies, ATTR-adapted conventional heart failure therapies and supportive care to manage CV complications, while heart transplantation remains the only approach able to restore cardiac function. Tafamidis (VYNDAQEL®, VYNDAMAX®), an oral TTR stabilizer, is the only approved treatment specifically targeting ATTR-CM, both wild-type and hereditary forms. Although tafamidis has shown to significatively improve survival rates and reduce number of CV hospitalizations over placebo, patients on tafamidis present progression of the disease over time, with benefits more clinically relevant in patients with mild to moderate disease severity than in those with more advanced stages of the disease. Additional therapies for TTR stabilization or TTR gene silencing are in late-stage clinical development for ATTR-CM. However, there is currently no treatment available to deplete ATTR amyloid from the heart and to revert cardiac dysfunction. Current treatment options for patients with ATTR-PN (including patients with a mixed phenotype) include liver transplantation, TTR-stabilizing therapy (tafamidis), and TTR silencing. Patients with mixed phenotypes are still underserved by the current best care due to lack of efficacy in a significant number of patients or progression of their dysfunctions while on standard therapies.

ALXN2220 (formerly known as NI006) is a recombinant human anti-ATTR IgG1 mAb that was generated based on a comprehensive immune repertoire analysis of memory B-cell complements of healthy elderly human subjects. ALXN2220 selectively binds amyloid conformations of both wild-type and mutant TTR, with no binding to physiologically folded TTR. The antibody acts as an ATTR depleter, by inducing antibody-mediated phagocytosis of ATTR fibrils and depletion of ATTR deposits from tissues (Michalon, 2021).

In nonclinical pharmacology studies (Michalon, 2021), ALXN2220 was shown to activate immune cells via binding to Fc gamma receptors. It has been demonstrated using different in vitro and in vivo models, that ALXN2220 triggers the elimination of ATTR fibrils from patient tissues through immune driven phagocytosis clearance in a dose- and time-dependent manner.

Safety, tolerability, PK, and efficacy of treatment with ALXN2220 were evaluated in a first-in-human Phase 1 study in adult participants with ATTR-CM (Study NI006-101, NCT04360434). Available data from Study NI006-101 indicates that ALXN2220 is generally well-tolerated in participants with ATTR-CM. Primary efficacy data from Cohort 1 to Cohort 6 showed that ALXN2220 reduced cardiac ATTR amyloid deposits in a dose- and time dependent-manner as measured by 2 independent imaging proxies of cardiac amyloid load (Garcia-Pavia, 2023).

ALXN2220's high selectivity toward TTR amyloid conformations results from its binding to a cryptic epitope in TTR, which is not accessible in the physiological protein conformation and becomes exposed only following misfolding of TTR. This is a relevant potential advantage over current treatments both approved or in development, as the absence of binding to physiological TTR avoids antibody sequestration on the physiological protein and preserves TTR's biological activity.

The present disclosure is based, in part, on the recognition that that the antibodies of the present disclosure promote the elimination of ATTR deposits in patients suffering from ATTR amyloidosis. In this regard, ALXN2220/NI006 is expected to restore organ function and structure. Specifically, in accordance with the present disclosure, treatment of patients with ALXN2220 is expected to decrease cardiac muscle stiffness, to improve heart contractility and elasticity, and to improve peripheral neurological functions. This effect is expected to result not only in symptom stabilization or regression of ATTR-CM but also improve organ function and survival of the patients undergoing treatment.

### 4.3 Dose

A new dosing regimen based on PK/PD modeling & simulation has been developed. The ALXN2220 Phase 3 study dose regimen of the present disclosure is a body weight-bracketed flat dose based on the patient's recent recorded body weight (within 30 days) administered IV q4w.

The proposed dose levels are based on nonclinical pharmacology studies, exposure, efficacy, and safety data from the ongoing Phase 1 Study NI006-101 in patients with ATTR-CM and PK/PD modeling and simulation of the Phase I data.

In nonclinical pharmacology studies (Michalon, 2021), ALXN2220 was shown to activate immune cells via binding to Fc gamma receptors. It has been demonstrated using different in vitro and in vivo models, that ALXN2220 triggers the elimination of ATTR fibrils from patient samples through immune driven phagocytosis clearance in a dose- and time-dependent manner.

In the Phase 1 study NI006-101, monthly ALXN2220 treatment was generally safe and well-tolerated in adult patients with ATTR-CM up to the highest dose tested (i.e., 60 mg/kg IV q4w). ALXN2220 PK profiles were seen to be dose-proportional, provide sustained ALXN2220 levels and to be compatible with monthly dosing. In this study, the amount of ATTR deposits in the heart was estimated using 2 different methods: quantification of cardiac tracer uptake in the heart by scintigraphy or quantification of ECV by cMRI. These 2 PD measurements are proxies for cardiac amyloid load and served to estimate baseline amyloid load and change over time during the clinical study. ALXN2220 showed dose- and time-dependent reductions in the cardiac amyloid load up to approximately 51% at 60 mg/kg at 12 months.

### Notable Clinical Safety and Biomarker Observations in Study NI006-101

ALXN2220 has been generally well tolerated in participants treated with 0.3 to 60 mg/kg in Study NI006-101 during the SAD/MAD and OLE phases. No participant experienced a protocol defined dose-limiting toxicity, and no SAEs were assessed as related to ALXN2220.

In the Phase 1 study, there were no safety observations in the SAD/MAD phase or the OLE which were supported further clinical investigation in Phase 3 at the highest dose. The current Phase 3 trial further includes appropriate risk mitigation and additional risk characterization features, as outlined below.

A transient, time- and dose-dependent increase in plasma CRP was observed during the initial treatment (mainly Cₘₐₓ dependent) with doses ≥10 mg/kg of ALXN2220 and was not accompanied by a broader release of proinflammatory cytokines (TNF-α, IFNg) or complement activation (C3, C4). Also, at 60 mg/kg (Cₘₐₓ>1400 μg/mL), transient increases of plasma NT-proBNP were observed 2 to 3 days following the first dose of ALXN2220. Such NT-proBNP increases were not observed following the fifth dose, indicating an effect that is most predominant with the first treatment administration. No long-term increases in CRP were observed. Over a treatment period of 12 months at 60 mg/kg, cTnT and NT-proBNP levels were reduced by median relative change of 21% and 72% compared to baseline, respectively.

### PK/PD Modeling and Simulation Analysis Overview

The current disclosure is based, in part, on identification and use of effective dosages of anti-TTR antibodies such as NI006/ALXN2220 in the treatment of ATTR-CM. In accordance with the present disclosure, a robust population PK/PD model was developed based on data from the Phase 1 study (NI006-101) of ALXN2220 in participants with ATTR-CM. Features of the modeling include, e.g., measurement of serum ALXN2220 concentrations and 2 proxy measurements of cardiac amyloid, short-axis ECV_Mid by MRI and HRWBR by scintigraphy.

PK of ALXN2220 was well described by a two-compartmental model with linear clearance from the central compartment and ATTR binding and ALXN2220:ATTR complex elimination in the peripheral compartment.

A linear PK model, with an estimated linear terminal half-life of 23.8 days, was used. Normal IgG1 PK and effect of target binding were able to explain the PK observations, making it a physiologically plausible model.

Body weight was found to be a significant covariate of the central and peripheral volumes of distribution.

A dose-finding approach along with dose formulation considerations resulted in the development of the following flat dose brackets, which optimized exposure for efficacy while maintaining safety across the anticipated weight range in the current study (Table 17):

TABLE 17

| Body Weight Range and Dose of ALXN2220 | |
|---|---|
| Body Weight Range | Dose |
| ≥40 kg to <60 kg | 2400 mg |
| ≥60 kg to <100 kg | 3200 mg |
| ≥100 kg | 4800 mg |

The above flat weight based ALXN2220 dosing regimen is expected to result in a median amyloid reduction between 30 and 60 mg/kg.

### 4.4 End-of-Study Definition

The study primary completion is defined as the date that the last surviving participant completes 24 months of the Blinded Treatment Period, i.e, Visit 30 [Week 104].

The end of the study is defined as the date the last participant completes the last scheduled procedure as indicated in the SoA (Tables 25 and 26).

### 5. Study Population

### 5.1 Inclusion Criteria

Participants are eligible to be included in the study only if all of the following criteria apply:

### Age

1. Male or female ≥18 years to ≤90 years of age at time of randomization

### Type of Participant and Disease Characteristics

2. Centrally confirmed diagnosis of ATTR-CM with either wild-type or variant TTR genotype based on:

Evidence of cardiac amyloidosis by echocardiography or cMRI and one of the following:
a. Endomyocardial biopsy with confirmatory TTR amyloid typing by either immunohistochemistry or mass spectrometry
OR
b. Grade 2 or 3 cardiac uptake on 99mTc scintigraphy (99mTc DPD, 99mTc PYP, or 99mTc HMDP) in the absence of monoclonal gammopathy
OR
c. Grade 2 or 3 cardiac uptake on 99mTc scintigraphy (99mTc DPD, 99mTc PYP, or 99mTc HMDP) AND confirmatory TTR amyloid typing by either immunohistochemistry or mass spectrometry in non-cardiac tissue in the presence of monoclonal gammopathy
3. Willing to be genetically tested for mutations in the TTR gene during screening, if genetic testing was not previously performed or if genetic results are not available
4. End-diastolic interventricular septal wall thickness ≥11 mm for women or ≥12 mm for men on echocardiography measured at Screening
5. NT-proBNP>2000 pg/mL measured by a central laboratory at Screening
6. Treatment with a loop diuretic for at least 30 days prior to Screening
7. History of heart failure as documented by one of the following events within 1 year prior to Screening:
   a. heart failure hospitalization
   b. urgent heart failure visit
   c. episode of volume overload documented by NT-proBNP>2000 pg/mL (or equivalent BNP)
8. NYHA Class II-IV at Screening
9. Life expectancy of ≥6 months as per the Investigator's judgment

### Sex

10. Male and/or female

### Informed Consent

11. Capable of giving signed informed consent as described in Section 10.1.3

### 5.2 Exclusion Criteria

Participants are excluded from the study if any of the following criteria apply:

### Medical Conditions

### Weight

### Prior/Concomitant Therapy

Section 6.9.2

### Prior/Concurrent Clinical Study Experience

### Diagnostic Assessments

### Other Exclusions

### 5.3 Lifestyle Considerations

### 5.4 Screen Failures

A screen failure occurs when a participant who has consented to participate in the clinical study is not subsequently assigned to study intervention.

### 5.5 Criteria for Temporarily Delaying Randomization

5.6 Participants with ATTR-CM

In participants with ATTRv-CM, PND score, FAP stage, sNfL and Norfolk QoL-DN will be recorded at designated timepoints (Table 25, footnote m and Table 26).

### 6. Study Intervention(s) and Concomitant Therapy

Study interventions are all prespecified, investigational and non-investigational medicinal products, medical devices and other interventions (e.g., surgical and behavioral) intended to be administered to the study participants during the study conduct.

### 6.1 Study Intervention(s) Administered

The study intervention composition and doses to be administered in this study are presented in Table 18.

TABLE 18
Study Intervention

| ARM Name | ALXN2220 | | Placebo |
|---|---|---|---|
| Study Intervention | ALXN2220 | | Placebo |
| Name | | | |
| Type | Biologics | | NA |
| Dose Formulation | ALXN2220 is formulated at 50 mg/mL in 20 mM | | 20 mM histidine buffer, 80 mg/mL |
| | histidine buffer, 80 mg/mL sucrose, 0.3 mg/mL | | sucrose, 0.3 mg/mL polysorbate |
| | polysorbate 80, at pH 5.8 | | 80, at pH 5.8 |
| Unit Dose Strength | 50 mg/mL | | NA |
| Dosage Level | Body weight range | Dose | Same number of vials and volume |
| | ≥40 kg to <60 kg | 2400 mg | as ALXN2220 |
| | ≥60 kg to <100 kg | 3200 mg | |
| | ≥100 kg | 4800 mg | |

| | | |
|---|---|---|
| Storage | 2-8° C.; protect from light | 2-8° C.; protect from light |
| Temperature | | |
| Route of | IV infusion over 2 hours for initial administration | IV infusion over 2 hours for initial |
| Administration | and over 1 hour for subsequent administrations | administration and over 1 hour for |
| | if no safety issues | subsequent administrations if no |
| | | safety issues |
| Use | Experimental | Placebo-comparator |

### 6.2 Preparation, Handling, Storage, and Accountability

The Investigator or designee must confirm appropriate conditions (e.g., temperature) have been maintained during transit for all study intervention received, and any discrepancies are reported and resolved before use of the study intervention.
Only participants enrolled in the study may receive study intervention, and only authorized site staff may supply, prepare, or administer study intervention.
All study intervention must be stored in a secure, environmentally controlled, and monitored (manual or automated) area in accordance with the labeled storage conditions with access limited to the Investigator and authorized site staff.
The Investigator or authorized site staff is responsible for study intervention accountability, reconciliation, and record maintenance (i.e., receipt, reconciliation, and final disposition records).
Site personnel responsible for drug storage/accountability and/or preparation are at risk of unblinding, and these personnel will not be involved in conducting study assessments. The site pharmacist should refer to the Pharmacy Manual for instructions on masking the study intervention.
Further guidance regarding preparation, handling, potential home-based infusions, storage, accountability, and information for the final disposition of unused study interventions are provided in the Pharmacy Manual.

### 6.3 Assignment to Study Intervention

All participants will be centrally assigned to randomized study intervention using an IVRS/IWRS. Participants will be randomized in a 2:1 ratio to ALXN2220 or placebo. Once a randomization number has been assigned it must not be reassigned.

Randomization will be stratified based on treatment with a disease modifying agent at Screening (TTR silencer±TTR stabilizer vs TTR stabilizer alone vs none), TTR genotype (variant vs wild-type), and disease severity based on cardiac biomarker at Screening (NT-proBNP>3000 pg/mL vs NT-proBNP≤3000 pg/mL).

For the purpose of stratified randomization, diflunisal will be considered a TTR stabilizer. Study intervention will be dispensed at the study visits as summarized in the SoA (Section 12). Returned study intervention should not be redispensed to the participants.

### 6.4 Blinding

Participants, investigative site personnel, Alexion staff, Alexion designees, and staff directly associated with the conduct of the study will be blinded to participant treatment assignments (except as indicated below in Section 6.4.2). To preserve the blinded treatment assignments, these individuals will also be blinded to any assessment results that have the potential to unblind treatment assignment, including PK results.

### 6.4.1 Unblinding

There will be up to 2 analysis milestones in the study that require unblinding:

### 1. Optional Interim Analysis

Study participants, study sites, and members of the Alexion study team who are in direct contact with the study sites will remain blinded (except as indicated below in Section 6.4.2). The IA data will be analyzed and interpreted by the IDMC and associated independent statistical center based on prespecified criteria (Section 9.4).

2. Final Unblinding after Completion of the Blinded Treatment Period

This will occur at the time of the primary analysis when the last surviving participant completes 24 months of study treatment. Study sites and all members of the Alexion study team will be unblinded. Further details will be provided in the Unblinding Plan.

### 6.4.2 Measures to Minimize Bias

The Pharmacy Manual will detail any investigative site personnel that may need to be unblinded for IV infusion preparation or investigational product accountability monitoring.

### 6.4.3 Emergency Unblinding

The IRT will be programmed with blind-breaking instructions. In the event that emergency unblinding is necessary, the IRT will provide clear step-by-step instructions for the Investigator to follow.

### 6.5 Study Intervention Compliance

When participants are dosed at the site, they will receive study intervention directly from the Investigator or designee, under medical supervision. The date and time of each dose administered in the clinic will be recorded in the source documents. The dose of study intervention and study participant identification will be confirmed at the time of dosing by a member of the study site staff other than the person administering the study intervention.

### 6.6 Dose Modification

The dose level in this study is based on the individual participant's weight as described in Section 4.3. If a participant's weight changes from one weight bracket to another during the study, the administered dose will change accordingly. For the dose determination of individual infusions, a weight measurement collected within 30 days prior to treatment administration has to be utilized.

If the weight of a participant drops below 39 kg (i.e., 40 kg-2.5%) continuation of treatment may only be permitted after discussion with the Sponsor's Medical Monitor.

Guidance for management of IRRs including adjustment in dose administration for safety reasons are located in Section 10.9.

6.7 Continued Access to Study Intervention after the End of the Study

Participants who complete the Blinded Treatment Period may be eligible to participate in an OLE study to be developed.

### 6.8 Treatment of Overdose

Any dose of ALXN2220 greater in amount than the planned monthly dose calculated according to the participant's weight (collected within 30 days prior to administration) and based on the specific weight brackets within a 4-week period (±time window according to SoA; Tables 25 and 26) will be considered an overdose.

In the absence of an antidote to ALXN2220, specific treatment for an overdose is not recommended and only general supportive measures are recommended.

### 6.9 Prior and Concomitant Therapy

In this study, participants in both treatment groups will receive standard therapy as concomitant therapy, which will be determined by the Investigator according to institutional practices and participant characteristics, unless prohibited in Section 6.9.2.

### 6.9.1 Allowed Medicine and Therapy

Participants are to be treated according to the standard therapy, as determined by treating and study physician(s). This includes ATTR-adapted conventional heart failure treatment (including diuretics) and may include approved disease modifying agents for ATTR amyloidosis.

Treatment with locally approved TTR gene silencing agent for ATTR amyloidosis is (except as listed in Section 6.9.2) permitted if given prior to enrollment in this study at a stable dose for at least 90 calendar days prior to signing the ICF. New treatment initiation of a TTR gene silencer is allowed at the locally approved dosage regimen, but not during the first 90 days after randomization.

### 6.9.2 Disallowed Medicine and Therapy

The following concurrent medications are prohibited during the study. If use during the study is medically required, the participant may be withdrawn, at least temporarily for the time of treatment with the disallowed medicine, from the administration of study intervention.
Inotersen. If previously used, a minimum wash-out period of 180 days prior to Day 1 has to be respected (Section 2.3.1).
ATTR amyloid depleter (i.e., monoclonal anti-ATTR antibody) approved or in clinical development.
Diflunisal unless prescribed as local standard therapy (for ATTR-CM, pain, or arthritis) under appropriate safety precautions as per Investigator's assessment.
Doxycycline or tauroursodeoxycholic acid (TUDCA). If previously used, a minimum washout period of 14 days prior to Day 1 is required. Short-term usage of doxycycline during the study for indications other than ATTR-CM (e.g., bacterial infection) may be permitted with the agreement of Alexion's Medical Monitor.
Non-dihydropyridine calcium channel blockers with conduction system effects (e.g., verapamil, diltiazem). If previously used, a minimum wash-out period of 14 days prior to Day 1 is required.
Treatment with systemic immunosuppressive or immune modulating drugs including, but not limited to, antimetabolites (e.g., mycophenolate mofetil, azathioprine), calcineurin inhibitors (e.g., cyclosporine, tacrolimus) and non-calcineurin inhibitors (e.g., everolimus, sirolimus), biologic agents and cytokine modulators (e.g., alemtuzumab, basiliximab, daclizumab, muromonab, rituximab, adalimumab, infliximab, etanercept, tocilizumab, stiltuximab). Treatment regimens for multiple myeloma and/or Light Chain (AL) amyloidosis such as cyclophosphamide, bortezomib and dexamethasone, and daratumumab are also not permitted. Short-term use of systemic corticosteroids (eg prednisone) may be permitted with the agreement of Alexion's Medical Monitor.
IV or subcutaneous human immunoglobulins (e.g., IVIG or SCIG).
Cytotoxic chemotherapy (e.g., cyclophosphamide).

### 7. Discontinuation of Study Participation and Participant Discontinuation/Withdrawal

### 7.1 Discontinuation of Study Intervention

In rare instances, it may be necessary for a participant to permanently discontinue study intervention. See the SoA (Section 12) for data to be collected at the time of discontinuation of study intervention and follow-up and for any further evaluations that need to be completed.

Discontinuation may follow inflammatory reaction (Section 10.10 and Section 10.11, respectively); pregnancy or planned pregnancy (Section 8.4.7); liver stopping criteria (Section 10.8); any other AE or other observation (e.g., laboratory abnormality) that would, in the opinion of the Investigator or Alexion, make continued participation in the study an unacceptable risk; use of disallowed medication (Section 6.9.2); and/or termination of the study.

### 7.1.1 Liver Chemistry Stopping Criteria

Study intervention will be discontinued for a participant if liver chemistry stopping criteria are met (Section 10.8).

### 7.1.2 Temporary Discontinuation

If the participant's average resting systolic BP is <90 mmHg at the preinfusion vital sign assessment (Section 8.3.2), study intervention administration should be delayed pending evaluation of the participant's clinical status. Guidance for treatment interruption and modification of study intervention administration for specific safety reasons is detailed in Section 10.8 through Section 10.11.

7.2 Participant Discontinuation/Withdrawal from the Study
See Section 12.

### 7.3 Lost to Follow-Up

A participant will be considered lost to follow-up if the participant repeatedly fails to return for scheduled visits and is unable to be contacted by the study site.

### 8. Study Assessment and Procedures

Study procedures and their timing are summarized in the SoA (Section 12). See Section 10.2 for a listing of clinical laboratory tests.

### 8.1 Administrative

### 8.1.1 Informed Consent

The Investigator, or qualified designee, must obtain a signed and dated informed consent/assent form from each participant prior to conducting any study procedures.

### 8.1.2 Inclusion/Exclusion Criteria

All inclusion (Section 5.1) and exclusion (Section 5.2) criteria must be reviewed by the Investigator or qualified designee to ensure the participant qualifies for study participation.

### For Inclusion Criterion 2 (Section 5.1):

The diagnosis of ATTR-CM will require confirmation by Medical Monitor.
Scintigraphy readings will require confirmation by a central imaging core laboratory according to the Imaging Manual.
If historical results are insufficient to assess monoclonal gammopathy, the required laboratory tests can be performed by the central laboratory during Screening.

### 8.1.3 Demographics

Demographic parameters, including age, sex, race, ethnicity, socioeconomic status, and disability.

### 8.1.4 Medical History

The Investigator will review the participant's history and diagnosis and document the following at the Screening Visit:

Medical history including all relevant medical/surgical history.

Any changes to medical history occurring during the Screening Period and prior to first dose of study intervention on Day 1 will be documented.

### Polyneuropathy Assessment

In centers previously identified with conditions for neurological assessment, participants with ATTRv-CM will have the option to be evaluated in a neurology consultation as described in Section 8.2.13.

### 8.2 Efficacy Assessments

### 8.2.1 Mortality

ACM, an efficacy endpoint for this study, and the events that lead to death will not be reported/collected as AEs except as detailed in Section 8.4.1. CV related mortality is one of the secondary endpoints.

All mortality events will be reviewed and adjudicated by an independent CEC and determined whether the cause of death meets the definition provided in the CEC Charter (Section 10.1.6.2).

### 8.2.2 Cardiovascular Clinical Events

CV clinical events are efficacy endpoints for this study and include:
Nonelective (unplanned) CV related hospitalizations, and
Unscheduled ambulatory visits for the treatment of heart failure (UHF visits)

Note: These clinical events will not be reported/collected as AEs or SAEs except as detailed in Section 8.4.1.

The Investigator is responsible for ensuring potential study endpoints, including dates of admissions and discharge, are collected and documented and for providing Investigator assessment whether the hospitalization is nonelective and CV related.

Subcategories of CV clinical events, including HF events (composite of HF hospitalizations and UHF visits) as well as hospitalizations for atrial fibrillation will also be assessed.

CV clinical events, including subcategories, will be reviewed and adjudicated by the CEC according to definitions and processes specified in the CEC charter (Section 10.1.6.2).

In addition, intensification of oral diuretic therapy (initiation of new or combination diuretic therapy, significant augmentation of existing diuretic therapy) is an exploratory endpoint of this study. Details of diuretic therapy will be proactively collected by site personnel at each study visit and reported on a specific eCRF page. These events will not be adjudicated by the CEC.

### 8.2.3 KCCQ

The KCCQ is a 23-item questionnaire developed to measure health status and health-related QoL in participants with heart failure. Items include heart failure symptoms, impact on physical and social functions, and how their heart failure impacts their QoL (Section 12). The KCCQ overall summary score, KCCQ-OS is a valid and reliable health status measure for patients with heart failure and has been employed in randomized controlled studies, including in ATTR-CM (Hanna, 2021; Maurer, 2018; Nativi-Nicolau, 2021).

### 8.2.4 6MWT

The 6MWT measures the distance a participant can quickly walk on a flat, hard surface in a period of 6 minutes. It evaluates the global and integrated response of all the systems involved during exercise. This is a self-paced test; participants choose their own exercise intensity and are allowed to stop and rest during the test (Crapo, 2002). If the participant has a need for a walking aid (e.g., cane) or supplemental oxygen at baseline, it must be consistently used at each subsequent 6MWT throughout the study.

### 8.2.5 Disease Stage

Several models using different surrogate markers of disease severity and/or prognosis in ATTR-CM are available (Garcia-Pavia, 2021). Although NYHA class has been shown to be prognostic in ATTR-CM, it lacks sensitivity as a standalone tool for detecting more subtle progression of ATTR-CM, and therefore should be interpreted in a multiparametric approach. The Mayo Clinic (Grogan, 2016), and the UK NAC (Gillmore, 2018) cardiac staging system for patients with ATTR-CM are 2 relatively simple applicable staging systems using universally employed biomarkers NT-proBNP and eGFR or cTnT. Another staging score for ATTR-CM is the Columbia score (Cheng, 2020) which includes NYHA and daily dose of diuretic therapy besides biomarker-based staging.

### 8.2.6 36-Item Short Form Survey

The SF-36 questionnaire (Ware, 1992) is the most widely used nondisease-specific PRO measure to assess health-related QoL and requires approximately 5 minutes to complete. It consists of 36 questions covering 8 domains: 4 on physical health and 4 on mental health, with a focus on the last 4 weeks. These 8 domains can be aggregated into 2 summary measures: PCS and MCS scores. A grade from 0 to 100 is assigned to each domain, with lower scores associated with worse QoL.

### 8.2.7 Euro QoL 5-Dimensions 5-Levels

The EQ-5D-5L (Oppe, 2014) is a widely used PRO measure evaluating health-related QoL. This includes the EQ-5D descriptive system and the EQ VAS. The descriptive system assesses mobility, self-care, usual activities, pain/discomfort, and anxiety/depression. Patients are asked to indicate the status of each dimension by selecting 1 of the 3 levels (no problems, some problems, and extreme problems). The responses are compiled to create a 5-digit number that describes the patient's health state. In the EQ VAS, the patient is asked to point their current health status on a 20-cm vertical scale ranging from 0 (‘the worst health you can imagine’) to 100 (‘the best health you can imagine’).

8.2.8 NT-proBNP and High Sensitivity cTnT

Natriuretic peptide (NT-proBNP, in pg/mL) and hs-cTnT in pg/mL, 2 widely established blood biomarkers of cardiac stress and cardiomyocyte damage with prognostic relevance (Gillmore, 2018; Grogan, 2016), will be used to assess ATTR-CM disease status at screening (part of the inclusion criteria) and to evaluate changes in cardiac structure and function over time in response to treatment with ALXN2220. See Section 10.2 for the list of clinical efficacy laboratory tests to be performed and the SoA (Section 12) for the timing and frequency.

### 8.2.9 Echocardiography

Cardiac structure and function in patients with ATTR-CM can be assessed non-invasively with echocardiography, and an increased left ventricular wall-thickness is a component in the diagnostic algorithm of ATTR-CM (Garcia-Pavia, 2021; Kittleson, 2023), and inclusion criterion for this study. Longitudinal changes in cardiac structure and function will be assessed using serial echocardiograms across all sites and following a harmonized acquisition protocol. Evaluation of all echocardiograms will be performed by a blinded central laboratory.

### 8.2.10 Cardiac Scintigraphy

Cardiac scintigraphy using 99mTc-DPD, 99mTc-HMDP and 99mTc-PYP tracers has been established as sensitive and specific tool to identify patients with cardiac ATTR amyloid deposits, has become a cornerstone in the diagnostic algorithm for ATTR-CM (Dorbala, 2020; Garcia-Pavia, 2021; Kittleson, 2023) and can be used as a surrogate marker for the cardiac amyloid load in patients with ATTR-CM (Garcia-Pavia, 2023). SPECT cardiac scintigraphy scans (±computed tomography, SPECT-CT, depending on availability at site) will be recorded according to a harmonized acquisition protocol across selected sites, during the Screening Period and Blinded Treatment Period, and evaluated by a blinded central laboratory. Image acquisition is to be performed 3 hours (±15 minutes) after tracer infusion, and tracer type should not be changed for each individual participant during the course of the study. If required for eligibility confirmation (Inclusion Criteria 2b and 2c), historical or current cardiac scintigraphy results need to be confirmed by the central laboratory.

8.2.12 cMRI

cMRI can be utilized to visualize and indirectly quantify the full continuum of amyloid deposition in the heart (Martinez-Naharro, 2017) using advanced cMRI protocols with contrast agents. ECV on cMRI correlates with other markers of ATTR disease status, is predictive of mortality (Martinez-Naharro, 2017) and can be considered a surrogate marker of cardiac amyloid load (Garcia-Pavia, 2023). In the current study, participants who provide separate informed consent will have optional serial cMRIs taken according to a harmonized acquisition protocol at selected sites with the required cMRI expertise and evaluated by a blinded central laboratory.

Dual imaging with cardiac scintigraphy and cMRI is encouraged at selected sites.

### 8.2.13 Cardiac, Salivary Gland or Skin Punch Biopsy (Optional)

Endomyocardial biopsy has historically been considered the gold standard for establishing the diagnosis of ATTR-CM and continues to be required in the diagnostic algorithm in case of inconclusive results on scintigraphy and hematological tests (Garcia-Pavia, 2021).

IENFD, SGNFD in skin punch has been shown to be accurate and reliable at evaluating small fiber neuropathy and has been used as an outcome measure in clinical studies for several neuropathies, including diabetic neuropathy, Fabry disease, and TTR neuropathy (Ebenezer, 2017; Leonardi, 2022; Masuda, 2017). The use of two biopsy samples has been shown to increase the sensitivity for the detection of amyloid by 86% (Freeman, 2022). These biopsies were used to quantify IENFD (sensory innervation) and SGNFD (autonomic innervation) based on blinded central analysis. Congo Red staining was used to serially assess dermal amyloid burden in the biopsies.

In the current study, if the participant has provided separate informed consent, cardiac, salivary or skin punch biopsies will be obtained according to a Laboratory Manual. Target engagement of ALXN2220, antibody-mediated amyloid depletion and related biological processes will be explored on tissue from cardiac or extracardiac biopsies.

### 8.2.13 Neurological Assessments

### 8.2.13.1 Neuropathy Impairment Score

The NIS is a composite score of clinical impairments that has been widely used to assess the severity of peripheral neuropathy objectively. This includes standard assessment of muscle weakness and groups of muscles, reflexes, and sensory modalities at specific sites on both sides of the body. The total NIS is graded on a scale of 0 to 244, with a higher score indicating more significant impairment; a 2-point change is considered the least degree of change a physician could recognize (Dyck, 1991; Dyck, 2019). In controlled clinical studies, NIS has been used to assess disease progression and response to treatment in a number of polyneuropathies and correlated with FAP stage, PND score, Norfolk QoL-DN, and disease duration (Adams, 2015; Coelho, 2017; Vinik, 2014) in patients with ATTR-PN.

### 8.2.13.2 Norfolk QoL-DN

The Norfolk QoL-DN is a self-administered 35-item questionnaire that quantifies the impact of neuropathy on patients' QoL. This instrument is a nerve fiber-specific, 5-domain tool validated in FAP patients (Vinik, 2014). The Norfolk QoL-DN consists of 1 composite score (TQoL) and 5 subdomain scores attributed to one of the following: physical functioning/large fiber neuropathy; activities of daily living (ADLs); symptoms: small fiber neuropathy: autonomic nerve function. The scores across the 5 domains are summed to provide the TQoL score and the total score ranging from −4 to 136 (the higher the score, the poorer the QoL). The Norfolk QoL-DN underwent linguistic validation for each country and language. The Norfolk QoL-DN was used in ATTR-PN studies as primary and secondary endpoints (Adams, 2018; Adams, 2023; Benson, 2018; Coelho, 2012; Coelho, 2023).

### 8.2.13.3 Polyneuropathy Disability Score

The PND score is a 6-stage scoring system to assess neuropathy in FAP patients (Yamamoto, 2007). Stage 0 indicates no impairment; Stage I indicates sensory disturbances with preserved walking capacity; Stage II indicates impaired walking capacity but the ability to walk without the need for a walking stick; a score Illa indicates 1 stick or crutch is required for walking; IIIb indicates 2 sticks or crutches are required for walking; and IV indicates the patient is wheelchair-bound or bedridden.

### Familial Amyloid Polyneuropathy Staging System

FAP staging system (Coutinho, 1980) is a system used to classify the severity of disability ATTR-PN patients. FAP staging encompasses 3 stages based on ambulatory status: Stage 1 does not require assistance with ambulation, Stage 2 requires assistance with ambulation, and Stage 3 is wheelchair-bound.

8.2.13.4 Serum Neurofilament light chain

The NfL is one of the neural cytoskeleton proteins with important roles in axonal and dendritic branching and growth. This protein can be measured in the blood and cerebrospinal fluid of patients and has been shown to be elevated in neurological diseases that involve neuronal damage of the CNS and PNS diseases (Gaetani, 2019). Recent data from observational studies and a clinical study support sNfL could be a promising multipurpose biomarker for neuropathy in hereditary transthyretin amyloidosis, namely for early diagnosis and to detect activity and evaluate treatment response (Kapoor, 2019; Maia, 2020; Ticau, 2021)

8.2.13.5 Nerve conduction studies

The nerve conduction tests involve five attributes: fibular CMAP, tibial CMAP, ulnar CMAP, SNAP, and ulnar SNAP amplitudes. The five attributes of nerve conduction results were reported as normal deviates (NDs) from percentiles derived from reference values of healthy study participants.

### 8.3.1 Physical Examinations

A complete physical examination will include, at a minimum, assessments of the following inspection of general appearance, skin, nose, ears, eyes, neck, throat, heart, abdomen, lungs, vascular system, nervous system, musculoskeletal system, and extremities. Height (at Screening only) and weight will also be measured and recorded.

An abbreviated physical examination will include general appearance, heart, lungs, skin, musculoskeletal system and extremities and other organs or body systems based upon Investigator's (or qualified designee) judgment and participant symptoms as specified in the SoA (Section 12).

### 8.3.2 Vital Signs

Vital signs will be measured in a resting position (reclining or supine) after 5 minutes rest as outlined in the SoA (Section 12) and will include temperature, systolic and diastolic BP, pulse, oxygen saturation (SpO₂, pulse oximetry), and respiratory rate. Three readings of BP and pulse will be taken. The first reading should be rejected. The second and third readings should be averaged to give the measurement to be recorded.

### 8.3.3 Electrocardiograms

Single 12-lead ECGs will be obtained as outlined in the SoA (Section 12) using an ECG machine that automatically calculates the heart rate and measures PR, QRS, QT, and QTc intervals. A blinded central laboratory may be used to perform specialized assessment, hence, electronic tracings may be stored centrally.

### 8.2.4 Clinical Safety Laboratory Tests

See Section 10.2 for the list of clinical safety laboratory tests to be performed and the SoA (Section 12) for the timing and frequency.
The Investigator must review the laboratory results, document this review, and record any clinically significant changes occurring during the study as an AE in the AE section of the CRF. The laboratory results must be retained with source documents.
All laboratory tests with values considered clinically significantly abnormal during participation in the study or up to 120 days after the last dose of study intervention should be repeated until the values return to normal or baseline or are no longer considered clinically significant by the Investigator or Alexion's Medical Monitor.
   If clinically significant values do not return to normal/baseline within a period of time judged reasonable by the Investigator, the etiology should be identified, and Alexion notified.
   All protocol-required laboratory tests, as defined in Section 10.2, must be conducted in accordance with the Laboratory Manual and the SoA (Section 12).
   If laboratory values from non-protocol-specified laboratory tests performed at the institution's local laboratory require a change in participant management or are considered clinically significant by the Investigator (e.g., SAE or AE or dose modification), then the results must be documented.
   During Screening and scheduled or unscheduled study visits, sites must obtain and ship samples for central lab testing regardless of sample collection for local labs. During Screening and scheduled or unscheduled study visits, central lab results prevail over local lab results when both are available.
   Delay in central lab results beyond the Screening Period will make local labs the basis for eligibility determination. In this case, even if the central lab result later shows the participant as ineligible, eligibility already determined based on local labs is acceptable.

Local labs without central lab results can be used during the study for safety testing, dose modification/withholding, or discontinuation from the study.

### 8.3.5 Pregnancy Testing

Refer to Section 5.1 Inclusion Criteria for pregnancy testing entry criteria.
Pregnancy testing (urine or serum as required by local regulations) should be conducted monthly ahead of infusion during intervention.
Pregnancy testing (urine or serum as required by local regulations) should be conducted during the same time frame as the contraceptive requirements (i.e., 5 months after the last dose of study intervention.
Additional serum or urine pregnancy tests may be performed, as determined necessary by the Investigator or required by local regulation, to establish the absence of pregnancy at any time during the participant's participation in the study.

### 8.3.6 Participant Safety Card

Before the first dose of study intervention a Participant Safety Card will be provided to participants to carry with them at all times until completion of the post-treatment safety follow-up visit or 7 months following the last dose of study intervention. The card describes the goal of the study, important information about study intervention, and provides contact information for the Investigator.

### 8.4 Adverse Events (AEs), Serious Adverse Events (SAEs), and Other Safety Reporting

The definitions of AEs and SAEs including protocol specific instructions are found in Section 10.3.

The Investigator and any qualified designees are responsible for detecting, documenting, and recording events that meet the definition of an AE or SAE and remain responsible for following up all AEs (see Section 7). This includes events reported by the participant (or, when appropriate, by a caregiver, surrogate, or the participant's legally authorized representative).

The method of recording, evaluating, and assessing causality of AEs and SAEs and the procedures for completing and transmitting SAE reports are provided in Section 10.3.

### 8.4.1 Clinical Events Referred to the Clinical Endpoint Committee

Clinical events suspected by the Investigator to reflect a component of the efficacy endpoints (any death, any CV clinical event), are to be recorded in the efficacy endpoint eCRFs for adjudication by the CEC.

Additional clinical events considered to be an efficacy endpoint by Alexion will also be routed for adjudication by the CEC.

When there is a difference in an event reported by an Investigator and the adjudicated CEC decision, the CEC's decision will be considered final. Positively adjudicated events will be analyzed and reported as efficacy parameters. Negatively adjudicated events (events not meeting prespecified efficacy endpoint criteria) will be routed back to the Investigator for recording as AEs or SAEs, using the definitions in Section 10.3.

The Investigator must report/record any suspected efficacy endpoint considered to be related to study intervention as an AE or SAE using the definitions in Section 10.3. Investigators must also record the relevant information on efficacy endpoint eCRFs for CEC adjudication. These AEs or SAEs are subject to regulatory reporting (Section 8.4.5) and will be analyzed and reported as safety parameters (Section 8.3).

### 8.4.2 Time Period and Frequency for Collecting AE and SAE Information

All AEs and SAEs will be collected from the signing of the ICF until the final post-treatment safety follow-up visit.

All SAEs will be recorded and reported to Alexion or designee immediately and under no circumstance should this exceed 24 hours, as indicated in Section 10.3.4. The Investigator will submit any updated SAE data to Alexion within 24 hours of it being available.

The Investigator is not obligated to actively seek new information on AEs or SAEs after conclusion of the study participation. However, if the Investigator learns of any SAE, including a death, at any time after a participant has concluded participation in the study, and the Investigator considers the event to be reasonably related to the study intervention or study participation, the Investigator must promptly notify Alexion.

### 8.4.3 Method of Detecting AEs and SAEs

The method of recording, evaluating, and assessing causality of AE and SAE and the procedures for completing and transmitting SAE reports are provided in Section 10.3.

Care will be taken not to introduce bias when detecting AEs and/or SAEs. Open-ended and nonleading verbal questioning of the participant is the preferred method to inquire about AE occurrences.

### 8.4.4 Follow-Up of AEs and SAEs

After the initial AE/SAE report, the Investigator is required to proactively follow each participant at subsequent visits/contacts. All SAEs will be followed until resolution, stabilization, the event is otherwise explained, or the participant is lost to follow-up (as defined in Section 7.3). Further information on follow-up procedures is provided in Section 10.3.

### 8.4.5 Regulatory Reporting Requirements for SAEs and Other Events

Section 10.3.4 and Section 10.3.5.

### 8.4.6 Medication Error, Drug Abuse, and Drug Misuse

Medication error, drug abuse, and drug misuse will be collected from signing of the ICF through the last scheduled procedure.

### 8.4.6.1 Timelines

If an event of medication error, drug abuse, or drug misuse occurs during the study, the Investigator or other site personnel will report to Alexion or designee immediately but no later than 24 hours of when they become aware of it.

The full definitions and examples of medication error, drug abuse, and drug misuse can be found in Section 10.4.

### 8.4.6.2 Medication Error

For the purposes of this clinical study a medication error is an unintended failure or mistake in the treatment process for an IMP that either causes harm to the participant or has the potential to cause harm to the participant.

### 8.4.6.3 Drug Abuse

Drug abuse is the persistent or sporadic intentional, non-therapeutic excessive use of IMP for a perceived reward or desired non-therapeutic effect.

### 8.4.6.4 Drug Misuse

Drug misuse is the intentional and inappropriate use of IMP for medicinal purposes outside of the authorized product information, or for unauthorized IMPs, outside the intended use as specified in the protocol, including deliberate administration of the product by the wrong route.

### 8.4.7 Pregnancy

Details of all pregnancies in female participants will be collected after the start of study intervention and until 5 months after the last dose of study intervention and until 7 months after the last dose of study intervention for female partners of male participants.

### 8.5 Pharmacokinetics

Blood samples of approximately 2 mL will be collected for measurement of serum concentrations of ALXN2220 as specified in the SoA (Section 12).
Instructions for the collection and handling of biological samples will be provided by Alexion. The actual date and time (24-hour clock time) of each sample will be recorded.
All efforts will be made to obtain the PK samples at the exact nominal time relative to dosing.
Study intervention concentration information that may unblind the study will not be reported to investigative sites or blinded personnel until the study has been unblinded.

### 8.6 Pharmacodynamics

PD measurements of interest include NT-proBNP, cardiac scintigraphy, echocardiography, cMRI as well as any safety cardiac markers.
Instructions for the collection and handling of biological samples will be provided by Alexion. The actual date and time (24-hour clock time) of each sample will be recorded.
All efforts will be made to obtain the PD samples at the exact nominal time relative to dosing. Out-of-window protocol deviation capture for PD samples follows that specified for PK sample collection (Section 8.5).
Samples collected for PK/PD analyses may be used for research purposes or to evaluate safety or efficacy aspects during or after the study.

### 8.7 Genetics

A single 2 mL blood sample for TTR genotyping will be collected from participants without available historic TTR genotype test results at Screening and after a signed informed consent has been obtained.

In the event of DNA extraction failure, a replacement genetic blood sample may be requested from the participant. Signed informed consent will be required to obtain a replacement sample unless it was included in the original consent.

In participants experiencing treatment-related AEs, and if separate signed informed consent is provided, additional evaluation of specific genetic polymorphisms associated with the occurrence of such AEs may be performed to generate hypotheses for further characterization and mitigation of adverse effects in populations at risk.

See Section 10.7 for information regarding genetic research. Details on processes for collection and shipment and destruction of these samples can be found in the Laboratory Manual.

### 8.8 Biomarkers

Serum and plasma samples will be collected for exploratory assessments. Biomarkers may include, but are not limited to, assessments of the following:
Complement factors C3 and C4, CRP
IL1b, IL6, IL8, IFNg, TNF-α (proinflammatory cytokines)
IL10, IL1RA (anti-inflammatory cytokines)
SAA, ferritin (positive acute phase proteins)
PICP
PIIINP
CITP
PRO-C6
Plasma NNTTR
Other proteomic assessments may be conducted based on scientific advancements and techniques

Samples will be collected according to the schedule described in the SoA (Section 12) and as detailed in Laboratory Manual provided separately to sites.

### 8.9 Immunogenicity Assessments

Serum samples for ADA will be collected at timepoints according to SoA (Section 12). All efforts will be made to obtain the immunogenicity samples at the exact nominal time relative to dosing. Out-of-window protocol deviations captured for immunogenicity samples will follow the same approach as specified for PK sample collection (Section 8.5).

The detection and characterization of ADA to ALXN2220 will be performed using validated assay methods by or under the supervision of Alexion.

ADA positive samples will be further characterized for antibody titer and presence of NAb. Samples may be stored for a maximum of 25 years (or according to local regulations) following the last participant's last visit for the study at a facility selected by Alexion. Additional analyses may be performed on collected ADA samples for further analysis or characterization.

### 8.10 Health Economics

For all participants throughout the study, the Investigator and study site personnel will collect data about health care resource utilization associated with medical encounters.

### 9. Statistical Considerations

This section is a summary of the planned statistical analyses of the most important endpoints including primary and secondary endpoints. The primary analysis will be conducted at the end of the Blinded Treatment Period (Section 4.4). After the primary analysis, supplemental analyses may be performed on the data collected during the Safety Follow-up Period to support submission requirements.

### 9.1 Statistical Hypotheses

The study is designed to test the following hypotheses for the primary and secondary efficacy endpoints:

### Primary Endpoint:

Composite endpoint of ACM and total CV clinical events during the Blinded Treatment Period.
   Null hypothesis: there is no treatment difference between the ALXN2220 and placebo groups in the rate of the composite endpoint of ACM and total CV clinical events.
   Alternative hypothesis: there is a treatment difference between ALXN2220 and placebo groups in the rate of the composite endpoint of ACM and total CV clinical events.

### Secondary Endpoints:

The following secondary endpoints will be assessed. The hierarchical testing procedure will be described in more detail in the SAP.
Composite of ACM and total HF events
   Null hypothesis: there is no treatment difference between ALXN2220 and placebo groups in the rate of the composite endpoint of ACM and total HF events.
   Alternative hypothesis: there is a treatment difference between ALXN2220 and placebo groups in the rate of the composite endpoint of ACM and total HF events.
Change from baseline in KCCQ-OS to 24 months
   Null hypothesis: there is no difference between ALXN2220 and placebo groups in the mean change in KCCQ-OS from baseline to 24 months.
   Alternative hypothesis: there is a treatment difference between ALXN2220 and placebo groups in the mean change in KCCQ-OS from baseline to 24 months.
Time to CV related mortality
   Null hypothesis: there is no treatment difference between ALXN2220 and placebo groups in the hazard rate of CV related mortality.
   Alternative hypothesis: there is a treatment difference between ALXN2220 and placebo groups in the hazard rate of CV related mortality.
Change from baseline in 6MWT to 24 months

Null hypothesis: there is no difference between ALXN2220 and placebo groups in the mean change in 6MWT from baseline to 24 months.

Alternative hypothesis: there is a treatment difference between ALXN2220 and placebo groups in the mean change in 6MWT from baseline to 24 months.
Rate of CV clinical events

Null hypothesis: there is no treatment difference between ALXN2220 and placebo groups in the rate of CV clinical events.

Alternative hypothesis: there is a treatment difference between ALXN2220 and placebo groups in the rate of CV clinical events.
Time to ACM
Null hypothesis: there is no treatment difference between ALXN2220 and placebo groups in the hazard rate of ACM.
Alternative hypothesis: there is a treatment difference between ALXN2220 and placebo groups in the hazard rate of ACM.

### 9.2 Analysis Sets

The following participant analysis sets are defined:

TABLE 19

| Participant Analysis Sets | |
|---|---|
| Population | Description |
| FAS | All participants who have been randomized and received at least 1 |
| | dose of study intervention. Participants will be analyzed according to |
| | the study intervention group to which they were randomized, |
| | regardless of the study intervention they actually received. |
| Safety Set | All participants who received at least 1 dose of study intervention. |
| | Participants will be analyzed according to the study intervention they |
| | actually received, regardless of the study intervention they were |
| | randomized to. |
| PPS | All participants who meet the definition of FAS and who are without |
| | major protocol deviations that would affect the interpretability of the |
| | efficacy outcomes (major protocol deviations will be described in the |
| | SAP). |
| PKAS | All participants who received at least 1 dose of ALXN2220 and have |
| | at least 1 measurable serum ALXN2220 concentration. |
| PDAS | All participants who receive at least 1 dose of study intervention and |
| | who have evaluable PD data. PD analyses will be based upon the |
| | study intervention received. |
| Immunogenicity | All randomized participants who receive at least 1 dose of the study |
| Analysis Set | intervention and who have at least 1 reportable post-first dose result |
| | in ADA assay. Participants will be analyzed according to the study |
| | intervention actually received. |

### 9.3 Statistical Analyses

Statistical methods described in this section will be further elaborated in a separate SAP. Summary statistics will be computed and displayed by treatment group and by visit, where applicable. Descriptive statistics for continuous variables will minimally include the number of participants, mean, SD, minimum, median, and maximum. For categorical variables, frequencies, and percentages will be presented. Graphical displays will be provided as appropriate. Analyses will be performed using the SAS® software Version 9.4 or higher.

### 9.3.1 General Considerations

Baseline is defined as the last measurement prior to study treatment administration on Day 1 of each part. If the Day 1 assessment is missing, invalid, or collected after administration of study treatment, the latest non-missing assessment prior to study treatment.

Continuous endpoints will be summarized using descriptive statistics, including number of observations and mean, SD, median, first and third quartile, minimum, and maximum values. Categorical variables will be summarized by frequency counts and percentage of participants.

All data and all outcomes derived from the data will be presented in detailed data listings or summary tabulations. Graphical displays may also be provided when appropriate.

### Continuous Endpoints

A MMRM will be used as the primary method to analyze continuous endpoints. The LS mean at each visit for each treatment group will be provided. The LS mean of treatment differences between ALXN2220 and placebo will also be provided with 95% CI and p-value.

### 9.3.2 Primary Endpoint(s) Analysis

### Derivation of Endpoint(s)

Analysis of the primary endpoint will be performed on the FAS.

The attributes of primary estimand for the primary endpoint are as follows:
a. Population: Adult (≥18 to ≤90 years of age) participants with symptomatic ATTR-CM (either variant or wild-type) and NT-proBNP>2000 pg/mL
b. Endpoint: Composite endpoint of ACM and total CV clinical events during the Blinded Treatment Period
c. Treatment conditions: ALXN2220 or placebo IV q4w
d. Handling of IEs and corresponding strategies: Summarized in Table 20
e. Population-level summary: Rate ratio from the Andersen-Gill model with robust variance estimation

TABLE 20
Handling of Intercurrent Events (IE) and Corresponding
Strategies for Composite Endpoint

| IE | Strategy Addressing the IE and Its Description |
|---|---|
| Study intervention | Treatment policy strategy - post-treatment |
| discontinuation | discontinuation data included in the analysis |
| Receive a heart | Composite strategy - IE will be treated as |
| transplant or a | death |
| permanent CMAD | |

### Multiplicity Adjustment

The Type I error rate will be controlled at the 0.05 two-sided level. A multiple testing strategy will be defined in the SAP for the primary and secondary efficacy endpoint hypothesis testing.

### Main Analytical Approach

The rates of the composite endpoint of ACM and total CV clinical events will be compared between ALXN2220 and placebo groups.

### Sensitivity Analyses

Details of all sensitivity analysis will be described in the SAP.

### 9.3.3 Secondary Endpoints Analysis

Analysis of secondary endpoints will be performed on the FAS. The hierarchical testing procedure will be described in more detail in the SAP.

### 9.3.3.1 Composite of ACM and Total HF Events

The attributes of the estimand for this secondary endpoint are as follows:
a. Population: Adult (≥18 to ≤90 years of age) participants with symptomatic ATTR-CM (either variant or wild-type) and NT-proBNP>2000 pg/mL
b. Endpoint: Composite of ACM and total HF events
c. Treatment conditions: ALXN2220 or placebo IV q4w
d. Handling of IEs: IEs and their corresponding strategies are similar to the CV clinical events component of the primary endpoint (Table 20).
e. Population-level summary: Rate ratio from the Andersen-Gill model with robust variance estimation

The rates of the composite endpoint of ACM and total HF events will be compared between ALXN2220 and placebo groups.

9.3.3.2 Change from Baseline in KCCQ-OS to 24 Months
The attributes of the estimand for this secondary endpoint are as follows:
a. Population: Adult (≥18 to ≤90 years of age) participants with symptomatic ATTR-CM (either variant or wild-type) and NT-proBNP>2000 pg/mL
b. Endpoint: Change from baseline in KCCQ-OS to 24 months
c. Treatment conditions: ALXN2220 or placebo IV q4w
d. Handling of IEs: IEs and their corresponding strategies as described in Table 21
e. Population-level summary: LS mean difference between ALXN2220 and placebo in change from baseline in KCCQ-OS to 24 months from the repeated measures mixed model
   The difference between the change from baseline to 24 months in KCCQ-OS between ALXN2220 and placebo groups will be analyzed.
   9.3.3.3 CV related Mortality
   The attributes of the estimand for this secondary endpoint are as follows:
a. Population: adult (≥18 to ≤90 years of age) participants with symptomatic ATTR-CM (either variant or wild-type) and NT-proBNP>2000 pg/mL
b. Endpoint: Time to CV related mortality
c. Treatment conditions: ALXN2220 or placebo IV q4w
d. Handling of IEs: IEs and their corresponding strategies are similar to the CV clinical events component of the primary endpoint (Table 20). In addition, non-CV death will be addressed based on composite strategy with observed data until the ICE will be analyzed.
e. Population-level summary: Hazard ratio from Cox proportional hazards model
   The hazard rates of CV related mortality will be compared between ALXN2220 and placebo groups.
   9.3.3.4 Change from Baseline in 6MWT to 24 Months
   The attributes of estimand for this secondary endpoint are as follows:
a. Population: Adult (≥18 to ≤90 years of age) participants with symptomatic ATTR-CM (either variant or wild-type) and NT-proBNP>2000 pg/mL
b. Endpoint: Change from baseline in 6MWT to 24 months
c. Treatment conditions: ALXN2220 or placebo IV q4w
d. Handling of IEs: IEs and their corresponding strategies as described in Table 21.
e. Population-level summary: LS mean difference between ALXN2220 and placebo in change from baseline in 6MWT to 24 months from the repeated measures mixed model
   The change from baseline to 24 months in 6MWT will utilize the same analytical approach as that used for change from baseline in KCCQ-OS.

### 9.3.3.5 Rate of CV Clinical Events

The attributes of the estimand for this secondary endpoint are as follows:
a. Population: Adult (≥18 to ≤90 years of age) participants with symptomatic ATTR-CM (either variant or wild-type) and NT-proBNP>2000 pg/mL
b. Endpoint: Rate of CV clinical events
c. Treatment conditions: ALXN2220 or placebo IV q4w
d. Handling of IEs: IEs and their corresponding strategies are similar to the CV clinical events component of the primary endpoint (Table 20).
e. Population-level summary: Rate ratio of CV clinical events from Andersen-Gill model with robust variance estimation
   The rates of the CV clinical events will be compared between ALXN2220 and placebo groups.

### 9.3.3.6 Time to ACM

The attributes of the estimand for this secondary endpoint are as follows:
a. Population: adult (≥18 to ≤90 years of age) participants with symptomatic ATTR-CM (either variant or wild-type) and NT-proBNP>2000 pg/mL
b. Endpoint: Time to of ACM
c. Treatment conditions: ALXN2220 or placebo IV q4w
d. Handling of IEs: IEs and their corresponding strategies are similar to the ACM component of the primary endpoint (Table 20).
e. Population-level summary: Hazard ratio from Cox proportional hazards model
   The hazard rates for ACM will be compared between ALXN2220 and placebo groups.

TABLE 21
Handling of Intercurrent Events and Corresponding Strategies for
KCCQ-OS and 6MWT

| IE | Strategy Addressing the IE and Its Description |
|---|---|
| Study treatment | Addressed based on treatment policy strategy with |
| discontinuation | post-treatment discontinuation data included in the |
| | analysis. |
| Death | Addressed based on composite strategy. Missing data |
| | post-death will use J2R imputation and sample from the |
| | bottom 50% of observed change from baseline values |
| | in the placebo participants, assuming MNAR. |
| Receive a heart | Treated as death and addressed based on composite |
| transplant | strategy. Assessment after the IE will be censored and |
| or a permanent | imputed by the same J2R approach as for death. |
| CMAD | |

### 9.3.4 Safety Analyses

All safety analyses will be made on the Safety set. The safety and tolerability of ALXN2220 in participants with ATTR-CM will be assessed based on TEAEs, SAEs, ECG abnormalities, clinical laboratory data, physical examinations, and vital sign measurements, and will be presented using descriptive statistics.

### 9.3.5 Immunogenicity Analyses

All immunogenicity analyses will be performed on the Immunogenicity Analysis Set.

### 9.3.6 Pharmacokinetic/Pharmacodynamic Analysis

The PK and PD analyses will be based on the PK Analysis Set and PD Analysis Set, respectively. Graphs of mean serum concentration-time profiles will be constructed. Graphs of serum concentration-time profiles for individual participants may also be provided. Boxplots of C_{trough} and Cₘₐₓ by study visit may be constructed. For a subset of Chinese population that provided additional PK samples, serum ALXN2220 concentrations and actual sampling dates and times will be used to derive the PK parameters by noncompartmental analyses methods.

Following PK parameters will be derived (data permitting):
Cₘₐₓ, AUCₜ, AUC_{∞}, t_{1/2}, CL, Vss.
Population PK analysis will be conducted according to a separate analysis plan.
Descriptive statistics will be presented for PD endpoints at each sampling time. The PD effects of ALXN2220 will be evaluated by assessing the absolute values, changes and percent changes from baseline values over time, as appropriate. Boxplots of absolute values, changes and percent changes by study visit may be constructed, as appropriate. Assessments of ALXN2220 PK/PD relationships may be explored using data from this study or in combination with data from other studies. PD parameters may be evaluated using MMRM using a similar analysis method as the primary endpoint (Section 9.3.2).

### Exploratory Endpoint Analyses

Exploratory endpoints will be analyzed by methods mentioned in Section 9.3.1.

### 9.3.8 Other Analyses

### 9.3.8.1 Examination of Subgroups

Subgroup analyses will be conducted for the primary and secondary efficacy endpoints, and NT-proBNP change from baseline, and will include, but not be limited to:
TTR genotype (variant vs wild-type)
Baseline NT-proBNP (NT-proBNP>3000 pg/mL vs NT-proBNP≤3000 pg/mL)
Baseline renal function (eGFR<45 mL/min/1.73 m² vs ≥45 mL/min/1.73 m²)
NYHA Classification (II vs III-IV)
Survival status (survived vs deceased)
Baseline LVEF (LVEF<40% [HFrEF] vs LVEF 40%-49% [HFmrEF] vs LVEF≥50% [HFpEF])
Treatment with disease modifying agents at Screening (TTR stabilizer vs TTR silencer vs TTR silencer/stabilizer combination vs none)
Any usage (during the study) of disease modifying agents (TTR stabilizer vs TTR silencer vs TTR silencer/stabilizer combination vs none)
Time on disease modifying agent
Weight (≥60 kg vs <60 kg).
   Detailed plans for the subgroup analyses will be provided in the SAP.

### 9.4 Interim Analysis

An interim analysis to test for early efficacy may be conducted.

### 9.5 Sample Size Determination

Approximately 1000 participants will be randomized in a 2:1 ratio to ALXN2220 or placebo.

### 10. Documentation and Operational Considerations

### 10.1 Regulatory, Ethical, and Study Oversight Considerations

### 10.1.1 Regulatory and Ethical Considerations

### 10.1.2 Financial Disclosure

### 10.1.3 Informed Consent Process

### 10.1.4 Recruitment Strategy

### 10.1.5 Data Protection

### 10.1.6 Committees Structure

### 10.1.6.1 Data Monitoring Committee

### 10.1.6.2 Clinical Events Adjudication Committee

### 10.1.7 Dissemination of Clinical Study Data

Study-related information and study results may be posted on publicly accessible clinical study databases

### 10.1.8. Data Quality Assurance

### 10.1.9 Source Documents

### 10.1.10 Study and Site Start and Termination/Closure

### Study Start

The study start date is the date on which the clinical study will be open for recruitment of participants. The first act of recruitment is the first site activation.

### Study/Site Termination

Study sites will be closed upon completion of the study. A study site is considered closed when all required documents and study supplies have been collected and a study-site closure visit has been performed. The Investigator may initiate study site closure at any time, provided there is reasonable cause and sufficient notice is given in advance of the intended termination.

### 10.1.11 Publication Policy

### 10.2 Clinical Laboratory Tests

The tests detailed in Table 22 will be performed by the central laboratory. Samples for clinical laboratory tests (Table 13) will be obtained according to the SoA (Tables 25 and 26) and sent to the central laboratory. Results from the central laboratory assessments obtained on dosing days are not required prior to dosing the participants.
Protocol-specific requirements for inclusion or exclusion of participants: See Section 5.
Additional laboratory tests may be performed at any time during the study as determined necessary by the Investigator or required by local regulations.
Pregnancy testing See Section 12.

TABLE 22
Protocol-required Laboratory Tests

| | |
|---|---|
| Laboratory Tests | Parameters |

Clinical Safety Laboratory Tests

| Screening only | HIV serology | Serum B6 |
|---|---|---|
| | Hepatitis B and C serology | Serum B12 |
| | Folic acid | sFLC^{a} |
| | | Serum IFE^{a} |
| | | Urine IFE^{a} |
| Hematology | Platelet count^{b} | |
| | RBC count | |
| | RBC indices: | Mean corpuscular volume |
| | | Mean corpuscular hemoglobin |
| | | Reticulocyte (absolute and percent) |
| | WBC count with differential: | Neutrophils, segmented |
| | | Lymphocytes |
| | | Monocytes |
| | | Eosinophils |
| | | Basophils |
| | Hemoglobin | |
| | Hematocrit | |
| Clinical chemistry^{c} | Alanine aminotransferase | Calcium |
| | Alkaline phosphatase | Magnesium |
| | Aspartate aminotransferase | Potassium |
| | Gamma-glutamyltransferase | Phosphate |
| | Total and direct bilirubin | Sodium |
| | Total protein | Chloride |
| | Albumin | Vitamin A |
| | Creatinine (including eGFR) | TTR |
| | Uric acid | TSH |
| | Blood urea nitrogen | fT4 |
| | Glucose (random) | T3 |
| | Carbon dioxide | Non-fasting lipid profile |
| Routine urinalysis | Bilirubin | pH |
| | Blood | Protein |
| | Glucose | Specific gravity |

Clinical Safety Laboratory Tests

| | |
|---|---|
| Ketones | Leukocyte esterase |
| | Nitrite |
| | Urobilinogen |

| | |
|---|---|
| Other study | Serum or urine human chorionic gonadotropin pregnancy test (as |
| specific tests | needed for WOCBP)^{d} |

Clinical Efficacy Laboratory Tests
hs-cTnT, NT-proBNP, CRP
Biomarker Laboratory Tests
CITP, PICP, PIIINP, PRO-C6, IL1b, IL6, IL8, INFg, TNF-α, IL10, IL1RA, C3, C4, SAA,
ferritin, sNfL
^{a}If historical results are not available or insufficient to exclude a monoclonal gammopathy for central eligibility confirmation, these tests can be performed as part of the Screening laboratory. These tests are not mandated in all participants.
^{b}Peripheral smear should be reviewed for any platelet count <50,000 × 10⁹/L.
^{c}Details of liver chemistry stopping criteria and required actions and follow-up are provided in Section 10.8: Liver Safety: Suggested Actions and Follow-up Assessments. All events of ALT ≥ 3 × ULN and total bilirubin ≥2 × ULN (>35% direct bilirubin) or ALT ≥3 × ULN and INR >1.5 (if INR measured), which may indicate severe liver injury (possible Hy's law), must be reported to Alexion in an expedited manner (excluding studies of hepatic impairment or cirrhosis).
^{d}Local urine testing will be standard for the protocol unless serum testing is required by local regulation or IRB/IEC

### 10.3 AEs and SAEs: Definitions and Procedures for Recording, Evaluating, Follow-up, and Reporting

### 10.3.1 Definition of AE

Section 8.2 and Section 8.4.1.
Section 8.4.1.

### 10.3.2 Definition of SAE

10.3.3 Recording and Follow-Up of AE and/or SAE

### 10.3.3.1 AE and SAE Recording

### 10.3.3.2 Assessment of Intensity

### 10.3.3.3 Assessment of Causality

### 10.3.3.4 Follow-Up of AEs and SAEs

Section 8.4.

### 10.3.4 Reporting of SAEs

### 10.3.5 Unexpected Events

### 10.4 Medication Error, Drug Abuse, and Drug Misuse

### Medication Error

For the purposes of this clinical study a medication error is an unintended failure or mistake in the treatment process for an IMP that either causes harm to the participant or has the potential to cause harm to the participant.

### Drug Abuse

For the purpose of this study, drug abuse is defined as the persistent or sporadic intentional, non-therapeutic excessive use of IMP for a perceived reward or desired non-therapeutic effect.

### Drug Misuse

Drug misuse is the intentional and inappropriate use of IMP for medicinal purposes outside of the authorized product information, or for unauthorized IMPs, outside the intended use as specified in the protocol, including deliberate administration of the product by the wrong route.

### 10.5 Contraceptive and Barrier Guidance

### 10.5.1 Definitions

### Woman of Childbearing Potential (WOCBP)

Women in the following categories are considered WOCBP (fertile):

### 10.5.2 Contraception Guidance

Female participants of childbearing potential are eligible to participate in this study (Section 5.1) if they agree to use a highly effective contraceptive method during the study intervention period and for a minimum of 5 months after the last dose of study intervention. Birth control methods that are considered highly effective are included in the table below.

Fertile male participants are eligible to participate in this study (Section 5.1) if they agree to use condoms during heterosexual intercourse with WOCBP during the study intervention period and for at least 7 months after the last dose of study intervention.

### 10.6 Handling of Human Biological Samples

All research and biological samples, including those for possible future research, are subject to national regulations and will only be conducted in a specified country if approved in that country.

### 10.6.1 Chain of Custody

### 10.6.2 Withdrawal of Informed Consent for Donated Biological Samples

### 10.7 Genetics Research

DNA samples will be utilized for TTR genotyping in participants without available historic test results, which will be required for the evaluation of ALXN2220 in the 2 genetics forms of ATTR-CM (wild-type or variant TTR) with different clinical features and natural history.

### 10.8 Liver Safety: Suggested Actions and Follow-up Assessments

### 10.9 Guidance for Management of Infusion-Related Reactions and Other Hypersensitivity Events

CTCAE Grade 1 (mild)
CTCAE Grade 2 (moderate)
CTCAE Grade 3 (severe) or Grade 4 (life threatening)
See Section 7.1.

### Anaphylaxis (CTCAE Grade 3 or Grade 4) or Other Severe Acute Hypersensitivity Event

See Section 7.1. In case of a suspected SAE of hypersensitivity or anaphylaxis, additional ADA samples may be collected during or in proximity of the event (Section 12).

### 10.10 Tissue Inflammation

See Section 7.1.

### 10.11 Decreased Platelet Count

See Section 7.1.

### 10.12 Medical Device AEs, ADEs, SAEs, SADEs, USADEs and Device Deficiencies: Definitions and Procedures for Recording, Evaluating, Follow-up, and Reporting in Medical Device Studies

### 10.13 COVID-19 Risk Assessment

ATTR-CM can cause irreversible morbidity and even mortality, if untreated. As such, the benefit a participant may receive from treatment with ALXN2220 is potentially significant.

The potential operational risks identified and the mitigation measures put in place in light of the COVID-19 pandemic are provided in Table 23.

TABLE 23
Potential Operational Risks and Mitigation Measures due to COVID-19

| | | |
|---|---|---|
| | Summary of Data/ | |
| Risks Category | Rationale for Risk | Mitigation Strategy |

Potential risks

| | | |
|---|---|---|
| Healthcare | COVID-19 pandemic may impact the workload | During the time that the |
| institution availability | of healthcare institutions globally and may | COVID-19 pandemic is |
| for non-COVID-19 | reduce staff availability to perform non-urgent | active, Alexion will not open |
| related activities | activities and non-COVID-19 related activities. | study sites or enroll new |
| | | participants at sites unless |
| | | the sites have the resourcing |
| | | and capabilities to implement |
| | | the study per protocol. |
| Data quality and | Lack of availability of site personnel to perform | During the time that the |
| integrity | study assessments and capture study specific | COVID19 pandemic is active, |
| | data in a timely manner and to maintain | Alexion will only open study |
| | adequate quality standards. | sites that report enough |
| | Lack of availability of site personnel to ensure | personnel capacity to |
| | adequate and continuous chain of custody, | sufficiently conduct clinical |
| | storage conditions, and monitoring for | study related activities. |
| | investigational product and biological samples. | During this timeframe, site |
| | Inability of study monitors and quality | capacity will be reviewed by |
| | personnel to conduct in-person visits | the Investigator and the |
| | to exercise adequate oversight of study | Sponsor's Medical Monitor |
| | execution at investigational sites. | prior to Screening. Each site |
| | Missing data (COVID-19 pandemic may impact | is also evaluated for the |
| | study visit schedules, and increase missed | capacity to perform remote |
| | visits and/or participant study discontinuations | monitoring visits and remote |
| | inadvertently resulting in missing data (e.g., for | source data verification. |
| | protocol-specified procedures). | During the time that the |
| | | COVID-19 pandemic is |
| | | active, it will be important to |
| | | capture specific information |
| | | in the CRF that explains the |
| | | reason the data is missing |
| | | (e.g., missed study visits or |
| | | participant study |
| | | discontinuations due to |
| | | COVID-19). |

### 10.14 COVID-19 Vaccine Risk Assessment

The majority of participants in Study NI006-1001 received COVID-19 vaccines during the study. There was no indication that safety or efficacy were altered in participants treated with ALXN2220 and COVID-19 vaccines. The potential operational risks identified and mitigation measures put in place in light of the COVID-19 vaccination rollout are provided in Table 24.

TABLE 24
Potential Operational Risks and Mitigation Measures due to COVID-19 Vaccine

| | | |
|---|---|---|
| Risks Category | Summary of Data/Rationale for Risk | Mitigation Strategy |

Potential risks

| | | |
|---|---|---|
| Data quality and | Missing data due to appointments for | Capture specific information in |
| integrity | COVID-19 vaccination or side effects of | the CRF that explains the |
| | COVID-19 vaccine may impact study visit | reason for missing data (e.g., |
| | schedules and increase missed visits and/or | missed study visits due to |
| | participant study discontinuations, | appointments for COVID-19 |
| | inadvertently resulting in missing data | vaccination or side effects of |
| | (e.g., for protocol-specified procedures). | COVID-19 vaccine). |

11. References: The Disclosure(s) in Following References are Incorporated by Reference in their Pertinent Parts
Adams D, Koike H, Slama M, Coelho T. Hereditary transthyretin amyloidosis: a model of medical progress for a fatal disease. Nat Rev Neurol. 2019; 15(7):387-404.
Adams D, Coelho T, Obici L, et al. Rapid progression of familial amyloidotic polyneuropathy: a multinational natural history study. Neurology. 2015; 85(8):675-682.
Adams D, Tournev I L, Taylor M S, et al. Efficacy and safety of vutrisiran for patients with hereditary transthyretin-mediated amyloidosis with polyneuropathy: a randomized clinical trial. Amyloid. 2023; 30(1):1-9.
Adams D, Gonzalez-Duarte A, O'Riordan W D, et al. Patisiran, an RNAi Therapeutic, for Hereditary Transthyretin Amyloidosis. N Engl J Med. 2018; 379(1):11-21.
Andersen P K, Gill R D. Cox's regression model for counting processes: a large sample study. Ann Statist. 1982; 10(4):1100-1120.
Ando Y, Coelho T, Berk J L, et al. Guideline of transthyretin-related hereditary amyloidosis for clinicians. Orphanet J Rare Dis. 2013; 8:31.
Ando Y, Adams D, Benson M D, et al. Guidelines and new directions in the therapy and monitoring of ATTRv amyloidosis. Amyloid. 2022; 29(3):143-155.
Benson M D, Waddington-Cruz M, Berk J L, et al. Inotersen Treatment for Patients with Hereditary Transthyretin Amyloidosis. N Engl J Med. 2018; 379(1):22-31.
Bezerra F, Saraiva M J, Almeida M R. Modulation of the Mechanisms Driving Transthyretin Amyloidosis. Front Mol Neurosci. 2020; 13:592644.
Cheng R K, Levy W C, Vasbinder A, et al. Diuretic Dose and NYHA Functional Class Are Independent Predictors of Mortality in Patients With Transthyretin Cardiac Amyloidosis. JACC CardioOncol. 2020; 2(3):414-424.
Coelho T, Vinik A, Vinik E J, Tripp T, Packman J, Grogan D R. Clinical measures in transthyretin familial amyloid polyneuropathy. Muscle Nerve. 2017; 55(3):323-332.
Coelho T, Maia L F, Martins da Silva A, et al. Tafamidis for transthyretin familial amyloid polyneuropathy: a randomized, controlled trial. Neurology. 2012; 79(8):785-792.
Coelho T, Waddington Cruz M, Chao C C, et al. Characteristics of Patients with Hereditary Transthyretin Amyloidosis-Polyneuropathy (ATTRv-PN) in NEURO-TTRansform, an Open-label Phase 3 Study of Eplontersen. Neurol Ther. 2023; 12(1):267-287.
Coutinho P, da Silva A L, Lopes J, et al. Forty years of experience with type I amyloid neuropathy. Review of 483 cases. Medicine. 1980.
Crapo. A. T. S. Committee on Proficiency Standards for Clinical Pulmonary Function Laboratories. ATS statement: guidelines for the six-minute walk test. Am J Respir Crit Care Med. 2002; 166(1):111-117.
Criddle R S, Lin H L, James I, Park J S, Hansen L D, Price J C. Proposing a minimal set of metrics and methods to predict probabilities of amyloidosis disease and onset age in individuals. Aging (Albany NY). 2020; 12(22):22356-22369.
Dispenzieri A, Coelho T, Conceicao I, et al. Clinical and genetic profile of patients enrolled in the Transthyretin Amyloidosis Outcomes Survey (THAOS): 14-year update. Orphanet J Rare Dis. 2022; 17(1):236.
Doessegger L, Banholzer M L. Clinical development methodology for infusion-related reactions with monoclonal antibodies. Clin Transl Immunology. 2015; 4(7):e39.
Dohrn M F, Ihne S, Hegenbart U, et al. Targeting transthyretin—Mechanism-based treatment approaches and future perspectives in hereditary amyloidosis. Journal of neurochemistry. 2021; 156(6):802-818.
Dorbala S, Ando Y, Bokhari S, et al. ASNC/AHA/ASE/EANM/HFSA/ISA/SCMR/SNMMI expert consensus recommendations for multimodality imaging in cardiac amyloidosis: Part 2 of 2-Diagnostic criteria and appropriate utilization. J Nucl Cardiol. 2020; 27(2):659-673.
Dyck P J, Kratz K M, Lehman K A, et al. The Rochester Diabetic Neuropathy Study: design, criteria for types of neuropathy, selection bias, and reproducibility of neuropathic tests. Neurology. 1991; 41(6):799-807.
Dyck P J B, Gonzalez-Duarte A, Obici L, et al. Development of measures of polyneuropathy impairment in hATTR amyloidosis: From NIS to mNIS+7. J Neurol Sci. 2019; 405:116424.
Ebenezer G J, Liu Y, Judge D P, et al. Cutaneous nerve biomarkers in transthyretin familial amyloid polyneuropathy. Ann Neurol. 2017; 82(1):44-56.
Eisele Y S, Monteiro C, Fearns C, et al. Targeting protein aggregation for the treatment of degenerative diseases. Nat Rev Drug Discov. 2015; 14(11):759-780.
Fontana M, Gilbertson J, Verona G, et al. Antibody-Associated Reversal of ATTR Amyloidosis-Related Cardiomyopathy. N Engl J Med. 2023; 388(23):2199-2201.
Freeman R, Gonzalez-Duarte A, Barroso F, et al. Cutaneous amyloid is a biomarker in early ATTRv neuropathy and progresses across disease stages. Annals of clinical and translational neurology. 2022; 9(9):1370-1383.
Gaetani L, Blennow K, Calabresi P, Di Filippo M, Parnetti L, Zetterberg H. Neurofilament light chain as a biomarker in neurological disorders. Journal of neurology, neurosurgery, and psychiatry. 2019; 90(8):870-881.
Garcia-Pavia P, Aus dem Siepen F, Donal E, et al. Phase 1 Trial of Antibody N1006 for Depletion of Cardiac Transthyretin Amyloid. N Engl J Med. 2023; 389(3):239-250.
Garcia-Pavia P, Rapezzi C, Adler Y, et al. Diagnosis and treatment of cardiac amyloidosis: a position statement of the ESC Working Group on Myocardial and Pericardial Diseases. Eur Heart J. 2021; 42(16):1554-1568.
Gertz M A, Benson M D, Dyck P J, et al. Diagnosis, Prognosis, and Therapy of Transthyretin Amyloidosis. J Am Coll Cardiol. 2015; 66(21):2451-2466.
Gillmore J D, Damy T, Fontana M, et al. A new staging system for cardiac transthyretin amyloidosis. Eur Heart J. 2018; 39(30):2799-2806.
Grogan M, Scott C G, Kyle R A, et al. Natural History of Wild-Type Transthyretin Cardiac Amyloidosis and Risk Stratification Using a Novel Staging System. J Am Coll Cardiol. 2016; 68(10):1014-1020.
Hanna M, Damy T, Grogan M, et al. Impact of Tafamidis on Health-Related Quality of Life in Patients With Transthyretin Amyloid Cardiomyopathy (from the Tafamidis in Transthyretin Cardiomyopathy Clinical Trial). Am J Cardiol. 2021; 141:98-105.
Huh Y, Riley S, Harnisch L, Nicholas T. Population pharmacokinetic modelling and simulation of tafamidis in healthy subjects and patients with transthyretin amyloidosis. Br J Clin Pharmacol. 2021; 87(9):3574-3587.
Kapoor M, Foiani M, Heslegrave A, et al. Plasma neurofilament light chain concentration is increased and correlates with the severity of neuropathy in hereditary transthyretin amyloidosis. J Peripher Nerv Syst. 2019; 24(4):314-319.
Kelly J W. Does protein aggregation drive postmitotic tissue degeneration? Sci Transl Med. 2021; 13(577).
Kenward M G, Roger J H. Small sample inference for fixed effects from restricted maximum likelihood. Biometrics. 1997; 53(3):983-997.
Kittleson M M, Ruberg F L, Ambardekar A V, et al. 2023 ACC Expert Consensus Decision Pathway on Comprehensive Multidisciplinary Care for the Patient With Cardiac Amyloidosis: A Report of the American College of Cardiology Solution Set Oversight Committee (Writing, Committee). J Am Coll Cardiol. 2023; 81(11):1076-1126.
Le Gal F, Gordien E, Affolabi D, et al. Quantification of hepatitis delta virus RNA in serum by consensus real-time PCR indicates different patterns of virological response to interferon therapy in chronically infected patients. J Clin Microbiol. 2005; 43(5):2363-2369.
Leonardi L, Adam C, Beaudonnet G, et al. Skin amyloid deposits and nerve fiber loss as markers of neuropathy onset and progression in hereditary transthyretin amyloidosis. Eur J Neurol. 2022; 29(5):1477-1487.
Lin D Y, Wei L J, Yang I, Ying Z. Semiparametric regression for the mean and rate functions of recurrent events. J R Stat Soc Series B Stat Methodol. 2002; 62(4):711-730.
Maia L F, Maceski A, Conceicao I, et al. Plasma neurofilament light chain: an early biomarker for hereditary ATTR amyloid polyneuropathy. Amyloid. 2020; 27(2):97-102. Martinez-Naharro A, Treibel T A, Abdel-Gadir A, et al. Magnetic Resonance in Transthyretin Cardiac Amyloidosis. J Am Coll Cardiol. 2017; 70(4):466-477.
Masuda T, Ueda M, Suenaga G, et al. Early skin denervation in hereditary and iatrogenic transthyretin amyloid neuropathy. Neurology. 2017; 88(23):2192-2197.
Maurer M S, Schwartz J H, Gundapaneni B, et al. Tafamidis treatment for patients with transthyretin amyloid cardiomyopathy. N Engl J Med. 2018; 379(11):1007-1016.
Michalon A, Hagenbuch A, Huy C, et al. A human antibody selective for transthyretin amyloid removes cardiac amyloid through phagocytic immune cells. Nat Commun. 2021; 12(1):3142.
Nativi-Nicolau J, Judge D P, Hoffman J E, et al. Natural history and progression of transthyretin amyloid cardiomyopathy: insights from ATTR-ACT. ESC Heart Fail. 2021; 8(5):3875-3884.
Oppe M, Devlin N J, van Hout B, Krabbe P F, de Charro F. A program of methodological research to arrive at the new international EQ-5D-5L valuation protocol. Value Health. 2014; 17(4):445-453.
Rapezzi C, Quarta C C, Obici L, et al. Disease profile and differential diagnosis of hereditary transthyretin-related amyloidosis with exclusively cardiac phenotype: an Italian perspective. Eur Heart J. 2013; 34(7):520-528.
Stewart M D, McCall B, Pasquini M, et al. Need for aligning the definition and reporting of cytokine release syndrome (CRS) in immuno-oncology clinical trials. Cytotherapy. 2022; 24(7):742-749.
Ticau S, Sridharan G V, Tsour S, et al. Neurofilament Light Chain as a Biomarker of Hereditary Transthyretin-Mediated Amyloidosis. Neurology. 2021; 96(3):e412-e422.
Vinik E J, Vinik A I, Paulson J F, et al. Norfolk QOL-DN: validation of a patient reported outcome measure in transthyretin familial amyloid polyneuropathy. J Peripher Nerv Syst. 2014; 19(2):104-114.
Ware J E, Jr., Sherbourne C D. The MOS 36-item short-form health survey (SF-36). I. Conceptual framework and item selection. Med Care. 1992; 30(6):473-483.
Yamamoto S, Wilczek H E, Nowak G, et al. Liver transplantation for familial amyloidotic polyneuropathy (FAP): a single-center experience over 16 years. Am J Transplant. 2007; 7(11):2597-2604.

### 12. Schedule of Activities

TABLE 25
Schedule of Activities up to 24 Months

| | Study Period | | | | | | | | | |
|---|---|---|---|---|---|---|---|---|---|---|
| | Screening | Blinded Treatment Period Up to 24 Months | | | | | | | | |
| | Visit | | | | | | | | | |
| | V1 | V 2 | V 3 | V 4 | V 5 | V 6 | V 7 | V 8 | V 9 | V 10 |
| | Week | | | | | | | | | |
| | −5 | 1 | 1 | 3 | 4 | 8 | 12 | 16 | 20 | 24 |
| | Day (Window ±3 for V4-10, ±7 for V11-FU2) | | | | | | | | | |
| | −35 | | | | | | | | | |
| Procedure | to −1 | D 1 | D 3 ^{c} (±1 d) | D 15 | D 29 | D 57 | D 85 | D 113 | D 141 | D 169 |

General assessments/procedures

| | | |
|---|---|---|
| Informed | X | |
| consent | | |
| Eligibility | X | |
| assessment | | |
| Demographics | X | |
| sFLC, sIFE, | X | |
| and uIFE^{d} | | |
| TTR | X | |
| genotyping^{e} | | |
| Medical | X | |
| history | | |
| Prior | X | |
| medications, | | |
| therapies, | | |
| and | | |
| procedures | | |
| Randomization | | X |

Study intervention

| | | | | | | | |
|---|---|---|---|---|---|---|---|
| Study | X^{f} | X | X | X | X | X | X |
| intervention | | | | | | | |
| infusion | | | | | | | |

Safety assessments

| | | | | | | | | | | |
|---|---|---|---|---|---|---|---|---|---|---|
| Weight | X | X | | X | X | X | X | X | X | X |
| Height | X | | | | | | | | | |
| Physical | X | X | | X | X | X | X | X | X | X |
| examination ^{g} | | | | | | | | | | |
| Pregnancy | X | X | | | X | X | X | X | X | X |
| test^{h} | | | | | | | | | | |
| Vital signsⁱ | X | X | | X | X | X | X | X | X | X |
| 12-lead ECG^{j} | X | X | | X | | | X | | | X |
| Clinical | X | X | X^{c} | X | X | X | X | | | X |
| laboratory | | | | | | | | | | |
| tests | | | | | | | | | | |

| | |
|---|---|
| Concomitant | X |
| medications, | |
| therapies, | |
| and | |
| procedures | |
| AEs | X |

Efficacy and QoL assessments

| | | | | | | | |
|---|---|---|---|---|---|---|---|
| NYHA | X | X | X | X | X | X | X |
| Functional | | | | | | | |
| Classification | | | | | | | |
| NT-proBNP | X | X | X | X | X | X | X |
| hs-cTnT | X | X | X | X | X | X | X |
| CRP | X | X | X | X | X | X | X |
| Echocardiogram^{k} | X | | | | | X | X |
| 6MWT^{k, l} | X | | | | | X | X |
| KCCQ | | X | | | | X | X |
| EQ-5D-5L | X | | | | | | X |
| and SF-36 | | | | | | | |
| sNfL^{m} | | X | | | | | X |
| PND score | X | | | | | | |
| and FAP | | | | | | | |
| stage^{k, m} | | | | | | | |
| Norfolk- | X | | | | | | |
| QoL-DN^{k, m} | | | | | | | |

PK/immunogenicity assessments

| | | | |
|---|---|---|---|
| PK samplesⁿ | X | X | X |
| Immunogenicity | X | X | X |
| samples^{o} | | | |
| Additional | X | X | X |
| biomarkers^{p} | | | |

Other assessments (optional at selected study sites)

| | | |
|---|---|---|
| Cardiac | X | X |
| scintigraphy^{k, q} | | |
| Cardiac MRI^{k, q} | X | X |
| Tissue biopsy^{k, r} | X | |
| Mixed | X | |
| phenotype | | |
| diagnosis^{s} | | |
| NC study^{k, s} | X | |
| NIS^{k, s} | X | |

| | Study Period | | | | | | | | | | |
|---|---|---|---|---|---|---|---|---|---|---|---|
| | Blinded Treatment Period Up to 24 Months | | | | | | | | | | |
| | Visit | | | | | | | | | | |
| | V 11 | V 12 | V 13 | V 14 | V 15 | V 16 | V 17 | V 18 | V 19 | V 20 | V 21 |
| | Week | | | | | | | | | | |
| | 28 | 32 | 36 | 40 | 44 | 48 | 52 | 56 | 60 | 64 | 68 |
| | Day (Window ±3 for V4-10, ±7 for V11-FU2) | | | | | | | | | | |
| Procedure | D 197 | D 225 | D 253 | D 281 | D 309 | D337 | D365 | D393 | D421 | D449 | D477 |
| | General assessments/procedures | | | | | | | | | | |
| Informed | | | | | | | | | | | |
| consent | | | | | | | | | | | |
| Eligibility | | | | | | | | | | | |
| assessment | | | | | | | | | | | |
| Demographics | | | | | | | | | | | |
| sFLC, sIFE, | | | | | | | | | | | |
| and uIFE^{d} | | | | | | | | | | | |
| TTR | | | | | | | | | | | |
| genotyping^{e} | | | | | | | | | | | |
| Medical | | | | | | | | | | | |
| history | | | | | | | | | | | |
| Prior | | | | | | | | | | | |
| medications, | | | | | | | | | | | |
| therapies, | | | | | | | | | | | |
| and | | | | | | | | | | | |
| procedures | | | | | | | | | | | |
| Randomization | | | | | | | | | | | |
| | Study intervention | | | | | | | | | | |
| Study | X | X | X | X | X | X | X | X | X | X | X |
| intervention | | | | | | | | | | | |
| infusion | | | | | | | | | | | |
| | Safety assessments | | | | | | | | | | |
| Weight | X | X | X | X | X | X | X | X | X | X | X |
| Height | | | | | | | | | | | |
| Physical | X | X | X | X | X | X | X | X | X | X | X |
| examination ^{g} | | | | | | | | | | | |
| Pregnancy | X | X | X | X | X | X | X | X | X | X | X |
| test^{h} | | | | | | | | | | | |
| Vital signsⁱ | X | X | X | X | X | X | X | X | X | X | X |
| 12-lead ECG^{j} | | | X | | | | X | | | X | |
| Clinical | | | X | | | | X | | | X | |
| laboratory | | | | | | | | | | | |
| tests | | | | | | | | | | | |
| Concomitant | X | | | | | | | | | | |
| medications, | | | | | | | | | | | |
| therapies, | | | | | | | | | | | |
| and | | | | | | | | | | | |
| procedures | | | | | | | | | | | |
| AEs | X | | | | | | | | | | |
| | Efficacy and QoL assessments | | | | | | | | | | |
| NYHA | | | X | | | | X | | | X | |
| Functional | | | | | | | | | | | |
| Classification | | | | | | | | | | | |
| NT-proBNP | | | X | | | | X | | | X | |
| hs-cTnT | | | X | | | | X | | | X | |
| CRP | | | X | | | | X | | | X | |
| Echocardiogram^{k} | | | | | | | | | | | |
| 6MWT^{k, l} | | | | | | | X | | | | |
| KCCQ | X | | | X | | | | X | | | |
| EQ-5D-5L | X | | | | | | | X | | | |
| and SF-36 | | | | | | | | | | | |
| sNfL^{m} | X | | | | | | | X | | | |
| PND score | | | | | | | | X | | | |
| and FAP | | | | | | | | | | | |
| stage^{k, m} | | | | | | | | | | | |
| Norfolk- | | | | | | | | X | | | |
| QoL-DN^{k, m} | | | | | | | | | | | |
| | PK/immunogenicity assessments | | | | | | | | | | |
| PK samplesⁿ | X | | | | | | | X | | | |
| Immunogenicity | X | | | | | | | X | | | |
| samples^{o} | | | | | | | | | | | |
| Additional | X | | | | | | | X | | | |
| biomarkers^{p} | | | | | | | | | | | |
| | Other assessments (optional at selected study sites) | | | | | | | | | | |
| Cardiac | X | | | | | | | X | | | |
| scintigraphy^{k, q} | | | | | | | | | | | |
| Cardiac MRI^{k, q} | X | | | | | | | X | | | |
| Tissue biopsy^{k, r} | | | | | | | | | | | |
| Mixed | | | | | | | | | | | |
| phenotype | | | | | | | | | | | |
| diagnosis^{s} | | | | | | | | | | | |
| NC study^{k, s} | | | | | | | | X | | | |
| NIS^{k, s} | | | | | | | | X | | | |

| | Study Period | | | | | | | | | | | |
|---|---|---|---|---|---|---|---|---|---|---|---|---|
| | | | | | | | | | | | Post- | |
| | | | | | | | | | | | Treatment | |
| | Blinded Treatment Period Up to 24 Months | | | | | | | | | | safety | |
| | Visit | | | | | | | | | | | |
| | V 22 | V 23 | V 24 | V 25 | V 26 | V 27 | V 28 | V 29 | V 30 | | FU1 | FU2 |
| | Week | | | | | | | | | | | |
| | 72 | 76 | 80 | 84 | 88 | 92 | 96 | 100 | 104 | | +8 | +17 |
| | Day (Window ±3 for V4-10, ±7 for V11-FU2) | | | | | | | | | | | |
| Procedure | D505 | D533 | D561 | D589 | D617 | D645 | D673 | D701 | D729 | ED^{a} | +60 d | +120 d |

General assessments/procedures

Informed
consent
Eligibility
assessment
Demographics
sFLC, sIFE,
and uIFE^{d}
TTR
genotyping^{e}
Medical
history
Prior
medications,
therapies,
and
procedures
Randomization

Study intervention

| | | | | | | | | | |
|---|---|---|---|---|---|---|---|---|---|
| Study | X | X | X | X | X | X | X | X | X |
| intervention | | | | | | | | | |
| infusion | | | | | | | | | |

Safety assessments

| | | | | | | | | | | | | |
|---|---|---|---|---|---|---|---|---|---|---|---|---|
| Weight | X | X | X | X | X | X | X | X | X | X | X | X |
| Height | | | | | | | | | | | | |
| Physical | X | X | X | X | X | X | X | X | X | X | X | X |
| examination ^{g} | | | | | | | | | | | | |
| Pregnancy | X | X | X | X | X | X | X | X | X | X | X | X |
| test^{h} | | | | | | | | | | | | |
| Vital signsⁱ | X | X | X | X | X | X | X | X | X | X | X | X |
| 12-lead ECG^{j} | | X | | | X | | | | X | X | X | X |
| Clinical | | X | | | X | | | | X | X | X | X |
| laboratory | | | | | | | | | | | | |
| tests | | | | | | | | | | | | |

| | | | |
|---|---|---|---|
| Concomitant | X | X | X |
| medications, | | | |
| therapies, | | | |
| and | | | |
| procedures | | | |
| AEs | X | X | X |

Efficacy and QoL assessments

| | | | | | | |
|---|---|---|---|---|---|---|
| NYHA | X | X | X | X | X | X |
| Functional | | | | | | |
| Classification | | | | | | |
| NT-proBNP | X | X | X | X | X | X |
| hs-cTnT | X | X | X | X | X | X |
| CRP | X | X | X | X | X | X |
| Echocardiogram^{k} | | | X | X | | |
| 6MWT^{k, l} | X | | X | X | | |
| KCCQ | X | | X | X | | [symbol] |
| EQ-5D-5L | X | | X | X | | [symbol] |
| and SF-36 | | | | | | |
| sNfL^{m} | | | X | X | | [symbol] |
| PND score | | | X | X | | [symbol] |
| and FAP | | | | | | |
| stage^{k, m} | | | | | | |
| Norfolk- | | | X | X | | [symbol] |
| QoL-DN^{k, m} | | | | | | |

PK/immunogenicity assessments

| | | | | | |
|---|---|---|---|---|---|
| PK samplesⁿ | X | X | X | X | X [symbol] |
| Immunogenicity | X | X | X | X | X [symbol] |
| samples^{o} | | | | | |
| Additional | X | X | X | X | X [symbol] |
| biomarkers^{p} | | | | | |

Other assessments (optional at selected study sites)

| | | | |
|---|---|---|---|
| Cardiac | X | X | [symbol] |
| scintigraphy^{k, q} | | | |
| Cardiac MRI^{k, q} | X | X | [symbol] |
| Tissue biopsy^{k, r} | X | X | [symbol] |
| Mixed | X | X | [symbol] |
| phenotype | | | |
| diagnosis^{s} | | | |
| NC study^{k, s} | X | X | [symbol] |
| NIS^{k, s} | X | X | [symbol] |

^{a}Participants who prematurely discontinue study intervention should be encouraged to complete all scheduled study visits and assessments (except study intervention infusion). Participants withdrawing from the study prior to completion of the Blinded Treatment Period, regardless of cause, will be asked to complete an ED Visit as soon as possible.
^{b}Safety Follow-up Visits are to be performed 60 days and 120 days after the last study intervention administration for all participants. Participants completing 24 months (Visit 30) before the last surviving participant has completed 24 months of blinded study treatment will immediately continue blinded treatment according to Table 26 without undergoing Safety Follow-up Visits according to this schedule. In participants withdrawing from the study prior to completion of the Blinded Treatment Period, the Safety Follow-up Visits should be performed 60 days and 120 days after the last study intervention administration.
^{c}On Day 3 ± 1 day (Visit 3), platelet count (only) is to be tested in a local laboratory and reported to the Investigator. Platelet count will be repeated as described in Section 10.11.
^{d}If historical results are not available or insufficient to exclude a monoclonal gammopathy for central eligibility confirmation, these tests can be performed as part of the Screening laboratory assessment. These tests are not required in all participants.
^{e}TTR genotype must be determined prior to randomization either through existing documentation or testing. If participants do not have confirmatory genotyping documentation prior to Screening, genotyping will be performed during Screening.
^{f}First dose of study intervention (ALXN2220 or placebo) must be administered within 24 hours of randomization.
^{g}A full physical examination includes inspection of general appearance, skin, nose, ears, eyes, neck, throat, heart, abdomen, lungs, vascular system, nervous system, musculoskeletal system, and extremities. Medical standard procedures will be applied. On treatment days, a full physical examination will be performed before the start of the infusion (up to −2 hours) and an abbreviated physical examination, inclusive of general appearance, heart, lungs, skin, musculoskeletal system and extremities and other organs or body systems as clinically indicated should be performed prior to the participant's discharge.
^{h}For WOCBP, serum hCG performed by the central laboratory is required at Screening. Prior to the first administration, a local serum pregnancy test has to be performed within 4 days prior to infusion. Subsequent pregnancy tests may be performed locally using serum (up to 4 days prior to infusion) or urine dipsticks (up to 1 day prior to infusion). A negative test result has to be available before infusion start.
ⁱVital sign measurements will be recorded at each visit and include systolic and diastolic BP, heart rate, respiratory rate, oxygen saturation, and body temperature. Vital signs should be measured before blood is drawn for laboratory tests. Measurements of systolic and diastolic BP, heart rate and respiratory rate should be made after the participant has been resting for at least 5 minutes. On treatment days, vital signs are to be measured within 15 minutes before of start of infusion, then every 15 minutes (±5 minutes) during the infusion and at the EOI. During the postinfusion observation period of at least 2 hours, vital signs are to be measured 30, 60, and 120 minutes after EOI (±5 minutes), and then periodically as clinically indicated, until the participant is discharged.
^{j}Both pre-infusion ECGs (to be performed within 2 hours prior to infusion) and postinfusion ECGs (to be performed up to 30 minutes post EOI) are to be performed after the participant has been resting for at least 5 minutes.
^{k}The time window is ±10 days from the visit date. This window does not apply for Screening Period.
^{l}On days with infusion, to be performed after blood draw for laboratory assessments and KCCQ, EQ-5D-5L and SF-36 assessments, but before start of infusion.
^{m}Only for participants with ATTRv-CM.
ⁿPK samples are to be taken predose (up to −2 hours) and at EOI (up to +30 minutes). EOI samples must be drawn from the opposite arm from the study intervention infusion. Additional PK sampling after first dose (24 ± 2 h, 168 ± 24 h, 336 ± 48 h, 672 ± 72 h [this sample must be taken prior to the second dose]) will be collected in a subset of willing Chinese participants.
^{o}Immunogenicity samples are to be taken predose (up to −2 hours). In case of suspected SAEs such as hypersensitivity or anaphylaxis, additional immunogenicity samples may be collected at or near the event.
^{p}Biomarker sampling will occur predose (up to −2 hours) on days of infusion (Section 8.8). Includes exploratory blood collection unless prohibited by local regulations. Stored as per the laboratory manual for follow up exploration of laboratory findings and/or AEs. Not for genetic research.
^{q}Cardiac scintigraphy and/or cardiac MRI may be performed in participants at selected study sites. If historic cardiac scintigraphy scans are not available or insufficient for central confirmation of the diagnosis, a cardiac scintigraphy may be repeated at any study site for central confirmation of eligibility.
^{r}Collection of an optional salivary gland biopsy, skin punch biopsy (in ATTRv participants) or cardiac biopsy at selected sites.
^{s}Only for participants with ATTRv-CM at study sites where neurological evaluation is possible. The phenotype is considered ‘mixed’ in the case of cardiac amyloidosis coexistent with sensorimotor neurological involvement documented by abnormal NC studies (during Screening or in historical NC study) or NIS ≥5 (during Screening or in historical documents (Section 5.6). [symbol]
indicates data missing or illegible when filed

TABLE 26
Schedule of Activities after 24 Months

| | Blinded Treatment Period | | | | | | | | |
|---|---|---|---|---|---|---|---|---|---|
| | After 24 Months up to 48 Months | | | | | | | Post-Treatment | |
| | (for participants who complete 24 months of | | | | | | | Safety | |
| | study treatment prior to the last participant) | | | | | | | Follow-up^{a} | |
| | Cycle^{b} (6 months duration) | | | | | | | | |
| | y, y + 1, y + 2, y + 3 | | | | | | | | |
| | Visit | | | | | | | | |
| | C_{y} V1 | C_{y} V2 | C_{y} V3 | C_{y} V4 | C_{y} V5 | C_{y} V6 | | FU1 | FU2 |
| | Week | | | | | | | | |
| | +4 | +8 | +12 | +16 | +20 | +24 | | +8 | +17 |
| Study Period | Day (Window: ±7) | | | | | | | | |
| Procedure | +28 | +56 | +84 | +112 | +140 | +168 | ED^{c} | +60 d | +120 d |

General assessments/procedures

| | | | | | | |
|---|---|---|---|---|---|---|
| Study intervention | X | X | X | X | X | X |
| infusion | | | | | | |

Safety assessments

| | | | | | | | | | |
|---|---|---|---|---|---|---|---|---|---|
| Weight | X | X | X | X | X | X | X | X | X |
| Physical examination^{d} | X | X | X | X | X | X | X | X | X |
| Pregnancy test^{e} | X | X | X | X | X | X | X | X | X |
| Vital signs^{f} | | X | X | X | X | X | X | X | X |
| 12-lead ECG^{g} | | | X | | | X | X | X | X |
| Clinical laboratory | | | X | | | X | X | X | X |
| tests | | | | | | | | | |
| Concomitant | | | X | | | | X | X | X |
| medications, | | | | | | | | | |
| therapies, and | | | | | | | | | |
| procedures | | | | | | | | | |
| AEs | | | X | | | | X | X | X |

Efficacy and QoL assessments

| | | | | | |
|---|---|---|---|---|---|
| NYHA Functional | X | X | X | X | X |
| Classification | | | | | |
| NT-proBNP | X | X | X | X | X |
| hs-cTnT | X | X | X | X | X |
| CRP | X | X | X | X | X |
| Echocardiogram^{h} | | X | X | | |
| 6MWT^{h, i} | | X | X | | |
| KCCQ | | X | X | | |
| EQ-5D-5L and SF-36 | | X | X | | |
| sNfL^{j} | | X | X | | |
| PND score and FAP | | X | X | | |
| stage^{h, j} | | | | | |
| Norfolk-QoL-DN^{h, j} | | X | X | | |

PK/immunogenicity assessments

| | | | | | |
|---|---|---|---|---|---|
| PK samples^{k} | X | X | X | X | X |
| Immunogenicity | X | X | X | X | X |
| samples^{l} | | | | | |
| Additional biomarkers^{m} | X | X | X | X | X |

Other assessments (optional at selected study sites)

| | | |
|---|---|---|
| Cardiac scintigraphy^{h, n} | X | X |
| Cardiac MRI^{h, n} | X | X |
| Nerve conduction | X | X |
| study^{h, o} | | |
| NIS^{h, o} | X | X |

^{a}Safety Follow-up Visits will be performed 60 and 120 days after the last dose of study intervention. In participants withdrawing from the study prior to completion of the Blinded Treatment Period, the safety follow-up visits should be performed 60 days and 120 days after the last study intervention administration.
^{b}The visit schedule will cycle every 6 months until the last surviving participant completes 24 months of blinded study treatment (Table 25). Cycles will be numbered in ascending order (1, 2, 3, 4) and visits will be named accordingly. Visits will be scheduled relative to the last study intervention administration at 24 months (Visit 30 in Table 25 for the first cycle) or in the previous cycle (C_{y} V6; for all subsequent cycles). When the last surviving participant completes 24 months of study treatment, no further blinded study intervention administrations will be performed and all ongoing participants will be scheduled for safety follow-up visits.
^{c}Participants who prematurely discontinue study intervention treatment should be encouraged to complete all scheduled study visits and assessments (except study intervention infusion). Participants withdrawing from the study prior to completion of the Blinded Treatment Period, regardless of cause, will be asked to complete an ED Visit as soon as possible.
^{d}A full physical examination includes inspection of general appearance, skin, nose, ears, eyes, neck, throat, heart, abdomen, lungs, vascular system, nervous system, musculoskeletal system, and extremities. Medical standard procedures will be applied. On treatment days, a full physical examination will be performed before the start of the infusion (up to −2 hours) and an abbreviated physical examination, inclusive of general appearance, heart, lungs, skin, musculoskeletal system and extremities and other organs or body systems as clinically indicated should be performed prior to the participant's discharge.
^{e}For WOCBP, pregnancy tests may be performed locally using serum (up to 4 days prior to infusion) or urine dipsticks (up to 1 day prior to infusion). A negative test result has to be available before infusion start.
^{f}Vital sign measurements will be recorded at each visit and include systolic and diastolic BP, heart rate, respiratory rate, oxygen saturation, and body temperature. Vital signs should be measured before blood is drawn for laboratory tests. Measurements of systolic and diastolic BP, heart rate and respiratory rate should be made after the participant has been resting for at least 5 minutes. On treatment days, vital signs are to be measured within 15 minutes before of start of infusion, then every 15 minutes (±5 minutes) during the infusion and at the EOI. During the postinfusion observation period of at least 2 hours, vital signs are to be measured 30, 60, and 120 minutes after EOI (±5 minutes), and then periodically as clinically indicated, until the participant is discharged.
^{g}Both preinfusion ECGs (to be performed within 2 hours prior to infusion) and postinfusion ECGs (to be performed up to 30 minute post EOI) are to be performed after the participant has been resting for at least 5 minutes.
^{h}The time window is ±10 days from the visit date.
ⁱOn days with infusion, to be performed after blood draw for laboratory assessments and KCCQ, EQ-5D-5L and SF-36 assessments, but before start of infusion.
^{j}Only for participants with ATTRv-CM.
^{k}PK samples are to be taken predose (up to −2 hours) and at EOI (up to +30 minutes). EOI samples must be drawn from the opposite arm from the study intervention infusion.
^{l}Immunogenicity samples are to be taken predose (up to −2 hours). In case of suspected SAEs such as hypersensitivity or anaphylaxis, additional immunogenicity samples may be collected at or near the event.
^{m}Biomarker sampling occurs predose (up to −2 hours) on days of infusion (Section 8.8). Includes exploratory blood collection unless prohibited by local regulations. Stored as per the laboratory manual for follow up exploration of laboratory findings and/or AEs. Not for genetic research.
ⁿCardiac scintigraphy and/or cardiac MRI may be performed in participants at selected study sites during the second and fourth cycle (after 36 months and 48 months of treatment).
^{o}Only for participants with ATTRv-CM at study sites where neurological evaluation is possible. The phenotype is considered ‘mixed’ in the case of cardiac amyloidosis coexistent with sensorimotor neurological involvement documented by abnormal NC studies (during Screening or in historical NC study) or NIS ≥5 (during Screening or in historical documents (Section 5.6).

List of Abbreviations

| Abbreviation or Term | Explanation |
|---|---|
| 6MWT | six-minute walk test |
| AA | serum amyloid A |
| ACM | all-cause mortality |
| ADA | antidrug antibody |
| ADL | activities of daily living |
| AE | adverse event |
| AL | amyloid light chain |
| ALT | alanine transaminase |
| AST | aspartate transaminase |
| ATTR | transthyretin amyloid protein |
| ATTR-CM | transthyretin amyloid cardiomyopathy |
| ATTR-PN | transthyretin amyloid polyneuropathy |
| ATTRv | variant transthyretin amyloid protein/genotype |
| ATTRv-CM | variant transthyretin amyloid cardiomyopathy |
| ATTRwt | wild-type transthyretin amyloid protein/genotype |
| AUC | area under the concentration-time curve |
| AUCα | area under the serum concentration-time curve from zero to infinity |
| AUCₜ | area under the serum concentration-time curve from time zero to the end of |
| | the dosing interval after the first dose |
| AUCₜₐᵤ | area under the concentration-time curve from time zero to the end of |
| | the dosing interval |
| B6 | vitamin B6 |
| B12 | vitamin B12 |
| BP | blood pressure |
| C3 | complement factor 3 |
| C4 | complement factor 4 |
| CEC | Clinical Events Adjudication Committee |
| CFR | Code of Federal Regulation |
| CI | confidence interval |
| CITP | collagen type I telopeptide |
| CIOMS | Council for International Organizations of Medical Sciences |
| CKD-Epi | Chronic Kidney Disease Epidemiology Collaboration |
| CL | serum clearance |
| CMAD | cardiac mechanical assist device |
| CMAP | compound muscle action potential |
| Cmax | maximum observed concentration |
| cMRI | cardiac magnetic resonance imaging |
| CNS | central nervous system |
| CONSORT | Consolidated Standards of Reporting Trials |
| COVID-19 | coronavirus disease 2019 |
| CRF | case report form |
| CRP | C-reactive protein |
| CSR | clinical study report |
| CTCAE | National Cancer Institute Common Terminology Criteria for Adverse Events |
| CTFG | Clinical Trial Facilitation Group |
| cTnT | cardiac troponin T |
| C_{trough} | minimum observed concentration |
| CV | cardiovascular |
| Cy | cycle |
| D/d | day |
| DILI | drug-induced liver injury |
| DPD | 3,3-diphosphono-1,2-propanodicarboxylic acid |
| ECG | electrocardiogram |
| eCRF | electronic case report form |
| ECV | extracellular volume |
| ECV_Mid | mid-ventricular extracellular volume |
| ED | early discontinuation |
| eGFR | estimated glomerular filtration rate |
| EQ-5D | EuroQol 5 dimensions |
| EQ-5D-5L | EuroQol 5 dimensions 5 levels quality of life questionnaire |
| EQ-VAS | European Quality of Life visual analogue scale |
| EU CTR | European Union Clinical Trials Regulation |
| EuroQoL | European Quality of Life |
| EU MDR | European Union Medical Device Regulation |
| FAP | familial amyloid polyneuropathy |
| FAS | Full Analysis Set |
| FSH | follicle-stimulating hormone |
| fT4 | free thyroxine |
| FU | follow-up |
| GCP | Good Clinical Practice |
| GLS | global longitudinal strain |
| GPS | Global Patient Safety |
| hATTR | hereditary ATTR |
| HBcAb | hepatitis B core antibody |
| HbsAg | hepatitis B surface antigen |
| hCG | human chorionic gonadotropin |
| HF | heart failure |
| HFrEF | heart failure reduced ejection fraction |
| HFmrEF | heart failure mid-range ejection fraction |
| HFpEF | heart failure preserved ejection fraction |
| HIV | human immunodeficiency virus |
| HMDP | hydroxymethylene diphosphonate |
| HRT | hormone replacement therapy |
| hs-cTnT | high sensitivity cardiac troponin T |
| HRWBR | heart-by-whole body retention ratio |
| IA | interim analysis |
| IB | Investigator's Brochure |
| ICF | informed consent form |
| ICH | International Council of Harmonisation of Technical Requirements for |
| | Pharmaceuticals for Human Use |
| ICMJE | International Committee of Medical Journal Editors |
| IDMC | independent data monitoring committee |
| IE | intercurrent event |
| IEC | independent ethics committee |
| IENFD | intra-epidermal nerve fiber density |
| IFE | immunofixation electrophoresis |
| IgG1 | immunoglobulin G1 |
| IL | interleukin |
| IMP | investigational medicinal product |
| INFg | interferon gamma |
| INR | international normalized ratio |
| IP | intellectual property |
| IQR | interquartile range |
| IRB | institutional review board |
| IRR | infusion-related reactions |
| IRT | interactive response technology |
| IV | intravenous(ly) |
| IVIG | intravenous immunoglobulin |
| IVRS/IWRS | interactive voice/web response system |
| J2R | jump to reference |
| KCCQ-OS | Kansas City Cardiomyopathy Questionnaire-Overall Score |
| KPS | Karnofsky Performance Status |
| LS | least squares |
| LVEF | left ventricular ejection fraction |
| mAb | monoclonal antibody |
| MAD | multiple ascending dose |
| MCS | mental component summary |
| MMRM | mixed model for repeated measures |
| MNAR | missing not at random |
| mNIS + 7 | Modified Neuropathy Impairment Score + 7 components |
| MRI | magnetic resonance imaging |
| NA | not applicable |
| NAb | neutralizing antibodies |
| NAC | National Amyloidosis Centre |
| NC | nerve conduction |
| ND | normal deviate |
| NfL | neurofilament light chain |
| NIS | Neuropathy Impairment Score |
| NNTTR | non-native TTR |
| Norfolk QoL-DN | Norfolk Quality of Life-Diabetic Neuropathy |
| NT-proBNP | N-terminal pro b-type natriuretic peptide |
| NYHA | New York Heart Association |
| OLE | open-label extension |
| PAG | patient advocacy group |
| PCS | Physical Component Summary |
| PD | pharmacodynamic(s) |
| PDAS | Pharmacodynamic Analysis Set |
| PICP | procollagen type I |
| PIIINP | serum amino-terminal propeptide of procollagen type III |
| PK | pharmacokinetic(s) |
| PKAS | Pharmacokinetic Analysis Set |
| PND | polyneuropathy disability |
| PNS | peripheral nervous system |
| PopPK | population pharmacokinetics |
| PPS | Per Protocol Set |
| PRO | patient-reported outcome |
| PRO-C6 | plasma propeptide of type VI collagen |
| PYP | pyrophosphate |
| q4w | every 4 weeks |
| QoL | quality of life |
| QoL-DN | Quality of Life-Diabetic Neuropathy |
| QTL | quality tolerance limits |
| RBC | red blood cell |
| RBP | retinol binding protein |
| RTSM | Randomization and Trial Supply Management |
| SAA | serum amyloid A protein |
| SAD | single ascending dose |
| SAE | serious adverse event |
| SAP | statistical analysis plan |
| SCIG | subcutaneous immunoglobulin |
| SD | standard deviation |
| SF-36 | Short Form 36 Health Survey |
| sFLC | serum free light chain |
| sIFE | serum immunofixation electrophoresis |
| SGNFD | sweat gland nerve fiber density |
| SNAP | sural sensory nerve action potential |
| sNfL | serum neurofilament light chain |
| SoA | schedule of activities |
| SpO₂ | peripheral oxygen saturation |
| SPECT | single photon emission computed tomography |
| SPEP | serum protein electrophoresis |
| SUSAR | suspected unexpected serious adverse reaction |
| t_{1/2} | terminal elimination half-life |
| T3 | triiodothyronine |
| T4 | thyroxine |
| TEAE | treatment-emergent adverse event |
| TNF-α | tumor necrosis factor-alpha |
| TSH | thyroid stimulating hormone |
| TTR | transthyretin |
| TQoL | total quality of life |
| TUDCA | tauroursodeoxycholic acid |
| UHF | urgent heart failure |
| uIFE | urine immunofixation electrophoresis |
| ULN | upper limit of normal |
| V | Visit |
| Vss | apparent volume of distribution at steady state |
| WBC | white blood cell |
| WOCBP | woman of childbearing potential |
| y | year |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the following items and the claims.

### ORDERED EMBODIMENTS A

[1] A human anti-transthyretin (TTR) antibody, which is capable of binding aggregated TTR species and does not substantially recognize physiological TTR species for use in a method of treating or effecting prophylaxis of a subject having or at risk of having a transthyretin-mediated amyloidosis (ATTR), wherein the method comprises administration of the antibody in a dosing regimen that results in a sustained plasma concentration of the antibody at about 1 μg/mL to about 1000 μg/mL in the subject or at an area under the curve (AUC) of about 2,000 μg*day/mL to about 100,000 μg*day/mL.
[2] The anti-TTR antibody for use according to [1], wherein the antibody binds aggregates of mutated and wild-type TTR species.
[3] The anti-TTR antibody for use according to [1] or [2], wherein the antibody does not bind to monomers and dimers of human native TTR.
[4] The anti-TTR antibody for use according to any one of [1] to [3], wherein the antibody binds to a TTR epitope which comprises or consists of the amino acid sequence EEEFVEGIY (SEQ ID NO: 49), GELHGLTTEEE (SEQ ID NO: 50), or WEPFA (SEQ ID NO: 51).
[5] The anti-TTR antibody for use according to any one of [1] to [4], wherein the anti-TTR antibody is administered at a dose of about 0.3 mg/kg to about 60 mg/kg.
[6] The anti-TTR antibody for use according to any one of [1] to [5], wherein the anti-TTR antibody thereof is administered at a dose of about 10 mg/kg.
[7] The anti-TTR antibody for use according to any one of [1] to [5], wherein the anti-TTR antibody is administered at a dose of about 30 mg/kg.
[8] The anti-TTR antibody for use according to any one of [1] to [5], wherein the anti-TTR antibody is administered at a dose of about 30 mg/kg to about 60 mg/kg.
[9] The anti-TTR antibody for use according to any one of [1] to [5] and [7] or [8], wherein the anti-TTR antibody is administered at a dose of about 30 mg/kg and results in a sustained plasma concentration of the antibody of about 50 μg/mL or an AUC of about 50,000 μg*day/mL in the subject.
[10] The anti-TTR antibody for use according to any one of [1] to [5] and [8], wherein the anti-TTR antibody is administered at a dose of about 60 mg/kg.
[11] The anti-TTR antibody for use according to any one of [1] to [5], wherein the anti-TTR antibody or is first administered at a starting dose of about 0.3 mg/kg to about 10 mg/kg.
[12] The anti-TTR antibody for use according to [11], wherein the anti-TTR antibody is further administered at a maintenance dose of about 10 mg/kg to about 60 mg/kg.
[13] The anti-TTR antibody for use according to any one of [1] to [4], wherein the anti-TTR antibody is administered at a maintenance dose of about 600 mg to about 7500 mg.
[14] The anti-TTR antibody for use according to [13], wherein the maintenance dose is about 2500 mg.
[15] The anti-TTR antibody for use according to [14], wherein the maintenance dose is 2400 mg.
[16] The anti-TTR antibody for use according to [13], wherein the maintenance dose is about 3000 mg.
[17] The anti-TTR antibody for use according to [13], wherein the maintenance dose is about 3500 mg.
[18] The anti-TTR antibody for use according to [17], wherein the maintenance dose is 3200 mg.
[19] The anti-TTR antibody for use according to any one of [13] to [18], wherein the anti-TTR antibody is further administered at a loading dose of about 600 mg to about 4000 mg.
[20] The anti-TTR antibody for use according to [19], wherein the loading dose is about 2500 mg.
[21] The anti-TTR antibody for use according to [20], wherein the loading dose is 2400 mg.
[22] The anti-TTR antibody for use according to [19], wherein the loading dose is about 3000 mg.
[23] The anti-TTR antibody for use according to [19], wherein the loading dose is about 3500 mg.
[24] The anti-TTR antibody for use according to [23], wherein the loading dose is 3200 mg.
[25] The anti-TTR antibody for use according to any one of [19] to [24], wherein the maintenance dose and/or the loading dose of the anti-TTR antibody is about 3000 mg.
[26] The anti-TTR antibody for use according to any one of [19] to [24], wherein the maintenance dose and/or the loading dose of the anti-TTR antibody is about 3500 mg.
[27] The anti-TTR antibody for use according to [26], wherein the maintenance dose and/or the loading dose of the anti-TTR antibody is 3200 mg.
[28] The anti-TTR antibody for use according to [13], wherein the anti-TTR antibody is administered at a maintenance dose of about 2000 mg to 2500 mg to a subject with a body weight of about 40 to about ≤60 kg.
[29] The anti-TTR antibody for use according to [28], wherein the anti-TTR antibody is administered at a maintenance dose of about 2000 mg to a subject with a body weight of about 40 to about ≤60 kg.
[30] The anti-TTR antibody for use according to [28], wherein the anti-TTR antibody is administered at a maintenance dose of about 2500 mg to a subject with a body weight of about 40 to about ≤60 kg.
[31] The anti-TTR antibody for use according to [30], wherein the anti-TTR antibody is administered at a maintenance dose of 2400 mg to a subject with a body weight of about 40 to about ≤60 kg.
[32] The anti-TTR antibody for use according to [13], wherein the anti-TTR antibody is administered at a maintenance dose of about 3000 mg to 3500 mg to a subject with a body weight of about ≥60 kg to about 100 kg.
[33] The anti-TTR antibody for use according to [32], wherein the anti-TTR antibody is administered at a maintenance dose of about 3000 mg to a subject with a body weight of about ≥60 kg to about 100 kg.
[34] The anti-TTR antibody for use according to [32], wherein the anti-TTR antibody is administered at a maintenance dose of about 3500 mg to a subject with a body weight of about ≥60 kg to about 100 kg.
[35] The anti-TTR antibody for use according to [34], wherein the anti-TTR antibody is administered at a maintenance dose of 3200 mg to a subject with a body weight of about ≥60 kg to about 100 kg.
[36] The anti-TTR antibody for use according to [13], wherein the anti-TTR antibody is administered at a maintenance dose of about 4000 mg to 5000 mg to a subject with a body weight of about ≤100 kg.
[37] The anti-TTR antibody for use according to [36], wherein the anti-TTR antibody is administered at a maintenance dose of about 4000 mg to a subject with a body weight of about ≤100 kg.
[38] The anti-TTR antibody for use according to [36], wherein the anti-TTR antibody is administered at a maintenance dose of about 4500 mg to a subject with a body weight of about ≤100 kg.
[39] The anti-TTR antibody for use according to [36], wherein the anti-TTR antibody is administered at a maintenance dose of about 5000 mg to a subject with a body weight of about ≤100 kg.
[40] The anti-TTR antibody for use according to [39], wherein the anti-TTR antibody is administered at a maintenance dose of 4800 mg to a subject with a body weight of about ≤100 kg.
[41] The anti-TTR antibody for use according to any one of [1] to [40], wherein the anti-TTR antibody is administered to the subject about once every 21 to 35 days.
[42] The anti-TTR antibody for use according to [41], wherein the anti-TTR antibody is administered to the subject about once every 28 to 35 days.
[43] The anti-TTR antibody for use according to [42], wherein the anti-TTR antibody is administered to the subject about once every 28 days.
[44] The anti-TTR antibody for use according to [42], wherein the anti-TTR antibody is administered to the subject about once every 35 days.
[45] The anti-TTR antibody for use according to any one of [1] to [44], wherein the anti-TTR antibody thereof is administered to the subject about once every 4 weeks (q4w).
[46] The anti-TTR antibody for use according to any one of [1] to [4], wherein the anti-TTR antibody is administered at a dose of about 2400 mg to a subject with a body weight of about 40 to about 560 kg once every 4 weeks (q4w).
[47] The anti-TTR antibody for use according to any one of [1] to [4], wherein the anti-TTR antibody is administered at a dose of about 3200 mg to a subject with a body weight of ≥60 kg to about 100 kg once every 4 weeks (q4w).
[48] The anti-TTR antibody for use according to any one of [1] to [4], wherein the anti-TTR antibody is administered at a dose of about 4800 mg to a subject with a body weight about ≤100 kg once every 4 weeks (q4w).
[49] The anti-TTR antibody for use according to any one of [1] to [48], wherein the anti-TTR antibody thereof is administered to the subject for about 4-30 months.
[50] The anti-TTR antibody for use according to [49], wherein the anti-TTR antibody is administered to the subject for about 12-18 months.
[51] The anti-TTR antibody for use according to [49], wherein the anti-TTR antibody is administered to the subject for about 4 months.
[52] The anti-TTR antibody for use according to [49], wherein the anti-TTR antibody is administered to the subject for about 11 months.
[53] The anti-TTR antibody for use according to any one of [49] to [52], wherein administration of the anti-TTR antibody is paused for about 1 week to about 1 year.
[54] The anti-TTR antibody for use according to any one of [7] to [9], wherein the anti-TTR antibody is administered to the subject at a dose of about 30 mg/kg for about 12-18 months.
[55] The anti-TTR antibody for use according to [30] or [46], wherein the anti-TTR antibody is administered at a dose of about 2400 mg to a subject with a body weight of about 40 to about ≤60 kg for about 24-48 months.
[56] The anti-TTR antibody for use according to [34] or [47], wherein the anti-TTR antibody is administered at a dose of about 3200 mg to a subject with a body weight of about ≥60 kg to about 100 kg for about 24-48 months.
[57] The anti-TTR antibody for use according to [39] or [48], wherein the anti-TTR antibody is administered at a dose of about 4800 mg to a subject with a body weight of about ≤100 kg for about 24-48 months.
[58] The anti-TTR antibody for use according to any one of [19] to [24], wherein the loading dose is administered once every other week for up to two months before administration of a maintenance dose.
[59] The anti-TTR antibody for use according to any one of [1] to [58], wherein the plasma concentration is sustained for about 1 day to about 30 days.
[60] The anti-TTR antibody for use according to [59], wherein the plasma concentration is sustained for about 1 day to about 1 week.
[61] The anti-TTR antibody for use according to any one of [1] to [60], wherein administration of the anti-TTR antibody is monitored by determining the level of one or more biomarkers.
[62] The anti-TTR antibody for use according to [61], wherein the biomarker is N-terminal pro-B-type natriuretic peptide (NT-proBNP), and wherein a decrease of the level of the biomarkers is indicative for efficacy of the treatment.
[63] The anti-TTR antibody for use according to [62] wherein the biomarkers further comprise cardiac troponin T (TnT).
[64] The anti-TTR antibody for use according to any one of [61] to [63], further comprising determining the level of C-reactive protein (CRP), wherein a dose dependent transient increase of CRP level is indicative for on-target immune activation and efficacy of the treatment.
[65] The anti-TTR antibody for use according to any one of [1] to [64], wherein administration of the anti-TTR antibody leads to reduction of cardiac amyloid burden.
[66] The anti-TTR antibody for use according to any one of [1] to [65], wherein administration of the anti-TTR antibody leads to cardiac mass reduction as measured by cardiac magnetic resonance imaging (MRI).
[67] The anti-TTR antibody for use according to any one of [1] to [66], wherein administration of the anti-TTR antibody leads to improved cardiovascular function as assessed by echocardiography.
[68] The anti-TTR antibody for use according to any one of [1] to [67], wherein the anti-TTR antibody is provided in an aqueous formulation at a concentration of about 25 to 125 mg/mL, which is diluted to about 1 to about 50 mg/mL prior to administration.
[69] The anti-TTR antibody for use according to any one of [1] to [68], wherein the subject has been previously treated with and/or is concurrently receiving a TTR tetramer stabilizer.
[70] The anti-TTR antibody for use according to [69], wherein the TTR tetramer stabilizer is selected from the group consisting of diflunisal, Tafamidis, and Acoramidis.
[71] The anti-TTR antibody for use according to any one of [1] to [70], wherein the antibody is administered to the subject by intravenous infusion in an aqueous formulation, wherein the aqueous formulation has a pH of about 5.0 to 6.5, comprises a histidine buffer, a sugar such as sucrose, and a surfactant such as polysorbate 80 (PS80), and wherein the antibody is present in the aqueous formulation at a concentration of about 25 to 125 mg/mL.
[72] The anti-TTR antibody for use according to [71], wherein the aqueous formulation has a pH of about 5.8 and comprises a 20 mM histidine buffer (L-histidine and L-histidine monohydrochloride), 6.5% weight per volume (w/v) sucrose, 0.03% PS80 w/v, and wherein the antibody is present in the aqueous formulation at a concentration of about 50 mg/mL.
[73] The anti-TTR antibody for use according to [71], wherein the aqueous formulation has a pH of about 5.8 and comprises a 20 mM histidine buffer (L-histidine and L-histidine monohydrochloride), 8% weight per volume (w/v) sucrose, 0.03% PS80 w/v, and wherein the antibody is present in the aqueous formulation at a concentration of about 50 mg/mL.
[74] The anti-TTR antibody for use according to any one of [71] to [73], wherein the antibody is administered to the subject in a diluted form comprising a diluent.
[75] The anti-TTR antibody for use according to [74], wherein the diluent is glucose or a polymer thereof, preferably glucose.
[76] The anti-TTR antibody for use according to [75], wherein the polymer is dextran.
[77] The anti-TTR antibody for use according to any one of [74] to [76], wherein an infusion line is flushed with the diluent before and after the intravenous infusion.
[78] The anti-TTR antibody for use according to any one of [74] to [76], wherein the intravenous infusion is performed with a syringe pump with an infusion syringe for a total antibody dose of up to 100 mg, or with an infusion pump for total antibody doses exceeding 100 mg, optionally using an infusion bag prefilled with the diluent.
[79] The anti-TTR antibody for use according to [78], wherein the infusion syringe includes a minimum administration volume of 10 mL of the aqueous formulation.
[80] The anti-TTR antibody for use according to any one of [74] to [78], wherein a total volume of the diluted form administered to the subject does not exceed 200 mL.
[81] The anti-TTR antibody for use according to any one of [71] to [80], wherein at least for a first infusion the aqueous formulation is administered over approximately 2 hours±10 minutes.
[82] The anti-TTR antibody for use according to [81], wherein the aqueous formulation is administered over approximately 1 hour±10 minutes.
[83] The anti-TTR antibody for use according to any one of [1] to [82], wherein the ATTR amyloidosis leads to Cardiomyopathy (CM).
[84] The anti-TTR antibody for use according to any one of [1] to [83], wherein the subject has ATTR amyloidosis with CM (ATTR-CM).
[85] The anti-TTR antibody for use according to [84], wherein the subject has been diagnosed with hereditary ATTR-CM for a known pathogenic TTR mutation.
[86] The anti-TTR antibody for use according to [84], wherein the subject has sporadic, WT-ATTR-CM and a negative genetic testing for a TTR mutation.
[87] The anti-TTR antibody for use according to any one of [83] to [86], wherein the subject has left ventricular ejection fraction (LVEF) 220% as measured, e.g., by echocardiography.
[88] The anti-TTR antibody for use according to any one of [83] to [87], wherein the subject has left ventricular wall thickness (LVWT) 212 mm as measured, e.g., by echocardiography.
[89] The anti-TTR antibody for use according to any one of [83] to [88], wherein the subject has an N-terminal pro b-type natriuretic peptide (NT-proBNP) level of about 300 μg/mL to about 20,000 μg/mL.
[90]. The anti-TTR antibody for use according to any one of [83] to [89], wherein the subject has an N-terminal pro b-type natriuretic peptide (NT-proBNP) level of >2000 μg/mL.
[91] The anti-TTR antibody for use according to any one of [1] to [90], wherein the anti-TTR antibody comprises a heavy chain variable (VH) region having the three complementary determining regions (CDRs) set forth in SEQ ID NOs: 1-3 and a light chain variable (VL) region having the three CDRs set forth in SEQ ID NOs: 4-6.
[92] The anti-TTR antibody for use according to any one of [1] to [91], wherein the VH region of the anti-TTR antibody comprises an amino acid sequence having at least 80% sequence identity to SEQ ID NO: 7 and the VL region comprises an amino acid sequence having at least 80% sequence identity to SEQ ID NO: 8.
[93] The anti-TTR antibody for use according to any one of [1] to [92], wherein the heavy chain variable region of the anti-TTR antibody comprises the amino acid sequence of SEQ ID NO: 7 and the light chain variable region comprises the amino acid of SEQ ID NO: 8.
[94] The anti-TTR antibody for use according to any one of [1] to [92], wherein the heavy chain variable region of the anti-TTR antibody comprises the amino acid sequence of SEQ ID NO: 11 and the light chain variable region comprises the amino acid of SEQ ID NO: 8.
[95] The anti-TTR antibody for use according to any one of [1] to [92], wherein the heavy chain variable region of the anti-TTR antibody comprises the amino acid sequence of SEQ ID NO: 11 and the light chain variable region comprises the amino acid of SEQ ID NO: 12.
[96] The anti-TTR antibody for use according to any one of [1] to [95], wherein the antibody comprises a human Ig constant region or an equivalent region capable of mediating phagocytosis.
[97] The anti-TTR antibody for use according to any one of [1] to [96], wherein the antibody is a human IgG.
[98] The anti-TTR antibody for use according to any one of [1] to [97], wherein the antibody is a human IgG1.
[99] The anti-TTR antibody for use according to any one of [1] to [98], wherein the antibody is a human IgG1m3 allotype.
[100] The anti-TTR antibody for use according to any one of [96] to [99], wherein the antibody comprises a kappa (c) light chain.
[101] The anti-TTR antibody for use according to any one of [1] to [100], wherein the antibody is NI006/ALXN2220.
[102] The anti-TTR antibody for use according to any one of [1] to [101], wherein the antibody comprises two heavy chains and two light chains, wherein each heavy chain is comprised of 450 amino acid residues having SEQ ID NO: 9, and each light chain is comprised of 214 amino acid residues having SEQ ID: 10.
[103] The anti-TTR antibody for use according to any one of [1] to [101], wherein the antibody comprises two heavy chains and two light chains, wherein each heavy chain is comprised of 449 amino acid residues having SEQ ID NO: 13, and each light chain is comprised of 214 amino acid residues having SEQ ID: 10.
[104] The anti-TTR antibody for use according to any one of [1] to [101], wherein the antibody comprises two heavy chains and two light chains, wherein each heavy chain is comprised of 449 amino acid residues having SEQ ID NO: 14, and each light chain is comprised of 214 amino acid residues having SEQ ID: 10.
[105] The anti-TTR antibody for use according to any one of [1] to [101], wherein the antibody comprises two heavy chains and two light chains, wherein each heavy chain is comprised of 448 amino acid residues having SEQ ID NO: 15, and each light chain is comprised of 214 amino acid residues having SEQ ID: 10.
[106] The anti-TTR antibody for use according to any one of [1] to [105], wherein the antibody is produced in CHO, preferably CHO-K1 host cells and purified from the cell culture.
[107] The anti-TTR antibody for use according to any one of [96] to [102], and [104] to [105], wherein the N-terminal glutamine in the amino acid sequence of the heavy chain of the antibody is modified to a pyroglutamate (pE).
[108] The anti-TTR antibody for use according to any one of [96] to [102], and [106] and [107], wherein the C-terminal lysine of the amino acid sequence of the heavy chain is cleaved off.
[109] The anti-TTR antibody for use according to any one of [96] to [108], which is N-glycosylated, preferably wherein the N-linked glycan is on N300 of the heavy chain.
[110] A TTR tetramer stabilizer for use in a method of treating or effecting prophylaxis of a subject having or at risk of ATTR-CM, wherein the subject has previously been and/or is concomitantly treated with the anti-TTR antibody as defined in any one of [1] to [109].
[111] The TTR tetramer stabilizer for use according to [110], wherein the TTR tetramer stabilizer is selected from the group consisting of diflunisal, Tafamidis, and Acoramidis.
[112] A method of treating ATTR, the method comprising administering to a human subject a human anti-TTR antibody at a dosage providing about 0.3 to about 100 mg/kg to the subject once every 3 to 42 days;
wherein the antibody comprises a heavy chain variable region having at least 80% sequence identity to SEQ ID NO: 7 and light chain variable region comprising an amino acid sequence having at least 80% sequence identity to SEQ ID NO: 8, wherein the heavy chain variable region comprises the CDRs set forth in SEQ ID NOs: 1-3 and the light chain variable region comprises the CDRs set forth in SEQ ID NOs: 4-6, and wherein the antibody is provided in a pharmaceutical composition at a concentration of about 10 mg/mL to about 125 mg/mL, which is diluted to about 1 mg/mL to about 50 mg/mL prior to administration.
[113] The method of [112], wherein the antibody comprises a human Ig constant region.
[114] The method of [112] or [113], wherein the antibody is a human IgG.
[115] The method of any one of [112] to [114], wherein the antibody is a human IgG1.
[116]. The method of any one of [112] to [115], wherein the antibody is a human IgG1m3 allotype.
[117] The method of any one of [112] to [116], wherein the antibody comprises a kappa (κ) light chain.
[118] The method of any one of [112] to [117], wherein the antibody is NI006/ALXN2220.
[119] The method of any one of [112] to [118], wherein the antibody comprises two heavy chains and two light chains, wherein each heavy chain is comprised of 450 amino acid residues having SEQ ID NO: 9, and each light chain is comprised of 214 amino acid residues having SEQ ID: 10.
[120] The method of any one of [112] to [118], wherein the antibody comprises two heavy chains and two light chains, wherein each heavy chain is comprised of 449 amino acid residues having SEQ ID NO: 13, and each light chain is comprised of 214 amino acid residues having SEQ ID: 10.
[121] The method of any one of [112] to [118], wherein the antibody comprises two heavy chains and two light chains, wherein each heavy chain is comprised of 449 amino acid residues having SEQ ID NO: 14, and each light chain is comprised of 214 amino acid residues having SEQ ID: 10.
[122] The method of any one of [112] to [118], wherein the antibody comprises two heavy chains and two light chains, wherein each heavy chain is comprised of 448 amino acid residues having SEQ ID NO: 15, and each light chain is comprised of 214 amino acid residues having SEQ ID: 10.
[123] The method of any one of [112] to [122], wherein the antibody is produced in CHO host cells and purified from the cell culture.
[124] The method any one of [118] to [119], and [121] to [123], wherein the N-terminal glutamine in the amino acid sequence of the heavy chain of the antibody is modified to a pyroglutamate (pE).
[125] The method of any one of [118] and [123] to [124], wherein the C-terminal lysine of the amino acid sequence of the heavy chain is cleaved off.
[126] The method of any one of [118] to [125], which is N-glycosylated, preferably wherein the N-linked glycan is on N300 of the heavy chain.
[127] The method of any one of [112] to [126], wherein the anti-TTR antibody is administered at a dose of about 0.3 mg/kg to about 60 mg/kg.
[128] The method of any one of [112] to [127], wherein the anti-TTR antibody is administered at a dose of about 10 mg/kg.
[129] The method of any one of [112] to [127], wherein the anti-TTR antibody is administered at a dose of about 30 mg/kg.
[130] The method of any one of [112] to [127], wherein the anti-TTR antibody is administered at a dose of about 30 mg/kg to about 60 mg/kg.
[131] The method of any one of [112] to [127] and [130], wherein the anti-TTR antibody is administered at a dose of about 30 mg/kg and results in a sustained plasma concentration of the antibody of about 50 μg/mL or an AUC of about 50,000 μg*day/mL in the subject.
[132] The method of any one of [112] to [127] and [130], wherein the anti-TTR antibody is administered at a dose of about 60 mg/kg.
[133] The method of any one of [112] to [127] wherein the anti-TTR antibody is first administered at a starting dose of about 0.3 mg/kg to about 10 mg/kg.
[134] The method of [133], wherein the anti-TTR antibody s further administered at a maintenance dose of about 10 mg/kg to about 60 mg/kg.
[135] The method of [134], wherein the anti-TTR antibody is administered at a maintenance dose of about 600 mg to about 7500 mg.
[136] The method of [135], wherein the maintenance dose is about 2500 mg.
[137] The method of [136], wherein the maintenance dose is about 2400 mg.
[138] The method of [135], wherein the maintenance dose is about 3000 mg.
[139] The method of [135], wherein the maintenance dose is about 3500 mg
[140] The method of [139], wherein the maintenance dose is about 3200 mg.
[141] The method of [135], wherein the anti-TTR antibody is further administered at a loading dose of about 600 mg to about 4000 mg.
[142] The method of [141], wherein the loading dose is about 2500 mg.
[143] The method of [142], wherein the loading dose is about 2400 mg.
[144] The method of [141], wherein the loading dose is about 3000 mg.
[145] The method of [141], wherein the loading dose is about 3500.
[146] The method of [145], wherein the loading dose is about 3200 mg.
[147] The method of [141], wherein the maintenance dose and/or the loading dose of the anti-TTR antibody is about 3000 mg.
[148] The method of [141], wherein the maintenance dose and/or the loading dose of the anti-TTR antibody is about 3500 mg.
[149] The method of [148], wherein the maintenance dose and/or the loading dose of the anti-TTR antibody is about 3200 mg.
[150] The method of [135], wherein the anti-TTR antibody is administered at a maintenance dose of about 2000 mg to 2500 mg to a subject with a body weight of about 40 to about ≤60 kg.
[151] The method of [150], wherein the anti-TTR antibody is administered at a maintenance dose of about 2000 mg to a subject with a body weight of about 40 to about ≤60 kg.
[152] The method of [150], wherein the anti-TTR antibody is administered at a maintenance dose of about 2500 mg to a subject with a body weight of about 40 to about ≤60 kg.
[153] The method of [152], wherein the anti-TTR antibody is administered at a maintenance dose of about 2400 mg to a subject with a body weight of about 40 to about ≤60 kg.
[154] The method of [135], wherein the anti-TTR antibody s administered at a maintenance dose of about 3000 mg to 3500 mg to a subject with a body weight of about ≥60 kg to about 100 kg.
[155] The method of [154], wherein the anti-TTR antibody is administered at a maintenance dose of about 3000 mg to a subject with a body weight of about ≥60 kg to about 100 kg.
[156] The method of [154], wherein the anti-TTR antibody is administered at a maintenance dose of about 3500 mg to a subject with a body weight of about ≥60 kg to about 100 kg.
[157] The method of [156], wherein the anti-TTR antibody is administered at a maintenance dose of about 3200 mg to a subject with a body weight of about ≥60 kg to about 100 kg.
[158] The method of [135], wherein the anti-TTR antibody is administered at a maintenance dose of about 4000 mg to 5000 mg to a subject with a body weight of about ≤100 kg.
[159] The method of [158], wherein the anti-TTR is administered at a maintenance dose of about 4000 mg to a subject with a body weight of about ≤100 kg.
[160] The method of [158], wherein the anti-TTR antibody is administered at a maintenance dose of about 4500 mg to a subject with a body weight of about ≤100 kg.
[161] The method of [158], wherein the anti-TTR antibody is administered at a maintenance dose of about 5000 mg to a subject with a body weight of about ≤100 kg.
[162] The method of [161], wherein the anti-TTR antibody is administered at a maintenance dose of about 4800 mg to a subject with a body weight of about ≤100 kg.
[163] The method of any one of [112] to [162], wherein the anti-TTR antibody is administered to the subject about once every 21 to 35 days.
[164] The method of any one of [112] to [163], wherein the anti-TTR antibody is administered to the subject about once every 28 to 35 days.
[165] The method of any one of [112] to [164], wherein the anti-TTR antibody is administered to the subject about once every 28 days.
[166] The method of any one of [112] to [164], wherein the anti-TTR antibody is administered to the subject about once every 35 days.
[167] The method of any one of [112] to [162], wherein the anti-TTR antibody is administered to the subject about once every 4 weeks (q4w).
[168] The method of any one of [112] to [167], wherein the anti-TTR antibody is administered to the subject for about 4-30 months.
[169] The method of [168], wherein the anti-TTR antibody is administered to the subject for about 12-18 months.
[170] The method of [168], wherein the anti-TTR is administered to the subject for about 4 months.
[171] The method of [168], wherein the anti-TTR antibody is administered to the subject for about 11 months.
[172] The method of [151], [155], or [160], wherein the anti-TTR antibody is administered to the subject for about 24-48 months.
[173] The method of any one of [168] to [172], wherein administration of the anti-TTR antibody is paused for about 1 week to about 1 year.
[174] The method of any one of [129] to [132], wherein the anti-TTR antibody is administered to the subject at a dose of about 30 mg/kg or 60 mg/kg for about 12-18 months.
[175] The method of any one of [141] to [146], wherein the loading dose is administered once every other week for up to two months before administration of a maintenance dose.
[176] The method of any one of [112] to [175], wherein administration of the anti-TTR antibody results in a sustained plasma concentration of the antibody at about 1 μg/mL to about 100 μg/mL in the subject or an AUC of about 2,000 μg*day/mL to about 100,000 μg*day/mL.
[177] The method of [176], wherein the plasma concentration is sustained for about 1 day to about 30 days.
[178] The method of [177], wherein the plasma concentration is sustained for about 1 day to about 1 week.
[179] The method of any one of [112] to [178], wherein administration of the anti-TTR antibody is monitored by determining the level of one or more biomarkers.
[180] The method of [179], wherein the biomarker is N-terminal pro-B-type natriuretic peptide (NT-proBNP), and wherein a decrease of the level of the biomarker is indicative of treatment efficacy.
[181] The method of [179], wherein the biomarkers further comprise cardiac troponin T (TnT).
[182] The method of any one of [179] to [181], further comprising determining the level of C-reactive protein (CRP), wherein a dose dependent transient increase of CRP level is indicative for on-target immune activation and efficiency of the treatment.
[183] The method of any one of [112] to [182], wherein administration of the anti-TTR antibody leads to reduction of cardiac amyloid burden.
[184] The method of any one of [112] to [183], wherein administration of the anti-TTR antibody leads to cardiac mass reduction as measured by cardiac magnetic resonance imaging (MRI).
[185] The method of any one of [112] to [184], wherein administration of the anti-TTR antibody leads to improved cardiovascular function as assessed by echocardiography.
[186] The method of any one of [112] to [185], wherein the anti-TTR antibody is provided in an aqueous formulation at a concentration of about 25 to 125 mg/mL, which is diluted to 1 mg/mL to about 50 mg/mL prior to administration.
[187] The method of any one of [112] to [186], wherein the subject has been previously treated with and/or is concurrently receiving a TTR tetramer stabilizer.
[188] The method of [187], wherein the TTR tetramer stabilizer is selected from the group consisting of diflunisal, Tafamidis, and Acoramidis.
[189] The method of any one of [112] to [188], wherein the antibody is administered to the subject by intravenous infusion in an aqueous formulation, wherein the aqueous formulation has a pH of about 5.0 to 6.5, comprises a histidine buffer, a sugar such as sucrose, and a surfactant such as polysorbate 80 (PS80), and wherein the antibody is present in the aqueous formulation at a concentration of about 25 to 125 mg/mL.
[190] The method of [189], wherein the aqueous formulation has a pH of about 5.8 and comprises 20 mM histidine hydrochloride, 6.5% weight per volume (w/v) sucrose, 0.03% PS80 w/v, and wherein the antibody is present in the aqueous formulation at a concentration of about 50 mg/mL.
[191] The method of [189], wherein the aqueous formulation has a pH of about 5.8 and comprises 20 mM histidine hydrochloride, 8% weight per volume (w/v) sucrose, 0.03% PS80 w/v, and wherein the antibody is present in the aqueous formulation at a concentration of about 50 mg/mL.
[192] The method of [189] to [191], wherein the antibody is administered to the subject in a diluted form comprising a diluent.
[193] The method of [192], wherein the diluent is glucose or a polymer thereof, preferably glucose.
[194] The method of [193], wherein the polymer is dextran.
[195] The method of any one of [192] to [194], wherein an infusion line is flushed with the diluent before and after the intravenous infusion.
[196] The method of any one of [192] to [195], wherein the intravenous infusion is performed with a syringe pump with an infusion syringe for a total antibody dose of up to 100 mg, or with an infusion pump for total antibody doses exceeding 100 mg, optionally using an infusion bag prefilled with the diluent.
[197] The method of [196], wherein the infusion syringe includes a minimum administration volume of 10 mL of the aqueous formulation.
[198] The method of any one of [192] to [197], wherein a total volume of the diluted form administered to the subject does not exceed 200 mL, including a flushing volume.
[199] The method of any one of [189] to [198], wherein at least for a first infusion the aqueous formulation is administered over approximately 2 hours±10 minutes.
[200] The method of [198], wherein the aqueous formulation is administered over approximately 1 hour±10 minutes.
[201] The method of any one of [112] to [200], wherein the ATTR amyloidosis leads to Cardiomyopathy (CM).
[202] The method of any one of [112] to [200], wherein the subject has ATTR amyloidosis with CM (ATTR-CM).
[203] The method of [202], wherein the subject has been diagnosed with hereditary ATTR-CM for a known pathogenic TTR mutation.
[204] The method of [202], wherein the subject has sporadic, WT-ATTR-CM and a negative genetic testing for a TTR mutation.
[205] The method of any one of [201] to [204], wherein the subject has left ventricular ejection fraction (LVEF) 220% as measured, e.g., by echocardiography.
[206] The method of any one of [201] to [205], wherein the subject has left ventricular wall thickness (LVWT) 212 mm as measured, e.g., by echocardiography.
[207] The method of any one of [201] to [206], wherein the subject has an N-terminal pro b-type natriuretic peptide (NT-proBNP) level of about 300 pg/mL to about 20,000 pg/mL.
[208] The anti-TTR antibody for use according to any one of [201] to [207], wherein the subject has an N-terminal pro b-type natriuretic peptide (NT-proBNP) level of >2000 pg/mL.
[209] The method of any one of [112] to [208], wherein the heavy chain variable region comprises the amino acid sequence of SEQ ID NO: 7 and the light chain variable region comprises the amino acid of SEQ ID NO: 8.
[210] The method of any one of [112] to [208], wherein the heavy chain variable region comprises the amino acid sequence of SEQ ID NO: 10 and the light chain variable region comprises the amino acid of SEQ ID NO: 8.
[211] The method of any one of [112] to [208], wherein the heavy chain variable region comprises the amino acid sequence of SEQ ID NO: 10 and the light chain variable region comprises the amino acid of SEQ ID NO: 11.
[212] The method of any one of [112] to [210], further comprising administering a TTR tetramer stabilizer to the subject.
[213] The method of [212], wherein the TTR tetramer stabilizer is selected from the group consisting of diflunisal, Tafamidis, and Acoramidis.
[214] A human anti-TTR antibody, which is capable of binding mutated, misfolded, misassembled and/or aggregated TTR species thereof and does not substantially recognize physiological TTR species, for use in treating or effecting prophylaxis of a subject having or at risk of having a transthyretin-mediated amyloidosis (ATTR) by administering the antibody in a dosing regimen that results in a sustained plasma concentration of the antibody at an AUC of about 2,000 μg*day/mL to about 100,000 μg*day/mL, preferably at least 30,000 μg*day/mL in the subject.
[215] The anti-TTR antibody according to [214], wherein the anti-TTR antibody is for administration at a dose of about 30 mg/kg and results in a sustained plasma concentration of the antibody at an AUC of about 2,000 μg*day/mL to about 100,000 μg*day/mL, preferably at least 50,000 μg*day/mL in the subject.
[216] Use of a human anti-TTR antibody, which is capable of binding mutated, misfolded, misassembled and/or aggregated TTR species and does not substantially recognize physiological TTR species, in the manufacture of a medicament for treating or effecting prophylaxis of a subject having or at risk of having ATTR, wherein the medicament is for administration to the subject in a dosing regimen that results in a sustained plasma concentration of the antibody of about 1 μg/mL to about 100 μg/mL in the subject.
[217] The use of [216], wherein the antibody is the antibody as defined in any one of the preceding embodiments.
[218] The use of [216] or [217], wherein administration of the medicament is performed as defined in any one of the preceding embodiments.
[219] The use of any one of [216] to [218], wherein the subject is the subject as defined in any one of the preceding embodiments.
[220] Use of a human anti-TTR antibody, which is capable of binding mutated, misfolded, misassembled and/or aggregated TTR species and does not substantially recognize physiological TTR species, in the manufacture of a medicament for treating or effecting prophylaxis of a subject having or at risk of having ATTR, wherein the medicament is for administration to the subject in a dosing regimen that results in a sustained plasma concentration of the antibody at an AUC of about 2,000 μg*day/mL to about 100,000 μg*day/mL, preferably at least 30,000 μg*day/mL in the subject.
[221] The use of [220], wherein the antibody is the antibody as defined in any one of the preceding embodiments.
[222] The use of [220] or [221], wherein administration of the medicament is performed as defined in any one of the preceding embodiments.
[223] The use of any one of [220] to [222], wherein the subject is the subject as defined in any one of the preceding embodiments.
[224] Use of a human anti-TTR antibody for the manufacture of a medicament for treating ATTR in a subject, wherein:
(a) the medicament is for administration to the subject at a dosage of about 0.3 to about 100 mg/kg of an anti-TTR antibody once every 3 to 42 days, preferably once every 4 weeks; and
(b) the comprises a heavy chain variable region having at least 80% sequence identity to SEQ ID NO: 7 and light chain variable region comprising an amino acid sequence having at least 80% sequence identity to SEQ ID NO: 8, wherein the heavy chain variable region comprises the CDRs set forth in SEQ ID NOs: 1-3 and the light chain variable region comprises the CDRs set forth in SEQ ID NOs: 4-6, and wherein the antibody is provided at a concentration of about 1 mg/mL to about 125 mg/mL, preferably of about 50 mg/mL.
[225] Use of a human anti-TTR antibody for the manufacture of a medicament for treating ATTR in a subject, wherein:
(a) the medicament is for administration to the subject at a dosage of about 2000 to 2500 mg, preferably of about 2400 mg to a subject with a body weight of about 40 to about ≤60 kg; of about 3000 to 3500 mg, preferably of about 3200 mg to a subject with a body weight of about ≥60 kg to about 100 kg; or about 4500 to 5000 mg, preferably of about 4800 mg to a subject with a body weight of about ≤100 kg once every 3 to 42 days, preferably once every 4 weeks; and
(b) the antibody comprises a heavy chain variable region having at least 80% sequence identity to SEQ ID NO: 7 and light chain variable region comprising an amino acid sequence having at least 80% sequence identity to SEQ ID NO: 8, wherein the heavy chain variable region comprises the CDRs set forth in SEQ ID NOs: 1-3 and the light chain variable region comprises the CDRs set forth in SEQ ID NOs: 4-6, and wherein the antibody is provided at a concentration of about 1 mg/mL to about 125 mg/mL, preferably of about 50 mg/mL.
[226] The use of [224] or [225], wherein the antibody is of the human IgG1m3 allotype comprising two heavy chains and two light chains, wherein each heavy chain has the amino acid sequence of SEQ ID NO: 9, and each light chain has the amino acid sequence of SEQ ID NO: 10.
[227] The use of any one of [224] to [226], wherein the heavy chain of the antibody lacks the C-terminal lysine, wherein the glutamine at the N-terminus of the heavy chain is modified as pyro-glutamic acid, and wherein the heavy chain is N-glycosylated.
[228] The use of any one of [224] to [227], wherein the antibody is administered to the subject by intravenous infusion in an aqueous formulation, wherein the aqueous formulation has a pH of about 5.0 to 6.5, comprises a histidine buffer, a sugar such as sucrose, and a surfactant such as polysorbate 80 (PS80), and wherein the antibody is present in the aqueous formulation at a concentration of about 25 to 125 mg/mL.
[229] The use of [228], wherein the aqueous formulation has a pH of about 5.8 and comprises 20 mM L-histidine buffer, preferably histidine and L-histidine monohydrochloride, 8% weight per volume (w/v) sucrose, 0.03% PS80 w/v, and wherein the antibody is present in the aqueous formulation at a concentration of about 50 mg/mL.
[230] A therapeutic kit comprising: (i) one or more container(s), wherein the container(s) comprise a formulation of an anti-TTR antibody in an amount of 2400 mg, 3200 mg or 4800 mg or multiple doses of any one thereof; and (ii) means for delivery of the antibody to a human, wherein the means optionally comprises an infusion bag and/or a syringe, wherein the anti-TTR antibody is capable of binding mutated, misfolded, misassembled and/or aggregated TTR species and does not substantially recognize physiological TTR species, preferably wherein the antibody is the antibody as defined in any one of the preceding embodiments.
[231] A therapeutic kit comprising: (i) one or more container(s), wherein the container(s) comprise a formulation of an anti-TTR antibody in an amount of 2400 mg, 3200 mg or 4800 mg or multiple doses of any one thereof; and (ii) means for delivery of the antibody to a human, wherein the means optionally comprises an infusion bag and/or a syringe, wherein the anti-TTR antibody is antibody NI006/ALXN2220.
[232] An article of manufacture comprising: one or more container(s) comprising (i) a formulation of an anti-TTR antibody in a total amount of 2400 mg, 3200 mg or 4800 mg or multiple doses of any one thereof; and (ii) a leaflet prescribing that the antibody is to be administered at a dosage of 400 mg to a subject with a body weight of 40 to ≤60 kg, of 3200 mg to a subject with a body weight of 360 kg to 100 kg; or of 4800 mg to a subject with a body weight of ≤100 kg once every 4 weeks, wherein the anti-TTR antibody is capable of binding mutated, misfolded, misassembled and/or aggregated TTR species and does not substantially recognize physiological TTR species, preferably wherein the antibody is the antibody as defined in any one of the preceding embodiments.
[233] An article of manufacture comprising: one or more container(s) comprising (i) a formulation of an anti-TTR antibody in a total amount of 2400 mg, 3200 mg or 4800 mg or multiple doses of any one thereof; and (ii) a leaflet prescribing that the antibody is to be administered at a dosage of 400 mg to a subject with a body weight of 40 to ≤60 kg, of 3200 mg to a subject with a body weight of ³60 kg to 100 kg; or of 4800 mg to a subject with a body weight of ≤100 kg once every 4 weeks, wherein the anti-TTR antibody is antibody NI006/ALXN2220.
[234] The kit of [230] or [231], or the article of manufacture of [232] or [233], wherein ALXN2220/NI006 is a recombinant human-derived immunoglobulin gamma 1 (IgG1) monoclonal antibody specifically recognizing aggregated wt and mutant forms of TTR and is expressed in a Chinese hamster ovary cell line.
[235] The kit of any one of [230], [231], and [234], or the article of manufacture of any one of
[232] to [233], wherein ALXN2220/NI006 has an approximate molecular weight of 150 kDa.
[236] The kit of any one of [230], [231], [234], and [235], or the article of manufacture of any one of [232] to [235], wherein ALXN2220/NI006 is a concentrate for solution for infusion that is presented as sterile, colorless to slightly yellow, clear to slightly opalescent liquid, essentially free of visible particles for intravenous use by infusion after dilution.
[237] The kit or article of manufacture of [236], wherein each mL of solution contains 50 mg/mL ALXN2220/NI006 in 20 mM histidine buffer, 80 mg/mL sucrose, 0.3 mg/mL polysorbate 80, at pH 5.8.
[238] The article of manufacture of any one of [232] to [237], wherein the recommended dosage of ALXN2220/NI006 on the label is 2400 mg for patients with weight of ≥40 kg to <60 kg, 3200 mg for ≥60 kg to <100 kg and 4800 mg for patients with weight of ≥100 kg that must be diluted then administered as an intravenous infusion over approximately one to two hours, once every four weeks.
[239] The kit of any one of [230], [231] and [234] to [237], or the article of manufacture of any one of [232] to [238], wherein prior to administration, ALXN2220/NI006 is diluted with 5% glucose solution.
[240] The kit of any one of [230], [231], and [234] to [237] and [239], or the article of manufacture of any one of [232] to [239], wherein ALXN2220/NI006 is a clear to opalescent and colorless to pale yellow solution, available as solution 100 mg/2 mL (50 mg/mL) in a single-dose vial.
[241] Use of C-reactive protein (CRP) as biomarker for monitoring the efficacy of the treatment of ATTR with a therapeutic agent which is capable of depleting amyloid deposition.
[242] The use of [241], wherein the therapeutic agent is an anti-TTR antibody.
[243] A method of monitoring the efficacy of the treatment of ATTR with a therapeutic agent which is capable of depleting amyloid deposition, wherein the method comprises determining the level of CRP in a sample from the subject, wherein the level of CRP is determined at a first timepoint before administration of the therapeutic agent and at least one second timepoint after administration of the therapeutic agent, wherein an increased level of CRP in the sample of the second timepoint in comparison to the sample of the first timepoint is indicative for the treatment efficiency.
[244] The method of [243], wherein the therapeutic agent is an anti-TTR antibody.
[245] A kit useful for monitoring the efficacy of the treatment of ATTR with a therapeutic agent which is capable of depleting amyloid deposition, said kit comprising at least a reagent for the detecting and/or capturing of CRP, and optionally further reagents and/or instructions for use.
[246] Use of the kit of [245] in a method of any one of the preceding embodiments.
[247] A human anti-transthyretin (TTR) antibody, which is a monoclonal antibody comprising
(a) a mature heavy chain variable region comprising three CDRs of SEQ ID NO:61 as set forth in WO 2019/108689 A1 and a mature light chain variable region comprising three CDRs of SEQ ID NO:70 as set forth in WO 2019/108689 A1, except that positions H52 and L26 by Kabat numbering can each be independently N or S, or a monoclonal antibody comprising a mature heavy chain variable region comprising three CDRs of SEQ ID NO: 1 as set forth in WO 2019/108689 A1 and a mature light chain variable region comprising three CDRs of SEQ ID NO:16 as set forth in WO 2019/108689 A1; and/or
(b) a mature heavy chain variable region comprising the amino acid sequence of SEQ ID NO:65 as set forth in WO 2019/108689 A1, and a mature light chain variable region comprising the amino acid sequence of SEQ ID NO:76 as set forth in WO 2019/108689 A1,
for use in a method of treating or effecting prophylaxis of a subject having or at risk of having a transthyretin-mediated amyloidosis (ATTR), wherein the method comprises administration of the antibody to the subject at a dosage of about 2000 to 2500 mg, preferably of about 2400 mg or 2500 mg to a subject with a body weight of about 40 to about ≤60 kg; of about 3000 to 3500 mg, preferably of about 3200 mg or 3500 mg to a subject with a body weight of about ≥60 kg to about 100 kg; or about 4500 to 5000 mg, preferably of about 4800 mg or 5000 mg to a subject with a body weight of about ≤100 kg once every 3 to 42 days, preferably once every 4 week.
[248] A method of treating ATTR, the method comprising administering to a human subject a human anti-TTR antibody at a dosage of about 2000 to 2500 mg, preferably of about 2400 mg or 2500 mg to a subject with a body weight of about 40 to about ≤60 kg; of about 3000 to 3500 mg, preferably of about 3200 mg or 3500 mg to a subject with a body weight of about ≥60 kg to about 100 kg; or about 4500 to 5000 mg, preferably of about 4800 mg or 5000 mg to a subject with a body weight of about ≤100 kg once every 3 to 42 days, preferably once every 4 week, wherein the antibody comprises:
(a) a mature heavy chain variable region comprising three CDRs of SEQ ID NO:61 as set forth in WO 2019/108689 A1 and a mature light chain variable region comprising three CDRs of SEQ ID NO:70 as set forth in WO 2019/108689 A1, except that positions H52 and L26 by Kabat numbering can each be independently N or S, or a monoclonal antibody comprising a mature heavy chain variable region comprising three CDRs of SEQ ID NO: 1 as set forth in WO 2019/108689 A1 and a mature light chain variable region comprising three CDRs of SEQ ID NO:16 as set forth in WO 2019/108689 A1; and/or
(b) a mature heavy chain variable region comprising the amino acid sequence of SEQ ID NO:65 as set forth in WO 2019/108689 A1, and a mature light chain variable region comprising the amino acid sequence of SEQ ID NO:76 as set forth in WO 2019/108689 A1.
[249] Use of a human anti-TTR antibody or for the manufacture of a medicament for treating ATTR in a subject, wherein:
(a) the medicament is for administration to the subject at a dosage of about 2000 to 2500 mg, preferably of about 2400 mg to a subject with a body weight of about 40 to about ≤60 kg; of about 3000 to 3500 mg, preferably of about 3200 mg to a subject with a body weight of about ≥60 kg to about 100 kg; or about 4500 to 5000 mg, preferably of about 4800 mg to a subject with a body weight of about ≤100 kg once every 3 to 42 days, preferably once every 4 weeks; and
(b) the antibody comprises
   (i) a mature heavy chain variable region comprising three CDRs of SEQ ID NO:61 as set forth in WO 2019/108689 A1 and a mature light chain variable region comprising three CDRs of SEQ ID NO:70 as set forth in WO 2019/108689 A1, except that positions H52 and L26 by Kabat numbering can each be independently N or S, or a monoclonal antibody comprising a mature heavy chain variable region comprising three CDRs of SEQ ID NO: 1 as set forth in WO 2019/108689 A1 and a mature light chain variable region comprising three CDRs of SEQ ID NO:16 as set forth in WO 2019/108689 A1; and/or
   (ii) a mature heavy chain variable region comprising the amino acid sequence of SEQ ID NO:65 as set forth in WO 2019/108689 A1, and a mature light chain variable region comprising the amino acid sequence of SEQ ID NO:76 as set forth in WO 2019/108689 A1.

### ORDERED EMBODIMENTS B

[1] An anti-transthyretin (TTR) antibody for use in a method of treating transthyretin-mediated amyloidosis (ATTR) in a subject in need of said treatment, wherein the method comprises administering the antibody at a dose of: 2000 mg to 2500 mg to a patient weighing equal or more than 40 kg (≥40 kg) to under 60 kg (<60 kg); (b) 3000 mg to 3500 mg to a patient weighing equal or more than 60 kg (≥60 kg) to under 100 kg (<100 kg); or (c) 4000 mg to 5000 mg to a patient weighing equal or more than 100 kg (≥100 kg), wherein the antibody comprises a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8.
[2] The antibody for use according to [1], wherein the antibody is administered at a dose of 2400 mg to a patient weighing equal or more than 40 kg (≥40 kg) to under 60 kg (<60 kg); (b) 3200 mg to a patient weighing equal or more than 60 kg (≥60 kg) to under 100 kg (<100 kg); or (c) 4800 mg to a patient weighing equal or more than 100 kg (≥100 kg).
[3] The antibody for use according to [1] or [2], wherein the antibody comprises a human Ig constant region.
[4] The antibody for use according to any one of [1] to [3], wherein the antibody comprises a human IgG constant region, preferably wherein the antibody comprises a human IgG1 constant region comprising an antibody heavy chain constant region of human IgG1m3 allotype.
[5] The antibody for use according to any one of [1] to [4], wherein the antibody comprises two heavy chains and two light chains, wherein each heavy chain comprises 450 amino acid residues comprising the sequence of SEQ ID NO: 9, and each light chain comprises 214 amino acid residues comprising the sequence of SEQ ID NO: 10.
[6] The antibody for use according to any one of [1] to [4], wherein the antibody comprises two heavy chains and two light chains, wherein each heavy chain comprises 449 amino acid residues comprising the sequence of SEQ ID NO: 13, and each light chain comprises 214 amino acid residues comprising the sequence of SEQ ID NO: 10.
[7] The antibody for use according to any one of [1] to [4], wherein the antibody comprises two heavy chains and two light chains, wherein each heavy chain comprises 449 amino acid residues comprising the sequence of SEQ ID NO: 14, and each light chain comprises 214 amino acid residues comprising the sequence of SEQ ID NO: 10.
[8] The antibody for use according to any one of [1] to [4]1-4, wherein the antibody comprises two heavy chains and two light chains, wherein each heavy chain comprises 448 amino acid residues comprising the sequence of SEQ ID NO: 15, and each light chain comprises 214 amino acid residues comprising the sequence of SEQ ID NO: 10.
[9] The antibody for use according to any one of [1] to [8], wherein the antibody is NI006/ALXN2220 or comprises a binding fragment thereof.
[10] The antibody for use according to any one of [1] to [9], wherein the antibody is a recombinant antibody produced in Chinese hamster ovary (CHO) cells, preferably wherein the antibody is produced in CHO-K1 cells.
[11] The antibody for use according to any one of [1] to [5] and [7] to [10], wherein the heavy chain of the antibody comprises a cyclized N-terminal glutaminyl residue comprising pyroglutamate (pyro-Q).
[12] The antibody for use according to any one of [1] to [5], [9], and[10], wherein the antibody heavy chain further comprises a clipped C-terminal lysine.
[13] The antibody for use according to any one of [1] to [12], wherein the antibody is N-glycosylated, preferably wherein the N-linked glycan is on N300 of the heavy chain of the antibody.
[14] The antibody for use according to any one of [1] to [13], wherein the subject has symptomatic ATTR-CM and/or NT-proBNP>2000 pg/mL.
[15] The antibody for use according to [14], wherein the subject has either variant ATTR-CM (ATTRv-CM/hATTR-CM) or wild-type ATTR-CM ((wATTR-CM).
[16] The antibody for use according to [14] or [15], wherein the subject is an adult human subject, preferably, a subject who is 18 years or older but less than 90 years old ((≥18 to ≤90 years of age).
[17] The antibody for use according to any one of [14] to [16], wherein the treatment lowers cardiac amyloid load within 12 months and/or composite of all-cause mortality (ACM) and total cardiovascular (CV) clinical events.
[18] The antibody for use according to any one of [14] to [17], wherein the treatment lowers composite of all-cause mortality (ACM) and heart failure (HF) events.
[19] The antibody for use according to any one of [14] to [18], wherein the treatment improves at least one of the following:
(a) symptoms, functionality, and health-related quality-of-life (QoL) as measured by the change from baseline in Kansas City Cardiomyopathy Questionnaire overall summary (KCCQ-OS) score;
(b) time to cardiovascular (CV)-related mortality;
(c) six-minute walk test (6MWT) score compared to baseline;
(d) rate of cardiovascular (CV) clinical events;
(e) time to all-cause mortality (ACM).
[20] The antibody for use according to any one of [14] to [19], wherein the treatment has at least one of the following effects:
(a) reduces NT-proBNP levels in the subject compared to baseline;
(b) reduces rate of heart failure (HF) events;
(c) reduces incidence of intensification of oral diuretic therapy, which optionally includes outpatient augmentation of oral diuretic therapy;
(d) reduces incidence of changes in disease modifying therapy;
(e) reduces incidence of hospitalization for atrial fibrillation;
(f) induces change from baseline in Kansas City Cardiomyopathy Questionnaire overall summary (KCCQ-OS) score beyond 24 months of study treatment;
(g) induces change from baseline in six-minute walk test (6MWT) beyond 24 months of study treatment;
(h) induces change from baseline in ATTR-CM disease severity based on the Mayo, NAC, and Columbia disease stage, and NYHA classification;
(i) induces change from baseline in GLS;
(j) induces change from baseline in stroke volume;
(k) induces change from baseline in echocardiography parameters of interest;
(l) induces change from baseline in hs-cTnT;
(m) induces change from baseline in DPD/PYP/HMDP cardiac scintigraphy cardiac uptake and/or cMRI-derived ECV, T1 and T2 mapping;
(n) induces change from baseline in eGFR
(o) induces change from baseline in EQ-5D-5L score and/or induces change from baseline in SF-36 scores;
(p) induces change from baseline in a marker selected from CRP, IL IL1b, IL6, IL8, IFNg, TNF-α, IL10, IL1RA, C3 and C4, serum amyloid A, and ferritin; preferably a marker which is CRP;
(q) induces change from baseline in a marker selected from serum carboxy-terminal PICP, PIIINP, serum CITP, and plasma PRO-C6;
(r) induces change from baseline in a marker selected from TTR (prealbumin), TSH, RBP and fT4;
(s) induces change from baseline in (1) PND score and FAP stage; (2) Norfolk QoL-DN total score; and/or (3) sNFL levels;
(t) induces change from baseline in NIS and/or induces a change in NC studies.
[21] The antibody for use according to any one of [1] to [20], wherein the anti-TTR antibody is administered intravenously (IV); preferably wherein the anti-TTR antibody is administered every four weeks (q4w) via IV infusion.
[22] The antibody for use according to any one of [1] to [21], wherein the anti-TTR antibody is administered for at least 24 months, preferably at least 48 months.
[23] The antibody for use according to any one of [1] to [22], wherein the anti-TTR antibody is administered in a body weight-bracketed flat dose based on the patient's recorded body weight, wherein said recording is made within 30 days of a scheduled administration, wherein said dose is administered intravenously (IV) via infusion every four weeks (q4w).
[24] The antibody for use according to any one of [1] to [23], wherein the anti-TTR antibody triggers the elimination of ATTR fibrils from patient samples through immune driven phagocytosis clearance in a dose- and time-dependent manner.
[25] The antibody for use according to [24], wherein the elimination of ATTR fibrils by the anti-TTR antibody treatment is measured via cardiac tracer uptake scintigraphy or quantification of ECV with cMRI.
[26] The antibody for use according to [24], wherein the patient's treatment with the anti-TTR antibody results in a dose- and time-dependent reduction in the cardiac amyloid load up to approximately 51% at 12 months, preferably wherein the dose corresponds to 60 mg/kg.
[27] The antibody for use according to [24], wherein the patient is a male or a female who
   (1) has a centrally confirmed diagnosis of ATTR-CM with either wild-type or variant TTR genotype based on evidence of cardiac amyloidosis by echocardiography or cMRI and one of the following:
a. Endomyocardial biopsy with confirmatory TTR amyloid typing by either immunohistochemistry or mass spectrometry; OR
b. Grade 2 or 3 cardiac uptake on 99mTc scintigraphy (99mTc DPD, 99mTc PYP, or 99mTc HMDP) in the absence of monoclonal gammopathy; OR
c. Grade 2 or 3 cardiac uptake on 99mTc scintigraphy (99mTc DPD, 99mTc PYP, or 99mTc HMDP) AND confirmatory TTR amyloid typing by either immunohistochemistry or mass spectrometry in non-cardiac tissue in the presence of monoclonal gammopathy;
   (2) is willing to be genetically tested for mutations in the TTR gene during screening, if genetic testing was not previously performed or if genetic results are not available
   (3) has end-diastolic interventricular septal wall thickness ≥11 mm for women or ≥12 mm for men on echocardiography measured at Screening
   (4) has NT-proBNP>2000 pg/mL, as measured by a central laboratory at screening
   (5) has treatment with a loop diuretic for at least 30 days prior to screening;
   (6) has a history of heart failure as documented by one of the following events within 1 year prior to screening:
a. heart failure hospitalization
b. urgent heart failure visit
c. episode of volume overload documented by NT-proBNP>2000 pg/mL (or equivalent BNP)
   (7) is New York Heart Association (NYHA) classification Class II-IV at screening; and/or
   (8) has a life expectancy of at least 6 months as per the clinician's judgment.
[28] The antibody for use according to any one of [1] to [27], wherein the anti-TTR antibody is NI006/ALXN2220 which is administered in a pharmaceutical formulation at 50 mg/mL in 20 mM histidine buffer, 80 mg/mL sucrose, 0.3 mg/mL polysorbate 80, at pH 5.8.
[29] The antibody for use according to any one of [1] to [28], wherein the anti-TTR antibody is NI006/ALXN2220 which is administered as an intravenous (IV) infusion in said patient over 2 hours for initial administration and over 1 hour for subsequent administrations.
[30] The antibody for use according to any one of [1] to [29], wherein the patient has been treated with a disease modifying agent selected from TTR silencer and TTR stabilizer, preferably wherein the disease modifying agent comprises tafamidis, diflunisal, or a TTR silencer.
[31] The antibody for use according to any one of [1] to [30], wherein the anti-TTR antibody is administered for up to 24 months in accordance with the intervention infusion schedule provided in Table 25.
[32] The antibody for use according to [31], wherein the anti-TTR antibody is administered, as follow-up, after the 24-month treatment period in accordance with the intervention infusion schedule of Table 26.
[33] The antibody for use according to any one of [1] to [32], wherein the treatment results in a median amyloid reduction when the dose corresponds to a dose between 30 and 60 mg/kg in the patient.
[34] The antibody for use according to any one of [1] to [33], wherein the patient is stratified based on:
(1) prior treatment with a disease modifying agent, wherein the disease modifying agent is selected from (a) TTR silencer optionally together with a TTR stabilizer; (b) TTR stabilizer alone; and (c) no TTR stabilizer or TTR silencer treatment;
(2) TTR genotype comprising ATTR variant (ATTRv) or ATTR wild-type (ATTRwt); or
(3) disease severity based on cardiac biomarker levels comprising NT-proBNP>3000 pg/mL vs NT-proBNP≤3000 pg/mL, optionally together with levels of high sensitivity cardiac troponin C (hs-cTnT) pre- and post-treatment.
[35] The antibody for use according to any one of [1] to [34], wherein the treatment efficacy is measured with endomyocardial biopsy comprising intra-epidermal nerve fiber density (IENFD) and/or sweat gland nerve fiber density (SGNFD).
[36] The antibody for use according to any one of [1] to [35], wherein serum biomarker levels are monitored pre and post treatment with the anti-TTR antibody, wherein the biomarkers are selected from: (a) complement factors selected from C3 and C4, together with CRP; (b) pro-inflammatory cytokines selected from IL1 b, IL6, IL8, IFNg, and TNF-α; (c) anti-inflammatory cytokines selected from IL10, IL1RA; (d) positive acute phase proteins selected from SAA and ferritin; (e) PICP; (f) PIIINP; (g) CITP; (h) PRO-C6; and (i) plasma NNTTR, or a combination thereof.
[37] The antibody for use according to any one of [1] to [36], wherein the anti-TTR antibody, when administered to the human patient, is safe and well-tolerated by the human patient.
[38] A method of treating ATTR in a human subject in need of said treatment, the method comprising, administering to said human subject, an anti-transthyretin (TTR) antibody comprising a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8, wherein the antibody is administered at a dose of: (a): 2000 mg to 2500 mg to a patient weighing equal or more than 40 kg (≥40 kg) to under 60 kg (<60 kg); (b) 3000 mg to 3500 mg to a patient weighing equal or more than 60 kg (≥60 kg) to under 100 kg (<100 kg); or (c) 4000 mg to 5000 mg to a patient weighing equal or more than 100 kg (≥100 kg).
[39] The method of [38], wherein the antibody is administered at a dose of 2400 mg to a patient weighing equal or more than 40 kg (≥40 kg) to under 60 kg (<60 kg); (b) 3200 mg to a patient weighing equal or more than 60 kg (≥60 kg) to under 100 kg (<100 kg); or (c) 4800 mg to a patient weighing equal or more than 100 kg (≥100 kg).
[40] The method of [38] or [39], wherein the antibody comprises a human Ig constant region.
[41] The method of any one of [38] to [40], wherein the antibody comprises a human IgG, preferably wherein the antibody comprises a human IgG1 comprising an antibody heavy chain constant region of human IgG1m3 allotype.
[42] The method of any one of [38] to [41], wherein the antibody comprises two heavy chains and two light chains, wherein each heavy chain comprises 450 amino acid residues comprising the sequence of SEQ ID NO: 9, and each light chain comprises 214 amino acid residues comprising the sequence of SEQ ID NO: 10.
[43] The antibody for use according to any one of [38] to [41], wherein the antibody comprises two heavy chains and two light chains, wherein each heavy chain comprises 449 amino acid residues comprising the sequence of SEQ ID NO: 13, and each light chain comprises 214 amino acid residues comprising the sequence of SEQ ID NO: 10.
[44] The antibody for use according to any one of [38] to [41], wherein the antibody comprises two heavy chains and two light chains, wherein each heavy chain comprises 449 amino acid residues comprising the sequence of SEQ ID NO: 14, and each light chain comprises 214 amino acid residues comprising the sequence of SEQ ID NO: 10.
[45] The antibody for use according to any one of [38] to [41], wherein the antibody comprises two heavy chains and two light chains, wherein each heavy chain comprises 448 amino acid residues comprising the sequence of SEQ ID NO: 15, and each light chain comprises 214 amino acid residues comprising the sequence of SEQ ID NO: 10.
[46] The method of any one of [38] to [45], wherein the antibody is NI006/ALXN222 or comprises a binding fragment thereof.
[47] The method of any one of [38] to [46], wherein the antibody is a recombinant antibody produced in Chinese hamster ovary (CHO) cells, preferably wherein the antibody is produced in CHO-K1 cells.
[48] The method of any one of [38] to [47], wherein the heavy chain of the antibody comprises a cyclized N-terminal glutaminyl residue comprising pyroglutamate (pyro-Q) and optionally wherein the antibody heavy chain further comprises a clipped C-terminal lysine.
[49] The method of any one of [38] to [48], wherein the antibody is N-glycosylated, preferably wherein the N-linked glycan is on N300 of the heavy chain of the antibody.
[50] The method of any one of [38] to [49], wherein the subject has symptomatic ATTR-CM and/or NT-proBNP>2000 pg/mL.
[51] The method of [50], wherein the subject has either variant ATTR-CM (ATTRv-CM/hATTR-CM) or wild-type ATTR-CM ((wATTR-CM).
[52] The method of [50] or [51], wherein the subject is an adult human subject, preferably, a subject who is 18 years or older but less than 90 years old ((≥18 to ≤90 years of age).
[53] The method of any one of [50] to [52], wherein the treatment lowers composite of all-cause mortality (ACM) and total cardiovascular (CV) clinical events.
[54] The method of any one of [50] to [53], wherein the treatment lowers composite of all-cause mortality (ACM) and heart failure (HF) events.
[55] The method of any one of [50] to [54], wherein the treatment improves at least one of the following:
(a) symptoms, functionality, and health-related quality-of-life (QoL) as measured by the change from baseline in Kansas City Cardiomyopathy Questionnaire overall summary (KCCQ-OS) score;
(b) time to cardiovascular (CV)-related mortality;
(c) six-minute walk test (6MWT) score compared to baseline;
(d) rate of cardiovascular (CV) clinical events;
(e) time to all-cause mortality (ACM).
[56] The method of any one of [50] to [55], wherein the treatment has at least one of the following effects:
(a) reduces NT-proBNP levels in the subject compared to baseline;
(b) reduces rate of heart failure (HF) events;
(c) reduces incidence of intensification of oral diuretic therapy, which optionally includes outpatient augmentation of oral diuretic therapy;
(d) reduces incidence of changes in disease modifying therapy;
(e) reduces incidence of hospitalization for atrial fibrillation;
(f) induces change from baseline in Kansas City Cardiomyopathy Questionnaire overall summary (KCCQ-OS) score beyond 24 months of study treatment;
(g) induces change from baseline in six-minute walk test (6MWT) beyond 24 months of study treatment;
(h) induces change from baseline in ATTR-CM disease severity based on the Mayo, NAC, and Columbia disease stage, and NYHA classification;
(i) induces change from baseline in GLS;
(j) induces change from baseline in stroke volume;
(k) induces change from baseline in echocardiography parameters of interest;
(l) induces change from baseline in hs-cTnT;
(m) induces change from baseline in DPD/PYP/HMDP cardiac scintigraphy cardiac uptake and/or cMRI-derived ECV, T1 and T2 mapping;
(n) induces change from baseline in eGFR
(o) induces change from baseline in EQ-5D-5L score and/or induces change from baseline in SF-36 scores;
(p) induces change from baseline in a marker selected from CRP, IL IL1b, IL6, IL8, IFNg, TNF-α, IL10, IL1RA, C3 and C4, serum amyloid A, and ferritin; preferably a marker which is CRP;
(q) induces change from baseline in a marker selected from serum carboxy-terminal PICP, PIIINP, serum CITP, and plasma PRO-C6;
(r) induces change from baseline in a marker selected from TTR (prealbumin), TSH, RBP and fT4;
(s) induces change from baseline in (1) PND score and FAP stage; (2) Norfolk QoL-DN total score; and/or (3) sNFL levels;
(t) induces change from baseline in NIS and/or induces a change in NC studies.
[57] The method of any one of [38] to [56], wherein the anti-TTR antibody is administered intravenously (IV); preferably wherein the anti-TTR antibody is administered every four weeks (q4w) via IV infusion.
[58] The method of any one of [38] to [57], wherein the anti-TTR antibody is administered for at least 24 months, preferably at least 48 months.
[59] The method of any one of [38] to [58], wherein the anti-TTR antibody is administered in a body weight-bracketed flat dose based on the patient's recorded body weight, wherein said recording is made within 30 days of a scheduled administration, wherein said dose is administered intravenously (IV) via infusion every four weeks (q4w).
[60] The method of any one of [38] to [59], wherein the anti-TTR antibody triggers the elimination of ATTR fibrils from patient samples through immune driven phagocytosis clearance in a dose- and time-dependent manner.
[61] The method of [60], wherein the elimination of ATTR fibrils by the anti-TTR antibody treatment is measured via cardiac tracer uptake scintigraphy or quantification of ECV with cMRI.
[62] The method of [60], wherein the patient's treatment with the anti-TTR antibody results in a dose- and time-dependent reduction in the cardiac amyloid load up to approximately 51% at 12 months, preferably wherein the dose corresponds to 60 mg/kg at 12 months.
[63] The method of [60], wherein the patient a male or a female who
   (1) has a centrally confirmed diagnosis of ATTR-CM with either wild-type or variant TTR genotype based on evidence of cardiac amyloidosis by echocardiography or cMRI and one of the following:
a. Endomyocardial biopsy with confirmatory TTR amyloid typing by either immunohistochemistry or mass spectrometry; OR
b. Grade 2 or 3 cardiac uptake on 99mTc scintigraphy (99mTc DPD, 99mTc PYP, or 99mTc HMDP) in the absence of monoclonal gammopathy; OR
c. Grade 2 or 3 cardiac uptake on 99mTc scintigraphy (99mTc DPD, 99mTc PYP, or 99mTc HMDP) AND confirmatory TTR amyloid typing by either immunohistochemistry or mass spectrometry in non-cardiac tissue in the presence of monoclonal gammopathy;
   (2) is willing to be genetically tested for mutations in the TTR gene during screening, if genetic testing was not previously performed or if genetic results are not available
   (3) has end-diastolic interventricular septal wall thickness ≥11 mm for women or ≥12 mm for men on echocardiography measured at Screening
   (4) has NT-proBNP>2000 pg/mL, as measured by a central laboratory at screening
   (5) has treatment with a loop diuretic for at least 30 days prior to screening;
   (6) has a history of heart failure as documented by one of the following events within 1 year prior to screening:
a. heart failure hospitalization
b. urgent heart failure visit
c. episode of volume overload documented by NT-proBNP>2000 pg/mL (or equivalent BNP) (7) is New York Heart Association (NYHA) classification Class II-IV at screening; and/or (8) has a life expectancy of at least 6 months as per the clinician's judgment.
[64] The method of any one of [38] to [63], wherein the anti-TTR antibody is NI006/ALXN2220 which is administered in a pharmaceutical formulation at 50 mg/mL in 20 mM histidine buffer, 80 mg/mL sucrose, 0.3 mg/mL polysorbate 80, at pH 5.8.
[65] The method of any one of [38] to [64], wherein the anti-TTR antibody is NI006/ALXN2220 which is administered as an intravenous (IV) infusion in said patient over 2 hours for initial administration and over 1 hour for subsequent administrations.
[66] The method of any one of [38] to [65], wherein the patient has been treated with a disease modifying agent selected from TTR silencer and TTR stabilizer, preferably wherein the disease modifying agent comprises tafamidis, diflunisal, or a TTR silencer.
[67] The method of any one of [38] to [66], wherein the anti-TTR antibody is administered for up to 24 months in accordance with the intervention infusion schedule provided in Table 25.
[68] The method of [67], wherein the anti-TTR antibody is administered, as follow-up, after the 24-month treatment period in accordance with the intervention infusion schedule of Table 26.
[69] The method of any one of [38] to [68], wherein the treatment results in a median amyloid reduction when the dose corresponds to a dose between 30 and 60 mg/kg in the patient.
[70] The method of any one of [38] to [69], wherein the patient is stratified based on:
(1) prior treatment with a disease modifying agent, wherein the disease modifying agent is selected from (a) TTR silencer optionally together with a TTR stabilizer; (b) TTR stabilizer alone; and (c) no TTR stabilizer or TTR silencer treatment;
(2) TTR genotype comprising ATTR variant (ATTRv) or ATTR wild-type (ATTRwt); or
(3) disease severity based on cardiac biomarker levels comprising NT-proBNP>3000 pg/mL vs NT-proBNP≤3000 pg/mL, optionally together with levels of high sensitivity cardiac troponin C (hs-cTnT) pre- and post-treatment.
[71] The method of any one of [38] to [70], wherein the treatment efficacy is measured with endomyocardial biopsy comprising intra-epidermal nerve fiber density (IENFD) and/or sweat gland nerve fiber density (SGNFD).
[72] The method of any one of [38] to [71], wherein serum biomarker levels are monitored pre and post treatment with the anti-TTR antibody, wherein the biomarkers are selected from: (a) complement factors selected from C3 and C4, together with CRP; (b) pro-inflammatory cytokines selected from IL1 b, IL6, IL8, IFNg, and TNF-α; (c) anti-inflammatory cytokines selected from IL 10, IL1RA; (d) positive acute phase proteins selected from SAA and ferritin; (e) PICP; (f) PIIINP; (g) CITP; (h) PRO-C6; and (i) plasma NNTTR, or a combination thereof.
[73] The method of any one of [38] to [72], wherein the anti-TTR antibody, when administered to the human patient, is safe and well-tolerated by the human patient.
[74] Use of an anti-TTR antibody, which comprises a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8, in the manufacture of a medicament for treating transthyretin-mediated amyloidosis (ATTR) in a subject in need of said treatment, wherein the treatment comprises administering the antibody at a dose of: 2000 mg to 2500 mg to a patient weighing equal or more than 40 kg (≥40 kg) to under 60 kg (<60 kg); (b) 3000 mg to 3500 mg to a patient weighing equal or more than 60 kg (≥60 kg) to under 100 kg (<100 kg); or (c) 4000 mg to 5000 mg to a patient weighing equal or more than 100 kg (≥100 kg).
[75] The use of [74], wherein the antibody is administered at a dose of 2400 mg to a patient weighing equal or more than 40 kg (≥40 kg) to under 60 kg (<60 kg); (b) 3200 mg to a patient weighing equal or more than 60 kg (≥60 kg) to under 100 kg (<100 kg); or (c) 4800 mg to a patient weighing equal or more than 100 kg (≥100 kg).
[76] The use of [74] or [75], wherein the antibody is the antibody as defined in any one of the preceding embodiments.
[77] The use of any one of [74] to [76], wherein the subject is the subject as defined in any one of the preceding embodiments.
[78] The use of any one of [74] to [77], wherein administration of the antibody is performed as defined in any one of the preceding embodiments.
[79] The use of any one of [74] to [78], wherein the treatment is performed as defined in any one of the preceding embodiments.
[80] A therapeutic kit comprising: (i) one or more container(s), wherein the container(s) comprise a formulation of an anti-TTR antibody in an amount of 2400 mg, 3200 mg or 4800 mg or multiple doses of any one thereof; and (ii) means for delivery of the antibody to a human, wherein the means optionally comprises an infusion bag and/or a syringe, wherein the antibody comprises a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8, preferably wherein the antibody is the as defined in any one of the preceding embodiments.
[81] A therapeutic kit comprising: (i) one or more container(s), wherein the container(s) comprise a formulation of an anti-TTR antibody in an amount of 2400 mg, 3200 mg or 4800 mg or multiple doses of any one thereof; and (ii) means for delivery of the antibody to a human, wherein the means optionally comprises an infusion bag and/or a syringe, wherein the anti-TTR antibody is antibody NI006/ALXN2220.
[82] An article of manufacture comprising: one or more container(s) comprising (i) a formulation of an anti-TTR antibody in a total amount of 2400 mg, 3200 mg or 4800 mg or multiple doses of any one thereof; and (ii) a leaflet prescribing that the antibody is to be administered at a dosage of 2400 mg to a subject with a body weight of 40 to ≤60 kg, of 3200 mg to a subject with a body weight of 360 kg to 100 kg; or of 4800 mg to a subject with a body weight of ≤100 kg once every 4 weeks, wherein the antibody comprises a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8, preferably wherein the antibody is the as defined in any one of the preceding embodiments.
[83] An article of manufacture comprising: one or more container(s) comprising (i) a formulation of an anti-TTR antibody in a total amount of 2400 mg, 3200 mg or 4800 mg or multiple doses of any one thereof; and (ii) a leaflet prescribing that the antibody is to be administered at a dosage of 2400 mg to a subject with a body weight of 40 to 560 kg, of 3200 mg to a subject with a body weight of 360 kg to 100 kg; or of 4800 mg to a subject with a body weight of ≤100 kg once every 4 weeks, wherein the anti-TTR antibody is antibody NI006/ALXN2220.
[84] The kit of [80] or [81], or the article of manufacture of [82] or [83], wherein ALXN2220/NI006 is a recombinant human-derived immunoglobulin gamma 1 (IgG1) monoclonal antibody specifically recognizing aggregated wild type and mutant forms of TTR and is expressed in a Chinese hamster ovary cell line, preferably in the CHO-K1 cell line.
[85] The kit of any one of [80], [81], and [84], or the article of manufacture of any one of [80] or
[83], wherein ALXN2220/NI006 has an approximate molecular weight of 150 kDa.
[86] The kit of any one of [80], [81], [84], and [85], or the article of manufacture of any one of
[82] to [85], wherein ALXN2220/NI006 is a concentrate for solution for infusion that is presented as sterile, colorless to slightly yellow, clear to slightly opalescent liquid, essentially free of visible particles for intravenous use by infusion after dilution.
[87] The kit or article of manufacture of [86], wherein each mL of solution contains 50 mg/mL ALXN2220/NI006 in 20 mM histidine buffer, 80 mg/mL sucrose, 0.3 mg/mL polysorbate 80, at pH 5.8.
[88] The article of manufacture of any one of [82] to [87], wherein the recommended dosage of ALXN2220/NI006 on the label is 2400 mg for patients with weight of ≥40 kg to <60 kg, 3200 mg for ≥60 kg to <100 kg and 4800 mg for patients with weight of ≥100 kg that must be diluted then administered as an intravenous infusion over approximately one to two hours, once every four weeks.
[89] The kit of any one of [80], [81], [84], and [85] to [87], or the article of manufacture of any one of [82] to [88], wherein prior to administration, ALXN2220/NI006 is diluted with 5% glucose solution.
[90] The kit of any one of [80], [81], [84], [85] to [87], and [89], or the article of manufacture of any one of [82] to [89], wherein ALXN2220/NI006 is a clear to opalescent and colorless to pale yellow solution, available as solution 100 mg/2 mL (50 mg/mL) in a single-dose vial.
[91] A method of treating ATTR, the method comprising administering to a human subject a human anti-TTR antibody, wherein the antibody is preferably an antibody as defined in any one of the preceding embodiments and administered at a dosage providing about 30 mg/kg or 60 mg/kg to the subject, preferably once every 28 days.
[92] The method of [91], where the method is performed as characterized in any of the preceding embodiments.
[93] An embodiment as characterized in any of the preceding embodiments.

1-93. (canceled)

94. A method of treating ATTR in a human subject in need of said treatment, the method comprising administering to said human subject:
(a) an anti-transthyretin (TTR) antibody comprising a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8, wherein the antibody is administered at a dose of: (a): 2000 mg to 2500 mg to a patient weighing equal or more than 40 kg (≥40 kg) to under 60 kg (≤60 kg); (b) 3000 mg to 3500 mg to a patient weighing equal or more than 60 kg (≥60 kg) to under 100 kg (≤100 kg); or (c) 4000 mg to 5000 mg to a patient weighing equal or more than 100 kg (≥100 kg); or
(b) a human anti-TTR antibody administered at a dosage providing about 30 mg/kg or 60 mg/kg to the subject, preferably once every 28 days.

95. The method of claim 94, wherein the antibody is administered at a dose of 2400 mg to a patient weighing equal or more than 40 kg (≥40 kg) to under 60 kg (≤60 kg); (b) 3200 mg to a patient weighing equal or more than 60 kg (≥60 kg) to under 100 kg (≤100 kg); or (c) 4800 mg to a patient weighing equal or more than 100 kg (≥100 kg).

96. The method of claim 94, wherein the antibody:
(a) comprises a human Ig constant region;
(b) comprises a human IgG1 comprising an antibody heavy chain constant region of human IgG1 m3 allotype;
(c) comprises two heavy chains and two light chains, wherein each heavy chain comprises 450 amino acid residues comprising the sequence of SEQ ID NO: 9, and each light chain comprises 214 amino acid residues comprising the sequence of SEQ ID NO: 10;
(d) comprises two heavy chains and two light chains, wherein each heavy chain comprises 449 amino acid residues comprising the sequence of SEQ ID NO: 13, and each light chain comprises 214 amino acid residues comprising the sequence of SEQ ID NO: 10;
(e) comprises two heavy chains and two light chains, wherein each heavy chain comprises 449 amino acid residues comprising the sequence of SEQ ID NO: 14, and each light chain comprises 214 amino acid residues comprising the sequence of SEQ ID NO: 10;
(f) two heavy chains and two light chains, wherein each heavy chain comprises 448 amino acid residues comprising the sequence of SEQ ID NO: 15, and each light chain comprises 214 amino acid residues comprising the sequence of SEQ ID NO: 10;
(g) antibody is NI006/ALXN222 or comprises a binding fragment thereof.

97. The method of claim 94, wherein the heavy chain of the antibody comprises a cyclized N-terminal glutaminyl residue comprising pyroglutamate (pyro-Q) and/or wherein the antibody heavy chain further comprises a clipped C-terminal lysine.

98. The method of claim 94, wherein the antibody is N-glycosylated, wherein the N-linked glycan is on N300 of the heavy chain of the antibody.

99. The method of claim 94, wherein the subject has symptomatic ATTR-CM and/or NT-proBNP>2000 μg/mL.

100. The method of claim 99, wherein the subject has either variant ATTR-CM (ATTRv-CM/hATTR-CM) or wild-type ATTR-CM ((wATTR-CM).

101. The method of claim 99, wherein the subject is an adult human subject who is 18 years or older but less than 90 years old ((≥18 to ≤90 years of age).

102. The method of claim 99, wherein the treatment lowers composite of all-cause mortality (ACM) and total cardiovascular (CV) clinical events.

103. The method of claim 99, wherein the treatment lowers composite of all-cause mortality (ACM) and heart failure (HF) events.

104. The method of claim 99, wherein the treatment improves at least one of the following:
(a) symptoms, functionality, and health-related quality-of-life (QoL) as measured by the change from baseline in Kansas City Cardiomyopathy Questionnaire overall summary (KCCQ-OS) score;
(b) time to cardiovascular (CV)-related mortality;
(c) six-minute walk test (6MWT) score compared to baseline;
(d) rate of cardiovascular (CV) clinical events; and
(e) time to all-cause mortality (ACM).

105. The method of claim 99, wherein the treatment has at least one of the following effects:
(a) reduces NT-proBNP levels in the subject compared to baseline;
(b) reduces rate of heart failure (HF) events;
(c) reduces incidence of intensification of oral diuretic therapy and/or includes outpatient augmentation of oral diuretic therapy;
(d) reduces incidence of changes in disease modifying therapy;
(e) reduces incidence of hospitalization for atrial fibrillation;
(f) induces change from baseline in Kansas City Cardiomyopathy Questionnaire overall summary (KCCQ-OS) score beyond 24 months of study treatment;
(g) induces change from baseline in six-minute walk test (6MWT) beyond 24 months of study treatment;
(h) induces change from baseline in ATTR-CM disease severity based on the Mayo, NAC, and Columbia disease stage, and NYHA classification;
(i) induces change from baseline in GLS;
(j) induces change from baseline in stroke volume;
(k) induces change from baseline in echocardiography parameters of interest;
(l) induces change from baseline in hs-cTnT;
(m) induces change from baseline in DPD/PYP/HMDP cardiac scintigraphy cardiac uptake and/or cMRI-derived ECV, T1 and T2 mapping;
(n) induces change from baseline in eGFR
(o) induces change from baseline in EQ-5D-5L score and/or induces change from baseline in SF-36 scores;
(p) induces change from baseline in a marker selected from CRP, IL IL1b, IL6, IL8, IFNg, TNF-α, IL10, IL1 RA, C3 and C4, serum amyloid A, and ferritin;
(q) induces change from baseline in a marker selected from serum carboxy-terminal PICP, PIIINP, serum CITP, and plasma PRO-C6;
(r) induces change from baseline in a marker selected from TTR (prealbumin), TSH, RBP and fT4;
(s) induces change from baseline in (1) PND score and FAP stage; (2) Norfolk QoL-DN total score; and/or (3) sNFL levels; and
(t) induces change from baseline in NIS and/or induces a change in NC studies.

106. The method of claim 94, wherein the anti-TTR antibody is administered intravenously (IV).

107. The method of claim 94, wherein the anti-TTR antibody is administered every four weeks (q4w) via IV infusion for at least 24 months.

108. The method of claim 94, wherein the anti-TTR antibody is administered in a body weight-bracketed flat dose based on the patient's recorded body weight, wherein said recording is made within 30 days of a scheduled administration, wherein said dose is administered intravenously (IV) via infusion every four weeks (q4w).

109. The method of claim 94, wherein the patient's treatment with the anti-TTR antibody results in a dose- and time-dependent reduction in the cardiac amyloid load up to approximately 51% at 12 months, and wherein the dose corresponds to 60 mg/kg at 12 months.

110. The method of claim 94, wherein the patient a male or a female who
(1) has a centrally confirmed diagnosis of ATTR-CM with either wild-type or variant TTR genotype based on evidence of cardiac amyloidosis by echocardiography or cMRI and one of the following:
a. Endomyocardial biopsy with confirmatory TTR amyloid typing by either immunohistochemistry or mass spectrometry; OR
b. Grade 2 or 3 cardiac uptake on 99mTc scintigraphy (99mTc DPD, 99mTc PYP, or 99mTc HMDP) in the absence of monoclonal gammopathy; OR
c. Grade 2 or 3 cardiac uptake on 99mTc scintigraphy (99mTc DPD, 99mTc PYP, or 99mTc HMDP) AND confirmatory TTR amyloid typing by either immunohistochemistry or mass spectrometry in non-cardiac tissue in the presence of monoclonal gammopathy;
(2) is willing to be genetically tested for mutations in the TTR gene during screening, if genetic testing was not previously performed or if genetic results are not available
(3) has end-diastolic interventricular septal wall thickness ≥11 mm for women or ≥12 mm for men on echocardiography measured at Screening
(4) has NT-proBNP>2000 μg/mL, as measured by a central laboratory at screening
(5) has treatment with a loop diuretic for at least 30 days prior to screening;
(6) has a history of heart failure as documented by one of the following events within 1 year prior to screening:
a. heart failure hospitalization
b. urgent heart failure visit
c. episode of volume overload documented by NT-proBNP>2000 μg/mL (or equivalent BNP)
(7) is New York Heart Association (NYHA) classification Class II-IV at screening; and/or
(8) has a life expectancy of at least 6 months as per the clinician's judgment.

111. The method of claim 94, wherein:
(a) the anti-TTR antibody is N1006/ALXN2220 which is administered in a pharmaceutical formulation at 50 mg/mL in 20 mM histidine buffer, 80 mg/mL sucrose, 0.3 mg/mL polysorbate 80, at pH 5.8;
(b) the anti-TTR antibody is N1006/ALXN2220 which is administered as an intravenous (IV) infusion in said patient over 2 hours for initial administration and over 1 hour for subsequent administrations;
(c) the patient has been treated with a disease modifying agent selected from TTR silencer and TTR stabilizer, preferably wherein the disease modifying agent comprises tafamidis, diflunisal, or a TTR silencer; or
(d) the anti-TTR antibody is administered for up to 24 months in accordance with the intervention infusion schedule provided in Table 25.

112. The method of claim 94, wherein the patient is stratified based on:
(i) prior treatment with a disease modifying agent, wherein the disease modifying agent is selected from (a) TTR silencer and/or a TTR stabilizer; (b) TTR stabilizer alone; and (c) no TTR stabilizer or TTR silencer treatment;
(ii) TTR genotype comprising ATTR variant (ATTRv) or ATTR wild-type (ATTRwt); or
(iii) disease severity based on cardiac biomarker levels comprising NT-proBNP>3000 μg/mL vs NT-proBNP≤3000 μg/mL, and/or together with levels of high sensitivity cardiac troponin C (hs-cTnT) pre- and post-treatment.

113. The method of claim 94, wherein the treatment efficacy is measured with endomyocardial biopsy comprising intra-epidermal nerve fiber density (IENFD) and/or sweat gland nerve fiber density (SGNFD).

114. The method of claim 94, wherein serum biomarker levels are monitored pre and post treatment with the anti-TTR antibody, wherein the biomarkers are selected from: (a) complement factors selected from C3 and C4, together with CRP; (b) pro-inflammatory cytokines selected from IL1b, IL6, IL8, IFNg, and TNF-α; (c) anti-inflammatory cytokines selected from IL10, IL1 RA; (d) positive acute phase proteins selected from SAA and ferritin; (e) PICP; (f) PIIINP; (g) CITP; (h) PRO-C6; and (i) plasma NNTTR, or a combination thereof.

115. A method of treating ATTR in a human subject in need of said treatment, the method comprising administering to said human subject an anti-transthyretin (TTR) antibody comprising a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8, wherein the antibody is administered at a dose of: (a): 2000 mg to 2500 mg to a patient weighing equal or more than 40 kg (≥40 kg) to under 60 kg (<60 kg); (b) 3000 mg to 3500 mg to a patient weighing equal or more than 60 kg (≥60 kg) to under 100 kg (<100 kg); or (c) 4000 mg to 5000 mg to a patient weighing equal or more than 100 kg (≥100 kg), wherein the anti-TTR antibody is administered every four weeks (q4w) via IV infusion for at least 24 months.

116. A therapeutic kit comprising:
(A) (i) one or more container(s), wherein the container(s) comprise a formulation of an anti-TTR antibody in an amount of 2400 mg, 3200 mg or 4800 mg or multiple doses of any one thereof; and (ii) a container, optionally an infusion bag and/or a syringe, for delivery of the antibody to a human, wherein the antibody comprises a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8; or
(B) (i) one or more container(s), wherein the container(s) comprise a formulation of an anti-TTR antibody in an amount of 2400 mg, 3200 mg or 4800 mg or multiple doses of any one thereof; and (ii) a container, optionally an infusion bag and/or a syringe, for delivery of the antibody to a human, wherein the anti-TTR antibody is antibody N1006/ALXN2220.

117. An article of manufacture comprising:
(A) one or more container(s) comprising (i) a formulation of an anti-TTR antibody in a total amount of 2400 mg, 3200 mg or 4800 mg or multiple doses of any one thereof; and (ii) a leaflet prescribing that the antibody is to be administered at a dosage of 2400 mg to a subject with a body weight of 40 to 560 kg, of 3200 mg to a subject with a body weight of 60 kg to 100 kg; or of 4800 mg to a subject with a body weight of ≤100 kg once every 4 weeks, wherein the antibody comprises a heavy chain variable region comprising complementary determining regions (CDRs) comprising heavy chain CDR1-3 whose sequences are set forth in SEQ ID NOs: 1-3, respectively, and light chain CDR1-3, whose sequences are set forth in SEQ ID NOs: 4-6, wherein the anti-TTR antibody comprises a heavy chain variable region comprising at least 80% sequence identity to SEQ ID NO: 7 and light chain variable region comprising an amino acid sequence having at least 80% sequence identity to SEQ ID NO: 8; or
(B) one or more container(s) comprising (i) a formulation of an anti-TTR antibody in a total amount of 2400 mg, 3200 mg or 4800 mg or multiple doses of any one thereof; and (ii) a leaflet prescribing that the antibody is to be administered at a dosage of 2400 mg to a subject with a body weight of 40 to 560 kg, of 3200 mg to a subject with a body weight of 60 kg to 100 kg; or of 4800 mg to a subject with a body weight of ≤100 kg once every 4 weeks, wherein the anti-TTR antibody is antibody N1006/ALXN2220.
